(19) United States
(12) Patent Application Publication
(10) Pub. No.: US 2005/0245503 A1
(43) Pub. Date: Nov. 3, 2005

(54) Therapeutic heterocycles

(76) Inventors: Yun-Xing Cheng, Quebec (CA); Xuehong Luo, Quebec (CA); Miroslaw Tomaszewski, Quebec (CA); Christopher Walpole, Quebec (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 10/498,864

(22) PCT Filed: Dec. 17, 2002
(86) PCT No.: PCT/SE02/02354

§ 371 Date: Jun. 16, 2004

(30) Foreign Application Priority Data
Dec. 19, 2001 (SE) 0104326-4

(51) Int. Cl. A61K 31/554; A61K 31/4743; A61K 31/502; A61K 31/38; A61K 31/4184; A61K 31/473

(52) U.S. Cl. 514/211.11; 514/248; 514/290; 514/291; 514/375; 514/393; 514/431; 540/547; 546/80; 546/93; 548/216; 548/302.1; 549/12

(57) ABSTRACT

Compounds of formula (I) or (II) wherein R₁, R₂, R₃, R₄, W, Q, G, X and Y are as defined in the specification, as well as salts, enantiomers thereof and pharmaceutical compositions including the compounds are prepared. They are useful in therapy, in particular in the management of pain.

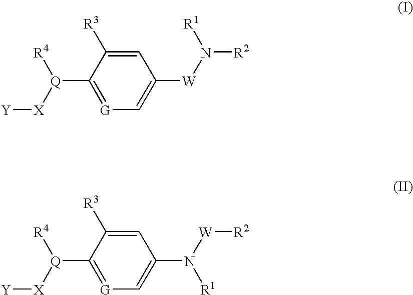

### BACKGROUND OF THE INVENTION

1. Field of the Invention

The present invention is directed to novel compounds, to processes for their preparation, their use and pharmaceutical compositions comprising the novel compounds. The compounds are useful in therapy, and in particular for the treatment of pain, septic shock, pancreatitis, edema, rhinitis, asthma, colitis, arthritis, hepatorenal syndrome, cancer, bacterial and viral infections, ulcerative colitis, and Alzheimer's Disease.

2. Discussion of Relevant Art

Two types of bradykinin receptor are known: The B1 receptor and the B2 receptor. A number of reports indicate an important role for the B2 receptor in the pathophysiology of pain. [e.g. Hall, J. M., Morton, I. K. M. The pharmacology and immunopharmacology of kinin receptors. In: Farmer SG (Ed). The kinin system. London: Academic Press, 1997; 9-44]. Hence, compounds that are B2 antagonists are useful in the relief of pain, including chronic pain and acute pain, e.g., chronic inflammatory pain, neuropathic pain, back pain, migraine, cancer pain, visceral pain, arthritis pain and post-operative pain.

### DETAILED DESCRIPTION OF THE INVENTION

Thus, the problem underlying the present invention was to find and obtain new compounds that are useful in treating pain.

Accordingly, in one aspect, the present invention provides compounds that are useful in treating pain.

In another aspect, the present invention provides compounds that are B2 antagonists.

Definitions

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

The term “Cₘ₋ₙ” or “Cₙ₋ₙ group” used alone or as a prefix, refers to any group having m to n carbon atoms, and having 0 to n multivalent heteroatoms selected from O, S, N and P, wherein m and n are 0 or positive integers, and n>m. For example, “C₁₋₆” would refer to a chemical group having 1 to 6 carbon atoms, and having 0 to 6 multivalent heteroatoms selected from O, S, N and P.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to monovalent straight or branched chain hydrocarbon radicals comprising 1 to about 12 carbon atoms.

The term “alkylene” used alone or as suffix or prefix, refers to divalent straight or branched chain hydrocarbon radicals comprising 1 to about 12 carbon atoms, which serves to links two structures together.

The term “alkenyl” used alone or as suffix or prefix, refers to a monovalent straight or branched chain hydrocarbon radical having at least one carbon-carbon double bond and comprising at least 2 up to about 12 carbon atoms.

The term “alkynyl” used alone or as suffix or prefix, refers to a monovalent straight or branched chain hydrocarbon radical having at least one carbon-carbon triple bond and comprising at least 2 up to about 12 carbon atoms.

The term “cycloalkyl,” used alone or as suffix or prefix, refers to a monovalent ring-containing hydrocarbon radical comprising at least 3 up to about 12 carbon atoms.

The term “cycloalkenyl” used alone or as suffix or prefix, refers to a monovalent ring-containing hydrocarbon radical having at least one carbon-carbon double bond and comprising at least 3 up to about 12 carbon atoms.

The term “cycloalkynyl” used alone or as suffix or prefix, refers to a monovalent ring-containing hydrocarbon radical having at least one carbon-carbon triple bond and comprising about 7 up to about 12 carbon atoms.

The term “aryl” used alone or as suffix or prefix, refers to a monovalent hydrocarbon radical having one or more polyunsaturated carbon rings having aromatic character, (e.g., 4n+2 delocalized electrons) and comprising 5 up to about 14 carbon atoms.

The term “arylene” used alone or as suffix or prefix, refers to a divalent hydrocarbon radical having one or more polyunsaturated carbon rings having aromatic character, (e.g., 4n+2 delocalized electrons) and comprising 5 up to about 14 carbon atoms, which serves to links two structures together.

The term “heterocycle” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroalkyl” used alon or as a suffix or prefix, refers to a radical formed as a result of replacing one or more carbon atom of an alkyl with one or more heteroatoms selected from N, O, P and S.

The term “heteroaromatic” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “heterocyclylene” used alone or as a suffix or prefix, refers to a divalent radical derived from a heterocycle by removing two hydrogens therefrom, which serves to links two structures together.

naming chemical structures. Optionally, a name of a compound may be generated using a chemical naming program: ACD/ChemSketch, Version 5.09/September 2001, Advanced Chemistry Development, Inc., Toronto, Canada.

The term “Cₘ₋ₙ” or “Cₘ₋ₙ group” used alone or as a prefix, refers to any group having m to n carbon atoms, and having 0 to n multivalent heteroatoms selected from O, S, N and P, wherein m and n are 0 or positive integers, and n>m. For example, “C₁₋₆” would refer to a chemical group having 1 to 6 carbon atoms, and having 0 to 6 multivalent heteroatoms selected from O, S, N and P.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to monovalent straight or branched chain hydrocarbon radicals comprising 1 to about 12 carbon atoms.

The term “alkylene” used alone or as suffix or prefix, refers to divalent straight or branched chain hydrocarbon radicals comprising 1 to about 12 carbon atoms, which serves to links two structures together.

The term “alkenyl” used alone or as suffix or prefix, refers to a monovalent straight or branched chain hydrocarbon radical having at least one carbon-carbon double bond and comprising at least 2 up to about 12 carbon atoms.

The term “alkynyl” used alone or as suffix or prefix, refers to a monovalent straight or branched chain hydrocarbon radical having at least one carbon-carbon triple bond and comprising at least 2 up to about 12 carbon atoms.

The term “cycloalkyl,” used alone or as suffix or prefix, refers to a monovalent ring-containing hydrocarbon radical comprising at least 3 up to about 12 carbon atoms.

The term “cycloalkenyl” used alone or as suffix or prefix, refers to a monovalent ring-containing hydrocarbon radical having at least one carbon-carbon double bond and comprising at least 3 up to about 12 carbon atoms.

The term “cycloalkynyl” used alone or as suffix or prefix, refers to a monovalent ring-containing hydrocarbon radical having at least one carbon-carbon triple bond and comprising about 7 up to about 12 carbon atoms.

The term “aryl” used alone or as suffix or prefix, refers to a monovalent hydrocarbon radical having one or more polyunsaturated carbon rings having aromatic character, (e.g., 4n+2 delocalized electrons) and comprising 5 up to about 14 carbon atoms.

The term “arylene” used alone or as suffix or prefix, refers to a divalent hydrocarbon radical having one or more polyunsaturated carbon rings having aromatic character, (e.g., 4n+2 delocalized electrons) and comprising 5 up to about 14 carbon atoms, which serves to links two structures together.

The term “heterocycle” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroalkyl” used alon or as a suffix or prefix, refers to a radical formed as a result of replacing one or more carbon atom of an alkyl with one or more heteroatoms selected from N, O, P and S.

The term “heteroaromatic” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “heterocyclylene” used alone or as a suffix or prefix, refers to a divalent radical derived from a heterocycle by removing two hydrogens therefrom, which serves to links two structures together.

The term “heteroaryl” used alone or as a suffix or prefix, refers to a heterocyclyl having aromatic character.

The term “heterocylcoalkyl” used alone or as a suffix or prefix, refers to a heterocyclyl that does not have aromatic character.

The term “heteroarylene” used alone or as a suffix or prefix, refers to a heterocyclylene having aromatic character.

The term “heterocycloalkylene” used alone or as a suffix or prefix, refers to a heterocyclylene that does not have aromatic character.

The term “six-membered” used as prefix refers to a group having a ring that contains six ring atoms.

The term “five-membered” used as prefix refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

The term “substituted” used as a prefix refers to a structure, molecule or group, wherein one or more hydrogens are replaced with one or more C₁₋₁₂hydrocarbon groups, or one or more chemical groups containing one or more heteroatoms selected from N, O, S, F, Cl, Br, I, and P. Exemplary chemical groups containing one or more heteroatoms include —NO₂, —OR, —Cl, —Br, —I, —F, —CF₃, —C(═O)R, —C(═O)OH, —NH₂, —SH, —NHR, —NR₂, —SR, —SO₃H, —SO₂R, —S(═O)R, —CN, —OH, —C(═O)OR, —C(═O)NR₂, —NRC(═O)R, oxo (═O), imino (═NR), thio (═S), and oximino (═N—OR), wherein each “R” is a C₁₋₁₂hydrocarbyl. For example, substituted phenyl may refer to nitrophenyl, methoxyphenyl, chlorophenyl, aminophenyl, etc., wherein the nitro, methoxy, chloro, and amino groups may replace any suitable hydrogen on the phenyl ring.

The term “substituted” used as a suffix of a first structure, molecule or group, followed by one or more names of chemical groups refers to a second structure, molecule or group, which is a result of replacing one or more hydrogens of the first structure, molecule or group with the one or more named chemical groups. For example, a “phenyl substituted by nitro” refers to nitrophenyl.

The term “optionally substituted” refers to both groups, structures, or molecules that are substituted and those that are not substituted.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” used alone or as a suffix or prefix, refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “amine” or “amino” used alone or as a suffix or prefix, refers to radicals of the general formula —NRR′, wherein R and R′ are independently selected from hydrogen or a hydrocarbon radical.

“Acyl” used alone, as a prefix or suffix, means —C(═O)—R, wherein R is an optionally substituted hydrocarbyl, hydrogen, amino or alkoxy. Acyl groups include, for example, acetyl, propionyl, benzoyl, phenyl acetyl, carboethoxy, and dimethylcarbamoyl.

Halogen includes fluorine, chlorine, bromine and iodine.

“Halogenated,” used as a prefix of a group, means one or more hydrogens on the group is replaced with one or more halogens.

“RT” or “rt” means room temperature.

A first ring group being “fused” with a second ring group means the first ring and the second ring share at least at least two atoms therebetween.

### DESCRIPTION OF PREFERRED EMBODIMENTS

In one aspect, the invention provides a compound of formula (I) or (II), pharmaceutically acceptable salts thereof, diasteriomers thereof, enantiomers thereof, or mixtures thereof: 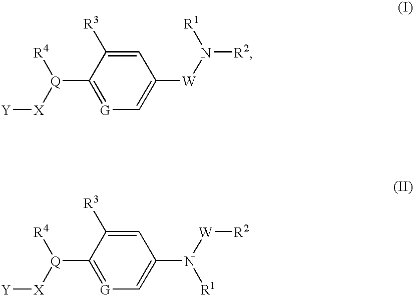
wherein
R¹ and R² are independently selected from hydrogen, optionally substituted C₁₋₁₂acyl, optionally substituted C₁₋₁₂alkyl-oxycarbonyl, optionally substituted C₁₋₁₂alkyl, optionally substituted C₁₋₁₂heteroalkyl, optionally substituted C₃₋₁₂cycloalkyl, optionally substituted C₆₋₁₂aryl, optionally substituted C₂₋₁₂heterocyclyl; optionally substituted aryl-C₁₋₆alkyl, and optionally substituted heterocyclyl-C₁₋₆alkyl;
W is a linking group that separates the groups linked thereto by one or two atoms;
G is N or CH;
R³ is halogen, hydrogen or C₁₋₆alkyl;
Q is N or CH;
R⁴ is —H or optionally substituted hydrocarbyl;
X is a divalent group including first nitrogen atom and a second nitrogen atom, wherein a first group linked to X is linked to the first nitrogen and a second group linked to X is linked to the second nitrogen atom, and wherein the first and second nitrogen atoms are separated by either one carbon atom, or two carbon atoms wherein said two carbon atoms form a double bond therebetween; and
Y is a group that includes an optionally substituted seven-membered ring and two optionally substituted aromatic rings, wherein each of the aromatic rings is independently fused with said seven-membered ring, and wherein each of said seven-membered ring and aromatic rings, independently, optionally, contains one or more heteroatoms.

In another aspect, the compounds of the present invention are those of formula (a) or (II), pharmaceutically acceptable salts thereof, diasteriomers thereof, enantiomers thereof, or mixtures thereof, wherein
R¹ and R² are independently selected from hydrogen, optionally substituted acyl, optionally substituted alkyl-oxycarbonyl, optionally substituted alkyl, optionally substituted heteroalkyl, optionally substituted cycloalkyl, optionally substituted aryl; optionally substituted heterocyclyl; optionally substituted aryl-C₁₋₆alkyl, and optionally substituted heterocyclyl-C₁₋₆alkyl;
W is a linking group selected from —C(═O)—, —C(═O)O— and —S(═O)₂—;
G is N or CH;
R³ is halogen, or hydrogen
Q is N or CH;
R⁴ is —H, or optionally substituted hydrocarbyl;
X is a divalent group including first nitrogen atom and a second nitrogen atom, wherein a first group linked to X is linked to the first nitrogen and a second group linked to X is linked to the second nitrogen atom, and wherein the first and second nitrogen atoms are separated by either one carbon atom, or two carbon atoms wherein said two carbon atoms form a double bond therebetween, more particularly X is selected from Formulas (i) and (ii), below 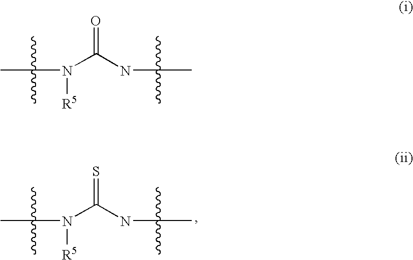
wherein R⁵ is —H, or optionally substituted C₁₋₆alkyl; and
Y is optionally subsitituted aryl, or optionally substituted heteroaryl; more particularly, Y is aryl optionally substituted by a C₁₋₁₂hydrocarbyl or heteroaryl optionally substituted by a C₁₋₁₂hydrocarbyl; most particularly, Y is C₁₋₆alkyl-cyclohexyl-phenyl.

In a further aspect, the compounds of the present invention are those of formula (I) or (II), pharmaceutically acceptable salts thereof, diasteriomers thereof, enantiomers thereof, or mixtures thereof, wherein
R¹ and R² are independently selected from hydrogen, optionally substituted acyl, optionally substituted alkyl-oxycarbonyl, optionally substituted alkyl, optionally substituted heteroalkyl, optionally substituted cycloakyl, optionally substituted aryl; optionally substituted heterocyclyl; optionally substituted aryl-C₁₋₆alkyl, and optionally substituted heterocyclyl-C₁₋₆alkyl;
W is a linking group selected from —C(═O)—, —C(═O)O— and S(═O)₂—;
G is N or CH;
R³ is halogen, or hydrogen
Q is N or CH;
R⁴ is —H, optionally substituted hydrocarbyl, a single bond, or a divalent group;
X is represented by (i), (ii), (iii), (iv), (v), (vi), (vii), (viii), (ix), (x), (xi), (xii), (xiii), (xiv), (xv), (xvi), or (xvii) below: 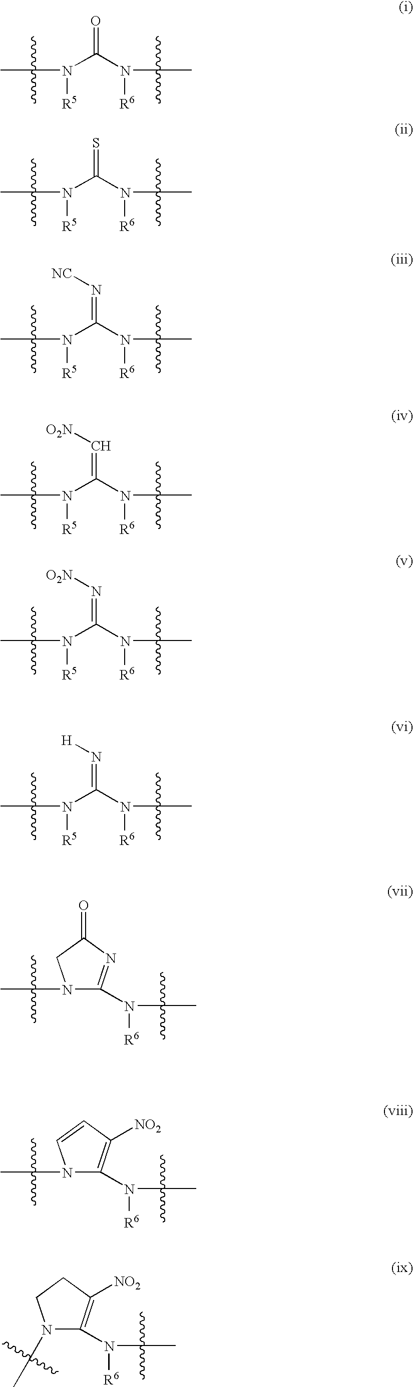 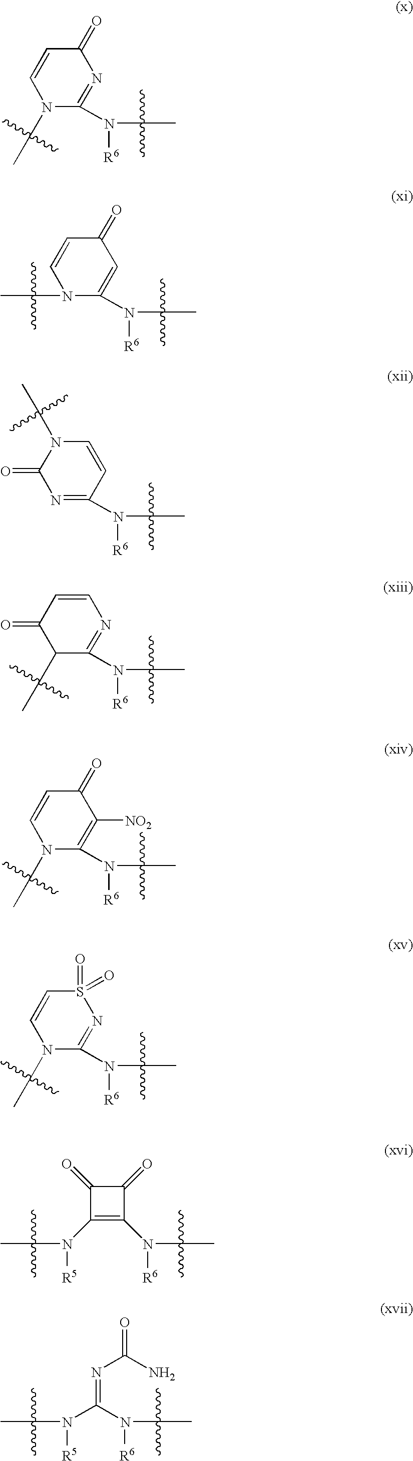
wherein R⁵ is selected from —H or optionally substituted alkyl, or a divalent C₀₋₆group together with R⁴ to form a portion of a ring, wherein said divalent C₀₋₆ group optionally contains one or more heteroatoms;
R⁶ is independently selected from —H or optionally substituted alkyl;
Y is represented by formula (III) below: 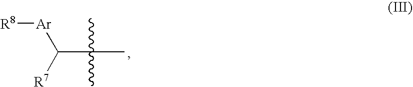
wherein
R⁷ is optionally substituted aryl, optionally substituted heteroaryl, optionally substituted aryl-C₁₋₆alkyl, optionally substituted heteroaryl-C₁₋₆alkyl, —C(═O)O—R⁹, —C(═O)NHR⁹, —C(═O)NR⁹R¹⁰, —SO₂NHR⁹, —SO₂NR⁹R¹⁰, —R¹¹NH₂, —R¹¹NHR¹², —R¹¹NR¹²R¹³, —R¹¹OH, —R¹¹OR¹², —R¹¹SH, —R¹¹SR¹², or a divalent C₀₋₆group which together with R⁸ forms a portion of a ring,
R⁸ is —H, halogen, optionally substituted R¹², —OR¹², —SR¹², —S(═O)R¹², —SO₂R¹², —C(═O)R¹², or a divalent C₀₋₆group which together with the divalent R⁷ forms the portion of the ring,
wherein R⁹ and R¹⁰ are independently C₁₋₁₂hydrocarbyl, R¹¹ is C₁₋₆alkylene, R¹² and R¹³ are independently C₁₋₆alkyl; and
Ar is optionally substituted arylene, optionally substituted heteroarylene, optionally substituted arylene-C₁₋₆alky, or optionally substituted heteroarylene-C₁₋₆alkyl.

Particularly, the compounds of the present invention are those of formula (I) or (II), pharmaceutically acceptable salts thereof, diasteriomers thereof, enantiomers thereof, or mixtures thereof, wherein
R¹ is selected from C₁₋₆alkyl, C₃₋₆cycloalkyl, aryl, aryl-C₁₋₆alkyl or heterocycyl, heterocyclyl-C₁₋₆alkyl, wherein said C₁₋₆alkyl and C₃₋₆cycloalkyl are optionally, independently, subsituted by R²⁰, —C(═O)R²⁰, oxo (═O), sulfo (═S), —OH, —OR²⁰, phenyl, halogen, heterocyclyl, —NH₂, —NHR²⁰, —NR²⁰R²¹, —C(═O)NH₂, —C(═O)NHR²⁰, —C(═O)NR²⁰R²¹ and —C(═O)OR²⁰, wherein said aryl is optionally substituted by —R²⁰, —C(═O)R²⁰, —OH, —OR²⁰, phenyl, halogen, heterocyclyl, —NH₂, —NHR²⁰, —NR²⁰R²¹, —C(═O)NH₂, —C(═O)NHR²⁰, —C(═O)NR²⁰R²¹ and —C(═O)OR²⁰, wherein said heterocyclyl is a five or six-membered heterocyclyl, wherein said heterocyclyl is optionally substituted by —R²⁰, aryl, heteroaryl, —NH₂, —NHR²⁰, —NR²⁰R²¹, —C(═O)NH₂, —C(═O)NHR²⁰, —C(═O)NR²⁰R²¹, —C(═O)OR²⁰, or oxo (═O);
R² is selected from C₁₋₆alkyl, C₃₋₆cycloalkyl, wherein said C₁₋₆alkyl and C₃₋₆cycloalkyl are optionally, independently, subsituted by R²⁰, —C(═O)R²⁰, oxo (═O), sulfo (═S), —OH, —OR²⁰, phenyl, halogen, heterocyclyl, —NH₂, —NR²⁰, —NR²⁰R²¹, —C(═O)NH₂, —C(═O)NHR²⁰, —C(═O)NR²⁰R²¹ and —C(═O)OR²⁰;
wherein R²⁰ and R²¹ are independently C₁₋₆alkyl;
R³ is selected from bromo, chloro and fluoro;
R⁴ is —H, optionally substituted (C₁-C₆)alkyl or optionally substituted alkenyl, or a divalent group together with R⁵ of X to form a portion of a ring;
W is —C(═O)—, or —S(═O)₂—;
G is N or CH;
Q is CH or N;
X is selected from Formulas (i) and (ii), below: 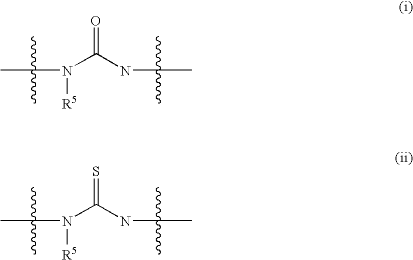
wherein R⁵ is —H, optionally substituted C₁₋₆alkyl, a bond or a divalent group wherein said bond or divalent group together with R⁴ forms the portion of the ring, wherein the ring is selected from optionally substituted Formula (a), (b) and (c), 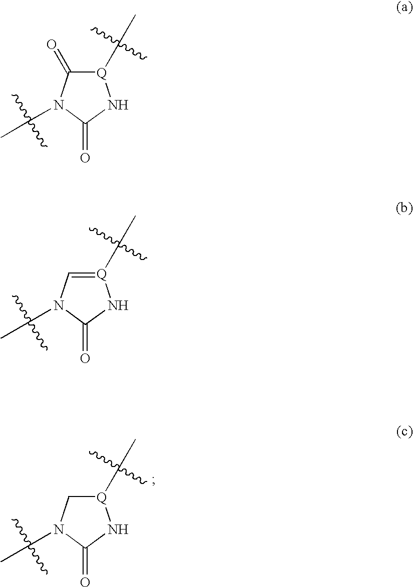
wherein when R⁴ or R⁵ is substituted, substituents thereof are preferably selected from: —OH, NH₂, —O—C₁₋₃alkyl, —CN, oxo (═O), —C(═O)O—C₁₋₄alkyl and halogen.

Y is represented by formula (III) below: 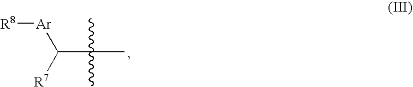
wherein R⁷ is optionally substituted aryl, optionally substituted heterocyclyl, or optionally substituted arylene which together with R⁸ forms a portion of a ring;
R⁸ is —R²², —OR²², —SR²², —S(═O)R²², —SO₂R²², —(═O)R²², or an optionally substituted divalent C₀₋₆group which together with the divalent R⁷ forms the portion of the ring;
wherein when R⁷ or R⁸ is substituted, preferable substituents thereof are halogen, nitro, cyano, R²², —C(═O)R²², —C(═O)OR²², —OH, —OR²², —C(═O)NH₂, —C(═O)NHR²², —C(═O)NR²²R²³, —SO₂NH₂, —SO₂NHR²² and —SO₂NR²²R²³;
Ar is optionally substituted arylene, optionally substituted heteroarylene, optionally substituted arylene-C₁₋₆alkyl, or optionally substituted heteroarylene-C₁₋₆alkyl; and
wherein R²² and R²³ are independently C₁₋₆alkyl.

More particularly, the compounds of the present invention are those of formula (I) or (II), pharmaceutically acceptable salts thereof, diasteriomers thereof, enantiomers thereof, or mixtures thereof, wherein
R¹ is selected from C₁₋₆alkyl, C₃₋₆cycloalkyl, aryl, aryl-C₁₋₆alkyl or heterocyclyl, heterocyclyl-C₁₋₆alkyl, wherein said C₁₋₆alkyl and C₃₋₆cycloalkyl and aryl are optionally, independently, subsituted by —OH, —C(═O)OR²⁴, —OR²⁴ and —NR²⁴R²⁵, wherein said heterocyclyl is derived from pyrrolidinone, five-membered lactone, five-membered thiolactone, pyrrolidine, tetrahyrofuran, thiophan, sulfolane, piperidine, piperazine, morpholine, thiomorpholine, dioxane, tetrahydropyran or tetrahydrothiopyran by removing a hydrogen therefrom, wherein said heterocyclyl is optionally substituted by oxo (═O);
R² is selected from C₁₋₆alkyl, C₃₋₆cycloalkyl, wherein said C₁₋₆alkyl and C₃₋₆cycloalkyl are optionally, independently, subsituted by —OH, —C(═O)OR²⁴, —OR²⁴ and —NR²⁴R²⁵,
wherein R²⁴ and R²⁵ are independently C₁₋₆alkyl;
When the compound of the present invention is represented by formula (I), most particularly, R¹ is a group derived from dihydrothiophene-2-one, pyrrolidinone, five-membered lactone, or five-membered thilactone by removing a hydrogen therefrom, wherein said group is optionally substituted by C₁₋₃alkyl or phenyl, or —CH₂C(═O)OC₂H₅; R² is —H or —CH₃;
When the compound is represented by formula (II), most particularly, R¹ is C₁₋₃alkyl and R² is C₁₋₆alkyl optionally substituted by halo or heteroaryl, or aryl optionally substituted by halo or heteroaryl;
R³ is chloro;
R⁴ is —H;
G is N;
Q is N or CH;
W is —C(═O)—;
X is selected from Formulas (i), and (ii), below: 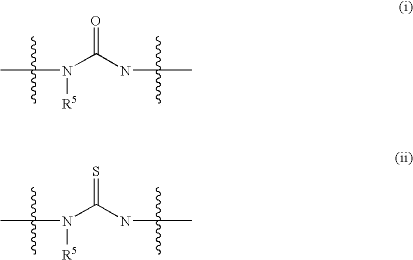
wherein R⁵ is —H or C₁₋₆alkyl. R⁵ is most particularly —H.

Y is selected from formulas (d), (e), (f), (g), (h), (j) and (k), below: 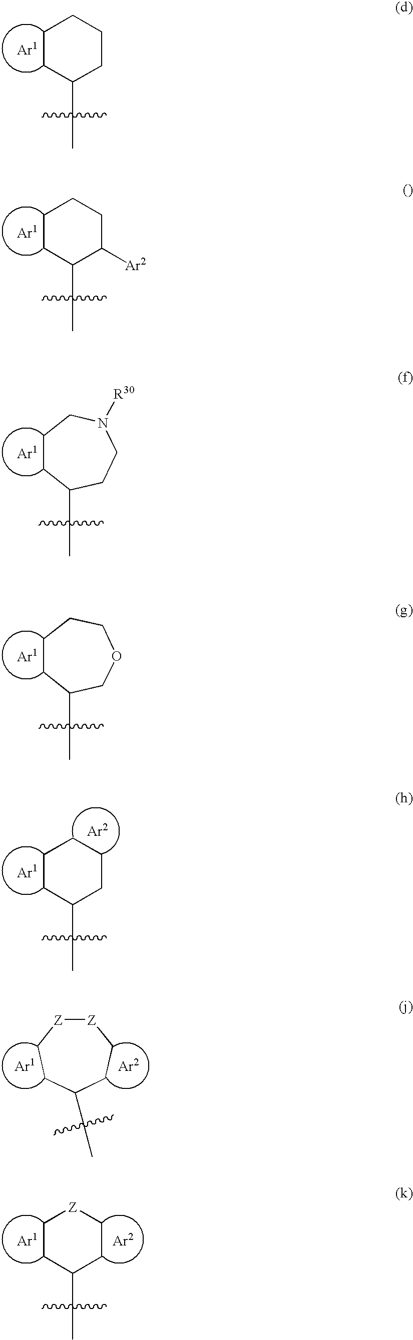
wherein Z is selected from —C—, —C(═O)—, —O—, —N(-alkyl)-, —NH—, —S—, —S(═O)— and —SO₂—; Ar¹ and Ar² are, independently, optionally substituted aryl, or optionally substituted heteraryl; R³⁰ is a C₁₋₆hydrocarbyl; when Ar¹ or Ar² is represented by a three-quarter cycle attached to a ring structure, Ar¹ or Ar² is fused with said ring structure.

Y is even more particularly represented by structure (l), (m), (n), (o), p), (q), (r), (s), (t), (u), (v), (w), (x), (y), (z), (a1), (b1), (c1), (d1), (e1), (f1), (g1), or (h1) below; Y is most particularly represented by structure (n), (s), (t), (z), (a1), (b1), (c1), (d1), (f1), (g1), or (h1) below. 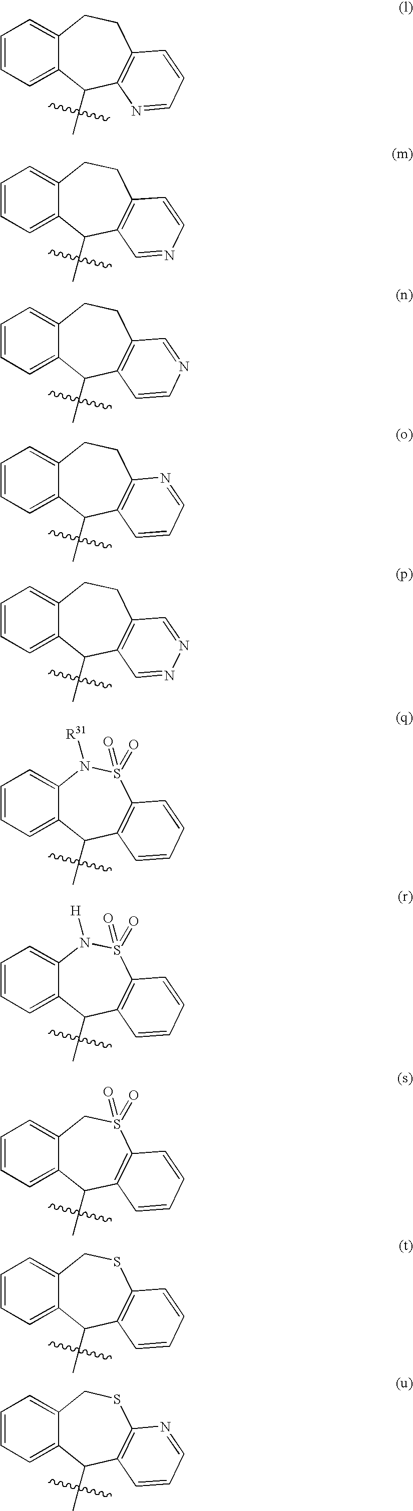 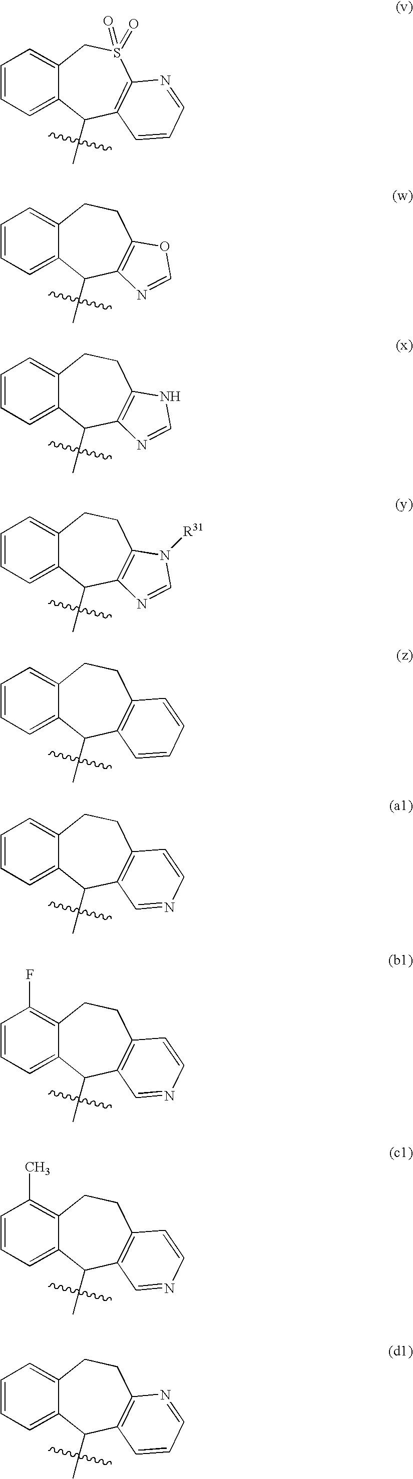 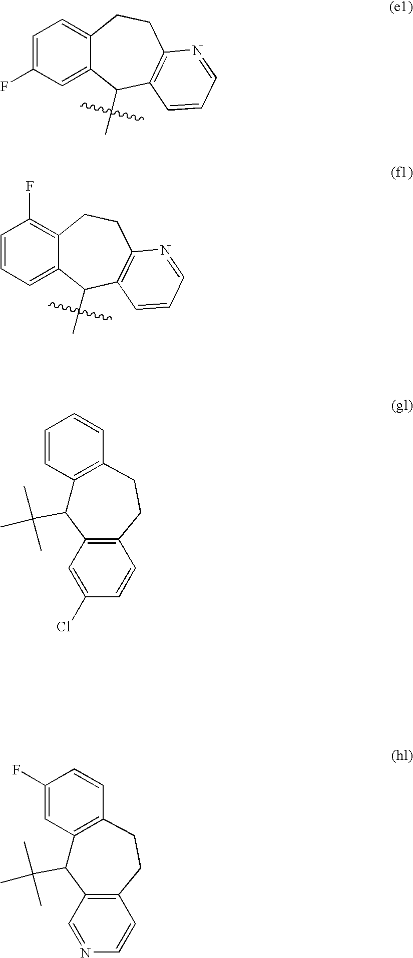
wherein R³¹ is a C₁₋₆alkyl.

Specific examples of compounds of the present invention that may be used in practicing the present invention are listed in Table 1, below.

TABLE 1
Compounds.

| | | | Mass |
|---|---|---|---|
| Cmpd | Chemical Structure | Chemical Name | Spec. |
| 1 | 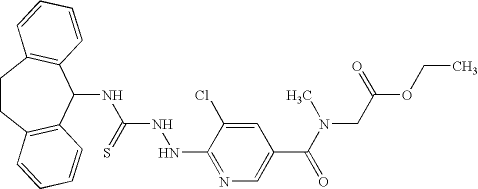 | N-[[5-chloro-6-12-[[(10,11- dihydro-5H-dibenzo[a,d]cyclo- hepten-5-yl)amino]thioxomethyl]- hydrazino]-3-pyridinyl]-carbonyl]- N-methyl-glycine ethyl ester | 538 |
| 2 | 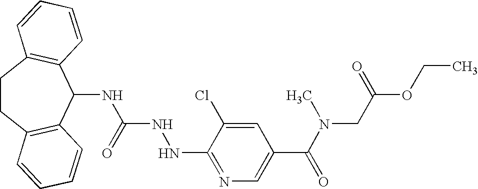 | N-[[(5-chloro-6-[2-[[10,11- dihydro-5H-dibenzo[a,d]cyclo- hepten-5-yl)amino]carbonyl]- hydrazino]-3-pyridinyl]carbonyl]- N-methyl-glycine ethyl ester | 522 |
| 3 | 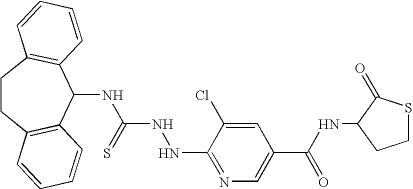 | 5-chloro-6-[2-[[(10,11-dihydro- 5H-dibenzo[a,d]-cyclohepten-5- yl)amino]thioxomethyl]hydrazino]- N-(tetrahydro-2-oxo-3-thienyl)-3- pyridine-carboxamide | 538 |
| 4 | 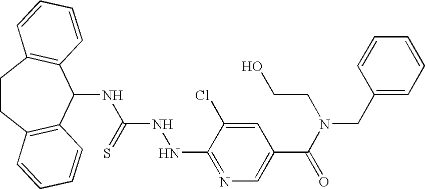 | 5-chloro-6-[2-[[(10,11-dihydro- 5H-dibenzo[a,d]-cyclohepten-5- yl)amino]thioxomethyl]hydrazino]- N-(2-hydroxyethyl)-N-(phenyl- methyl)-3-pyridinecarboxamide | 572 |
| 5 | 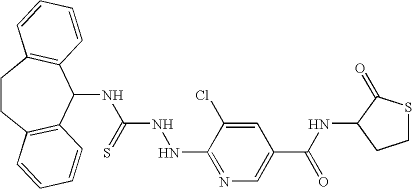 | 5-chloro-6-[2-[[(10,11-dihydro- 5H-dibenzo[a,d]-cyclohepten-5- yl)amino]carbonyl)hydrazino]-N- (tetrahydro-2-oxo-3-thienyl)-3- pyridinecarboxamide | 522 |
| 6 | 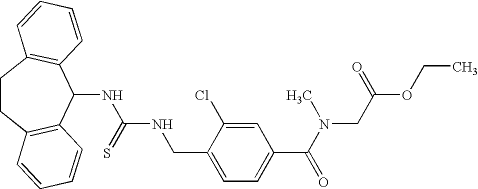 | N-[3-chloro-4-[[[[(10,11-dihydro- 5H-dibenzo[a,d]cyclohepten-5- yl)amino]carbonyl)amino]methyl]benzoyl]-N-methyl-glycine ethyl ester | 520 |
| 7 | 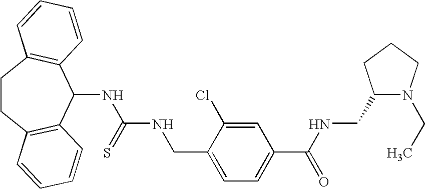 | 3-chloro-4-[[[[(10,11-dihydro-5H- dibenzo[a,d]cyclo-hepten-5- yl)amino]carbonyl]amino]methyl]- N-[[(2S)-1-ethyl-2-pyrrolidinyl]- methyl]-benzamide | 531 |
| 8 | 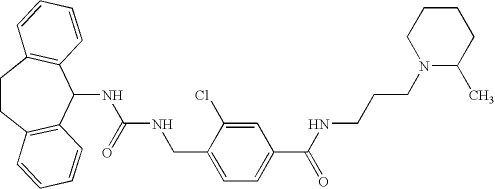 | 3-chloro-4-[[[[(10,11-dihydro-5H- dibenzo[a,d]cyclo-hepten-5-yl- amino]carbonyl]amino]methyl]-N- [3-(2-methyl-1-piperidinyl)- propyl]-benzamide | 559 |
| 9 | 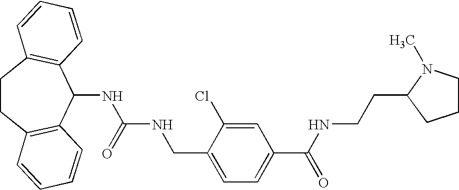 | 3-chloro-4-[[[[(10,11-dihydro-5H- dibenzo[a,d]-cyclohepten-5- yl)amino]carbonyl]amino]methyl]- N-[2-(1-methyl-2-pyrrolidinyl)- ethyl]-benzamide | 531 |
| 10 | 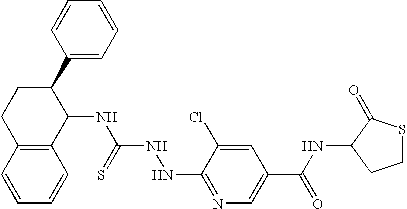 | 5-chloro-N-(tetrahydro-2-oxo-3- thienyl)-6-[2-[[[(1S,2R)-1,2,3,4- tetrahydro-2-phenyl-1- naphthalenyl]amino]thioxomethyl]hydrazino]-3-pyridinecarboxamide | 552 |
| 11 | 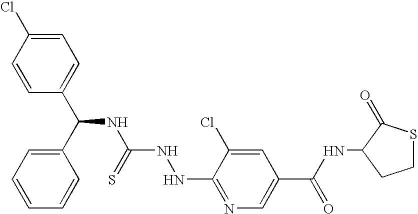 | 5-Chloro-6-[2-[[[(4-chloro- phenyl)-phenyl-methyl]-amino]- thioxomethyl]-hydrazino]-N- (tetrahydro-2-oxo-3-thienyl)-3- pyridine-carboxamide | 546 |
| 12 | 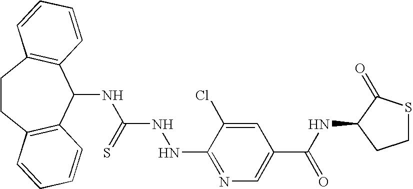 | 5-chloro-6-[2-[[(10,11-dihydro- 5H-dibenzo[a,d]cyclohepten-5- yl)amino]thioxomethyl]hydrazino]- N-[(3R)-tetrahydro-2-oxo-3- thienyl]-3-pyridinecarboxamide | 538 |
| 13 | 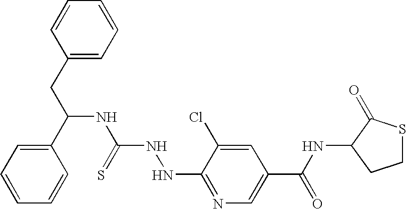 | 5-chloro-6-[2-[[(1,2-diphenyl- ethyl)amino]thioxomethyl]hydrazino]- N-(tetrahydro-2-oxo-3- thienyl)-3-pyridinecarboxamide | 526 |
| 14 | 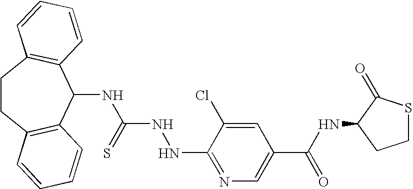 | 5-chloro-6-[2-[[(10,11-dihydro- 5H-dibenzo[a,d]cyclohepten-5- yl)amino]thioxomethyl]hydrazino]- N-((3S)-tetrahydro-2-oxo-3- thienyl)-3-pyridinecarboxamide | 538 |
| 15 | 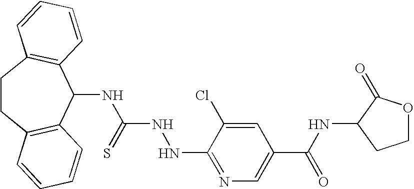 | 5-chloro-6-[2-[[(10,11-dihydro- 5H-dibenzo[a,d]cyclohepten-5- yl)amino]thioxomethyl]hydrazino]- N-(tetrahydro-2-oxo-3-furanyl)-3- pyridinecarboxamide | 522 |
| 16 | 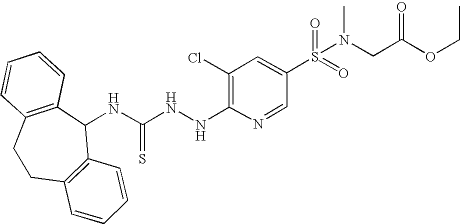 | N-[[5-chloro-6-[2-[[(10,11- dihydro-5h-dibenzo[a,d]cyclo- hepten-5-yl)amino]thioxomethyl]- hydrazino]-3-pyridinyl]sulfonyl]- n-methyl-glycine, ethyl ester | 574 |
| 17 | 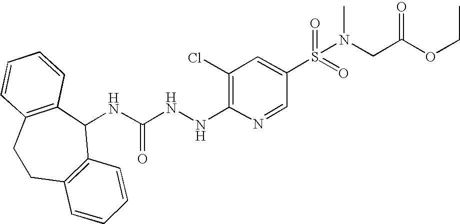 | N-[[5-chloro-6-[2-[[(10,11- dihydro-5H-dibenzo[a,d]cyclo- hepten-5-yl)amino]carbonyl]- hydrazino]-3-pyridinyl]sulfonyl]- N-methyl-glycine, ethyl ester | 558 |
| 18 | 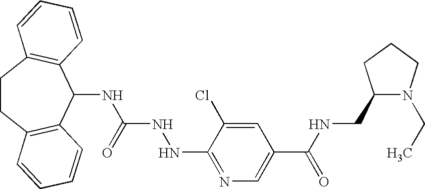 | 5-chloro-6-[2-[[(10,11-dihydro- 5H-dibenzo[a,d]cyclohepten-5- yl)amino]carbonyl]hydrazino]-N- [[(2S)-1-ethyl-2-pyrrolidinyl]- methyl]-3-pyridinecarboxamide | 533 |
| 19 | 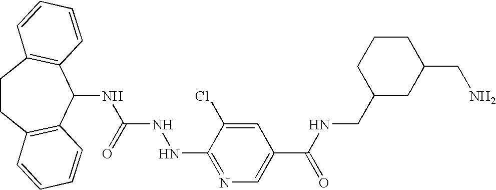 | N-[[3-(aminomethyl)cyclohexyl]- methyl]-5-chloro-6-[2-[[(10,11- dihydro-5H-dibenzo[a,d]cyclo- hepten-5-yl)amino]carbonyl]- hydrazino]-3-pyridinecarboxamide | 547 |
| 20 | 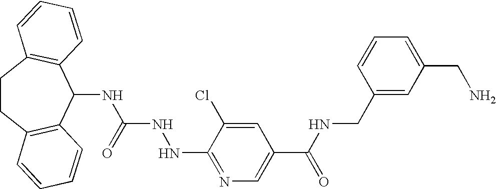 | N-[[3-(aminomethyl)phenyl]- methyl]-5-chloro-6-[2-[[(10,11- dihydro-5H-dibenzo[a,d]cyclo- hepten-5-yl)amino]carbonyl]- hydrazino]-3-pyridinecarboxamide | 541 |
| 21 | 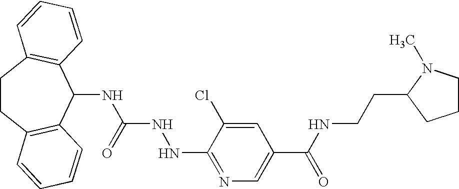 | 5-chloro-6-[2-[[(10,11-dihydro- 5H-dibenzo[a,d]cyclohepten-5- yl)amino]carbonyl]hydrazino]-N- [2-(1-methyl-2-pyrrolidinyl)ethyl]- 3-pyridinecarboxamide | 533 |
| 22 | 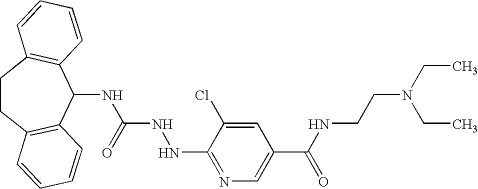 | 5-chloro-N-[2-(diethylamino)- ethyl]-6-[2-[[(10,11-dihydro-5H- dibenzo[a,d]cyclohepten-5-yl)- amino]carbonyl]hydrazino]-3- pyridinecarboxamide | 521 |
| 23 | 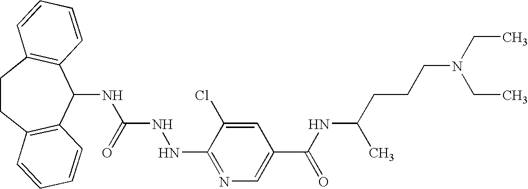 | 5-chloro-N-[4-(diethylamino)-1- methylbutyl]-6-[2-[[(10,11- dihydro-5H-dibenzo[a,d]cyclo- hepten-5-yl)amino]carbonyl]- hydrazino]-3-pyridinecarboxamide | 563 |
| 24 | 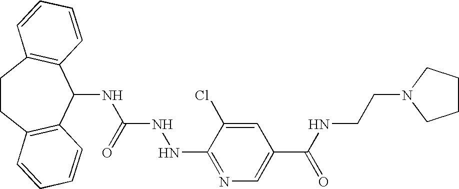 | 5-chloro-6-[2-[[(10,11-dihydro- 5H-dibenzo[a,d]cyclohepten-5-yl)- amino]carbonyl]hydrazino]-N-[2- (1-pyrrolidinyl)ethyl]-3-pyridine- carboxamide | 519 |
| 25 | 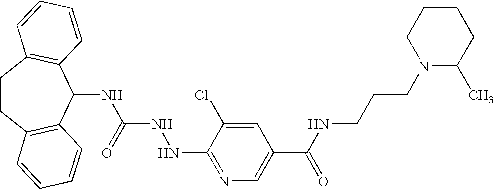 | 5-chloro-6-[2-[[(10,11-dihydro- 5H-dibenzo[a,d]cyclohepten-5- yl)amino]carbonyl]hydrazino]-N- [3-(2-methyl-1-piperidinyl)- propyl]-3-pyridinecarboxamide | 561 |
| 26 | 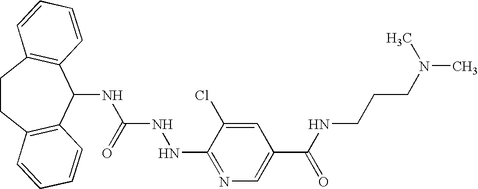 | 5-chloro-6-[2-[[(10,11-dihydro- 5H-dibenzo[a,d]cyclohepten-5- yl)amino]carbonyl]hydrazino]-N- [3-(dimethylamino)propyl]-3- pyridinecarbox.amide | 507 |
| 27 | 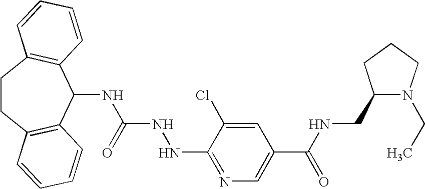 | 5-chloro-6-[2-[[(10,11-dihydro- 5H-dibenzo[a,d]cyclohepten-5- yl)amino]carbonyl]hydrazino]-N- [[(2R)-1-ethyl-2-pyrrolidinyl]- methyl]-3-pyridinecarboxamide | 533 |
| 28 | 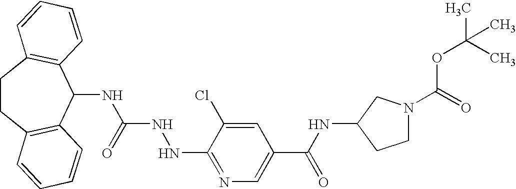 | 3-[[[5-chloro-6-[2-[[(10,11- dihydro-5H-dibenzo[a,d]cyclo- hepten-5-yl)amino]carbonyl]- hydrazino]-3-pyridinyl]carbonyl]- amino]-1-pyrrolidinecarboxylic acid,1,1-dimethylethyl ester | 591 |
| 29 | 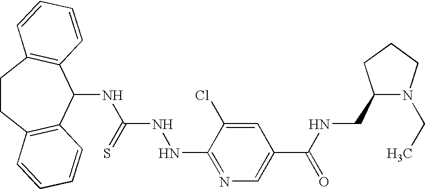 | 5-chloro-6-[2-[[(10,11-dihydro- 5H-dibenzo[a,d]cyclohepten-5- yl)amino]thioxomethyl]hydrazino]- N-[[(2S)-1-ethyl-2-pyrrolidinyl]- methyl]-3-pyridinecarboxamide | 549 |
| 30 | 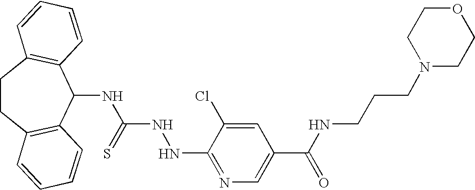 | 5-chloro-6-[2-[[(10,11-dihydro- 5H-dibenzo[a,d]cyclohepten-5- yl)amino]thioxomethyl]hydrazino]- N-[3-(4-morpholinyl)propyl]-3- pyridinecarboxamide | 565 |
| 31 | 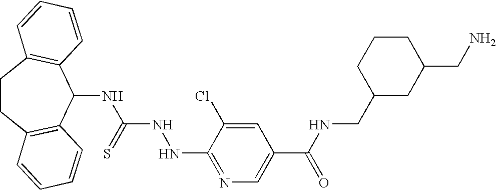 | N-[[3-(aminomethyl)cyclohexyl]- methyl]-5-chloro-6-[2-[[(10,11- dihydro-5H-dibenzo[a,d]cyclo- hepten-5-yl)amino]thioxomethyl]- hydrazino]-3-pyridine- carboxamide | 563 |
| 32 | 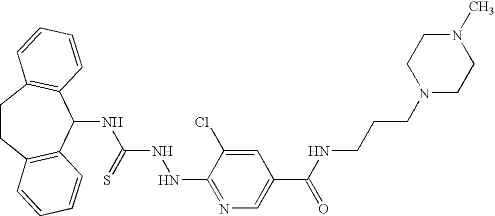 | 5-chloro-6-[2-[[(10,11-dihydro- 5H-dibenzo[a,d]cyclohepten-5- yl)amino]thioxomethyl]hydrazino]- N-[3-(4-methyl-1-piperazinyl)- propyl]-3-pyridinecarboxamide | 578 |
| 33 | 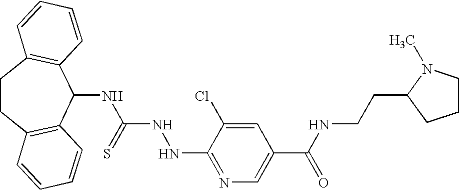 | 5-chloro-6-[2-[[(10,11-dihydro- 5H-dibenzo[a,d]cyclohepten-5- yl)amino]thioxomethyl]hydrazino]- N-[2-(1-methyl-2-pyrrolidinyl)- ethyl]-3-pyridinecarboxamide | 549 |
| 34 | 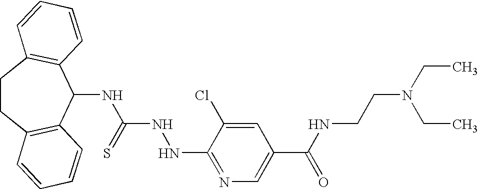 | 5-chloro-N-[2-(diethylamino)- ethyl]-6-[2-[[(10,11-dihydro-5H- dibenzo[a,d]cyc1ohepten-5-yl)- amino]thioxomethyl]hydrazino]- 3-pyridinecarboxamide | 537 |
| 35 | 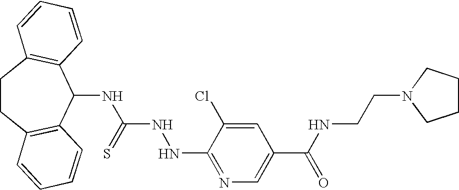 | 5-chloro-6-[2-[[(10,11-dihydro- 5H-dibenzo[a,d]cyclohepten-5- yl)amino]thioxomethyl]hydrazino]- N-[2-(1-pyrrolidinyl)ethyl]-3- pyridinecarboxamide | 535 |
| 36 | 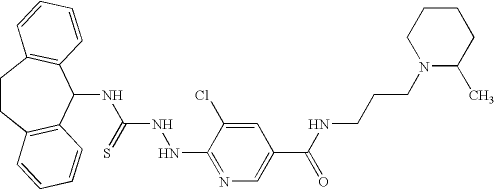 | 5-chloro-6-[2-[[(10,11-dihydro- 5H-dibenzo[a,d]cyclohepten-5- yl)amino]thioxomethyl]hydrazino]- N-[3-(2-methyl-1-piperidinyl)- propyl]-3-pyridinecarboxamide | 577 |
| 37 | 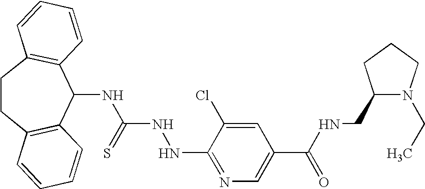 | 5-chloro-6-[2-[[(10,11-dihydro- 5H-dibenzo[a,d]cyclohepten-5- yl)amino]thioxomethyl]hydrazino]- N-[((2R)-1-ethyl-2-pyrrolidinyl)- methyl]-3-pyridinecarboxamide | 549 |
| 38 | 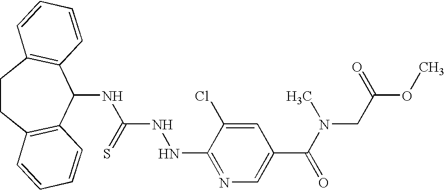 | N-[[5-chloro-6-[2-[[(10,11- dihydro-5H-dibenzo[a,d]cyclo- hepten-5-yl)amino]thioxomethyl]- hydrazino]-3-pyridinyl]carbonyl]- N-methyl-glycine, methyl ester | 524 |
| 39 | 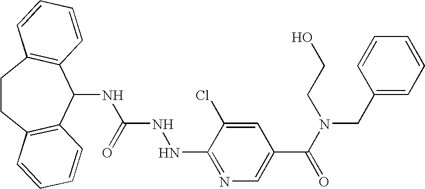 | 5-chloro-6-[2-[[(10,11-dihydro- 5H-dibenzo[a,d]cyclohepten-5- yl)amino]carbonyl]hydrazino]-N- (2-hydroxyethyl)-N-(phenyl- methyl)-3-pyridinecarboxamide | 556 |
| 40 | 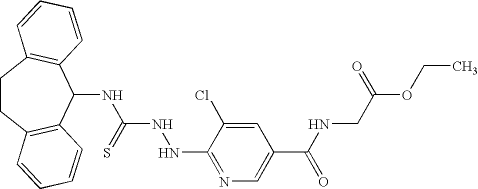 | N-[[5-chloro-6-[2-[[(10,11- dihydro-5H-dibenzo[a,d]cyclo- hepten-5-yl)amino]thioxomethyl]- hydrazino]-3-pyridinyl]carbonyl]- glycine, ethyl ester | 524 |
| 41 | 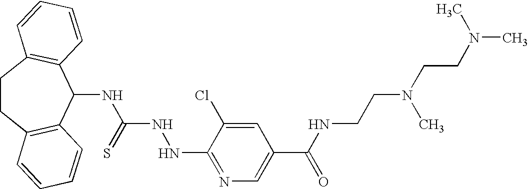 | 5-chloro-6-[2-[[(10,11-dihydro- 5H-dibenzo[a,d]cyclohepten-5- yl)amino]thioxomethyl]hydrazino]- N-[2-[[2-(dimethylamino)ethyl]- methylamino]-ethyl]-3-pyridine- carboxamide | 566 |
| 42 | 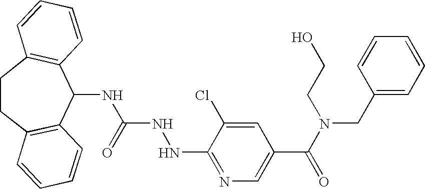 | 2-[2-chloro-4-[[(2-hydroxyethyl)- (phenylmethyl)amino]carbonyl]phenyl]-N-(10,11-dihydro-5H- dibenzo[a,d]cyclohepten-5-yl)- hydrazinecarboxamide | 555 |
| 43 | 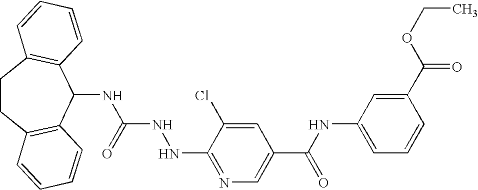 | 3-[[[5-chloro-6-[2-[[(10,11- dihydro-5H-dibenzo[a,d]cyclo- hepten-5-yl)amino]carbonyl]- hydrazino]-3-pyridinyl]-carbonyl]- amino]-benzoic acid, ethyl ester | 570 |
| 44 | 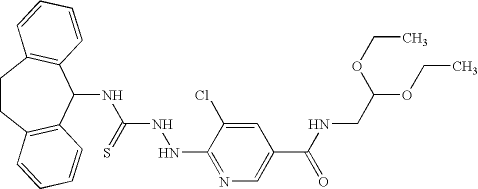 | 5-chloro-N-(4,4-diethoxybutyl)-6- [2-[[(10,11-dihydro-5H-dibenzo- [a,d]cyclohepten-5-yl)amino]- carbonyl]hydrazino]-3-pyridine- carboxamide | 566 |
| 45 | 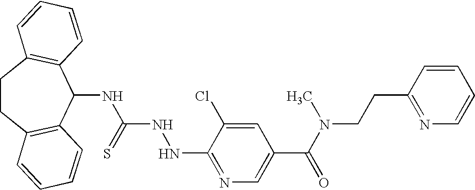 | 3-pyridinecarboxamide, 5-chloro- 6-[2-[[(10,11-dihydro-5H-dibenzo- [a,d]cyclohepten-5-yl)amino]- thioxomethyl]hydrazino]-N- methyl-N-[2-(2-pyridinyl)ethyl]- | 557 |
| 46 | 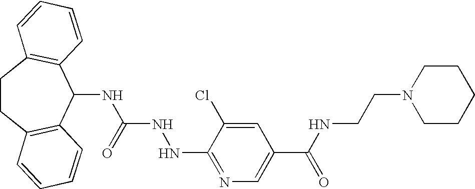 | 5-chloro-6-[2-[[(10,11-dihydro- 5H-dibenzo[a,d]cyclohepten-5- yl)amino]carbonyl]hydrazino]-N- [2-(1-piperidinyl)ethyl]-3- pyridinecarboxamide | 533 |
| 47 | 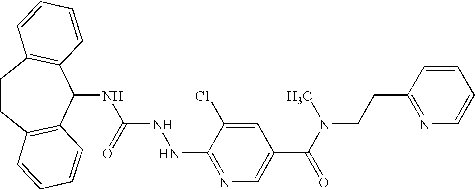 | 3-pyridinecarboxamide, 5-chloro- 6-[2-[[(10,11-dihydro-5H-dibenzo- [a,d]cyclohepten-5-yl)amino]- carbonyl]hydrazino]-N-methyl-N- [2-(2-pyridinyl)ethyl]- | 541 |
| 48 | 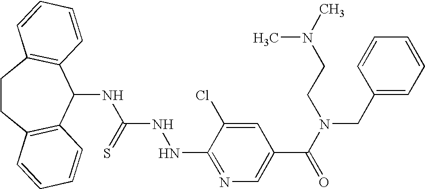 | 5-chloro-6-[2-[[(10,11-dihydro- 5H-dibenzo[a,d]cyclohepten-5-yl)- amino]carbonyl]hydrazino]-N-[2- (dimethylamino)ethyl]-N-(phenyl- methyl)-3-pyridinecarboxamide | 583 |
| 49 | 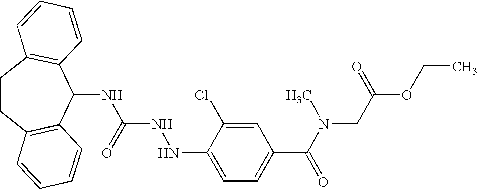 | N-[3-chloro-4-[2-[[(10,11- dihydro-5H-dibenzo[a,d]cyclo- hepten-5-yl)amino]carbonyl]- hydrazino]benzoyl]-N-methyl- glycine, ethyl ester | 521 |
| 50 | 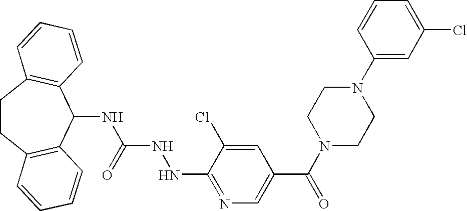 | 2-[3-chloro-5-[[4-(3-chloro- phenyl)-1-piperazinyl]carbonyl]-2- pyridinyl]-N-(10,11-dihydro-5H- dibenzo[a,d]cyclohepten-5-yl)- hydrazinecarboxamide | 601 |
| 51 | 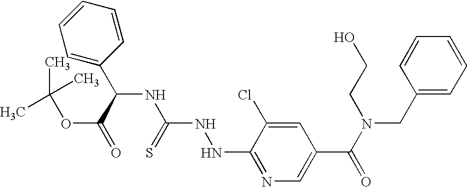 | (α^{a}S)-α-[[[2-(3-chloro-5-[[(2- hydroxyethyl)(phenylmethyl)amino]carbonyl]-2-pyridinyl]- hydrazino]thioxomethyl]amino]- benzeneacetic acid, 1,1- dimethylethyl ester | 570 |
| 52 | 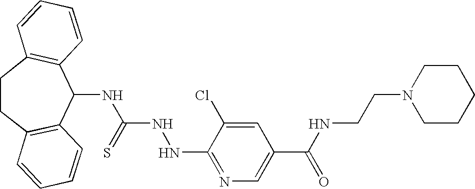 | 5-chloro-6-[2-[((10,11-dihydro- 5H-dibenzo[a,d]cyclohepten-5- yl)amino]thioxomethyl]hydrazino]- N-[2-(1-piperidinyl)ethyl]-3- pyridinecarboxamide | 549 |
| 53 | 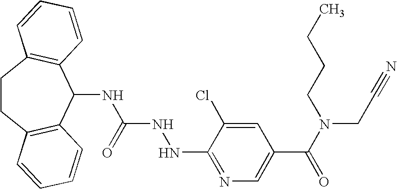 | N-butyl-5-chloro-N-(cyano- methyl)-6-[2-[[(10,11-dihydro-5H- dibenzo[a,d]cyclohepten-5-yl)- amino]carbonyl]hydrazino]-3- pyridinecarboxamide | 517 |
| 54 | 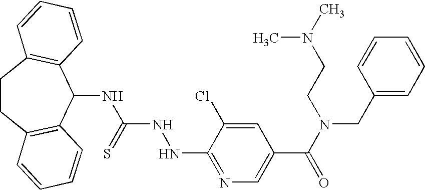 | 5-chloro-6-[2-[[(10,11-dihydro- 5H-dibenzo[a,d]cyclohepten-5- yl)amino]thioxomethyl]hydrazino]- N-[2-(dimethylamino)ethyl]-N- (phenylmethyl)-3-pyridine- carboxamide | 599 |
| 55 | 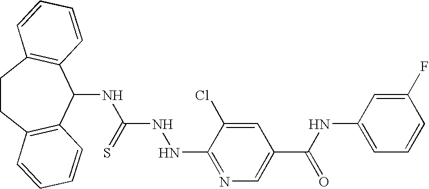 | 5-chloro-6-[2-[[(10,11-dihydro- 5H-dibenzo[a,d]cyclohepten-5- yl)amino]thioxomethyl]hydrazino]- N-(3-fluorophenyl)-3-pyridine- carboxamide | 532 |
| 56 | 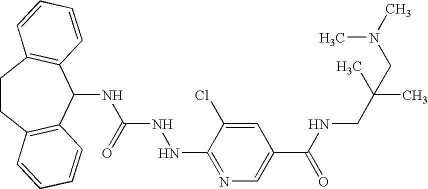 | 5-chloro-6-[2-[[(10,11-dihydro- 5H-dibenzo[a,d]cyclohepten-5- yl)amino]carbonyl]hydrazino]-N- [3-(dimethylamino)-2,2-dimethyl- propyl]-3-pyridinecarboxamide | 535 |
| 57 | 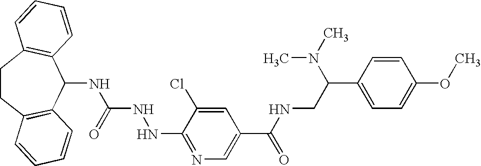 | 5-chloro-6-[2-[[(10,11-dihydro- 5H-dibenzo[a,d]cyclohepten-5-yl)- amino]carbonyl]hydrazino]-N-[2- (dimethylamino)-2-(4-methoxy- phenyl)ethyl]-3-pyridine- carboxamide | 599 |
| 58 | 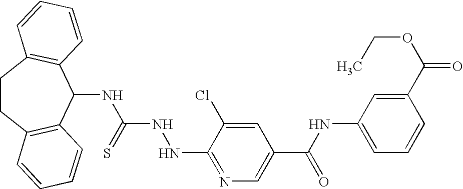 | 3-[[[5-chloro-6-[2-[[(10,11- dihydro-5H-dibenzo[a,d]cyclo- hepten-5-yl)amino]thioxomethyl]- hydrazino]-3-pyridinyl]carbonyl]- amino]-benzoic acid, ethyl ester | 586 |
| 59 | 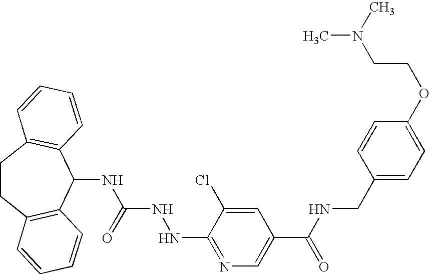 | 5-chloro-6-[2-[[(10,11-dihydro- 5H-dibenzo[a,d]cyclohepten-5- yl)amino]carbonyl]hydrazino]-N- [[4-[2-(dimethylamino)ethoxy]- phenyl]methyl]-3-pyridine- carboxamide | 599 |
| 60 | 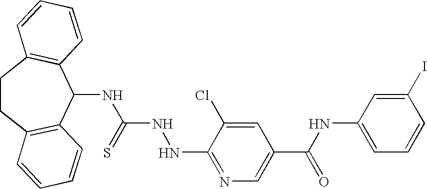 | 5-chloro-6-[2-[[(10,11-dihydro- 5H-dibenzo[a,d]cyclohepten-5- yl)amino]thioxomethyl]hydrazino]- N-(3-iodophenyl)-3-pyridine- carboxamide | 640 |
| 61 | 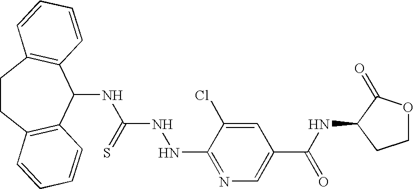 | 5-chloro-6-[2-[[(10,11-dihydro- 5H-benzo[a,d]cyclohepten-5- yl)amino]thioxomethyl]hydrazino]- N-[(3R)-tetrahydro-2-oxo-3- fuanyl]-3-pyridinecarboxamide | 522 |
| 62 | 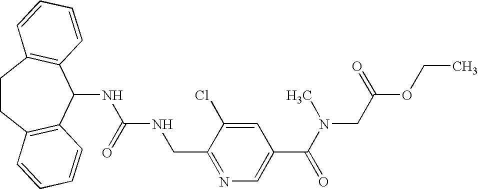 | N-[[5-Chloro-6-[[[[(10,11- dihydro-5H-dibenzo[a,d]cyclo- hepten-5-yl)amino]carbonyl]- amino]methyl]-3-pyridinyl]- carbonyl]-N-methyl-glycine ethyl ester | 521 |
| 63 | 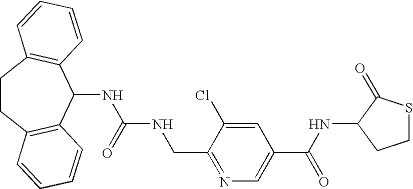 | 5-Chloro-6-[[[[(10,11-dihydro-5H- dibenzo[a,d]cyclohepten-5-yl)- amino]carbonyl]amino]methyl]-N- (tetrahydro-2-oxo-3-thienyl)-3- pyridinecarboxamide | 521 |
| 64 | 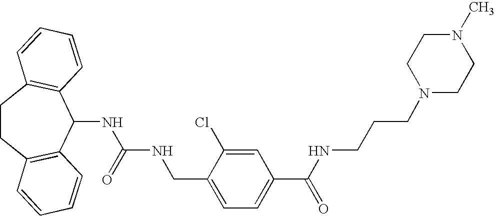 | 3-Chloro-4-[[[[(10,11-dihydro-5H- dibenzo[a,d]cyclohepten-5-yl)- amino]carbonyl]amino]methyl]-N- [3-(4-methyl-1-piperazinyl)- propyl]-benzamide | 560 |
| 65 | 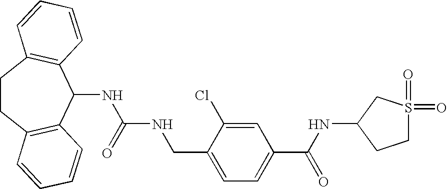 | 3-Chloro-4-[[[[(10,11-dihydro-5H- dibenzo[a,d]cyclohepten-5-yl)- amino]carbonyl]amino]methyl]-N- (tetrahydro-1,1-dioxido-3-thienyl)- benzamide | 538 |
| 66 | 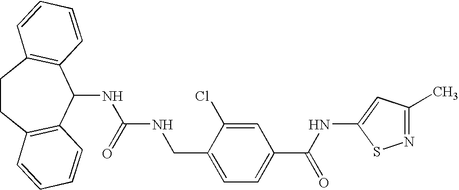 | 3-Chloro-4-[[[[(10,11-dihydro-5H- dibenzo[a,d]cyclohepten-5-yl)- amino]carbonyl]amino]methyl]-N- (3-methyl-5-isothiazolyl)- benzamide | 517 |
| 67 | 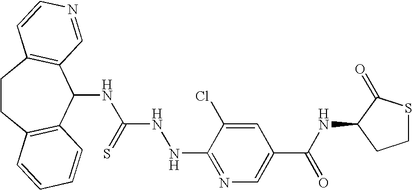 | 5-chloro-6-[2-[[(6,11-dihydro-5H- benzo[5,6]cyclohepta[1,2- c]pyridin-11- yl)amino]thioxomethyl]hydrazino]- N-[(3R)-tetrahydro-2-oxo-3- thienyl]-3-pyridinecarboxamide | 539 |
| 68 | 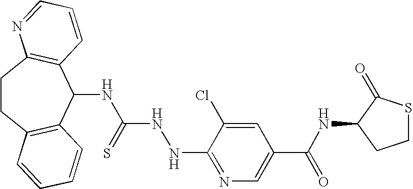 | 5-chloro-6-[2-[[(10,11-dihydro- 5H-benzo[4,5]cyclohepta[1,2- b]pyridin-5- yl)amino]thioxomethyl]hydrazino]- N-[(3R)-tetrahydro-2-oxo-3- thienyl]-3-pyridinecarboxamide | 539 |
| 69 | 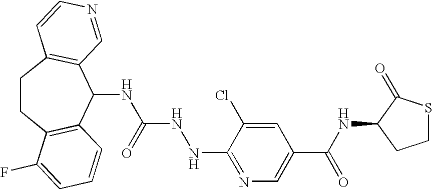 | 5-chloro-6-[2-[[(7-fluoro-6,11- dihydro-5H- benzo[5,6]cyclohepta8 1,2- c]pyridin-11- yl)amino]thioxomethyl]hydrazino]- N-[(3R)-tetrahydro-2-oxo-3- thienyl]-3-pyridinecarboxamide | 557 |
| 70 | 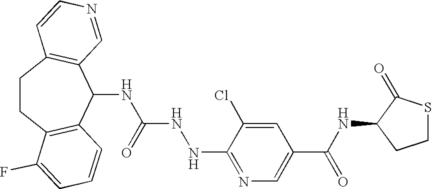 | 5-chloro-6-[2-[[(7-fluoro-6,11- dihydro-5H- benzo[5,6]cyclohepta[1,2- c]pyridin-11- yl)amino]carbonyl]hydrazino]-N- [(3R)-tetrahydro-2-oxo-3-thienyl]- 3-pyridinecarboxamide | 541 |
| 71 | 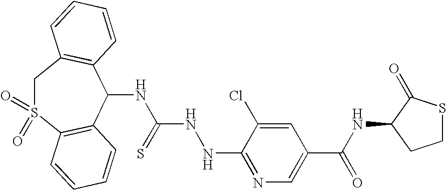 | 5-chloro-6-[2-[[(6,11-dihydro-5,5- dioxidodibenzo[b,e]thiepin-11- yl)amino]thioxomethyl]hydrazino]- N-[(3R)-tetrahydro-2-oxo-3- thienyl]-3-pyridinecarboxamide | 588 |
| 72 | 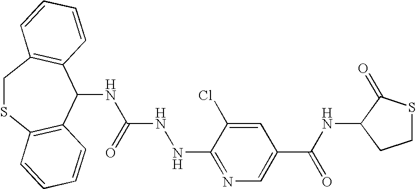 | 5-chloro-6-[2-[[(6,11- dihydrodibenzo[b,e]thiepin-11- yl)amino]thioxomethyl]hydrazino]- N-(tetrahydro-2-oxo-3-thienyl)- 3-pyridinecarboxamide | 556 |
| 73 | 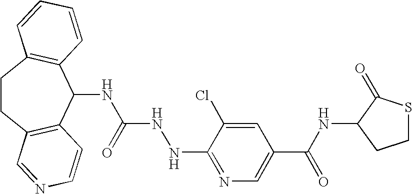 | 5-chloro-6-[2-[[(10,11-dihydro- 5H-benzo[4,5]cyclohepta[1,2- c]pyridin-5- yl)amino]carbonyl]hydrazino]-N- (tetrahydro-2-oxo-3-thienyl)-3- pyridinecarboxamide | 523 |
| 74 | 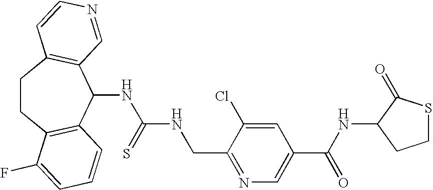 | 5-chloro-6-[[[[(7-fluoro-6,11- dihydro-5H- benzo[5,6]cyclohepta[1,2- c]pyridin-11- yl)amino]thioxomethyl]amino]methyl]-N-[(3R)-tetrahydro-2-oxo-3- thienyl]-3-pyridinecarboxamide | 556 |
| 75 | 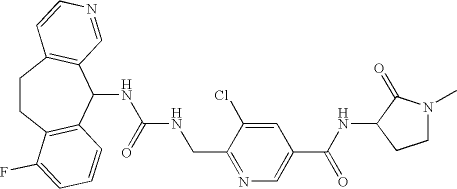 | 5-chloro-6-{[[[(7-fluoro-6,11- dihydro-5H- benzo[5,6]cyclohepta[1,2- c]pyridin-11- yl)amino]thioxomethyl]amino]methyl]-N-[(3R)-1-methyl-2-oxo-3- pyrrolidinyl]-3- pyridinecarboxamide | 553 |
| 76 | 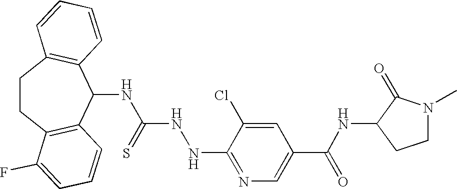 | 5-chloro-6-[2-[[(7-fluoro-6,11- dihydro-5H- benzo[5,6]cyclohepta[1,2- c]pyridin-11- yl)amino]thioxomethyl]hydrazino]- N-[(3R)-1-methyl-2-oxo-3- pyrrolidinyl]-3- pyridinecarboxamide | 554 |
| 77 | 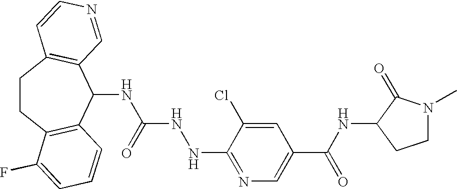 | 5-chloro-6-[2-[[(7-fluoro-6,11- dihydro-5H- benzo[5,6]cyclohepta[1,2- c]pyridin-11- yl)amino]carbonyl]hydrazino]-N- [(3R)-1-methyl-2-oxo-3- pyrrolidinyl]-3- pyridinecarboxamide | 538 |
| 78 | 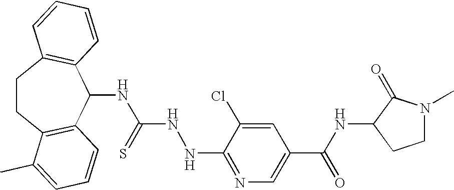 | 5-chloro-6-[2-[[(6,11-dihydro-7- methyl-5H- benzo[5,6]cyclohepta[1,2- c]pyridin-11- yl)amino]thioxomethyl]hydrazino]- N-[(3R)-1-methyl-2-oxo-3- pyrrolidinyl]-3- pyridinecarboxamide | |
| 79 | 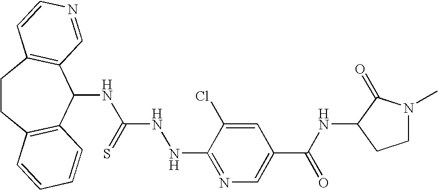 | 5-chloro-6-[2-4[(6,11-dihydro-5H- benzo[5,6]cyclohepta[1,2- c]pyridin-11- yl)amino]thioxomethyl]hydrazino]- N-[(3R)-1-methyl-2-oxo-3- pyrrolidinyl]-3- pyridinecarboxamide | 536 |
| 80 | 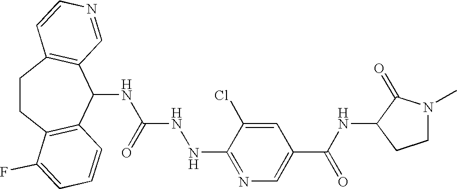 | 5-chloro-6-[([[(7-fluoro-6,11- dihydro-5H- benzo[5,6]cyclohepta[1,2- c]pyridin-11- yl)amino]carbonyl]amino]methyl]- N-[(3R)-1-methyl-2-oxo-3- pyrrolidinyl]-3- pyridinecarboxamide | 537 |
| 81 | 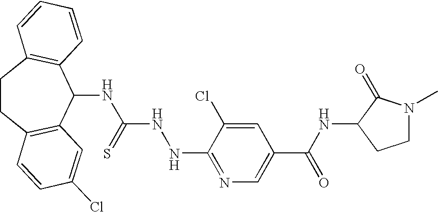 | 5-chloro-6-[2-[[(3-chloro-10,11- dihydro-5H- dibenzo[a,d]cyclohepten-5- yl)amino]thioxomethyl]hydrazino]- N-[(3R)-1-methyl-2-oxo-3- pyrrolidinyl]-3- pyridinecarboxamide | 569 |
| 82 | 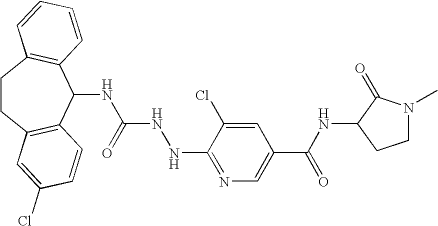 | 5-chloro-6-[2-[[(2-chloro-10,11- dihydro-5H- dibenzo[a,d]cyclohepten-5- yl)amino]thioxomethyl]hydrazino]- N-[(3R)-1-methyl-2-oxo-3- pyrrolidinyl]-3- pyridinecarboxamide | 569 |
| 83 | 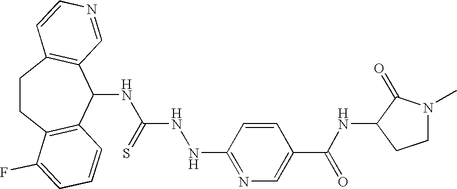 | 6-[2-[[(7-fluoro-6,11-dihydro-5H- benzo[5,6]cyclohepta[1,2- c]pyridin-11- yl)amino]thioxomethyl]hydrazino]- N-[(3R)-1-methyl-2-oxo-3- pyrrolidinyl]-3- pyridinecarboxamide | 520 |
| 84 | 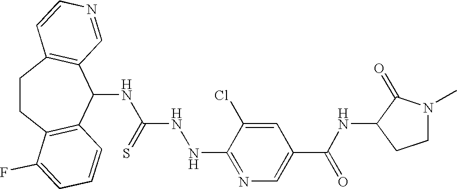 | 5-chloro-6-[2-[[(7-fluoro-6,11- dihydro-5H- benzo[5,6]cyclohepta[1,2- c]pyridin-11- yl)amino]thioxomethyl]-1- methylhydrazino]-N-(3R)-1- methyl-2-oxo-3-pyrrolidinyl]-3- pyridinecarboxamide | 568 |
| 85 | 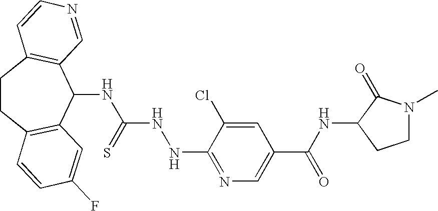 | 5-chloro-6-[2-[[(9-fluoro-6,11- dihydro-5H- benzo[5,6]cyclohepta[1,2- c]pyridin-11- yl)amino]thioxomethyl]hydrazino]- N-[(3R)-1-methyl-2-oxo-3- pyrrolidinyl]-3- pyridinecarboxamide | 554 |
| 86 | 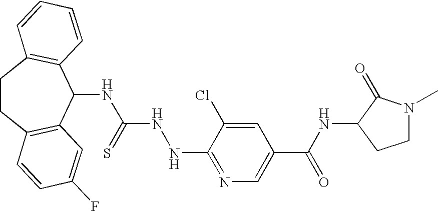 | 5-chloro-6-[2-[[(7-fluoro-10,11- dihydro-5H- benzo[4,5]cyclohepta[1,2- b]pyridin-5- yl)amino]thioxomethyl]hydrazino]- N-[(3R)-1-methyl-2-oxo-3- pyrrolidinyl]-3- pyridinecarboxamide | 554 |
| 87 | 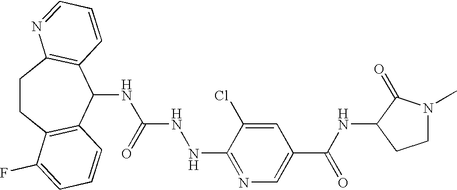 | 5-chloro-6-[2-[[(9-fluoro-10,11- dihydro-5H- benzo[4,5]cyclohepta[1,2- b]pyridin-5- yl)amino]carbonyl]hydrazino]-N- [(3R)-1-methyl-2-oxo-3- pyrrolidinyl]-3- pyridinecarboxamide | 538 |
| 88 | 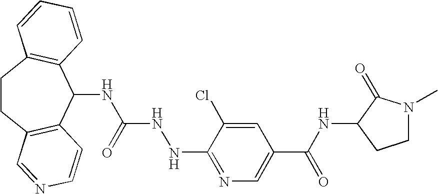 | 5-chloro-6-[2-[[(10,11-dihydro- 5H-benzo[4,5]cyciohepta[1,2- c]pyridin-5- yl)amino]carbonyl]hydrazino]-N- (1-methyl-2-oxo-3-pyrrolidinyl)- 3-pyridinecarboxamide | 520 |
| 89 | 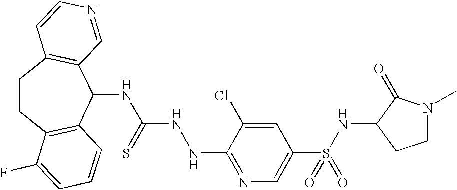 | 2-[3-chloro-5-[[[(3R)-1-methyl-2- oxo-3- pyrrolidinyl]amino]sulfonyl]-2- pyridinyl]-N-(7-fluoro-6,11- dihydro-5H- benzo[5,6]cyclohepta[1,2- c]pyridin-11-yl)- hydrazinecarbothioamide | 590 |
| 90 | 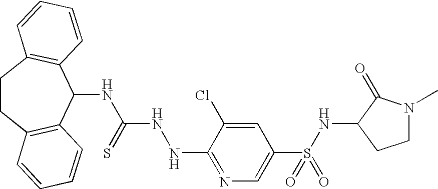 | 2-[3-chloro-5-[[[(3R)-1-methyl-2- oxo-3- pyrrolidinyl]amino]sulfonyl]-2- pyridinyl]-N-(10,11-dihydro-5H- dibenzo[a,d]cyclohepten-5-yl)- hydrazinecarbothioamide | 571 |
| 91 | 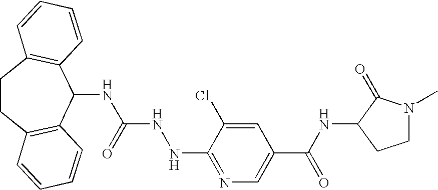 | 5-chloro-6-[2-[[(10,11-dihydro- 5H-dibenzo[a,d]cyclohepten-5- yl)amino]carbonyl]hydrazino]-N- [(3R)-1-methyl-2-oxo-3- pyrrolidinyl]-3- pyridinecarboxamide | 519 |
| 92 | 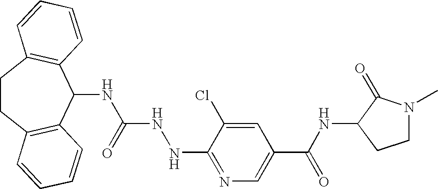 | 5-chloro-6-[2-[[(10,11-dihydro- 5H-dibenzo[a,d]cyclohepten-5- yl)amino]thioxomethyl]hydrazino]- N-[(3R)-1-methyl-2-oxo-3- pyrrolidinyl]-3- pyridinecarboxamide | 535 |
| 93 | 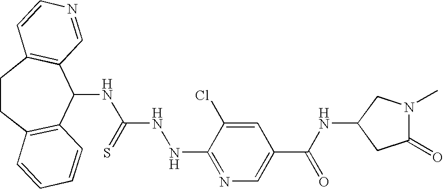 | 5-chloro-6-[2-[[(6,11-dihydro-5H- benzo[5,6]cyclohepta[1,2- c]pyridin-11- yl)amino]thioxomethyl]hydrazino]- N-[(3R)-1-methyl-5-oxo-3- pyrrolidinyl]-3- pyridinecarboxamide | 536 |
| 94 | 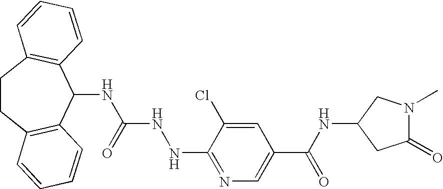 | 5-chloro-6-[2-[[(6,11-dihydro-5H- benzo[5,6]cyclohepta[1,2- c]pyridin-11- yl)amino]thioxomethyl]hydrazino]- N-[(3S)-1-methyl-5-oxo-3- pyrrolidinyl]-3- pyridinecarboxamide | 536 |
| 95 | 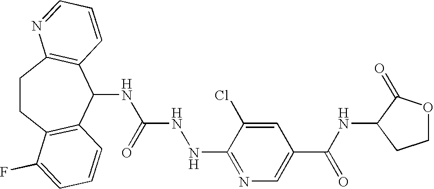 | 5-chloro-6-[2-[[(9-fluoro-10,11- dihydro-5H- benzo[4,5]cyclohepta[1,2- b]pyridin-5- yl)amino]carbonyl]hydrazino]-N- [(3R)-tetrahydro-2-oxo-3-furanyl]- 3-pyridinecarboxamide | 525 |
| 96 | 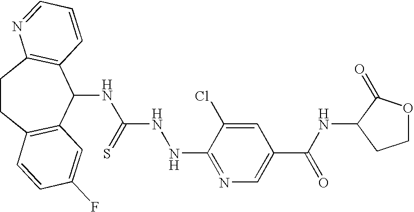 | 5-chloro-6-[2-[[(7-fluoro-10,11- dihydro-5H- benzo[4,5]cyclohepta[1,2- b]pyridin-5- yl)amino]thioxomethyl]hydrazino]- N-[(3R)-tetrahydro-2-oxo-3- furanyl]-3-pyridinecarboxamide | 541 |
| 97 | 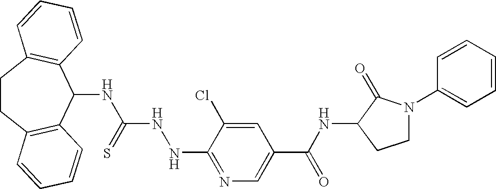 | 5-chloro-6-[2-[[(10,11-dihydro- 5H-dibenzo[a,d]cyclohepten-5- yl)amino]thioxomethyl]hydrazino]- N-(2-oxo-1-phenyl-3- pyrrolidinyl)-3- pyridinecarboxamide | 597 |
| 98 | 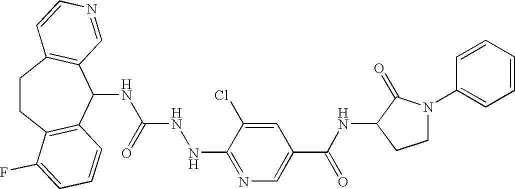 | 5-chloro-6-[2-[[(7-fluoro-6,11- dihydro-5H- benzo[5,6]cyclohepta[1,2- c]pyridin-11- yl)amino]thioxomethyl]hydrazino]- N-[(3R)-2-oxo-1-phenyl-3- pyrrolidinyl]-3- pyridinecarboxamide | 616 |
| 99 | 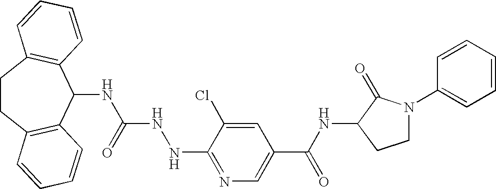 | 5-chloro-6-[2-[[(10,11-dihydro- 5H-dibenzo[a,d]cyclohepten-5- yl)amino]carbonyl]hydrazino]-N- (2-oxo-1-phenyl-3-pyrrolidinyl)- 3-pyridinecarboxamide | 581 |
| 100 | 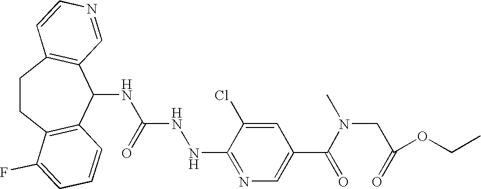 | N-[(5-chloro-6-[[[[(7-fluoro-6,11- dihydro-5H- benzo[5,6]cyclohepta[1,2- c]pyridin-11- yl)amino]thioxomethyl]amino]methyl]-3-pyridinyl]carbonyl]-N- methyl-glycine, ethyl ester | 556 |
| 101 | 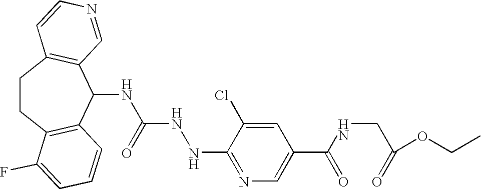 | N-[[5-chloro-6-[2-[[(7-fluoro- 6,11-dihydro-5H- benzo[5,6]cyclohepta[1,2- c]pyridin-11- yl)amino]thioxomethyl]hydrazino]- 3-pyridinyl]carbonyl]-glycine, ethyl ester | 543 |
| 102 | 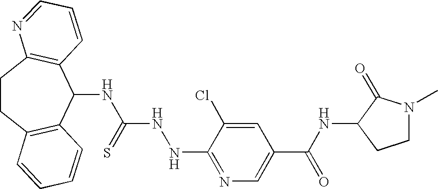 | 5-chloro-6-[2-[[(10,11-dihydro- 5H-benzo[4,5]cyclohepta[1,2- b]pyridin-5- yl)amino]thioxomethyl]hydrazino]- N-((3R)-1-methyl-2-oxo-3- pyrrolidinyl]-3- pyridinecarboxamide | 536 |
| 103 | 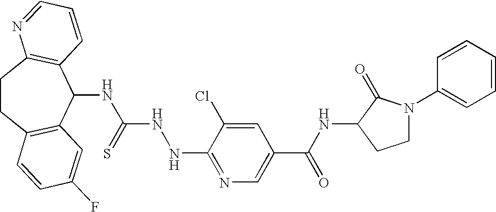 | 5-chloro-6-[2-[[(7-fluoro-10,11- dihydro-5H- benzo[4,5]cyclohepta[1,2- b]pyridin-5- yl)amino]thioxomethyl]hydrazino]- N-[(3R)-2-oxo-1-phenyl-3- pyrrolidinyl]-3- pyridinecarboxamide | 616 |
| 104 | 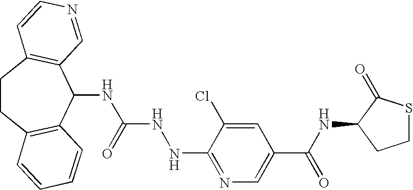 | 5-chloro-6-[2-[[(6,11-dihydro-5H- benzo[5,6]cyclohepta[1,2- c]pyridin-11- yl)amino]carbonyl]hydrazino]-N- [(3R)-tetrahydro-2-oxo-3-thienyl]- 3-pyridinecarboxamide | 523 |
| 105 | 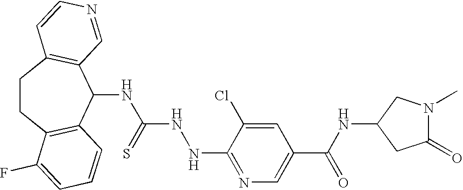 | 5-chloro-6-[2-[[(7-fluoro-6,11- dihydro-5H- benzo[5,6]cyclohepta[1,2- c]pyridin-11- yl)amino]thioxomethyl]hydrazino]- N-[(3R)-1-methyl-5-oxo-3- pyrrolidinyl]-3- pyridinecarboxamide | 554 |
| 106 | 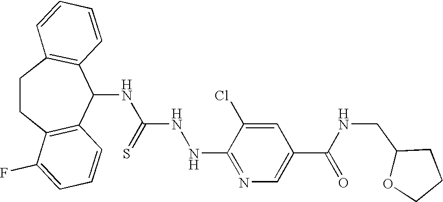 | 5-chloro-6-[2-[[(7-fluoro-6,11- dihydro-5H- benzo[5,6]cyclohepta[1,2- c]pyridin-11- yl)amino]thioxomethyl]hydrazino]- N-[[(2R)-tetrahydro-2- furanyl]methyl]-3- pyridinecarboxamide | 541 |
| 107 | 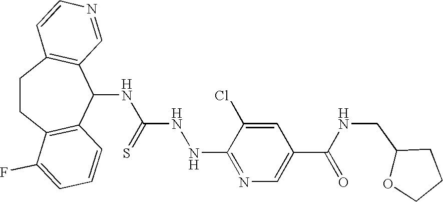 | 5-chloro-6-[2-[[(7-fluoro-6,11- dihydro-5H- benzo[5,6]cyclohepta[1,2- c]pyridin-11- yl)amino]thioxomethyl]hydrazino]- N-[[(2S)-tetrahydro-2- furanyl]methyl]-3- pyridinecarboxamide | 541 |
| 108 | 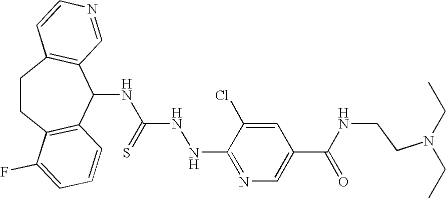 | 5-chloro-N-[2- (diethylamino)ethyl]-6-[2-[[7- fluoro-6,11-dihydro-5H- benzo[5,6]cyclohepta[1,2- c]pyridin-11- yl)amino]thioxomethyl]hydrazino]- 3-pyridinecarboxamide | 556 |
| 109 | 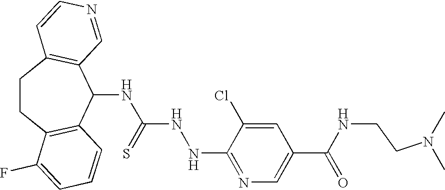 | 5-chloro-N-[2- (dimethylamino)ethyl]-6-[2-[((7- fluoro-6,11-dihydro-5H- benzo[5,6]cyclohepta(1,2- c]pyridin-11- yl)amino]thioxomethyl]hydrazino]- 3-pyridinecarboxamide | 528 |
| 110 | 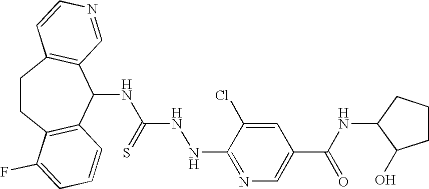 | 5-chloro-6-[2-[[(7-fluoro-6,11- dihydro-5H- benzo[5,6]cyclohepta[1,2- c]pyridin-11- yl)amino]thioxomethyl]hydrazino]- N-[(1R*,2R*)-2- hydroxycyclopentyl]-3- pyridinecarboxamide | 541 |
| 111 | 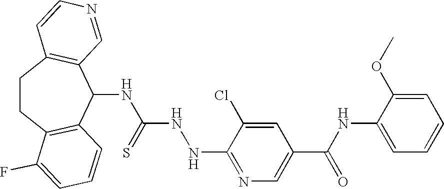 | 5-chloro-6-[2-[[(7-fluoro-6,11- dihydro-5H- benzo[5,6]cyclohepta[1,2- c]pyridin-11- yl)amino]thioxomethyl]hydrazino]- N-(2-methoxyphenyl)-3- pyridinecarboxamide | 563 |
| 112 | 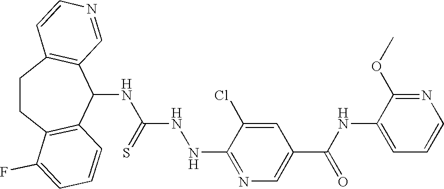 | 5-chloro-6-[2-[[(7-fluoro-6,11- dihydro-5H- benzo[5,6]cyclohepta[1,2- c]pyridin-11- yl)amino]thioxomethyl]hydrazino]- N-(2-methoxy-3-pyridinyl)-3- pyridinecarboxamide | 564 |
| 113 | 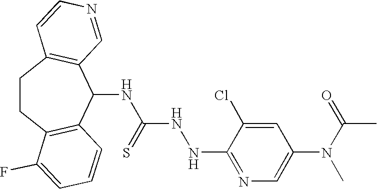 | N-[5-chloro-6-[2-[[(7-fluoro-6,11- dihydro-5H- benzo[5,6]cyclohepta[1,2- c]pyridin-11- yl)amino]thioxomethyl]hydrazino]- 3-pyridinyl]-N-methyl-acetamide | 485 |
| 114 | 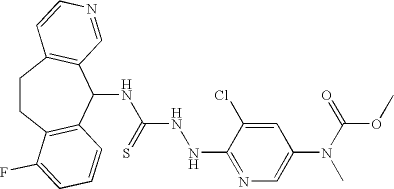 | [5-chloro-6-[2-[[(7-fluoro-6,11- dihydro-5H- benzo[5,6]cyclohepta[1,2- c]pyridin-11- yl)amino]thioxomethyl]hydrazino]- 3-pyridinyl]methyl-carbamic acid, methyl ester | 501 |
| 115 | 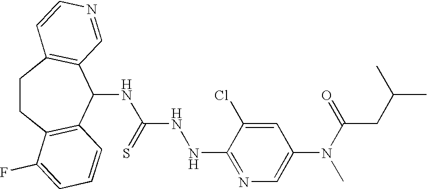 | N-[5-chloro-6-[2-[[(7-fluoro-6,11- dihydro-5H- benzo[5,6]cyclohepta[1,2- benzo[5,6]cyclohepta[1,2- c]pyridin-11- yl)amino]thioxomethyl]hydrazino]- 3-pyridinyl]-N,3-dimethyl- butanamide | 527 |
| 116 | 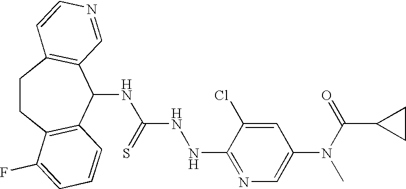 | N-[5-chloro-6-[2-[[[7-fluoro-6,11- dihydro-5H- benzo[5,6]cyclohepta[1,2- c]pyridin-11- yl)amino]thioxomethyl]hydrazino]- 3-pyridinyl]-N-methyl- cyclopropanecarboxamide | 511 |
| 117 | 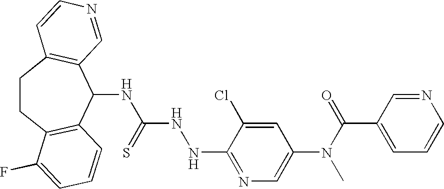 | N-[5-chloro-6-[2-[[(7-fluoro-6,11- dihydro-5H- benzo[5,6]cyclohepta[1,2- c]pyridin-11- yl)amino]thioxomethyl]hydrazino]- 3-pyridinyl]-N-methyl-3- pyridinecarboxamide | 548 |
| 118 | 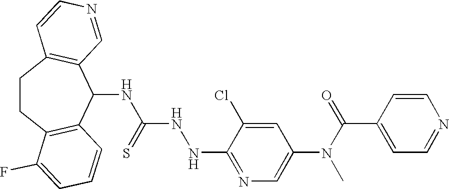 | N-[5-chloro-6-[2-[[(7-fluoro-6,11- dihydro-5H- benzo[5,6]cyclohepta[1,2- c]pyridin-11- yl)amino]thioxomethyl]hydrazino]- 3-pyridinyl]-N-methyl-4- pyridinecarboxamide | 548 |
| 119 | 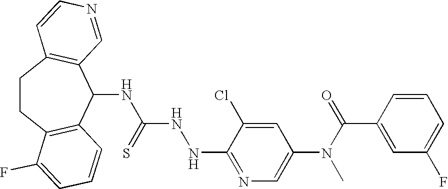 | N-[5-chloro-6-[2-[[(7-fluoro-6,11- dihydro-5H- benzo[5,6]cyclohepta[1,2- yl)amino]thioxomethyl]hydrazino]- 3-pyridinyl]-3-fluoro-N-methyl- benzamide | 565 |
| 120 | 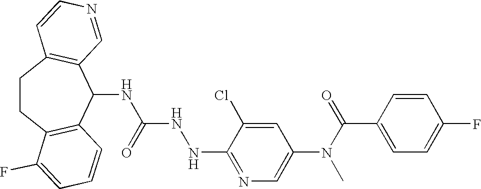 | N-[5-chloro-6-[2-[[(7-fluoro-6,11- dihydro-5H- benzo[5,6]cyclohepta[1,2- c]pyridin-11- yl)amino]thioxomethyl]hydrazino]- 3-pyridinyl]-4-fluoro-N-methyl- benzamide | 565 |
| 121 | 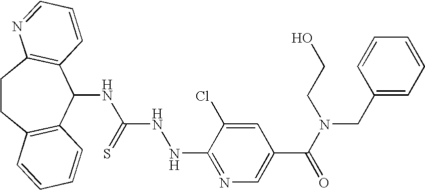 | 5-chloro-6-[2-[[(10,11-dihydro- 5H-benzo[4,5]cyclohepta[1,2- b]pyridin-5- yl)amino]thioxomethyl]hydrazino]- N-(2-hydroxyethyl)-N- (phenylmethyl)-3- pyridinecarboxamide | 573 |
| 122 | 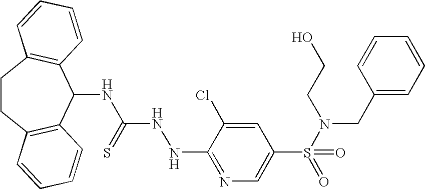 | 2-[3-chloro-5-[[(2- hydroxyethyl)(phenylmethyl)amino]sulfonyl]-2-pyridinyl]-N-(10,11- dihydro-5H- dibenzo[a,d]cyclohepten-5-yl)- hydrazinecarbothioamide | 608 |
| 123 | 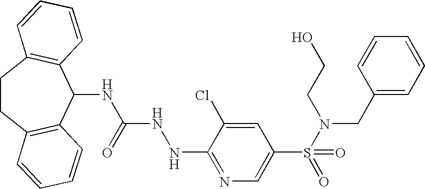 | 2-[3-chloro-5-[[(2- hydroxyethyl)(phenylmethyl)amino]sulfonyl]-2-pyridinyl]-N-(10,11- dihydro-5H- dibenzo[a,d]cyclohepten-5-yl)- hydrazinecarboxamide | 592 |
| 124 | 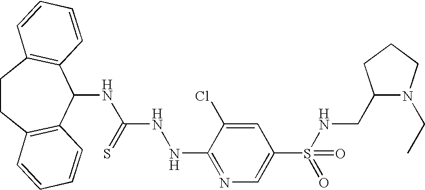 | 2-[3-chloro-5-{[[(1-ethyl-2- pyrrolidinyl)methyl]amino]sulfonyl]- 2-pyridinyl]-N-[7-ethenyl-8,9- dihydro-6-[(1Z)-1-propenyl]-5H- benzocyclohepten-5-yl)- hydrazinecarbothioamide | 585 |
| 125 | 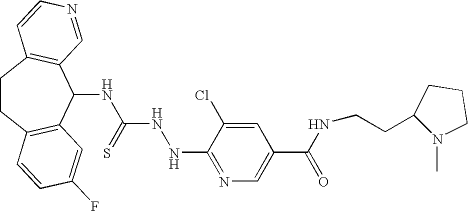 | 5-chloro-6-[2-[[(9-fluoro-6,11- dihydro-5H- benzo[5,6]cyclohepta[1,2- c]pyridin-11- yl)amino]thioxomethyl]hydrazino]- N-[2-(1-methyl-2- pyrrolidinyl)ethyl]-3- pyridinecarboxamide | 568 |
| 126 | 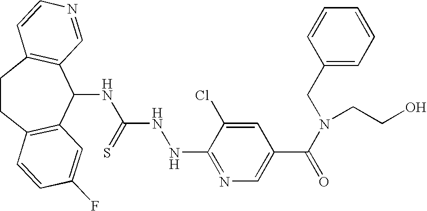 | 5-chloro-6-[2-[[(9-fluoro-6,11- dihydro-5H- benzo[5,6]cyclohepta[1,2- c]pyridin-11- yl)amino]thioxomethyl]hydrazino]- N-(2-hydroxyethyl)-N- (phenylmethyl)-3- pyridinecarboxamide | 591 |
| 127 | 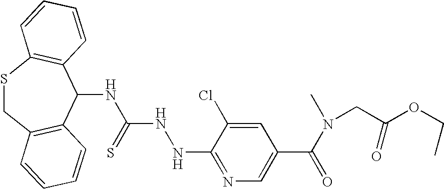 | N-[[5-chloro-6-[2-[[(6,11- dihydrodibenzo[b,e]thiepin-11- yl)amino]thioxomethyl]hydrazino]- 3-pyridinyl]carbonyl]-N-methyl- glycine, ethyl ester | 556 |
| 128 | 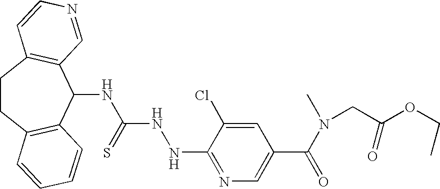 | N-[[5-chloro-6-[2-[[(6,11-dihydro- 5H-benzo[5,6]cyclohepta[1,2- c]pyridin-11- yl)amino]thioxomethyl]hydrazino]- 3-pyridinyl]carbonyl]-N-methyl- glycine, ethyl ester | 539 |
| 129 | 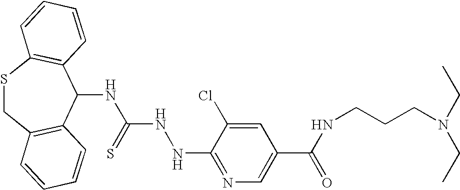 | 5-chloro-N-[2- (diethylamino)ethyl]-6-[2-[[(6,11- dihydrodibenzo[b,e]thiepin-11- yl)amino]thioxomethyl]hydrazino]- 3-pyridinecarboxamide | 555 |
| 130 | 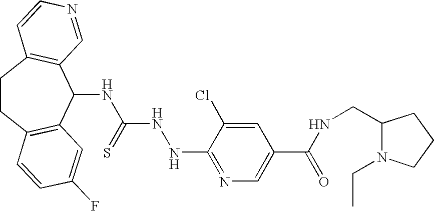 | 5-chloro-N-[(1-ethyl-2- pyrrolidinyl)methyl]-6-[2-[[(9- fluoro-6,11-dihydro-5H- benzo[5,6]cyclohepta[1,2- c]pyridin-11- yl)amino]thioxomethyl]hydrazino]- 3-pyridinecarboxamide | 568 |
| 131 | 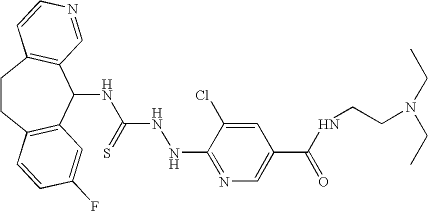 | 5-chloro-N-[2- (diethylamino)ethyl]-6-[2-[[(9- fluoro-6,11-dihydro-5H- benzo[5,6]cyclohepta[1,2- c]pyridin-11- yl)amino]thioxomethyl]hydrazino]- 3-pyridinecarboxamide | 556 |
| 132 | 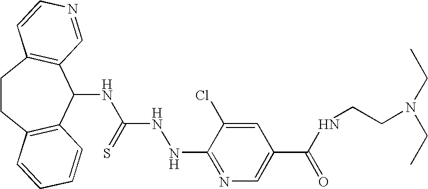 | 5-chloro-N-[2- (diethylamino)ethyl]-6-[2-[[(6,11- dihydro-5H- benzo[5,6]cyclohepta[1,2- c]pyridin-11- yl)amino]thioxomethyl]hydrazino]- 3-pyridinecarboxamide | 538 |
| 133 | 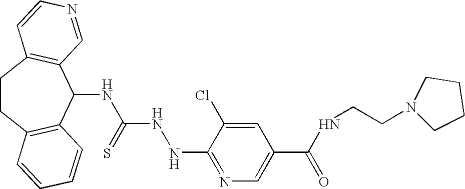 | 5-chloro-6-[2-[[(6,11-dihydro-5H- benzo[5,6]cyclohepta[1,2- c]pyridin-11- yl)amino]thioxomethyl]hydrazino]- N-[2-(1-pyrrolidinyl)ethyl]-3- pyridinecarboxamide | 536 |
| 139 | 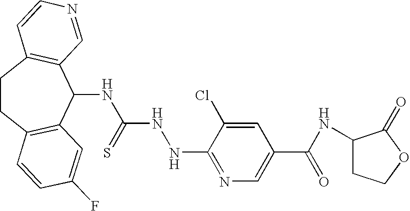 | 5-chloro-6-[2-[[(9-fluoro-6,11- dihydro-5H- benzo[5,6]cyclohepta[1,2- c]pyridin-11- yl)amino]thioxomethyl]hydrazino]- 5N-(tetrahydro-2-oxo-3-furanyl)- 3-pyridinecaxboxamide | 541 |
| 140 | 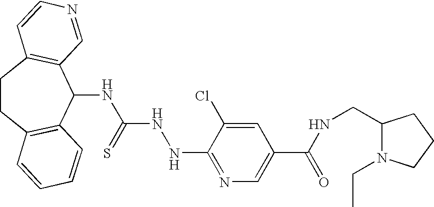 | 5-chloro-6-[2-[[(6,11-dihydro-5H- benzo[5,6]cyclohepta[1,2- c]pyridin-11- yl)amino]thioxomethyl]hydrazinol]- N-[(1-ethyl-2- pyrrolidinyl)methyl]-3- pyridinecarboxamide | 550 |
| 141 | 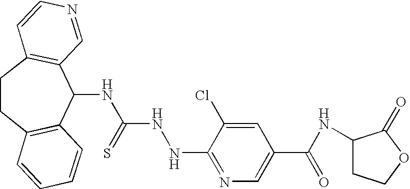 | 5-chloro-6-[2-[[(6,11-dihydro-5H- benzo[5,6]cyclohepta[1,2- c]pyridin-11- yl)amino]thioxomethyl]hydrazino]- N-(tetrahydro-2-oxo-3-furanyl)- 3-pyridinecarboxamide | 523 |
| 142 | 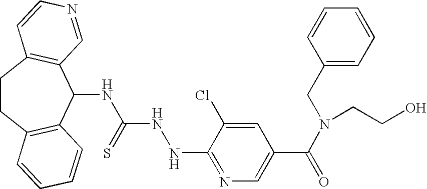 | 5-chloro-6-[2-[[(6,11-dihydro-5H- benzo[5,6]cyclohepta[1,2- c]pyridin-11- yl)amino]thioxomethyl]hydrazino]- N-(2-hydroxyethyl)-N- (phenylmethyl)-3- pyridinecarboxamide | 573 |
| 143 | 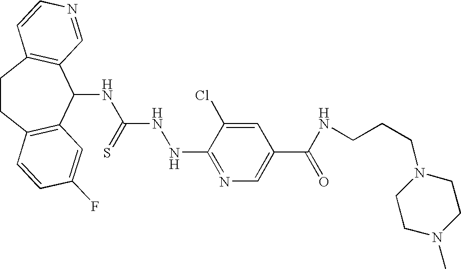 | 5-chloro-6-[2-[[(9-fluoro-6,11- dihydro-5H- benzo[5,6]cyclohepta[1,2- c]pyridin-11- yl)amino]thioxomethyl]hydrazino]- N-[3-(4-methyl-1- piperazinyl)propyl]-3- pyridinecarboxamide | 597 |
| 144 | 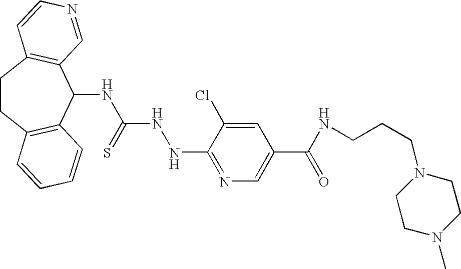 | 5-chloro-6-[2-[[(6,11-dihydro-5H- benzo[5,6]cyclohepta[1,2- c]pyridin-11- yl)amino]thioxomethyl]hydrazino]- N-[3-(4-methyl-1- piperazinyl)propyl]-3- pyridinecarboxamide | 579 |
| 145 | 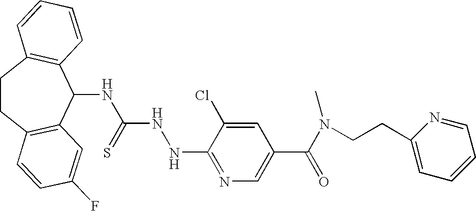 | 5-chloro-6-(2-{[(9-fluoro-6,11- dihydro-5H- benzo[5,6]cyclohepta[1,2- c]pyridin-11- yl)amino]thioxomethyl]hydrazino]- N-methyl-N-[2-(2- pyridinyl)ethyl]-3- pyridinecarboxamide | 576 |
| 146 | 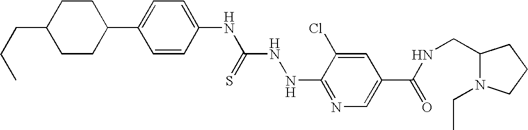 | 5-chloro-N-[[(2S)-1-ethyl-2- pyrrolidinyl]methyl]-6-[2-[[[4-(4- propylcyclohexyl)phenyl]amino]thioxomethyl]hydrazino]-3- pyridinecarboxaniide | 557 |
| 147 | 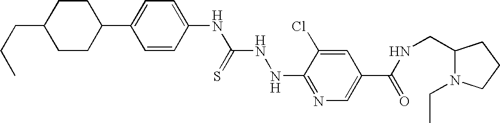 | 5-chloro-N-[[(2R)-1-ethyl-2- pyrrolidinyl]methyl]-6-[2-[[[4-(4- propylcyclohexyl)phenyl]amino]thioxomethyl]hydrazino]-3- pyridinecarboxamide | 557 |
| 148 | 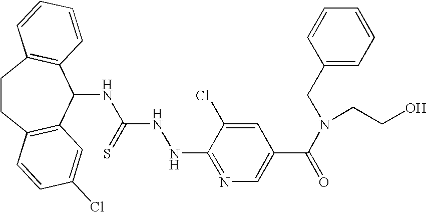 | 5-chloro-6-[2-[[(9-chloro-6,11- dihydro-5H- benzo[5,6]cyclohepta[1,2- c]pyridin-11- yl)amino]thioxomethyl]hydrazino]- N-(2-hydroxyethyl)-N- (phenylmethyl)-3- pyridinecarboxamide | 607 |
| 149 | 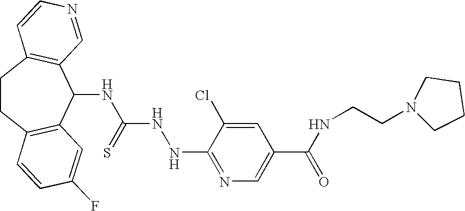 | 5-chloro-6-[2-[[(9-fluoro-6,11- dihydro-5H- benzo[5,6]cyclohepta[1,2- c[pyridin-11- yl)amino]thioxomethyl]hydrazino]- N-[2-(1-pyrrolidinyl)ethyl]-3- pyridinecarboxamide | 554 |
| 150 | 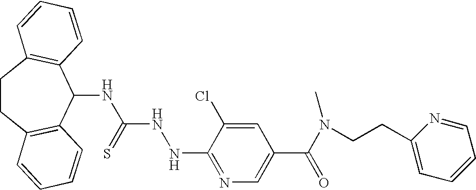 | 5-chloro-6-[2-[[(6,11-dihydro-5H- benzo[5,6]cyclohepta[1,2- c]-pyridin-11- yl)amino]thioxomethyl]hydrazino]- N-methyl-N-[2-(2- pyridinyl)ethyl]-3- pyridinecarboxamide | 558 |
| 151 | 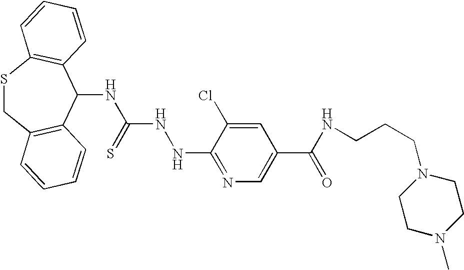 | 5-chloro-6-[2-[[(6,11- dihydrodibenzo[b,e]thiepin-11- yl)amino]thioxomethyl]hydrazino]- N-[3-(4-methyl-1- piperazinyl)propyl]-3- pyridinecarboxamide | 596 |
| 152 | 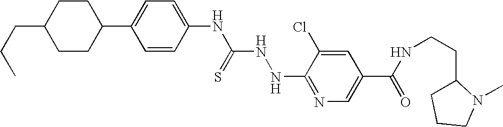 | 5-chloro-N-[2-(1-methyl-2- pyrrolidinyl)ethyl]-6-[2-[[[4-(4- propylcyclohexyl)phenyl]amino]thioxomethyl]hydrazino]-3- pyridinecarboxamide | 557 |
| 153 | 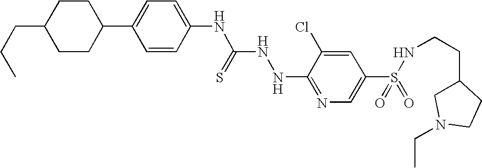 | 2-[3-chloro-5-[[[(1-ethyl-2- pyrrolidinyl)methyl]amino]sulfonyl]-2-pyridinyl]-N-[4-(4- propylcyclohexyl)phenyl]- hydrazinecarbothioamide | 593 |
| 154 | 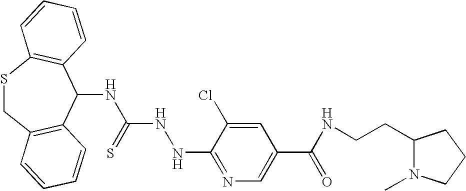 | 5-chloro-6-[2-[[(6,11- dihydrodibenzo[b,e]thiepin-11- yl)amino]thioxomethyl]hydrazino]- N-[2-(1-methyl-2- pyrrolidinyl)ethyl]-3- pyridinecarboxamide | 567 |
| 155 | 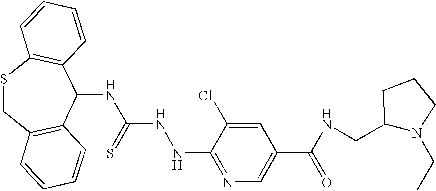 | 5-chloro-6-[2-[[(6,11- dihydrodibenzo[b,e]thiepin-11- yl)amino]thioxomethyl]hydrazino]- N-[(1-ethyl-2- pyrrolidinyl)methyl]-3- pyridinecarboxamide | 567 |
| 156 | 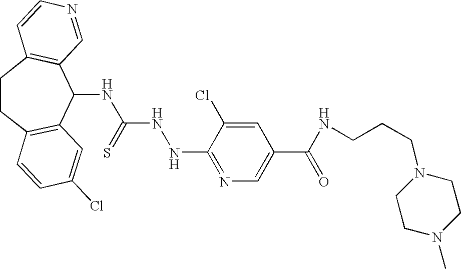 | 5-chloro-6-{2-[[(9-chloro-6,11- dihydro-5H- benzo[5,6]cyclohepta[1,2- c]pyridin-11- yl)amino]thioxomethyl]hydrazino]- N-[3-(4-methyl-1- piperazinyl)propyl]-3- pyridinecarboxamide | 613 |
| 157 | 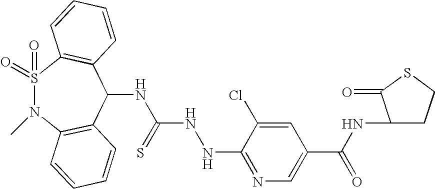 | 5-chloro-6-[2-[[(6,11-dihydro-6- methyl-5,5- dioxidodibenzo[c,f][1,2]thiazepin-11- yl)amino]thioxomethyl]hydrazino]- N-(tetrahydro-2-oxo-3-thienyl)- 3-pyridinecarboxamide | 603 |
| 158 | 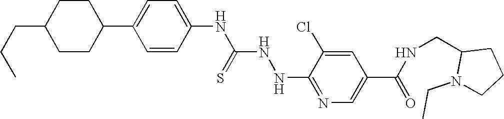 | 5-chloro-N-[(1-ethyl-2- pyrrolidinyl)methyl]-6-[2-[[[4-(4- propylcyclohexyl)phenyl]amino]thioxomethyl]hydrazino]-3- pyridinecarboxamide | 557 |
| 159 | 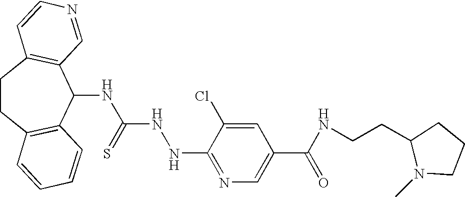 | 5-chloro-6-[2-[[(6,11-dihydro-5H- benzo[5,6]cyclohepta[1,2- c]pyridin-11- yl)amino]thioxomethyl]hydrazino]- N-[2-(1-methyl-2- pyrrolidinyl)ethyl]-3- pyridinecarboxamide | 550 |
| 160 | 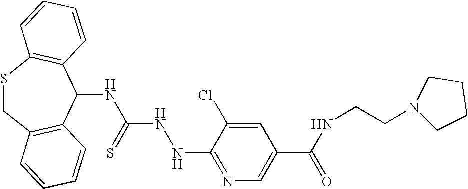 | 5-chloro-6-[2-[[(6,11- dihydrodibenzo[b,e]thiepin-11- yl)amino]thioxomethyl]hydrazino]- N-[2-(1-pyrrolidinyl)ethyl]-3- pyridinecarboxamide | 553 |
| 161 | 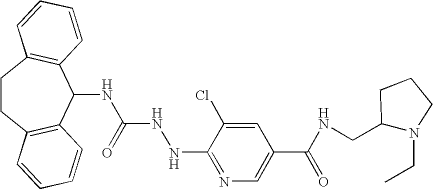 | 2-[3-chloro-5-[[[(1-ethyl-2- pyrrolidinyl)methyl]amino]sulfonyl]- 2-pyridinyl]-N-(10,11-dihydro- 5H-dibenzo[a,d]cyclohepten-5-yl)- hydrazinecarboxamide | 569 |
| 162 | 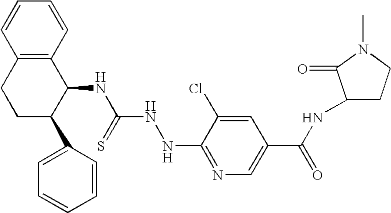 | 5-chloro-N-((3R)-1-methyl-2-oxo- 3-pyrrolidinyl]-6-[2-[[[(1R,2R)- 1,2,3,4-tetrahydro-2-phenyl-1- naphthalenyl]amino]thioxomethyl]hydrazino]-3- pyridinecarboxamide | 549 |
| 163 | 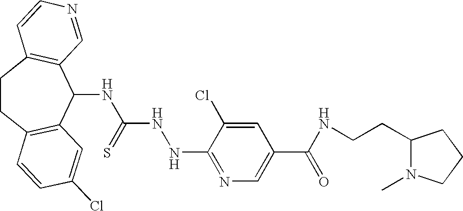 | 5-chloro-6-[2-[[(9-chloro-6,11- dihydro-5H- benzo[5,6]cyclohepta[1,2- c]pyridin-11- yl)amino]thioxomethyl]hydrazino]- N-[2-(1-methyl-2- pyrrolidinyl)ethyl]-3- pyridinecarboxamide | 584 |
| 164 | 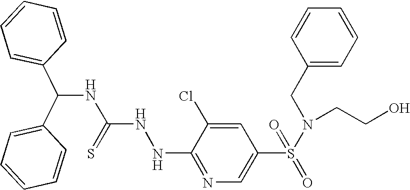 | 2-[3-chloro-5-[[(2- hydroxyethyl)(phenylmethyl)amino]sulfonyl]-2-pyridinyl)-N- (diphenylmethyl)- hydrazinecarbothioamide | 582 |
| 165 | 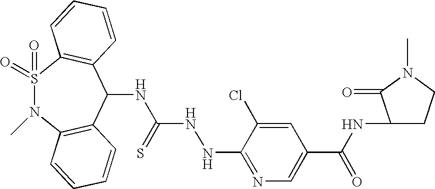 | 5-chloro-6-[2-[[(6,11-dihydro-6- methyl-5,5- dioxidodibenzo[c,f][1,2]thiazepin-11- yl)amino]thioxomethyl]hydrazino]- N-(1-methyl-2-oxo-3- pyrrolidinyl)-3- pyridinecarboxamide | 600 |

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I or II. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I or II. It will further be understood that the present invention encompasses tautomers of the compounds of the formula I or E.

It will also be understood that certain compounds of the present invention may exist in solvated, for example hydrated, as well as unsolvated forms. It will further be understood that the present invention encompasses all such solvated forms of the compounds of the formula I or II.

Within the scope of the invention are also salts of the compounds of the formula I or II. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of formula I or II above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

The novel compounds of the present invention are useful in therapy, especially for the treatment of various pain conditions such as chronic pain, neuropathic pain, acute pain, cancer pain, pain caused by rheumatoid arthritis, migraine, visceral pain etc. This list should however not be interpreted as exhaustive.

Compounds of the invention are useful in disease states where degeneration or dysfunction of Bradykinin receptors is present or implicated in that paradigm. This may involve the use of isotopically labelled versions of the compounds of the invention in diagnostic techniques and imaging applications such as positron emission tomography (PET).

Compounds of the invention are useful for the treatment of septic shock, pancreatitis, edema, rhinitis, asthma, colitis, arthritis, hepatorenal syndrome, cancer, (including but not restricted to SCLC, prostrate cancer), bacterial and viral infections, ulcerative colitis, and Alzheimer's Disease.

Compounds of the invention are useful as an analgesic agent for use during general anaesthesia and monitored anaesthesia care. Combinations of agents with different properties are often used to achieve a balance of effects needed to maintain the anaesthetic state (e.g. amnesia, analgesia, muscle relaxation and sedation). Included in this combination are inhaled anaesthetics, hypnotics, anxiolytics, neuromuscular blockers and opioids.

Also within the scope of the invention is the use of any of the compounds according to the formula I or II above, for the manufacture of a medicament for the treatment of any of the conditions discussed above.

A further aspect of the invention is a method for the treatment of a subject suffering from any of the conditions discussed above, whereby an effective amount of a compound according to the formula I or II above, is administered to a patient in need of such treatment.

Thus, the invention provides a compound of formula I or II, or pharmaceutically acceptable salt or solvate thereof, as hereinbefore defined for use in therapy.

In a further aspect, the present invention provides the use of a compound of formula I or II, or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be contrued accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: acute pain, chronic pain, neuropathic pain, acute pain, back pain, cancer pain, and visceral pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints.

In one embodiment of the invention, the route of administration may be orally, intravenously or intramuscularly.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (percent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of formula I or II as defined above for the manufacture of a medicament.

Also within the scope of the invention is the use of any compound of formula I or II for the manufacture of a medicament for the therapy of pain.

Additionally provided is the use of any compound according to Formula I or II for the manufacture of a medicament for the therapy of various pain conditions including, but not limited to: acute pain, chronic pain, neuropathic pain, acute pain, back pain, cancer pain, and visceral pain.

A further aspect of the invention is a method for therapy of a subject suffering from any of the conditions discussed above, whereby an effective amount of a compound according to the formula I or II above, is administered to a patient in need of such therapy.

Additionally, there is provided a pharmaceutical composition comprising a compound of Formula I or II, or a pharmaceutically acceptable salt thereof, in association with a pharmaceutically acceptable carrier.

Particularly, there is provided a pharmaceutical composition comprising a compound of Formula I or II, or a pharmaceutically acceptable salt thereof, in association with a pharmaceutically acceptable carrier for therapy, more particularly for therapy of pain.

Further, there is provided a pharmaceutical composition comprising a compound of Formula I or II, or a pharmaceutically acceptable salt thereof, in association with a pharmaceutically acceptable carrier use in any of the conditions discussed above.

In a further aspect, the present invention provides a method of preparing a compound of formula I or II.

Compounds of formula I or II have been prepared as single compound syntheses and/or using parallel synthetic protocols.

In one embodiment, the present invention provides a process for preparing compounds of Formula I wherein X is represented by formula (i) or (ii), comprising reacting a compound of general formula IV, 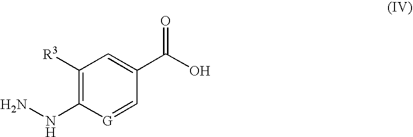
wherein G is CH or N and R³ is halogen, with an isocyanate (Y—NCO) or thioisocyanate (Y—NCS), wherein Y is as defined above; to give a compound of general formula V, wherein T is S or O; 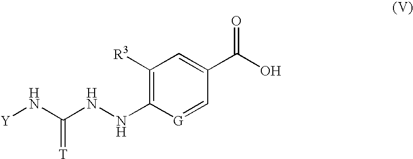
which is further coupled with a primary or secondary amine HNR¹R², wherein R¹ and R² are as defined above, using a standard amide coupling reagent such as HATU and an acid scavenger such as diisopropylethyl amine (DIPEA), to yield a compound of formula I, wherein W is —C(═O)—.

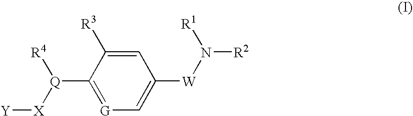

In another embodiment, the present invention provides a method of preparing a compound of formula VI, wherein the method includes the step of reacting a compound of formula VII with a compound of Y—NCO or Y—NCS: 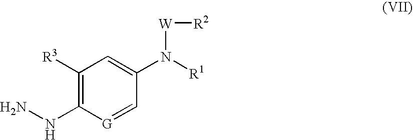
to form the compound of formula VI: 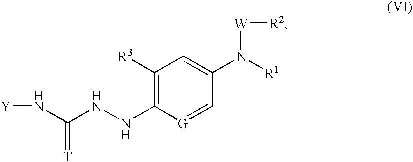
wherein T is O or S; and W, Y, G, R¹ and R² are as defined above.

In an even further aspect, the present invention provides useful reaction intermediates Y—NCS, Y—NCO, and the compounds of formulas (V) and (VII), wherein W, Y, G, R¹ and R² are as defined above.

Compounds of the present invention may also be prepared by combinatorial methods.

General combinatorial protocol for plates: The corresponding acid (0.04 m in DMA, 0.5 ml, 20 mol), 80 different amines (0.5 m in DMA, 50 μl, 25 μmol), and DIPEA (1 m in DMA, 50 μl, 50 μmol) are successively distributed to a 96-well format plate, then HATU (0.25 m in DMA, 100 μl, 25 μmol) is added to the wells. The plate is shaken overnight at room temperature and worked up by removing DMA under reduced pressure, and adding dichloromethane (500 μl) to the wells, and washing with H₂O (3×500 μl), then dichloromethane is evaporated in vacuo to provide a ˜10 mg per well plate with most of the compound purity in the range of 50-90%.

It will be understood by those of ordinary skill in the art that a chemical reaction which fails to efficiently yield the desired product within the context of a combinatorial protocol may nonetheless efficiently yield the desired product when the reaction is performed in a single reaction or parallel reaction format, without undue experimentation on the part of the chemist. In this regard, several of the compounds which were not prepared efficiently in the combinatorial array, were subsequently prepared in separate syntheses.

Particularly, the compounds of the present invention can be prepared according to the synthetic routes as exemplified in Schemes 1-9 and further detailed in the Examples, wherein Y and R¹ and R² are as defined above. 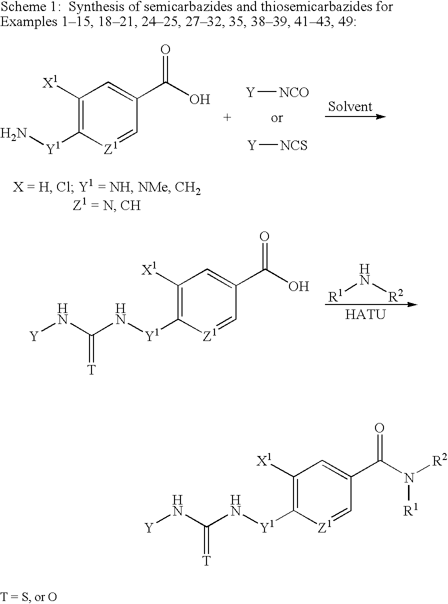 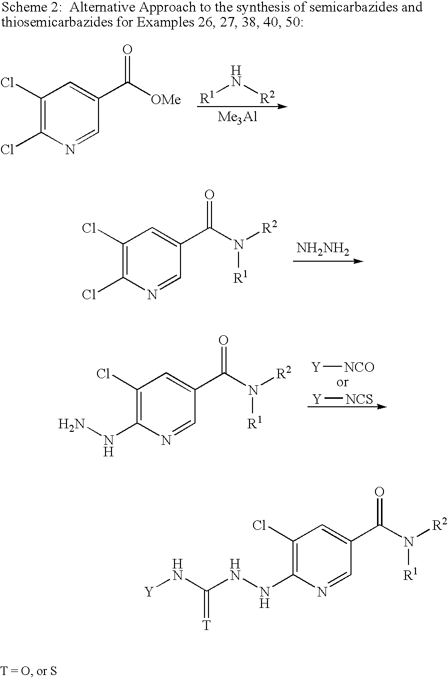 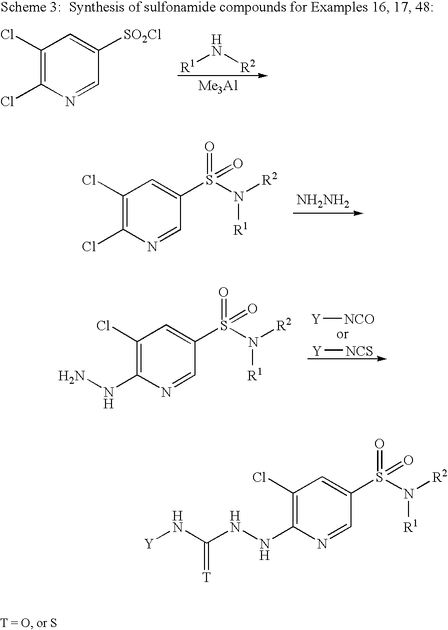 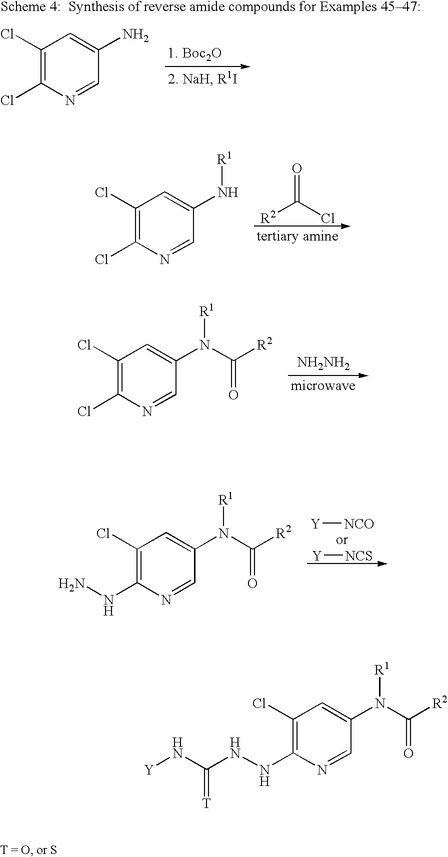 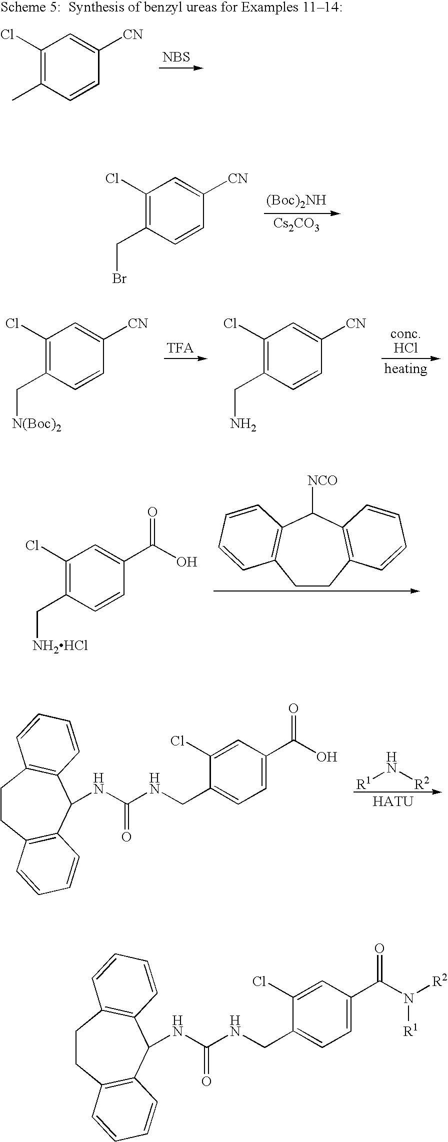 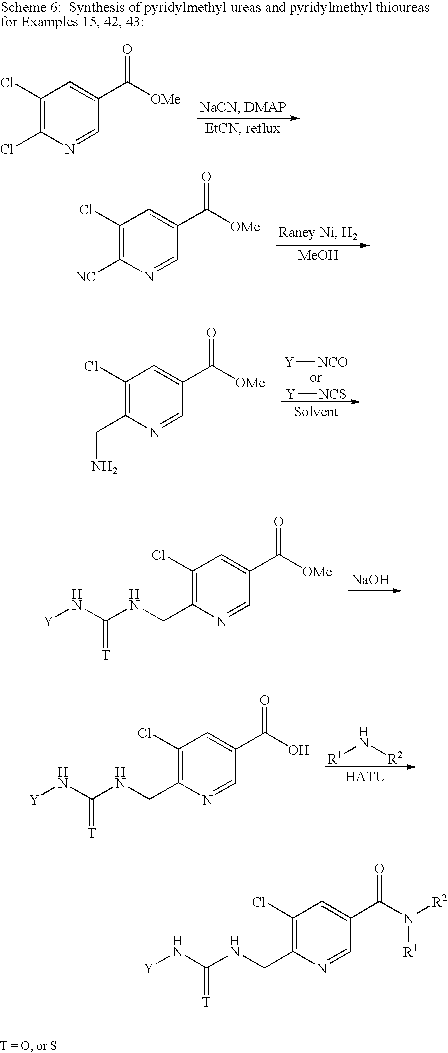 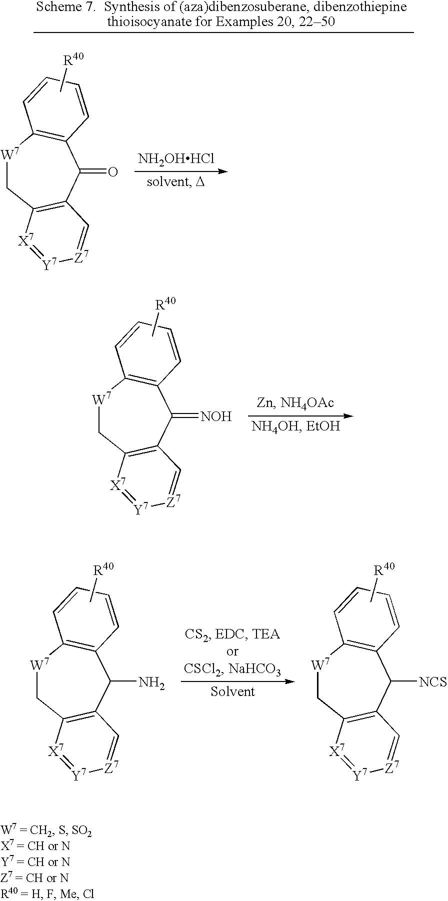 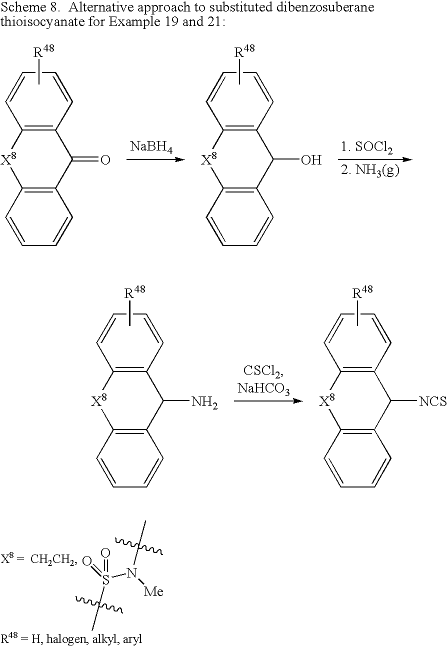 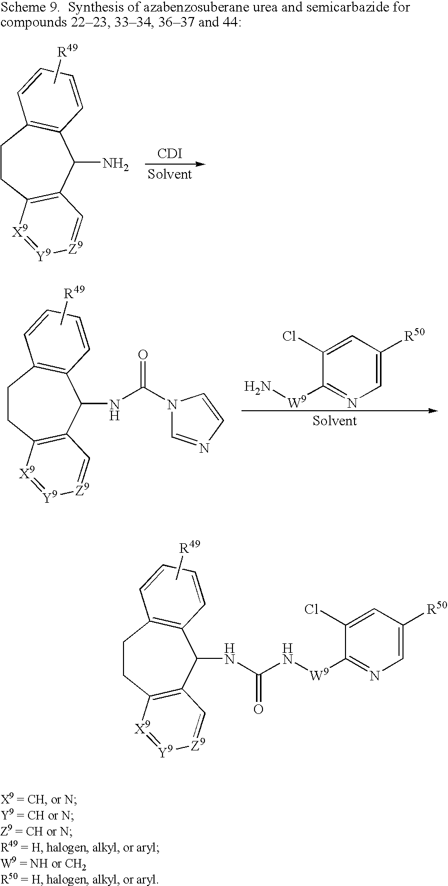
Biological Evaluation
1. B2 Bradykinin
A. Human Bradykinin B2 (hB2) Receptor Expression and Membrane Preparation

The cloned human Bradykinin B2 (hB2) receptor in the pCIN vector was purchased from Receptor Biology. The hB2 receptor was stably transfected into HEK 293 S cells and a clonal cell line was generated. Cells were grown in T-flasks with DMEM culture media containing 10% FBS, 2 mM glutamine, 600 μg/ml neomycin and an antibiotic cocktail (100 IU penicillin, 100 μg/ml streptomycin, 0.25 μg/ml amphotericin B). Membranes, expressing the hB2 receptor, were prepared from this cell line according this protocol: Cells are harvested at 1 to 1.2 million cells/ml, pelleted, and resuspended in ice-cold lysis buffer (50 mM Tris, pH 7.0, 2.5 mM EDTA, with PMSF added just prior to use to 0.5 mM from a 0.5 M stock in DMSO. After lysis on ice for 15 min, the cells are homogenized with a polytron for 10 sec. The suspension is spun at 1000 g for 10 min at 4° C. The supernatant is saved on ice and the pellets resuspended and spun as before. The supernatants from both spins are combined and spun at 46,000 g for 10-30 min. The pellets are resuspended in cold Tris buffer (50 mM Tris/Cl, pH 7.0) at a dilution of 0.2-1 ml per 40 million cells and spun again. The final pellets are resuspended in membrane buffer (50 mM Tris, 0.32 M sucrose, pH 7.0). Aliquots are frozen in dry ice/ethanol and stored at −70° C. until use. The protein concentrations are determined by a modified Lowry with SDS.

B. hB2 Receptor Binding

Membranes expressing the hB2 receptor are thawed at 37° C., passed 3 times through a 25-gauge blunt-end needle, diluted in the bradykinin binding buffer (50 mM Tris, 3 mM MgCl₂, and 1 mg/ml BSA, pH 7.4, 0.02 mg/ml Phenantholine, 0.25 mg/ml Pefabloc) and 80 μL aliquots containing the appropriate amount of protein (final concentration of 0.25 μg/ml) are distributed in 96-well polystyrene plates (Treff Lab). The IC₅₀ of compounds are evaluated from 10-point dose-response curves, where the serial dilutions are done on a final volume of 150 μL, with 70 μL of ¹²⁵I-Desamino-TyrHOE140 (Kd=0.05) at 50,000 to 60,000 dpm per well (0.03-0.04 nM) in a final volume of 300 μl. The total and non-specific binding are determined in the absence and presence of 0.1 μM (150 μL) of Bradykinin respectively. The plates are vortexed and incubated for 60 minutes at room temperature, filtered through Unifilters-96 GF/B (Canberra Packard), which were presoaked in 0.1% polyethyleneimine, with a harvester using 3 ml of wash buffer (50 mM Tris, pH 7.0, 3 mM MgCl₂). The filters are dried for 1 hour at 55° C. The radioactivity (cpm) is counted in a TopCount (Canberra Packard) after adding 65 μl/well of MS-20 scintillation liquid (Canberra Packard). Compounds of the present invention have demonstrated hB2 receptor binding at concentrations less than 10 μM.

Based on the above assays, the dissociation constant (Ki) for a particular compound of the invention towards a particular receptor is determined using the following equation:
Ki=IC₅₀/(1+[rad]/Kd),

Wherein IC₅₀ is the concentration of the compound of the invention at which 50% displacement has been observed;
[rad] is a standard or reference radioactive ligand concentration at that moment; and
Kd is the dissociation constant of the radioactive ligand towards the particular receptor.
   GTP[γ]³⁵S Binding Experiments on Bradykinin (B2) Receptors
   A. General Information

The procedures below describe how to perform and interpret GTP[γ]³⁵S binding experiments designed to determine the activity of new compounds on the human B2 receptor.

B. General Procedure of the Assay

Human Bradykinin-2 GTP[γ]³⁵S Binding

Human Bradykinin-2 membranes (hB2 293s) are thawed at 37° C., passed 3 times through a 25-gauge blunt-end needle and diluted in the GTPγS binding buffer for the assay (50 mM Hepes, pH 7.4; 200 mM NaCl; 1 mM EDTA; 5 mM MgCl₂. To this added freshly prepared 1 mM DTT, 0.5% BSA, 1 μM GDP. The EC50 and Emax of compounds are evaluated from 10-point dose-response curves done in 300 μl with the appropriate amount of membrane protein and 100,000-120,000 dpm of GTPγ³⁵S per well (0.11-0.14 nM). Bradykinin (1-9) is used as the standard agonist at hB2. The ranges of concentrations tested should include a maximal concentration of 0.1 μM bradykinin in order to establish the Eₘₐₓ.

The plates are vortexed and incubated for 60 minutes at room temperature, filtered on GF/B Unifilters (presoaked in water) with the Packard harvester using 4 ml/well of wash buffer (50 mM Tris, 5 mM MgCl₂, 50 mM NaCl, pH 7.0), minimum. The filters are dried for 1 hour at 55° C. The radioactivity (cpm) is counted in a TopCount (Packard) after adding 65 μl/well of MS-20 scintillation liquid.

Antagonist reversal studies are done in a similar manner except that the compound dose-response curve's are performed in the presence of a constant concentration of agonist (approx. 80% bradykinin Eₘₐₓ; ˜5 nM). A standard B2 Antagonist is used as the reference antagonist at hB2. The ranges of antagonist concentrations tested should include a maximal concentration of 3 μM of the standard B2 Antagonist in order to establish the maximal displacement (Dₘₐₓ).

C. Radioligand: Preparation of GTP[γ]³⁵S GTP[γ]³⁵S is acquired from Perkin-Elmer (250 μCi/20 μl. It is diluted from with 10 mM DTT, 50 mM Tris, pH 7 (dilute in 2 ml, 1.0 mCI/20μ). Sonicate the solution, filter through a 0.45 μm filter, and freeze aliquots at −70° C. For the experiment, use ˜0.3 nM dilution of this tracer in the GTP binding buffer.

D. Data Analysis

The EC₅₀ and Eₘₐₓ of compounds are evaluated from 10-point dose-response curves done in 300 μl with the appropriate amount of membrane protein and GTPγ³⁵S per well and are calculated in Activity base with ExcelFit. The basal and maximal stimulated binding are determined in absence and presence of standard reference compounds, respectively.

The stimulation (Stim) in the presence of compounds is expressed as the percentage of Dₘₐₓ of the reference antagonist. Values of IC₅₀, Ki′ and Dₘₐₓ for ligands capable of competing for agonist stimulated binding are calculated in Activity Base. Mean±S.E.M. values of IC₅₀, Ki′ and % Dₘₐₓ are reported for ligands tested in at least three dose-response curves.

Biological data for certain compounds of the invention are listed in Table 2 below.

TABLE 2

| Compound Nos. | Ki (hB2) (nM) |
|---|---|
| 1-165 | 5-5000 |

### EXAMPLES

The invention will further be described in more detail by the following Examples which describe methods whereby compounds of the present invention may be prepared, purified, analyzed and tested, and which are not to be construed as limiting the invention.

### Example 1

### 5-Chloro-6-[2-[[(10,11-dihydro-5H-dibenzo[a,d]cyclohepten-5-yl)amino]-thioxomethyl]hydrazino]-N-[(3R)-tetrahydro-2-oxo-3-thienyl]-3-pyridinecarboxamide

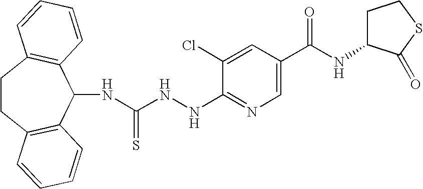

### 1A: 10,11-dihydro-5-isothiocyanato-5H-dibenzo[a,d]cycloheptene

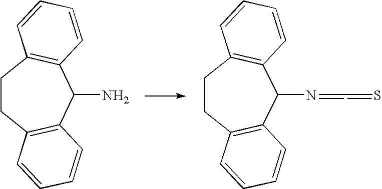

CS₂ (51.7 mL, 860 mmol) and EDC (30.2 g, 157.5 mmol) was added to the suspension of 10,11-dihydro-5H-dibenzo[a,d]cyclohepten-5-amine (30.0 g, 143.3 mmol) (prepared according to P. Melloni et al J. Med. Chem. 1979, 22, 183-191; WO 0160826) in Et₂O (900 mL) at −10° C. The reaction mixture was stirred at this temperature for 15 min, then Et₃N (22 mL, 157 mmol) was added at such rate that the temperature was maintained between −10 and −5° C. The resulting mixture was stirred at −10° C. for 3 hrs, and then room temperature overnight. The reaction mixture was then filtered and the filtrate was concentrated. The residue was dissolved in EtOAc (600 mL), and washed sequentially with 5% HCl (50 mL), H₂O (50 mL), 5% aq. NaHCO₃ (50 mL), and brine (2×50 mL). The organic phase was dried over MgSO₄, filtered and concentrated in vacuo. The residue was recrystallized in hexanes/EtOAc to produce the title compound (24.7 g, 69%).

### 1B: 5-chloro-6-[2-[[(10,11-dihydro-5H-dibenzo[a,d]cyclohepten-5-yl)amino]thioxo-methyl]hydrazino]pyridinecarboxylic Acid

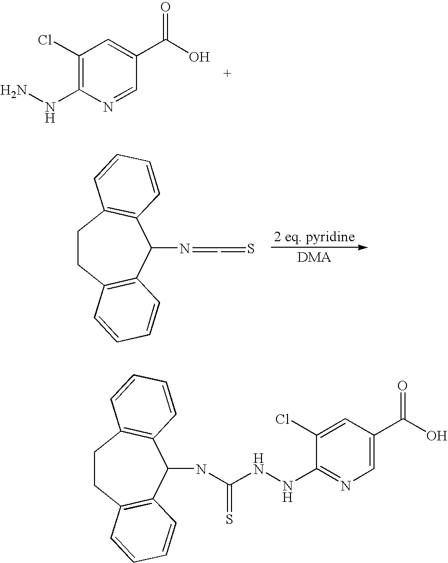

A mixture of 5-chloro-6-hydrazino-3-pyridinecarboxylic acid (1.91 g, 10.02 mmol) (prepared from commercial 5,6-dichloronicotinic acid and hydrazine according to: Graf J. Prakt. Chem. 1933, 138, 244-256, which is incorporated by reference herein for its disclosure in the preparation of 5-chloro-6-hydrazino-3-pyridinecarboxylic acid) and 10,11-dihydro-5-isothiocyanato-5H-dibenzo[a,d]cycloheptene (2.57 g, 10.2 mmol) in DMA (80 mL) containing 1.7 mL of pyridine was stirred at room temperature overnight. The solvent was removed in vacuo, and the residue was triturated with Et₂O (30 mL). The white solid was collected and dried in vacuo to afford a white solid (5.30 g, quantitative) as a 1:1 complex of the title compound with DMA. ¹HNMR (DMSO-d6): δ 13.00 (s, 1H), 9.67 (s, 1H), 9.41 (s, 1H), 8.93 d, J=8.8 Hz, 1H), 8.57 (s, 1H), 8.03 (s, 1H), 7.52 (brs, 1H), 7.27 (d, J=6 Hz, 2H), 7.09 (br, 4H), 7.01 (br, 2H), 3.24 (m, 2H), 2.94 (m, 2H), 2.91 (s, 3H, DMA), 2.75 (s, 3H, DMA), 1.92 (s, 3H, DMA) ppm. MS (ESI) (M+1)⁺=439.

A small fraction of the product was recrystallized in EtOH to give DMA-free title compound. ¹HNMR (DMSO-d6): δ 13.00 (s, 1H), 9.67 (s, 1H), 9.41 (s, 1H), 8.93 d, J=8.8 Hz, 1H), 8.57 (s, 1H), 8.03 (s, 1H), 7.52 (brs, 1H), 7.27 (d, J=6 Hz, 2H), 7.09 (br, 4H), 7.01 (br, 2H), 3.24 (m, 2H), 2.94 (m, 2H) ppm.

### 1C: 5-chloro-6-[2-[[(10,11-dihydro-5H-dibenzo[a,d]cyclohepten-5-yl)amino]-thioxomethyl]hydrazino]-N-[(3R)-tetrahydro-2-oxo-3-thienyl]-3-pyridinecarboxamide

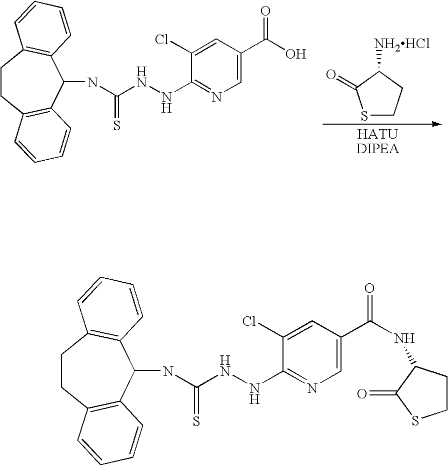

HATU (190 mg, 0.50 mmol) was added to a mixture of 5-chloro-6-[2-[[(10,11-dihydro-5H-dibenzo[a,d]cyclohepten-5-yl)amino]thioxo-methyl]hydrazino]-3-pyridinecarboxylic acid.DMA (180 mg, 0.34 mmol), D-homocysteine thiolactone hydrochloride (65 mg, 0.42 mmol) and DIPEA (0.30 mL) in DMA (5 mL) at room temperature. The reaction mixture was stirred at room temperature for 2 h, and then DMA was removed under reduced pressure. Water (5 mL) was added to the residue and the solid was collected and dried in vacuo to give crude product (215 mg). The product was recrystallized twice in EtOH to provide the title compound (60 mg, 33%) as light gray solid. [α]_{D} +23.7° (c 0.23, DMA). ¹HNMR (DMSO-d6): δ 9.64 (s, 1H), 9.27 (s, 1H), 8.89 (brs, 1H), 8.69 (d, J=6.8 Hz, 1H), 8.54 (s, 1H), 8.10 (s, 1H), 7.48 (br, 1H), 7.26 (d, J=6.4 Hz, 2H), 7.20-6.90 (m, 6H), 4.81 (m, 1H), 3.43 (m, 1H), 3.32 (m, 1H), 2.93 (m, 1H), 2.52 (m, 4H), 2.23 (m, 1H) ppm. MS (ESI) (M+1)⁺=538. Anal. Calcd for C₂₆H₂₄ClN₅O₂S₂.H₂O: C, 56.16; H, 4.67; N, 12.59. Found: C, 56.33; H, 4.39; N, 12.41.

### Example 2

### 5-Chloro-6-[2-[[(10,11-dihydro-5H-dibenzo[a,d]cyclohepten-5-yl)amino]-thioxomethyl]hydrazino]-N-[(3S)-tetrahydro-2-oxo-3-thienyl]-3-pyridinecarboxamide

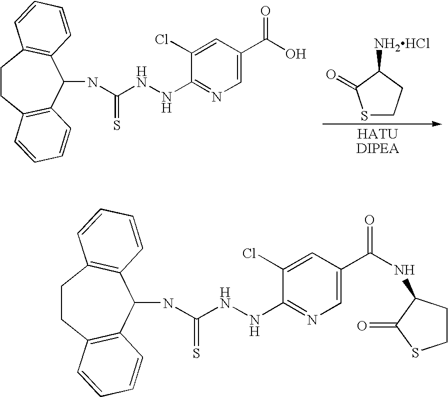

Following the general HATU coupling procedure of Example 1C: the title compound (110 mg, 23%) was prepared from 5-chloro-6-[2-[[(10,11-dihydro-5H-dibenzo[a,d]cyclohepten-5-yl)amino]thioxo-methyl]hydrazino]-3-pyridinecarboxylic acid.DMA (460 mg) and L-homocysteine thiolactone hydrochloride (180 mg). ¹HNMR (DMSO-d6): δ 9.64 (s, 1H), 9.27 (s, 1H), 8.89 (brs, 1H), 8.69 (d, J=6.8 Hz, 1H), 8.54 (s, 1H), 8.10 (s, 1H), 7.48 (br, 1H), 7.26 (d, J=6.4 Hz, 2H), 7.20-6.90 (m, 6H), 4.81 (m, 1H), 3.43 (m, 1H), 3.32 (m, 1H), 2.93 (m, 1H), 2.52 (m, 4H), 2.23 (m, 1H) ppm. MS (ESI) (M+1)⁺=538. Anal. Calcd for C₂₆H₂₄ClN₅O₂S₂.1.25H₂O: C, 55.71; H, 4.76; N, 12.49. Found: C, 55.71; H, 4.40; N, 12.55. [α]_{D} −22.8° (c 0.52, DMA).

### Example 3

### 5-chloro-6-[2-[[(10,11-dihydro-5H-dibenzo [a,d]cyclohepten-5-yl)amino]thioxomethyl]hydrazino]-N-(tetrahydro-2-oxo-3-furanyl)-3-pyridinecarboxamide

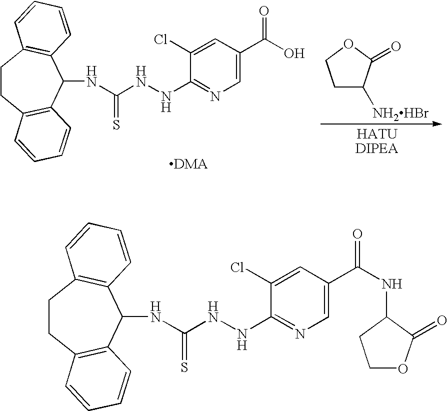

Following general HATU coupling procedure of Example 1C: The title compound (50 mg, 55%) was obtained from 5-chloro-6-[2-[[(10,11-dihydro-5H-dibenzo[a,d]cyclohepten-5-yl)amino]thioxo-methyl]hydrazino]-3-pyridinecarboxylic acid.DMA (92 mg, 0.175 mmol) and α-amino-γ-butyrolactone hydrobromide (40 mg, 0.22 mmol). ¹HNMR (DMSO-d6): δ 9.65 (9.29) (brs, 1H), 8.94 (br, 2H), 8.55 (s, 1H), 8.11 (s, 1H), 7.49 (br, 1H), 7.29 (br, 2H), 7.20-6.90 (m, 7H), 4.80-4.66 (m, 1H), 4.44-4.36 (m, 1H), 4.30-4.18 (m, 1H), 3.35-3.16 (br, 2H), 3.02-2.86 (br, 2H), 2.50-2.40 (m, 1H), 2.36-2.24 (m, 1H) ppm. MS (ESI) (M+1)⁺=522. Anal. Calcd for C₂₆H₂₄ClN₅O₃S.1.75H₂O: C, 56.42; H, 5.01; N, 12.65. Found: C, 56.40; H, 4.72; N, 12.42.

### Example 4

### N-[[5-chloro-6-[2-[[(10,11-dihydro-5H-dibenzo[a,d]cyclohepten-5-yl)amino]-thioxomethyl]hydrazino]-3-pyridinyl]carbonyl]-N-methylglycine Ethyl Ester

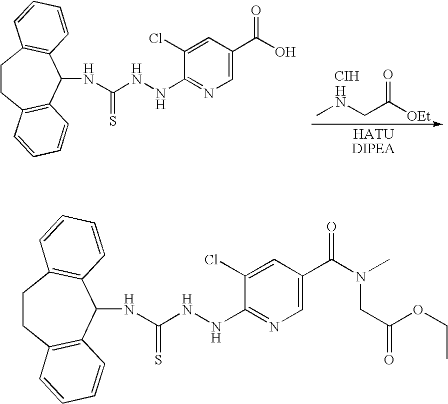

Following general procedure of HATU coupling of Example 1C: HATU (40 mg, 0.105 mmol) was added to a mixture of 5-chloro-6-[2-[[(10,11-dihydro-5H-dibenzo[a,d]cyclohepten-5-yl)amino]thioxo-methyl]hydrazino]-3-pyridinecarboxylic acid.DMA (50 mg, 0.095 mmol), sarcosine ethyl ester hydrochloride (17 mg, 1.10 mmol), and DIPEA (0.25 mmol) in DMA (4 mL) at room temperature. The reaction mixture was stirred at room temperature for 2 h, and then DMA was removed under reduced pressure. Water (5 mL) was added to the residue and the solid was collected and dried in vacuo to give crude product. The crude product was purified by flash chromatography on silica gel (EtOAc:CH₂Cl₂ 1:4) to provide the title compound (30 mg, 59%). ¹HNMR (DMSO-d6): δ 9.60 (9.18) (br, 1H), 8.20 (8.05) (br, 1H), 7.79 (7.67) (br, 1H), 7.49 (br, 1H), 7.26 (br, 2H), 7.20-6.90 (m, 7H), 4.16 (s, 2H), 4.10 (m, 2H), 3.05 (s, 3H), 2.94 (m, 4H), 1.18 (m, 3H) ppm. MS (ESI) (M+1)⁺: 538.

### Compound 5: 5-chloro-6-[2-[[(10,11-dihydro-5H-dibenzo[a,d]cyclohepten-5-yl)amino]thioxomethyl]hydrazino]-N-(2-hydroxyethyl)-N-(phenylmethyl)-3-pyridinecarboxamide

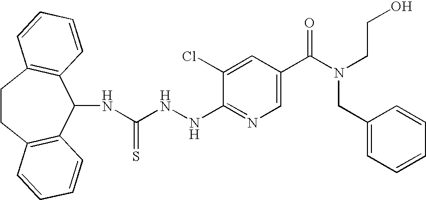

HATU (42 mg, 0.11 mmol) was added to a mixture of 5-chloro-6-[2-[[(10,11-dihydro-5H-dibenzo[a,d]cyclohepten-5-yl)amino]thioxo-methyl]hydrazino]-3-pyridinecarboxylic acid.DMA (53 mg, 0.10 mmol), N-benzylethanolamine (17 mg, 0.11 mmol), and DIPEA (39 mg, 0.3 mmol) in DMA (4 mL) at room temperature. The reaction mixture was stirred at room temperature for 2 h, worked up by adding H₂O (10 mL), extracted with EtOAc (2×30 mL). The extracts were washed with H₂O (10 mL), saturated NaHCO₃ (5 mL), brine (5 mL), and dried over sodium sulfate. Removal of solvent gave a yellow semi-solid which was triturated with H₂O to give a yellow solid. The solid was collected and dried in vacuo and then purified by preparative TLC plate (EtOAc:CH₂Cl₂ 1:1) to give the title compound (12 mg, 21%). IMR (DMSO-d6): δ 9.56 (9.08) (s, 1H), 8.87 (br, 1H), 8.22 (brs, 1H), 7.88 (brs, 1H), 7.46 (br, 1H), 7.40-6.80 (m, 14H), 4.66 (s, 2H), 3.60-3.40 (n, 2H), 3.38-3.10 (m, 4H, overlap with H₂O in DMSO), 3.00-2.80 (m, 2H) ppm. MS (ESI) (M+1)⁺: 572.

### Example 6

### 5-chloro-6-[2-[[(10,11-dihydro-5H-dibenzo[a,d]cyclohepten-5-yl)amino]carbonyl]-hydrazino]-N-(tetrahydro-2-oxo-3-thienyl)-3-pyridinecarboxamide

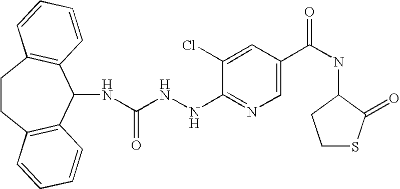

### 6A: 5-chloro-6-[2-[[(10,11-dihydro-5H-dibenzo[a,d]cyclohepten-5-yl)amino]carbonyl]-hydrazino]-3-pyridinecarboxylic Acid

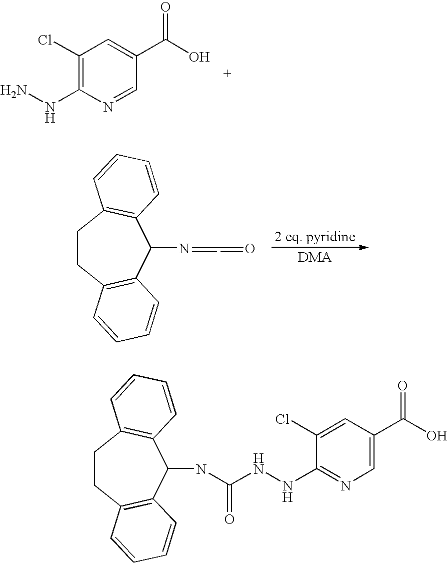

A mixture of 5-chloro-6-hydrazino-3-pyridinecarboxylic acid (1.88 g, 10.01 mmol) and 10,11-dihydro-5-isocyanato-5H-dibenzo[a,d]cycloheptene (2.38 g, 10.11 mmol) (prepared according to: M. A. Davis et al J. Med. Chem. 1964, 7, 88-94, which is incorporated by reference herein for its disclosure of the preparation of 10,11-dihydro-5-isocyanato-5H-dibenzo[a,d]cycloheptene) and pyridine (1.65 mL, 20 mmol) in DMA (60 mL) was stirred at room temperature overnight. The solvent was removed in vacuo, and the residue was triturated with MeOH (3 mL) and washed with Et₂O (50 mL) and dried to give a white solid (4.60 g, 90.4%) as a 1:1 complex of the title compound and DMA. ¹HNMR (DMSO-d6): δ 9.01 (brs, 1H), 8.48 (s, 1H), 7.95 (s, 1H), 7.94 (brs, 1H), 7.58-7.52 (m, 1H), 7.33 (d, J=7.6 Hz, 2H), 7.18-7.05 (m, 6H), 6.24 (br, 1H), 3.24-3.04 (m, 4H), 2.91 (s, 3H, DMA), 2.75 (s, 3H, DMA), 1.92 (s, 3H, DMA) ppm. MS (ESI) (M+1)⁺: 423.

### 6B: 5-chloro-6-[2-[[(10,11-dihydro-5H-dibenzo[a,d]cyclohepten-5-yl)amino]carbonyl]-hydrazino]-N-(tetrahydro-2-oxo-3-thienyl)-3-pyridinecarboxamide

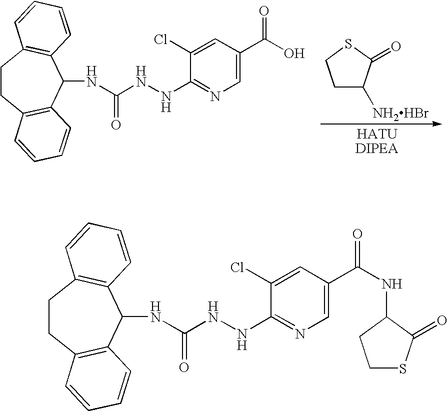

Following general HATU coupling procedure of Example 1C: HATU (76 mg, 0.20 mmol) was added to a mixture of 5-chloro-6-[2-[[(10,11-dihydro-5H-dibenzo[a,d]cyclohepten-5-yl)amino]carbonyl]-hydrazino]-3-pyridinecarboxylic acid. DMA complex (82 mg, 0.16 mmol), homocysteine thiolactone hydrochloride (31 mg, 0.20 mmol), and DIPEA (0.15 mL) in DMA (4 mL) at room temperature. The reaction mixture was stirred at room temperature for 2 h, and then DMA was removed under reduced pressure. Water (10 mL) was added to the residue and the solid was collected. The product was recrystallized in EtOH and EtOAc to provide the title compound (54 mg, 64%). ¹HNMR (DMSO-d6): δ 8.88 (7.92) (s, 1H), 8.65 (d, J=8.0 Hz, 1H), 8.47 (d, J=1.2 Hz, 1H), 8.05 (s, 1H), 7.60-7.50 (m, 1H), 7.34 (d, J=6.0 Hz, 2H), 7.13 (br, 6H), 6.25 (br, 1H), 4.85-4.76 (m, 1H), 3.48-3.38 (m, 1H), 3.34-3.26 (m, 1H, overlap with H₂O in DMSO-d6), 3.22-3.06 (m, 4H), 2.50-2.40 (m, 1H, overlap with DMSO), 2.28-2.16 (m., 1H) ppm. MS (ESI) (M+1)⁺: 522. Anal. Calcd for C₂₆H₂₄ClN₅O₃S.0.25H₂O: C, 59.30; H, 4.70; N, 13.29. Found: C, 59.39; H, 4.67; N, 13.19.

### Example 7

### N-[[5-chloro-6-[2-[[(10,11-dihydro-5H-dibenzo[a,d]cyclohepten-5-yl)amino]carbonyl]-hydrazino]-3-pyridinyl]carbonyl]-N-methyl-glycine Ethyl Ester

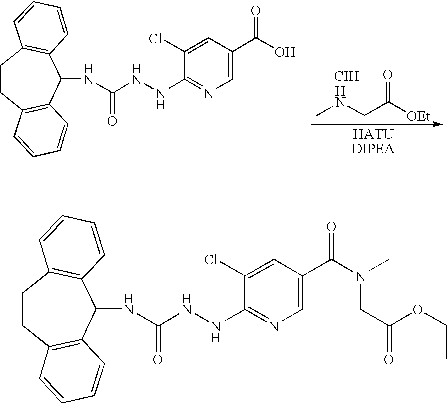

Following general HATU coupling procedure of Example 1C: HATU (40 mg, 0.11 mmol) was added to a mixture of 5-chloro-6-[2-[[(10,11-dihydro-5H-dibenzo[a,d]cyclohepten-5-yl)amino]carbonyl]-hydrazino]-3-pyridinecarboxylic acid DMA complex (51 mg, 0.10 mmol), homocysteine thiolactone hydrochloride (17 mg, 0.11 mmol), and DIPEA (50 ILL) in DMA (4 mL) at room temperature. The reaction mixture was stirred at room temperature for 2 h, and then DMA was removed under reduced pressure. Water (10 mL) was added to the residue and the solid was collected. The crude product was purified by preparative TLC (MeOH:CH₂Cl₂ 1:9) to provide the title compound (15 mg, 29%). ¹HNMR (DMSO-d6): δ 8.75 (s, 1H), 8.09 (s, 0.75H), 7.95 (s, 0.25H), 7.86 (s, 1H), 7.71 (s, 0.75H), 7.57 (s, 0.25H), 7.51 (d, J=8 Hz, 1H), 7.31 (d, J=7.2 Hz, 2H), 7.18-7.04 (m, 6H), 6.25 (s, 1H), 4.12 (s, 2H), 4.06 (q, J=7.2 Hz, 2H), 3.20-2.85 (m, 7H), 1.25-1.05 (m, 3H, combination of two sets of triplets from two rotamers). MS (ESI) (M+1)⁺: 522.

### Example 8

### 5-chloro-N-(tetrahydro-2-oxo-3-thienyl)-6-[2-[[[(1R*,2S*)-1,2,3,4-tetrahydro-2-phenyl-4-naphthalenyl]amino]thioxomethyl]hydrazino]-3-pyridinecarboxamide

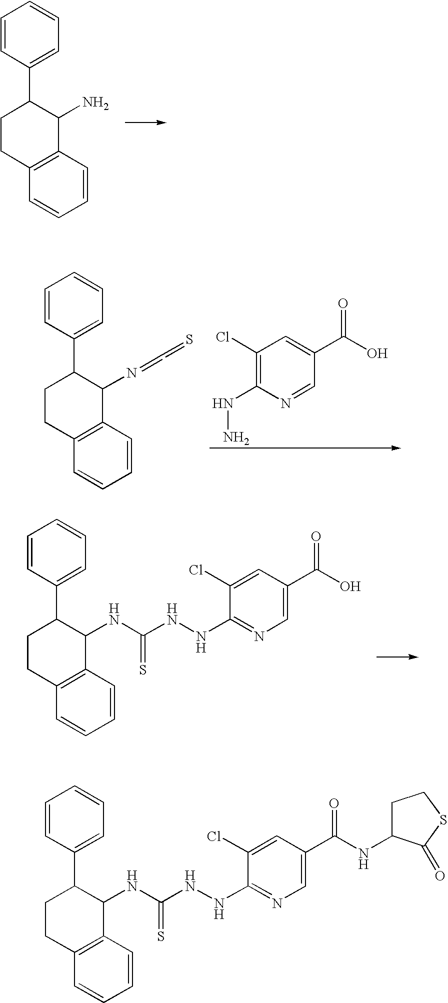

### 8A: (1R*,2S*)-1,2,3,4-tetrahydro-1-isothiocyanato-2-phenyl-naphthalene

To the vigorously stirred reaction mixture of (1R*,2S*)-1,2,3,4-tetrahydro-2-phenyl-1-naphthalenamine hydrochloride (52 mg, 0.20 mmol) (prepared according to: E. J. Pribyl et al, U.S. Pat. No. 3,388,121 and J. W. Clader et al, Eur. Pat. Appl. No. EP 508425 A1, which are incorporated by reference herein for their disclosures in the to preparation of (1R*,2S*)-1,2,3,4-tetrahydro-2-phenyl-1-naphthalenamine hydrochloride) in 1,2-dichloroethane (3 mL) and saturated aqueous NaHCO₃ solution (3 mL) was added thiophosgene (0.10 mL) at room temperature. The reaction mixture was stirred at room temperature for 1 h, diluted with EtOAc (30 mL). The organic layer was separated and washed with saturated NaHCO₃ (10 mL), brine (10 mL), and dried over Na₂SO₄. Removal of solvent and excess thiophosgene in vacuo gave the title compound, which was used without purification in the next step.

### 8B: 5-chloro-6-[2-[[[(1R*,2S*)-1,2,3,4-tetrahydro-2-phenyl-1-naphthalenyl]amino]thioxomethyl]hydrazino]-3-pyridinecarboxylic Acid

5-chloro-6-hydrazino-3-pyridinecarboxylic acid (37 mg, 0.20 mmol) was added to the solution of (1R*,2S*)-1,2,3,4-tetrahydro-1-isothiocyanato-2-phenyl-naphthalene in DMA (4 mL), and the reaction mixture was stirred at room temperature overnight. The resulting solution was used in the next step without further purification.

### 8C: 5-chloro-N-(tetrahydro-2-oxo-3-thienyl)-6-[2-[[[(1R*,2S*)-1,2,3,4-tetrahydro-2-phenyl-1-naphthalenyl]amino]thioxomethyl]hydrazino]-3-pyridinecarboxamide

To 5-chloro-6-[2-[[[(1R*,2S*)-1,2,3,4-tetrahydro-2-phenyl-1-naphthalenyl]amino]-thioxomethyl]hydrazino]-3-pyridinecarboxylic acid (0.20 mmol) in DMA (4 mL) was added homocysteine thiolactone hydrochloride (38 mg, 0.25 mmol), DIPEA (0.15 mL), followed by HATU (95 mg, 0.25 mmol). The reaction mixture was stirred at room temperature for 2 h, and concentrated in vacuo. Added H₂O (5 mL) to the residue and the solid was collected and recrystallized from EtOH to give the title compound (50 mg, 45% in 3 steps). ¹HNMR (DMSO-d6): δ 9.29 (s, 1H), 8.87 (s, 1H), 8.67 (d, J=7.2 Hz, 1H), 8.43 (s, 1H), 8.00 (s, 1H), 7.39 (brs, 1H), 7.25-6.75 (m, 9H), 6.00 (m, 1H), 4.79 (m, 1H), 3.45-3.36 (m, 1H), 3.34-3.18 (m, 3H, overlap with H₂O in DMSO), 2.80-2.64 (m, 1H), 2.30-2.15 (m, 1H), 2.08-1.88 (m, 2H) ppm. MS (ESI) (M+1)⁺: 552.

### Example 9

### 5-chloro-6-[2-[[[(4-chlorophenyl)phenylmethyl]amino]thioxomethyl]-hydrazino]-N-(tetrahydro-2-oxo-3-thienyl)-3-pyridinecarboxamide

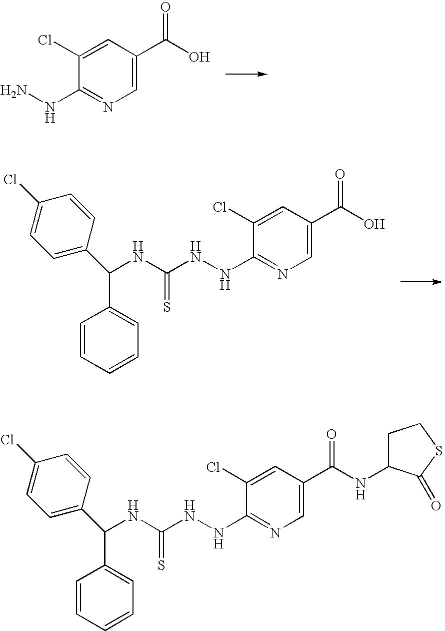

### 9A: 5-chloro-6-[2-[[[(4-chlorophenyl)phenylmethyl]amino]-thioxomethyl]hydrazino]-3-pyridinecarboxylic Acid

Following the general procedure of Example 8B: 5-chloro-6-hydrazino-3-pyridinecarboxylic acid (37 mg, 0.20 mmol) was added to the solution of 1-chloro-4-(isothiocyanatophenylmethyl)benzene (0.20 mmol) [prepared from 4-chloro-a-phenyl-benzenemethanamine HCl salt and thiophosgene in dichloroethane and saturated NaHCO₃ following general procedure Example 8A] in DMA (4 mL), and the mixture was stirred at room temperature overnight. The pyridinecarboxylic acid solution was used in the next step without further purification.

### 9B: 5-chloro-6-[2-[[[(4-chlorophenyl)phenylmethyl]amino]thioxomethyl]hydrazino]-N-(tetrahydro-2-oxo-3-thienyl)-3-pyridinecarboxamide

To the above prepared 5-chloro-6-[2-[[[(4-chlorophenyl)phenylmethyl]amino]-thioxomethyl]hydrazino]-3-pyridinecarboxylic acid (0.20 mmol) in DMA (4 mL) from Example 9A was added homocysteine thiolactone hydrochloride (38 mg, 0.25 mmol), DIPEA (0.15 mL), followed by HATU (95 mg, 0.25 mmol). The reaction mixture was stirred at room temperature for 2 h, and concentrated in vacuo. H₂O (5 mL) was added to the residue and the solid was collected and recrystallized from EtOH to give the title compound (51 mg, 47% in two steps). ¹HNMR (DMSO-d6): δ 9.54 (brs, 1H), 9.09 (brs, 1H), 0.85-8.72 (m, 1H), 8.68 (d, J=8 Hz, 1H), 8.55 (s, 1H), 8.05 (s, 1H), 7.40-7.05 (m, 9H), 6.95 (d, J=8.8 Hz, 1H), 4.85-4.75 (m, 1H), 3.46-3.39 (m, 1H), 3.35-3.20 (m, 1H, overlap with H2O in DMSO) 2.50-2.40 (m, 1H, overlap with DMSO), 2.30-2.16 (m, 1H) ppm. MS (ESI) (M+1)⁺: 546.

### Example 10

### 5-chloro-6-[2-[[(1,2-diphenylethyl)amino]thioxomethyl]-hydrazino]-N-(tetrahydro-2-oxo-3-thienyl)-3-pyridinecarboxamide

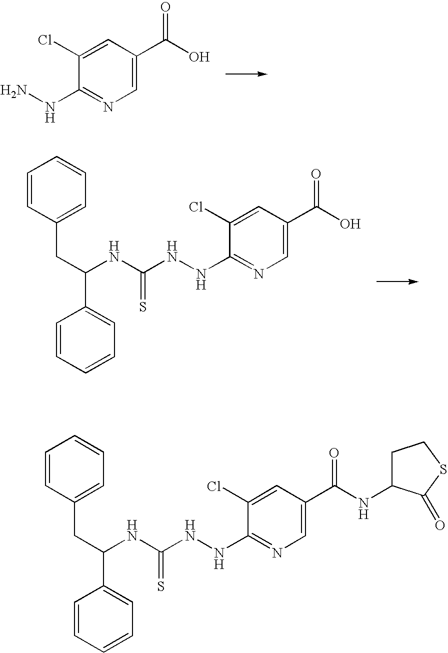

### 10A: 5-chloro-6-[2-[[(1,2-diphenylethyl)amino]thioxomethyl]hydrazino]-3-pyridinecarboxylic Acid

Following the general procedure of Example 8B: The solution of 5-chloro-6-hydrazino-3-pyridinecarboxylic acid (37 mg, 0.20 mmol) and 1,2-diphenylethylisothiocyanate (0.20 mmol) [prepared from 1,2-diphenylethylamine and thiophosgene in dichloroethane and saturated NaHCO₃ following general procedure Example 8A] in DMA (4 mL) was stirred at room temperature overnight. The pyridinecarboxylic acid solution was used in the next step without further purification.

### 10B: 5-chloro-6-[2-[[(1,2-diphenylethylamino]thioxomethyl]hydrazino]-N-(tetrahydro-2-oxo-3-thienyl)-3-pyridinecarboxamide

To the above prepared 5-chloro-6-[2-[[(1,2-diphenylethyl)amino]thioxomethyl]-hydrazino]-3-pyridinecarboxylic acid (0.20 mmol) in DMA (4 mL) from Step 10A was added homocysteine thiolactone hydrochloride (38 mg, 0.25 mmol), DIPEA (0.15 mL), followed by HATU (95 mg, 0.25 mmol). The reaction mixture was stirred at room temperature for 2 h, and concentrated in vacuo. H₂O (5 mL) was added to the residue and the solid was collected and recrystallized from EtOH to give the title compound (36 mg, 34% in two steps). ¹HNMR (DMSO-d6): δ 9.32 (d, J=3.2 Hz, 1H), 9.09 (brs, 1H), 8.72 (d, J=8.0 Hz, 1H), 8.48 (s, 1H), 8.19 (brs, 1H), 8.11 (s, 1H), 7.35-7.05 (m, 10H), 5.76-5.66 (m, 1H), 4.88-4.78 (m, 1H), 3.50-3.40 (m, 1H), 3.36-3.26 (m, 1H, overlap with H₂O in DMSO), 3.13 (dd, J=9.0, 14.0. Hz, 1H), 2.96 (dd, J=6.0, 14.0 Hz, 1H), 2.52-2.42 (m, 1H, overlap with DMSO), 2.34-2.20 (m, 1H) ppm. MS (ESI) (M+1)⁺: 526. Anal. Calcd for C₂₅H₂₄ClN₅O₂S₂.0.25H₂O: C, 56.59; H, 4.65; N, 13.20. Found: C, 56.52; H, 4.33; N, 13.23.

### Example 11

### N-[3-Chloro-4-[[[[(10,11-dihydro-5H-dibenzo[a,d]cyclohepten-5-yl)amino]carbonyl]amino]methyl]benzoyl]-N-methyl-glycine, Ethyl Ester

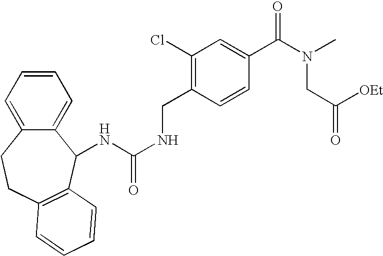

### 11A: 4-(Bromomethyl)-3-chlorobenzonitrile

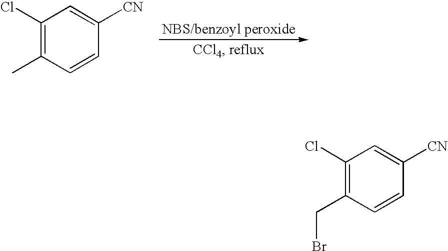

To a stirred suspension of 3-chloro-4-methylbenzonitrile (4.55 g, 30 mmol) and NBS (5.52 g, 31 mmol) in CCl₄ (40 mL) was added benzoyl peroxide (110 mg). The reaction mixture was heated to reflux for 3 h, allowed to cool to room temperature and filtered through Cetite. The Celite pad was washed with 10 mL of CCl₄, and the filtrate was concentrated in vacuo. The product was purified by recrystallization from ethanol and hexanes to yield the title compound as white crystals (3.46 g, 50%). ¹H NMR (CDCl₃): δ 7.69 (d, J=1.5 Hz, 1H), 7.62-7.54 (m, 2H), 4.58 (s, 2H) ppm.

### 11B: 1,1-dimethylethyl-[(2-chloro-4-cyanophenyl)methyl]-[(1,1dimethylethoxy]-carbamate

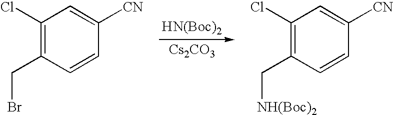

To a stirred solution of 4-(bromomethyl)-3-chlorobenzonitrite (1.1 g, 4.76 mmol) in DMF (15 mL) was added HN(Boc)₂ (1.14 g, 5.24 mmol) followed by Cs₂CO₃ (1.71 g, 5.24 mmol). The resulting mixture was stirred at room temperature overnight. The light brown reaction mixture was then concentrated in vacuo, and the residue was taken up into EtOAc (50 mL). The organic phase was washed with water (2×10 mL), brine (1×10 mL) and then concentrated in vacuo to provide the title compound as a light brown oil (1.67 g, 96%). This material was used in the step without further purification. ¹H-NMR (CDCl₃): δ 7.49 (s, 1H), 7.41 (d, J=6.3 Hz, 1H), 7.14 (d, J=6.3 Hz, 1H), 4.77 (s, 2H), 1.30 (s, 18H) ppm.

### 11C: 4-(Aminomethyl)-3-chlorobenzonitrite

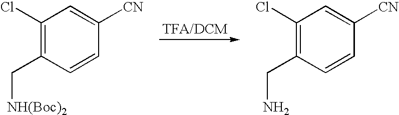

To a solution of 1,1-dimethylethyl-[(2-chloro-4-cyanophenyl)methyl][(1,1-dimethylethoxy]carbamate (1.67 g, 4.55 mmol) in CH₂Cl₂ (5 mL) was added TFA (2.8 mL, 36.4 mmol). The resulting mixture was stirred at room temperature overnight. The reaction mixture was then concentrated in vacuo, and the residue was taken up into DCM (80 mL). The organic phase was washed with saturated NaHCO₃ (2×20 mL), brine (1×20 mL), dried over Na₂SO₄, filtered, and concentrated in vacuo to provide the title compound as a light brown oil (0.76 g, quantitative). This material was used in the following step without further purification. ¹H-NMR (CD₃OD): δ 7.89 (s, 1H), 7.76-7.70 (m, 2H), 5.29 (s, 2H) ppm.

### 11D: 4-(Aminomethyl)-3-chloro Benzoic Acid Hydrochloride

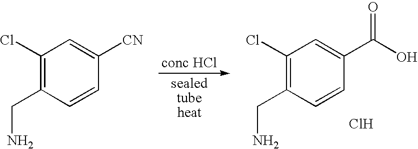

4-(Aminomethyl)-3-chlorobenzonitrile (0.76 g, 4.55 mmol) was suspended in conc. HCl (3 mL) and the mixture was stirred at 150° C. in a sealed tube for 8 h, allowed to cool to room temperature. The solid was collected and washed with Et₂O to afford the title compound (0.87 g, 87%). This material was used in the step without further purification. MS (ESI) (M+H)⁺=186.

### 11E: 3-Chloro-4-[[[[(10,11-dihydro-5H-dibenzo[a,d]cyclohepten-5-yl)amino]carbonyl]amino]methyl]-benzoic Acid

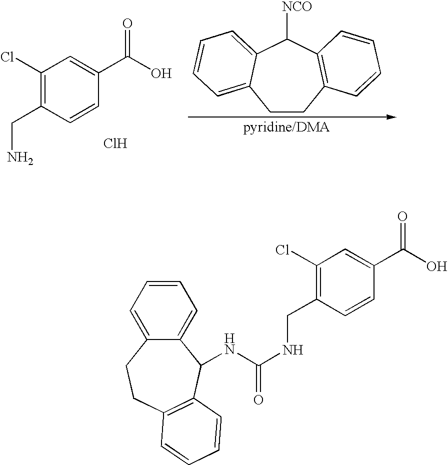

Pyridine (1.21 mL, 15 mmol) was added to a solution of 4-(Aminomethyl)-3-chloro benzoic acid hydrochloride (666 mg, 3.0 mmol) in DMA (25 mL). The mixture was stirred at room temperature for 30 min, and then 10,11-dihydro-5-isocyanato-5H-dibenzo[a,d]cycloheptene (777 mg, 3.3 mol) was added and the mixture was stirred at room temperature overnight. The reaction mixture was concentrated in vacuo, and water (10 mL) was added. The precipitate was isolated and washed with ethyl acetate, diethyl ether, and dried to give the title compound (1.02 g, 82%). This material was used in the next step without further purification. MS (ESI) (M+H)⁺=421.

### 11F: N-[3-Chloro-4-[[[[(10,11-dihydro-5H-dibenzo[a,d]cyclohepten-5-yl)amino]-carbonyl]amino]methyl]benzoyl]-N-methyl-glycine Ethyl Ester

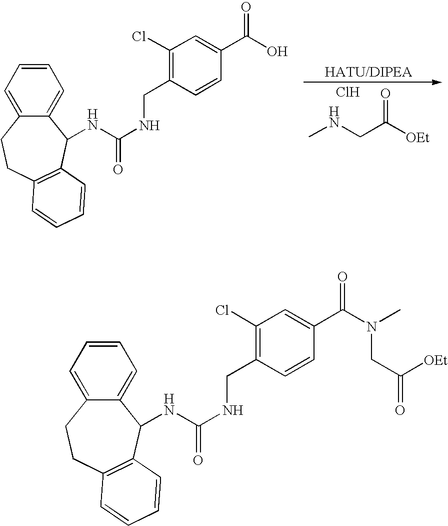

To a stirred solution of 3-Chloro-4-[[[[(10,11-dihydro-5H-dibenzo[a,d]cyclohepten-5-yl)amino]carbonyl]amino]methyl]-benzoic acid (60 mg, 0.143 mmol) in DMA (2 mL) was added HATU (65 mg, 0.172 mmol) followed by DIPEA (34 μL, 0.172 mmol). The mixture was stirred at room temperature for 10 min, and then sarcosine ethyl ester hydrochloride (26.4 mg, 0.172 mmol) was added. The resulting mixture was stirred at room temperature for 4 h, and concentrated in vacuo. The residue was taken up in CH₂Cl₂ (20 mL), and washed with saturated NaHCO₃ (2×10 mL) and then brine (1×10 mL). The material obtained after removal of the solvent was purified by reverse-phase HPLC (20˜70% MeCN in water). Isolated fractions containing the pure product were treated with NaHCO₃ (excess) powder. The supernatants were all combined, concentrated in vacuo and extracted with ethyl acetate (2×20 mL). The ethyl acetate extract was washed with brine, dried over Na₂SO₄ and concentrated to provide the title compound (23.8 mg, 32%). ¹H-NMR (CD₃OD): δ 7.45-7.35 (m, 1H), 7.34-7.26 (m, 4H), 7.14-7.05 (m, 6H), 6.20 (s, 1H), 4.84 (s, 2H), 4.40 (s, one rotamer, 1.5H), 4.38 (s, one rotamer, 0.5H), 4.22-4.10 (m, 2H), 3.28-3.08 (m, 4H), 3.03 (s, one rotamer, 1H), 2.96(s, one rotamer, 2H), 1.26 (t, one rotamer, J=7.6 Hz, 2H), 1.17 (t, one rotamer, J=7.6 Hz, 1H) ppm. MS (ESI) (M+H)⁺=520.

### Example 12

### 3-Chloro-4-[[[[(10,11-dihydro-5H-dibenzo[a,d]cyclohepten-5-yl)amino]carbonyl]amino]methyl]-N-[[(2R)-1-ethyl-2-pyrrolidinyl]methyl]-benzamide

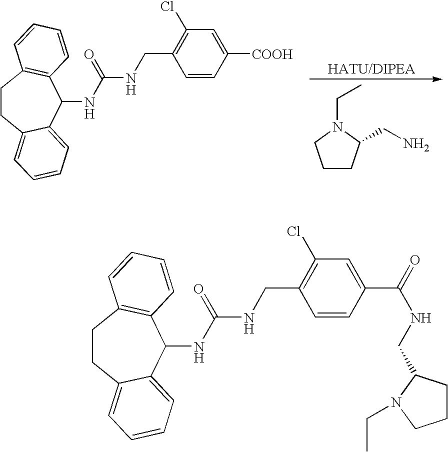

Following general procedure 11F: To a stirred solution of 3-Chloro-4-[[[[(10,11-dihydro-5H-dibenzo[a,d]cyclohepten-5-yl)amino]carbonyl]amino]methyl]-benzoic acid (60 mg, 0.143 mmol) in DMA (2 mL) was added HATU (65 mg, 0.172 mmol) followed by DIPEA (34 μL, 0.172 mmol). The mixture was stirred at room temperature for 10 min, then (S)-(−)-2-aminomethyl-1-ethylpyrrolidine (22.0 mg, 0.172 mmol) was added. The resulting mixture was stirred at room temperature for 4 h. After work-up, the crude product was purified by reverse-phase HPLC (20-70% MeCN in water) affording the title compound (29.6 mg, 37%). ¹H-NMR (CD₃OD): δ 7.74 (s, 1H), 7.49 (d, J=6.0 Hz, 1H), 7.37 (d, J=6.0 Hz, 2H), 7.29-7.24 (m, 3H), 7.22-7.11 (m, 4H), 5.93 (d, J=4.2 Hz, 1H), 5.41 (s, br, 1H), 5.11 (s, br, 1H), 4.41 (d, J=4.8 Hz, 2H), 3.66-3.59 (m, 1H), 3.45-3.26 (m, 4H), 3.11-2.99 (m, 2H), 2.95-2.76 (m, 2H), 2.43-2.25 (m, 2H), 1.84-1.70 (m, 2H), 1.16 (t, J=5.4 Hz, 3H) ppm. MS (ESI) (M+H)⁺=531.

### Example 13

### 3-Chloro-4-[[[[(10,11-dihydro-5H-dibenzo[a,d]cyclohepten-5-yl)-amino]carbonyl]amino]methyl]-N-[2-(1-methyl-2-pyrrolidinyl)ethyl]-benzamide

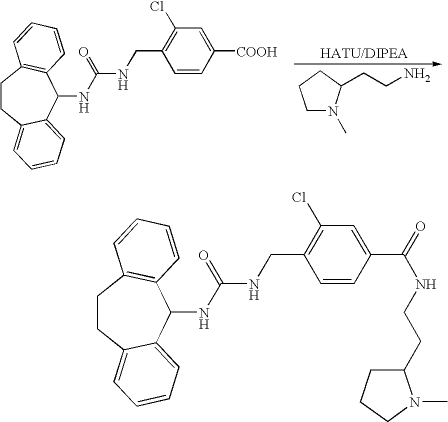

Following general procedure Example 11F: To a stirred solution of 3-Chloro-4-[[[[(10,11-dihydro-5H-dibenzo[a,d]cyclohepten-5-yl)amino]carbonyl]amino]methyl]-benzoic acid (60 mg, 0.143 mmol) in DMA (2 mL) was added HATU (65 mg, 0.172 mmol) followed by DIPEA (34 μL, 0.172 mmol). The mixture was stirred at room temperature for 10 min, then 2-(2-aminoethyl)-1-methylpyrrolidine (22.0 mg, 0.172 mmol) was added. The resulting mixture was stirred at room temperature for 4 h. After work-up, the crude product was purified by reverse-phase HPLC (20˜70% MeCN in water) to provide the title compound (16.4 mg, 21%). ¹H-NMR (CD₃OD): δ 9.00 (d, J=4.2 Hz, 1H), 7.50 (d, J=1.5 Hz, 1H), 7.38 (d, J=5.7 Hz, 2H), 7.26 (m, 1H), 7.14-7.02 (m, 6H), 6.64 (d, J=6.0 Hz, 1H), 6.24 (d, J=6.0 Hz, 1H), 6.06 (t, J=4.8 Hz, 1H), 4.31 (d, J=4.8 Hz, 2H), 3.58-3.48 (m, 1H), 3.33-3.18 (m, 2H), 3.15-3.03 (m, 2H), 2.44-2.37 (m, 1H), 2.32 (s, 3H), 2.23-2.15 (m, 1H), 1.98 (s, br, 1H), 1.91-1.78 (m, 1H), 1.77-1.58 (m, 4H) ppm. MS (ESI) (M+H)⁺=531.

### Example 14

### 3-Chloro-4-[[[[(10,11-dihydro-5H-dibenzo[a,d]cyclohepten-5-yl)amino]carbonyl]amino]methyl]-N-[3-(2-methyl-1-piperidinyl)propyl]-benzamide

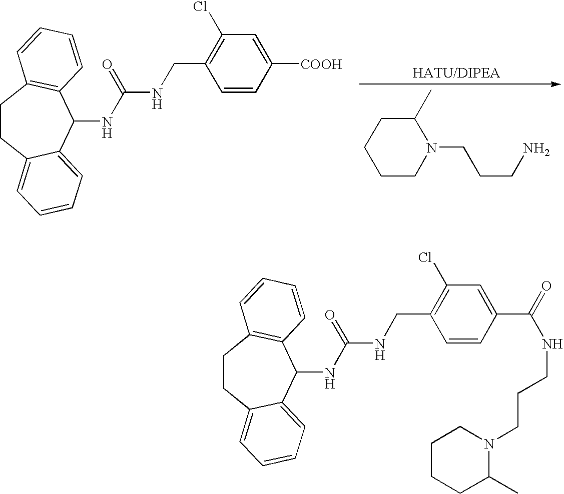

Following general procedure Example 11F: To a stirred solution of 3-Chloro-4-[[[[(10,11-dihydro-5H-dibenzo[a,d]cyclohepten-5-yl)amino]carbonyl]amino]methyl]-benzoic acid (60 mg, 0.143 mmol) in DMA (2 mL) was added HATU (65 mg, 0.172 mmol) followed by DIPEA (34 mL, 0.172 mmol). The mixture was stirred at room temperature for 10 min, and then 1-(3-aminopropyl)-2-pipecoline (26.9 mg, 0.172 mmol) was added. The resulting mixture was stirred at room temperature for 4 h. After work-up, the crude product was purified by reverse-phase HPLC (20-70% MeCN in water) to provide the title compound (24.8 mg, 31%). ¹H-NMR (CD₃OD): δ 9.14 (s, 1H), 7.57 (d, J=1.2 Hz, 1H), 7.42-7.34 (m, 3H), 7.15-7.01 (m, 6H), 6.91 (d, J=6.0 Hz, 1H), 6.29 (m, 2H), 4.34 (d, J=4.5 Hz, 2H), 3.54-3.43 (m, 1H), 3.26-3.06 (m, 4H), 2.94-2.84 (m, 2H), 2.31-2.17 (m, 2H), 2.04-1.94 (m, 1H), 1.79-1.51 (m, 4H), 1.49-1.38 (m, 1H), 1.36-1.21 (m, 2H), 1.02 (d, J=4.8 Hz, 3H) ppm. MS (ESI) (M+H)⁺=559.

### Example 15

### N-[[5-Chloro-6-[[[[(10,11-dihydro-5H-dibenzo[a,d]cyclohepten-5-yl)amino]carbonyl]amino]methyl]-3-pyridinyl]carbonyl]-N-methyl-glycine Ethyl Ester

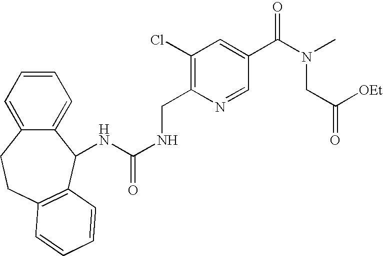

### 15A: Methyl 5,6-dichloro-3-pyridinecarboxylate

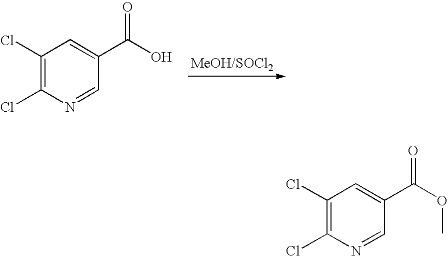

To a stirred solution of 5,6-dichloro-3-pyridinecarboxylic acid (1.92 g, 10 mmol) in MeOH (30 mL) cooled to 0° C., thionyl chloride (0.726 mL, 10 mmol) was added dropwise and the resulting mixture was stirred at room temperature overnight. The reaction mixture was then concentrated in vacuo, and the residue was taken up in CH₂Cl₂ (50 mL). The organic phase was washed with saturated NaHCO₃ (2×10 mL) and brine (1×10 mL), dried over MgSO₄, filtered, and concentrated in vacuo to provide the title compound as a white solid (1.39 g, 67%). This material was used in the next step without further purification. ¹H-NMR (CDCl₃): δ 8.89 (s, 1H), 8.37 (s, 1), 3.98 (s, 3H) ppm.

### 15B: Methyl 5-chloro-6-cyano-3-pyridinecarboxylate

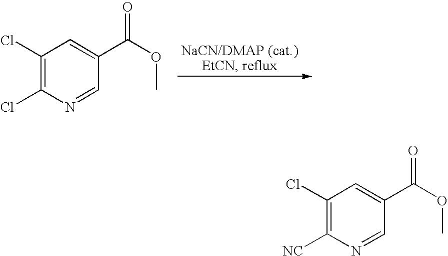

To a stirred solution of methyl 5,6-dichloro-3-pyridinecarboxylate (824 mg, 4.0 mmol) in EtCN (40 mL) was added NaCN (294 mg, 6.0 mmol) and then DMAP (80 mg, 10% w.t.). The resulting mixture was stirred at 99° C. overnight and cooled to room temperature. The reaction mixture was then concentrated in vacuo, water (30 mL) was added and extracted with CH₂Cl₂ (3×20 mL). The organic layers were combined, washed with water (2×10 mL), brine (1×10 mL), dried over MgSO₄, and concentrated in vacuo. The product was purified by flash chromatography (10˜15% EtOAc in Hexanes) to provide the title compound as a white solid (508 mg, 65%). ¹H-NMR (CDCl₃): δ 9.17 (s, 1H), 8.46 (s, 1H), 4.03 (s, 3H) ppm. MS (ESI) (M+H)⁺=197.

### 15C: Methyl 6-(aminomethyl)-5-chloro-3-pyridinecarboxylate

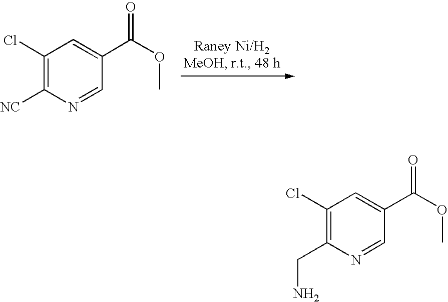

Raney nickel (slurry in water, cat.) was added to a solution of methyl 5-chloro-6-cyano-3-pyridinecarboxylate (393 mg, 2.0 mmol) in MeOH (40 mL). The resulting mixture was hydrogenated at 40 psi until the reaction was complete by LC/MS (48 h). After filtration, the green filtrate was concentrated in vacuo, and the residue was used directly for the next step without further purification. MS (ESI) (M+H)⁺=201.

### 15D: Methyl 5-chloro-6-[[[[(10,11-dihydro-5H-dibenzo[a,d]cyclohepten-5-yl)amino]carbonyl]amino]methyl]-3-pyridinecarboxylate

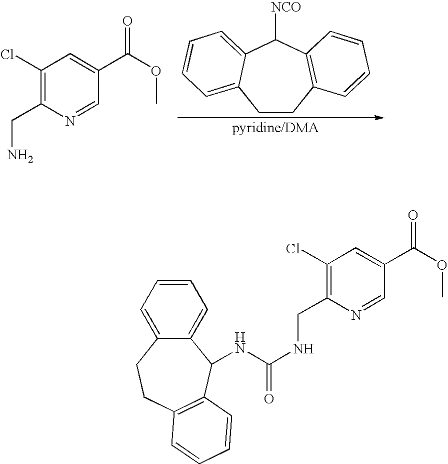

Following general procedure Example 11E: To a solution of the above crude methyl 6-(aminomethyl)-5-chloro-3-pyridinecarboxylate (from 2.0 mmol of precursor) in DMA (10 mL) was added pyridine (0.81 mL, 10 mmol). The mixture was stirred at room temperature for 30 min, and then 10,11-dihydro-5-isocyanato-5H-dibenzo[a,d]cycloheptene (565 mg, 2.4 mol) was added and mixture was stirred at room temperature overnight. The reaction mixture was then concentrated in vacuo, and triturated with water (10 mL). The precipitate was collected by filtration, washed with methanol, and then dried to give the title compound as a white solid (620 mg, 72%). This material was used for the next step without further purification. MS (ESI) (M+H)⁺=436.

### 15F: 5-chloro-6-[[[[(10,11-dihydro-5H-dibenzo[a,d]cyclohepten-5-yl)amino]carbonyl]-amino]methyl]-3-pyridinecarboxylic Acid

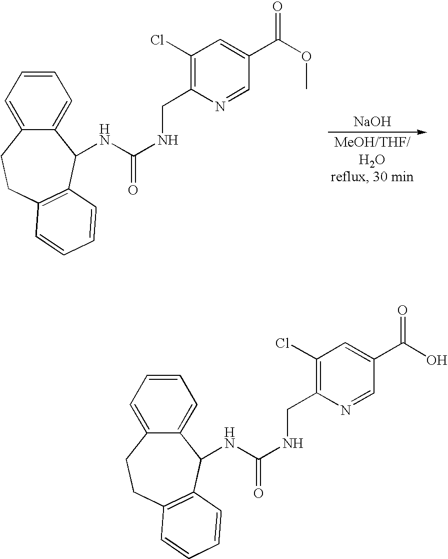

To a stirred suspension of methyl 5-chloro-6-[[[[(10,11-dihydro-5H-dibenzo[a,d]-cyclohepten-5-yl)amino]carbonyl]amino]methyl]-3-pyridinecarboxylate (620 mg, 1.43 mmol) in MeOH/THF/H₂O (1:1:1, 45 mL) was added NaOH (284 mg, 7.1 mmol). The resulting mixture was refluxed for 30 min during which time the solution became clear. After cooling of the reaction mixture to room temperature, the organic solvents were removed in vacuo, and the aqueous slurry was neutralized with 10% acetic acid to pH=6˜7. The precipitate was collected by filtration, washed with water and dried to give the title compound as a white solid (580 mg, 97%). This material was used in the next step without further purification. MS (ESI) (M+H)⁺=422.

### 15G: N-[[5-Chloro-6-[[[[(10,11-dihydro-5H-dibenzo[a,d]cyclohepten-5-yl)amino]-carbonyl]amino]methyl]-3-pyridinyl]carbonyl]-N-methyl-glycine Ethyl Ester

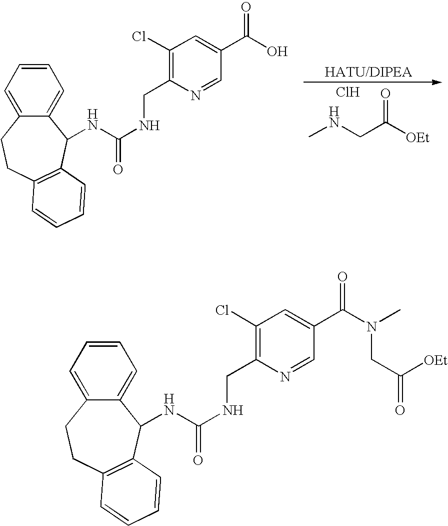

Following general procedure Example 11F: To a stirred solution of 5-chloro-6-[[[[(10,11-dihydro-5H-dibenzo[a,d]cyclohepten-5-yl)amino]carbonyl]amino]methyl]-3-pyridinecarboxylic acid (84 mg, 0.20 mmol) in DMA (3 mL) was added HATU (91 mg, 0.24 mmol) followed by DIPEA (47 μL, 0.24 mmol). The mixture was stirred at room temperature for 10 min, and then sarcosine ethyl ester hydrochloride (36.8 mg, 0.24 mmol) was added. The resulting mixture was stirred at room temperature for 4 h. After work-up, the crude product was purified by preparative TLC (5% Hexanes in EtOAc) to provide the title compound (8.8 mg, 8.5%). ¹H-NMR (CDCl₃): δ 8.40 (s, 0.75H, one rotamer), 8.34 (s, 0.25H, one rotamer), 7.74 (s, 0.75H, one rotamer), 7.66 (s, 0.25H, one rotamer), 7.40 (d, J=6.0 Hz, 2H), 7.18-7.07 (m, 6H), 5.97 (s, br, 1H), 5.90 (s, br, 1H), 5.58 (s, br, 1H), 4.60-4.53 (m, 2H), 4.25-4.16 (m, 4H), 3.48-3.37 (m, 2H), 3.10-3.04 (m, 2H), 3.03 (s, one rotamer, 1H), 3.01 (s, one rotamer, 2H), 1.30-1.24 (m, 3H) ppm. MS (ESI) (M+H)⁺=521.

### Example 16

### N-[[5-chloro-6-[2-[[(10,11-dihydro-5H-dibenzo[a,d]cyclohepten-5-yl)amino]thioxomethyl]hydrazino]-3-pyridinyl]sulfonyl]-N-methyl-glycine, Ethyl Ester

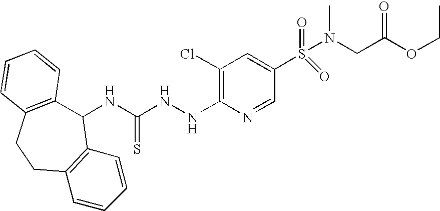

### 16A: N-[(5,6-dichloro-3-pyridinyl)sulfonyl]-N-methyl-glycine Ethyl Ester

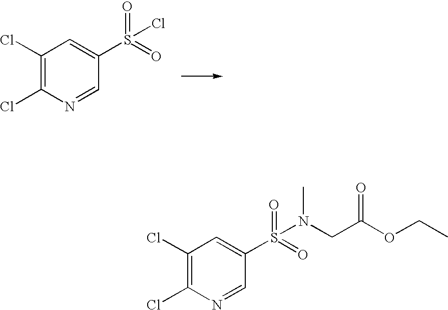

A solution of 5,6-dichloro-3-pyridinesulfonyl chloride (Prepared according to: M. W. Crawley, European Patent Application EP0147105A2 (1985), which is incorporated by reference herein for its disclosure in the preparation of 5,6-dichloro-3-pyridinesulfonyl chloride) (246 mg, 1.00 mmol) in CH₂Cl₂ (5 mL) was added to a mixture of sarcosine ethyl ester hydrochloride (191 mg, 1.25 mmol) and DIPEA (0.60 mL, 3.5 mmol) in CH₂Cl₂ at room temperature. The reaction mixture was stirred at room temperature for 3 h, diluted with EtOAc (50 mL), washed with 5% HCl, H₂O, and brine, dried over Na₂SO₄. Removal of solvent gave the title compound (302 mg, 92%), MS(ESI) (M+1)⁺: 327.

### 16B: N-[(5-chloro-6-hydrazino-3-pyridinyl)sulfonyl]-N-methyl-glycine, Ethyl Ester

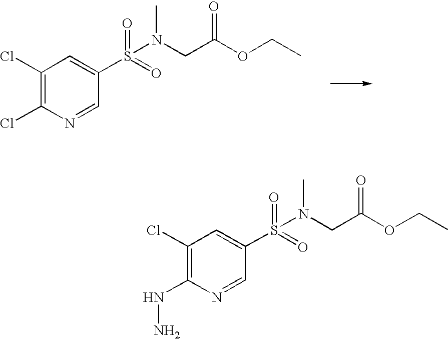

1 M hydrazine (2 mL, 2 mmol) in EtOH was added to the solution of N-[(5,6-dichloro-3-pyridinyl)sulfonyl]-N-methyl-glycine ethyl ester (302 mg) in EtOH (5 mL), and the mixture was heated to reflux for 3 h, allowed to cool to room temperature, and concentrated in vacuo. The residue was triturated with H₂O (3 mL), and the light yellow solid (230 mg, 77%) was collected and dried. MS (ESI) (M+1)⁺=323.

### 16C: N-[[5-chloro-6-[2-[[(10,11-dihydro-5H-dibenzo[a,d]cyclohepten-5-yl)amino]thioxomethyl]hydrazino]-3-pyridinyl]sulfonyl]-N-methyl-glycine, Ethyl Ester

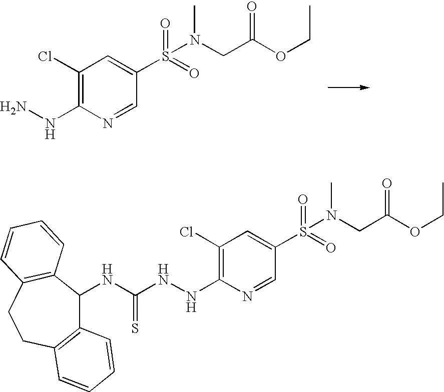

The mixture of the hydrazine compound from 16B (52 mg, 0.16 mmol) and 10,11-dihydro-5-isothiocyanato-5H-dibenzo[a,d]cycloheptene (50 mg, 0.20 mmol), pyridine (50 μL) in DMA (2 mL) was stirred at room temperature overnight. The solvent was evaporated and the residue was triturated with Et₂O (5 mL). The solid was collected and recrystallized in MeOH to the title compound (65 mg, 71%). ¹HNMR (DMSO-d6): δ 9.74 (9.60) (s, 1H), 9.02 (br, 1H), 8.45 (s, 1H), 8.08 (s, 1H), 7.55 (br, 1H), 7.40-7.05 (m, 8H), 7.03 (brs, 1H), 4.05 (s, 2H), 4.05 (q, J=6.8 Hz, 2H), 3.40-3.10 (m, 2H, overlap with H₂O in DMSO), 3.00-2.85 (m, 2H), 2.79 (s, 3H), 1.14 (t, J=6.8 Hz, 3H) ppm. MS (ESI) (M+1)⁺: 574.

### Example 17

### N-[[5-chloro-6-[2-[[(10,11-dihydro-5H-dibenzo[a,d]cyclohepten-5-yl)amino]carbonyl]hydrazino]-3-pyridinyl]sulfonyl]-N-methyl-glycine, Ethyl Ester

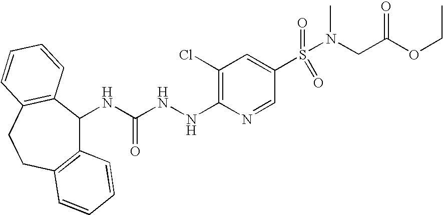

The mixture of N-[(5-chloro-6-hydrazino-3-pyridinyl)sulfonyl]-N-methyl-glycine, ethyl ester from Example 16B (32 mg, 0.10 mmol) and 10,11-dihydro-5-isocyanato-5H-dibenzo[a,d]cycloheptene (28 mg, 0.12 mmol), pyridine (50 μL) in DMA (2 mL) was stirred at room temperature overnight. The solvent was evaporated and the residue was triturated with Et₂O (5 mL). The solid was collected and recrystallized in MeOH twice to the title compound (25 mg, 45%). ¹HNMR (DMSO-d6): δ 9.19 (s, 1H), 8.32 (d, J=2 Hz, 1H), 7.99 (s, 1H), 7.61 (d, J=4 Hz, 1H), 7.59 (d, J=4 Hz, 1H), 7.33 (d, J=7.6 Hz, 2H), 7.20-7.05 (m, 6H), 6.26 (br, 1H), 4.03 (s, 2H), 3.98 (q, J=7.2 Hz, 2H), 3.25-3.05 (m, 4H), 2.76 (s, 3H), 1.12 (t, J=7.2 Hz, 3H) ppm. MS (ESI) (+1)⁺: 558.

### Example 18

### 5-Chloro-6-[2-[[(10,11-dihydro-5H-dibenzo[a,d]cyclohepten-5-yl)amino]thioxomethyl]hydrazino]-N-[(3R)-1-methyl-2-oxo-3-pyrrolidinyl]-3-pyridinecarboxamide

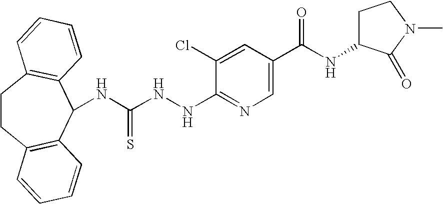

### 18A: [(1R)-1-[(methylamino)carbonyl]-3-(methylthio)propyl]-carbamic Acid, 1,1-dimethylethyl Ester

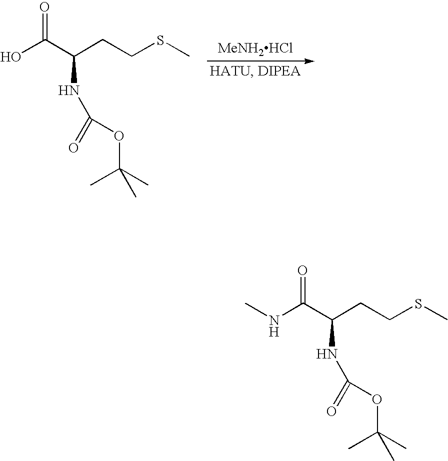

HATU (9.203 g, 24.22 mmol) was added in one portion to a solution of Boc-D-methionine (5.023 g, 20.15 mmol), MeNH₂.HCl (2.035 g, 3014 mmol), and DIPEA (11 ml, 63.8 mmol) in DMF (100 ml) at 0° C. The mixture was then stirred at room temperature for 2 h, and concentrated in vacuo to remove DMF and excess DIPEA, and H₂O (100 ml) was added. The mixture was then extracted with CH₂Cl₂ (2×150 ml), the combined extracts were washed with saturated NaHCO₃ (30 ml) and brine (30 ml), dried over Na₂SO₄. Removal of solvent produced product which was purified by flash chromatography on silica gel pretreated with 1% Et₃N in CH₂Cl₂ (eluent MeOH:CH₂Cl₂=1:30) to give the title compound (3.40 g, 63%).

### 18B: [(3R)-1-methyl-2-oxo-3-pyrrolidinyl]-carbamic Acid, 1,1-dimethylethyl Ester

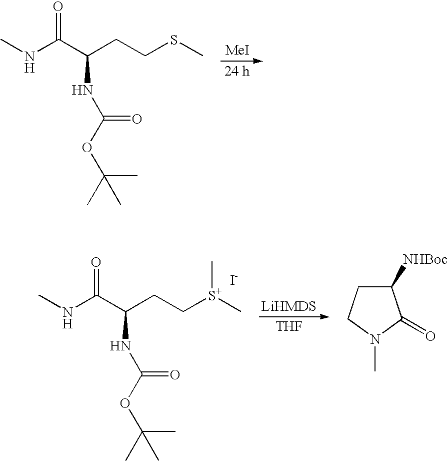

[(1R)-1-[(methylamino)carbonyl]-3-(methylthio)propyl]-carbamic acid, 1,1-dimethylethyl ester from step 18A (1.521 g, 5,80 mmol) was dissolved MeI (20 ml), and the mixture was stirred at room temperature overnight. Excess MeI was removed in vacuo, and the residue was kept under vacuum pump for 2 h to give the intermediate [(3R)-3-[[(1,1-dimethylethoxy)carbonyl]amino]4-(methylamino)-4-oxobutyl]dimethyl-sulfonium, iodide salt (2.65 g, quantitatively). The sulfonium salt (816 mg, 2.02 mmol) was dissolved in anhydrous THF (40 ml), and the solution was cooled to 0° C. in ice-bath. LiHMDS (1M, 2.05 ml, 2.05 mmol) was added dropwise to the above cold solution. The reaction mixture was stirred at room temperature for 2 h, and H₂O (5 ml) was added. The majority of THF was evaporated, and the residue was dissolved CH₂Cl₂ (100 ml) and washed with H₂O (20 ml), the aqueous phase were extracted with CH₂Cl₂ (50 ml). The combined CH₂Cl₂ solution were dried and concentrated in vacuo. The residue was purified on silica gel pretreated with 1% Et₃N in CH₂Cl₂ (eluent MeOH:CH₂Cl₂ 1:25) to afford a light yellow solid. The light yellow solid was washed with Et₂O to produce a white solid (220 mg). ¹HNMR (CDCl₃): δ 5.12 (br, 1H), 4.14 (m, 1H), 3.28-3.38 (m, 2H), 2.89 (s, 3H), 2.60-2.70 (m, 1H), 1.82-1.93 (m, 1H), 1.45 (s, 9H) ppm.

### 18C: (3R)-amino-1-methyl-2-pyrrolidinone Monohydrochloride

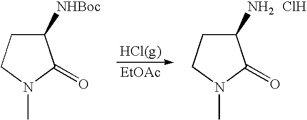

[(3R)-1-methyl-2-oxo-3-pyrrolidinyl]-carbamic acid, 1,1-dimethylethyl ester (220 mg, 1.02 mmol) from Example 18B was dissolved in EtOAc (20 ml), and HCl(g) was bubbled into the solution for 10 min, the mixture was then stirred at room temperature for 2 h, and the solvent was evaporated to give the compound (3R)-amino-1-methyl-2-pyrrolidinone, HCl salt (103 mg).

### 18D: 5-chloro-6-[2-[[(10,11-dihydro-5H-dibenzo[a,d]cyclohepten-5-yl)amino]thioxomethyl]hydrazino]-N-[(3R)-1-methyl-2-oxo-3-pyrrolidinyl]-3-pyridinecarboxamide

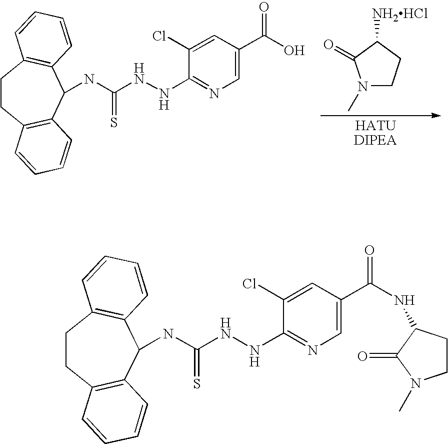

Following the general HATU coupling procedure of Example 1C: the title compound (110 mg, 23%) was prepared from 5-chloro-6-[2-[[(10,11-dihydro-5H-dibenzo[a,d]cyclohepten-5-yl)amino]thioxo-methyl]hydrazino]-3-pyridinecarboxylic acid.DMA (460 mg) and (3R)-amino-1-methyl-2-pyrrolidinone, HCl salt (180 mg). ¹HNMR (DMSO-d6): δ 9.67 (s, 1H), 9.25(brs, 1H), 8.89 (brs, 1H), 8.69 (d, J=6.8 Hz, 1H), 8.57(s, 1H), 8.13 (s, 1H), 7.51 (br, 1H), 7.29 (d, J=6.4 Hz, 2H), 7.20-6.90 (m, 6H), 4.45-4.60 (m, 1H), 3.10-3.40 (m, 4H), 2.96 (m, 2H, 2.75 (s, 3H), 2.31 (br, 1H), 1.90 (m, 1H) ppm. MS (ESI) (M+1)⁺=535.17. Anal. Calcd for C₂₇H₂₇ClN₆O₂S.H₂O: C, 58.63; H, 5.28; N, 15.15. Found: C, 58.76; H, 5.15; N, 14.83.

### Example 19

### 5-chloro-6-[2-[[(3-chloro-10,11-dihydro-5H-dibenzo[a,d]cyclohepten-5-yl)amino]thioxomethyl]hydrazino]-N-[(3R)-1-methyl-2-oxo-3-pyrrolidinyl]-3-pyridinecarboxamide

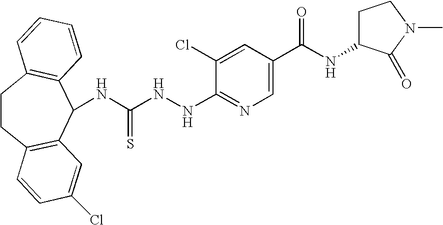

### 19A: 2-[2-(4-chlorophenyl)ethyl]-benzoic Acid

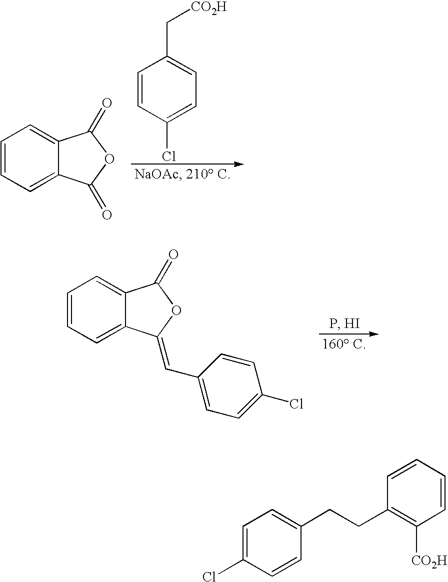

The mixture of Phthalic anhydride (10.80 g, 72.97 mmol) and 4-Cl-phenylacetic acid (9.40 g, 55.10 mmol), NaOAc (0.56 g, 6.83 mmol) was heated at 210-230° C. for 2 h, allowed to cool to 80-90° C., EtOH (50 ml) was added, the solid was collected and washed with EtOH (30 ml), followed by heane/EtOH (9:1, 20 ml), dried to give the intermediate (11.50 g).

The above intermediate (11.50 g) was added to 57% HI (60 ml) and red phosphorus (4.50 g) was added the mixture was then heated at 160-165° C. for 16 h, allowed to cool to room temperature, poured into crushed ice (400 g), then neutralized with 50% KOH to pH˜14. The solid was filtered off, the filtrate was acidified with 12 N HCl to pH˜1, the white solid was collected and dried to give the title compound (6.50 g). MS (ESI) (M+1+CH₃CN)⁺=302.

### 19B: 3-chloro-10,11-dihydro-5H-dibenzo[a,d]cyclohepten-5-one

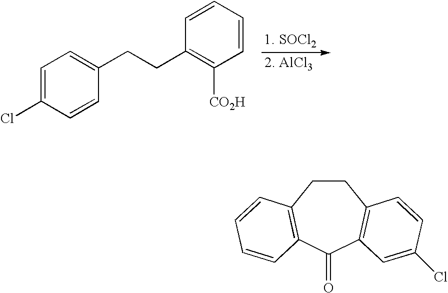

Thionyl chloride (10 ml) was added to a solution of 2-[2-(4-chlorophenyl)ethyl]-benzoic acid (3.50 g) in CH₂Cl₂ (100 ml). The mixture was stirred at reflux overnight, and the solvent and excess SOCl₂ was evaporated in vacuo. The residue was re-dissolved in CH₂Cl₂ (100 ml) and AlCl₃ (4.50 g) was added, the reaction mixture was stirred at room temperature for 3 h, ice water (30 ml) was added. After stirring for another 20 min, the organic phase was separated and washed with H₂O (20 ml), 1N NaOH (2×20 ml), brine (20 ml), dried over Na₂SO₄. The solvent was removed in vacuo and the residue was chromatographed on silica gel (EtOAc: CH₂Cl₂ 1:4) to afford the title compound (2.65 g).

### 19C: 3-chloro-10,11-dihydro-5H-dibenzo[a,d]cyclohepten-5-ol

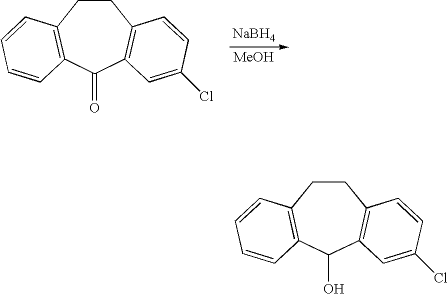

NaBH₄ (86 mg, 2.26 mmol) was added portionwise to a solution of 3-chloro-10,11-dihydro-5H-dibenzo[a,d]cyclohepten-5-one (590 mg, 2.43 mmol) in MeOH (20 ml) at room temperature overnight. MeOH was evaporated and H₂O (20 ml) was added, the solid was collected and washed with H₂O (20 ml), dried to give the title compound (590 mg).

### 19D: 3-chloro-10,11-dihydro-5H-dibenzo[a,d]cyclohepten-5-amine

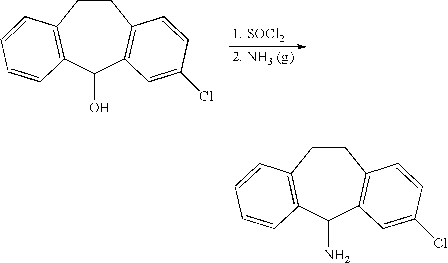

SOCl₂ (1 ml) was added to a solution of 3-chloro-10,11-dihydro-5H-dibenzo[a,d]cyclohepten-5-ol (590 mg, 2.42 mmol) in CH₂Cl₂ (10 ml), and the mixture was heated at reflux for 1 h, and CH₂Cl₂ and excess SOCl₂ was evaporated in vacuo. The residue was re-dissolved in CHCl₃, and NH₃ (g) was bubbled into the solution for 20 min. and the reaction mixture was stirred at room temperature for 2 h. CHCl₃ was evaporated in vacuo and the residue was dissolved in 1N HCl (5 ml) and washed with Et₂O (20 ml). Then the aqueous solution was neutralized with 1 N NaOH to pH˜9-10, extracted with Et₂O (50 ml), and the Et₂O solution was washed brine (10 ml), dried over Na₂SO₄ and concentrated in vacuo to produce the title compound (281 mg, 48%).

### 19E: 3-chloro-10,11-dihydro-5-isothiocyanato-5H-dibenzo[a,d]cycloheptene

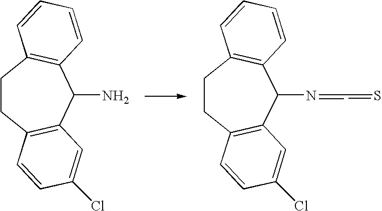

CSCl₂ (0.5 ml) was added in one portion to a vigorously stirred solution of 3-chloro-10,11-dihydro-5H-dibenzo[a,d]cyclohepten-5-amine (281 mg) in CH₂Cl₂ (15 ml) and saturated NaHCO₃ (5 ml) at room temperature, the mixture was stirred at room temperature for 2 h and diluted with CH₂Cl₂ (30 ml), then the organic phase was separated and washed with brine (10 ml), dried over Na₂SO₄, and concentrated in vacuo to give the title compound (246 mg, 75%).

### 19F: 5-chloro-6-[2-[[(3-chloro-10,11-dihydro-5H-dibenzo[a,d]cyclohepten-5-yl)amino]thioxomethyl]hydrazino]-3-pyridinecarboxylic Acid

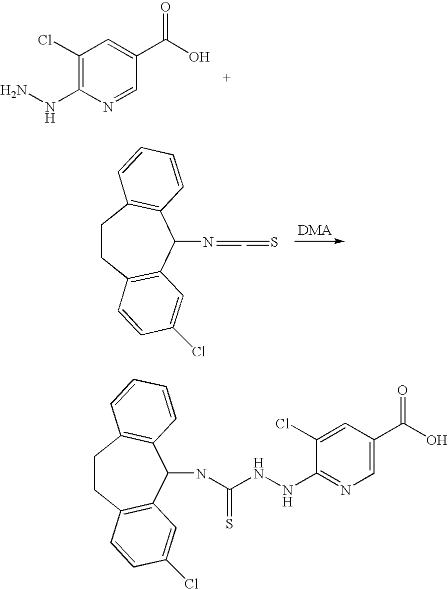

A mixture of 5-chloro-6-hydrazino-3-pyridinecarboxylic acid (37 ml, 0.20 mmol) and 3-chloro-10,11-dihydro-5-isothiocyanato-5H-dibenzo[a,d]cycloheptene (58 mg, 0.20 mmol) in DMA (3 ml) was stirred at room temperature overnight. The title compound (0.20 mmol) as its DMA solution was ready for use.

### 19G: 5-chloro-6-[2-[[(3-chloro-10,11-dihydro-5H-dibenzo[a,d]cyclohepten-5-yl)amino]thioxomethyl]hydrazino]-N-[(3R)-1-methyl-2-oxo-3-pyrrolidinyl]-3-pyridinecarboxamide

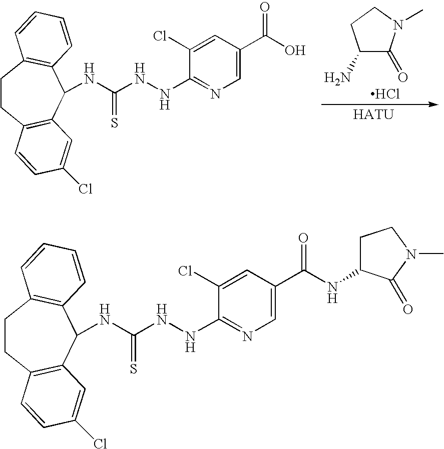 (3R)-amino-1-methyl-2-pyrrolidinone, monohydrochloride (33 mg, 0.22 mmol) from Example 18C, followed by DIPEA (0.2 ml) and HATU (5 mg, 0.25 mmol) was added the solution of 5-chloro-6-[2-[[(3-chloro-10,11-dihydro-5H-dibenzo[a,d]cyclohepten-5-yl)amino]thioxomethyl]hydrazino]-3-pyridinecarboxylic acid (0.20 mmol) in DMA (3 ml) from Example 19F. The reaction mixture was stirred at room temperature for 2 h and then H₂O (10 ml) was added, the precipitate was collected and recrystallized from MeOH to produce the title compound (56 mg). ¹HNMR (DMSO-d6): δ 9.74 (s, 1H), 9.31 (s, 1H), 9.18 (d, J=9.2 Hz, 1H), 8.73 (d, J=8.2 Hz, 1H), 8.63 (s, 1H), 8.16 (s, 1H), 7.67 (d, J=8.2 Hz, 1H), 7.25-7.45 (m, 2H), 5.95-7.25 (m, 5H), 4.50-4.65 (m, 1H), 3.34 (br, 4H), 2.95-3.00 (m, 2H), 2.76 (s, 3H), 2.24-2.39 (m, 1H), 1.83-1.89 (m, 1H) ppm. MS(ESI)(M+1)⁺=569. HRMS(M+1)+ Calcd for C₂₇H₂₆Cl₂N₆O₂S: 569.1293; Found: 569.1205.

### Example 20

### 5-chloro-6-[2-[[(6,11-dihydrodibenzo[b,e]thiepin-11-yl)amino]thioxomethyl]hydrazino]-N-[(3R)-tetrahydro-2-oxo-3-thienyl]-3-pyridinecarboxamide

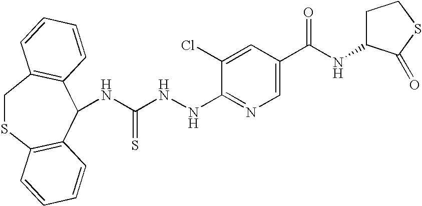

### 20A: 6,11-dihydro-dibenzo[b,e]thiepin-11-amine

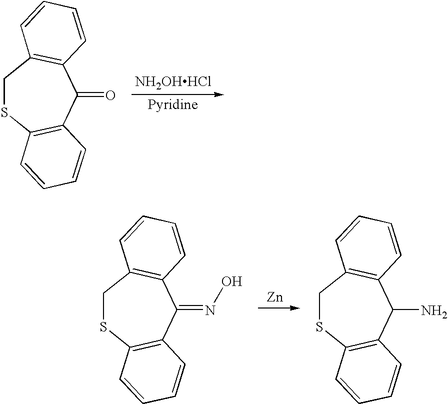

NH₂OH.HCl (1.20 g) was added to a solution of dibenzo[b,e]thiepin-[(6H)-one (1.20 g) in pyridine (20 ml), and the mixture was heated at reflux for 3 days, allowed to cool to room temperature, and pyridine was evaporated, and the residue was dissolved in CH₂Cl₂ (100 ml), and washed with 2N HCl (2×20 ml), saturated NaHCO₃ (20 ml), brine, dried over Na₂SO₄. Evaporation of solvent gave the oxime intermediate (605 mg).

The oxime intermediate (520 mg) was dissolved in EtOH (20 ml), and 5N NH₄OH (20 ml), NH₄OAc and Zn (1.10 g) were added. The reaction mixture was heated at reflux overnight, allowed to cool to room temperature, diluted with EtOH (100 ml), and then filtered through celite. The solvent was evaporated and the residue was dissolved in Et₂O (200 ml), washed with 1N NaOH (20 ml), brine, dried over Na₂SO₄. The solvent was removed the in vacuo and the crude product was re-dissoved in Et₂O (50 ml), and 1 N HCl in Et₂O was added until no more precipitae was formed. The solid was collected to give the title compound (260 mg) as its HCl salt.

### 20B: 6,11-dihydro-11-isothiocyanato-dibenzo[b,e]thiepin

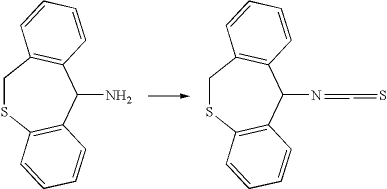

Following the general procedure of Example 19E: CSCl₂ (0.15 ml) was added in one portion to a vigorously stirred solution of 6,11-dihydro-dibenzo[b,e]thiepin-11-amine, HCl salt (86 mg) in CH₂Cl₂ (5 ml) and saturated NaHCO₃ (5 ml) at room temperature, the mixture was stirred at room temperature for 2 h and diluted with CH₂Cl₂ (20 ml), then the organic phase was separated and washed with brine (10 ml), dried over Na₂SO₄, and concentrated in vacuo to give the title compound (91 mg).

### 20C: 5-chloro-6-[2-[[(6,11-dihydrodibenzo[b,e]thiepin-11-yl)amino]thioxomethyl]hydrazino]-3-pyridinecarboxylic Acid

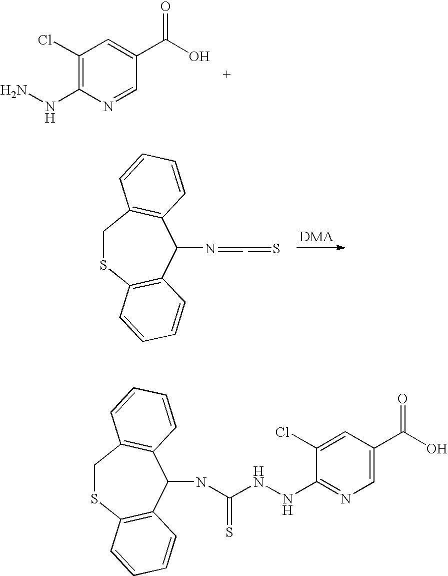

A mixture of 5-chloro-6-hydrazino-3-pyridinecarboxylic acid (37 ml, 0.20 mmol) and 6,11-dihydro-11-isothiocyanato-dibenzo[b,e]thiepin (53 mg, 0.20 mmol), in DMA (3 ml) was stirred at room temperature overnight. The title compound (0.20 mmol) as its DMA solution was ready for use.

### 20D: 5-chloro-6-[2-[[(6,11-dihydrodibenzo[b,e]thiepin-11-yl)amino]thioxomethyl]hydrazino]-N-[(3R)-tetrahydro-2-oxo-3-thienyl]-3-pyridinecarboxamide

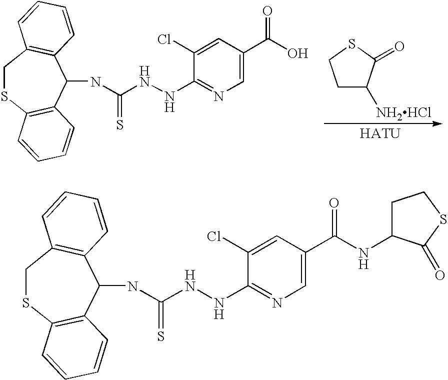

Following the general HATU coupling procedure of Example 1C: the title compound (30 mg) was prepared from 5-chloro-6-[2-[[(6,11-dihydrodibenzo[b,e]thiepin-11-yl)amino]thioxomethyl]hydrazino]-3-pyridinecarboxylic acid (0.1 mmol) and racemic homocysteine thiolactone hydrochloride (17 mg). ¹HNMR(DMSO-d6): δ 9.78 (s, 1H), 9.29 (s, 1H), 8.87 (m, 1H), 8.71 (m, 1H), 8.49 (s, 1H), 8.14 (s, 1H), 7.00-7.60 (m, 9H), 4.75-4.85 (m, 1H), 4.68 (d, J=14.1 Hz, 1H), 4.02 (d, J=14.1 Hz, 1H), 3.35-3.50 (m, 1H), 3.25-3.35 (m, 1H), 2.40-2.50 (m, 1H), 2.15-2.30 (m, 1H) ppm. MS(M+1)⁺=556. HRMS (M+1)⁺ Calcd for C₂₅H₂₂ClN₅O₂S₃: 556.0702. Found: 556.0795.

### Example 21

### 5-Chloro-6-[2-[[(6,11-dihydro-6-methyl-5,5-dioxidodibenzo[c,f][1,2]thiazepin-11-yl)amino]thioxomethyl]hydrazino]-N-[(3R)-tetrahydro-2-oxo-3-thienyl]-3-pyridinecarboxamide

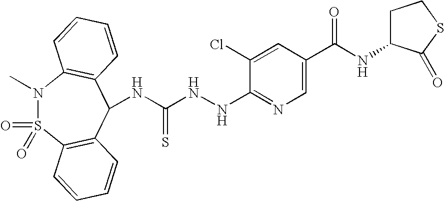

### 21A: 6,11-Dihydro-6-methyl-5,5-dioxide-dibenzo[c,f][1,2]thiazepin-11-ol

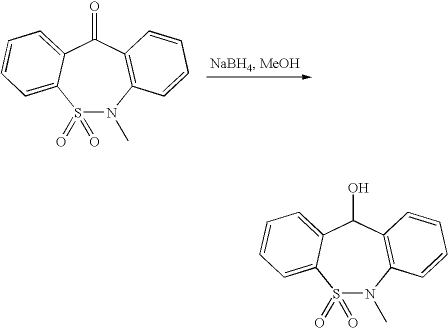

NaBH₄ (0.5 g, 13.16 mmol) was added to a solution of 6-methyl-dibenzo[c,f][1,2]thiazepin-11(6H)-one, 5,5-dioxide (1.5 g, 5.49 mmol) (prepared according to the method described in Abramovitch, R. A., Azogu, C. I., McMaster, I. T. and Vanderpool, D. P., J. Org. Chem., 1978, 43, 1218, which is incorporated by reference herein for its disclosure in the preparation of 6-methyl-dibenzo[c,f][1,2]thiazepin-11(6H)-one, 5,5-dioxide) in MeOH/CH₂Cl₂ (50/15 ml) and the reaction was stirred for 2 hours. The solvents were then removed under reduced pressure and the residue was diluted in CH₂Cl₂ and was washed with saturated NaHCO₃, water, and brine, dried and concentrated under reduced pressure to provide alcohol which was used directly for the next step (1.5 g). ¹H NMR (400 MHz, CDCl₃): δ 7.98 (d, J=7.6 Hz, 1H), 7.70-7.58 (m, 3H), 7.55-7.49 (m, 1H), 7.42-7.25 (m, 3H), 5.91 (d, J=10.3 Hz, 1H), 4.42 (d, J=10.3 Hz, 1H), 3.13 (s, 3H) ppm. MS (ESI (M+1)⁺: 276.

### 21B: 6,11-Dihydro-6-methyl-5,5-dioxide-dibenzo[c,f][1,2]thiazepin-11-amine

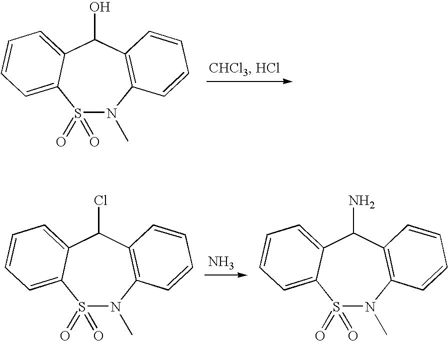

Following the same procedure of Example 19D: the title compound (200 mg, 13% in two steps) was prepared from 6,11-dihydro-6-methyl-5,5-dioxide-dibenzo[c,f][1,2]thiazepin-11-ol (1.5 g, 5.49 mmol). ¹H NMR (400 MHz, CDCl₃): δ 7.98 (d, J=7.8 Hz, 1H), 7.62-7.51 (m, 2H), 7.49 (d, J=7.4 Hz, 1H), 7.44 (t, J=7.2 Hz, 1H), 7.38-7.24 (m, 3H), 5.45 (s, 1H), 3.26 (s, 3H), 2.23 (bs, 2) ppm. MS (ESI) (+1)⁺: 275.

### 21C: 6,11-Dihydro-11-isothiocyanato-6-methyl-5,5-dioxide-dibenzo[c,f][1,2]thiazepine

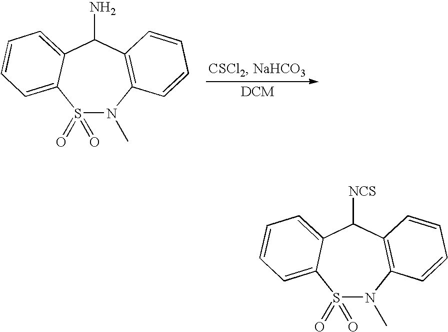

Following the general procedure of Example 19E: To a virgorously stirred solution of 6,11-dihydro-6-methyl-5,5-dioxide-dibenzo[c,f][1,2]thiazepin-11-amine (0.16 g, 0.58 mmol) in CH₂Cl₂ (8.7 ml) and saturated NaHCO₃ solution (8.7 ml) was added thiophosgene (0.29 mL, 3.8 mmol) in one portion at room temperature. The mixture was stirred for 2 hours and then was diluted in EtOAc. Aqueous phase was then separated and the organic phase was washed several times with saturated NaHCO₃ and then with brine, dried and concentrated under reduced pressure. The product was directly used for the next step.

### 21D: 5-Chloro-6-[2-[[(6,11-dihydro-6-methyl-5,5-dioxidodibenzo[c,f][1,2]thiazepin-11-yl)amino]thioxomethyl]hydrazino]-3-pyridinecarboxylic Acid

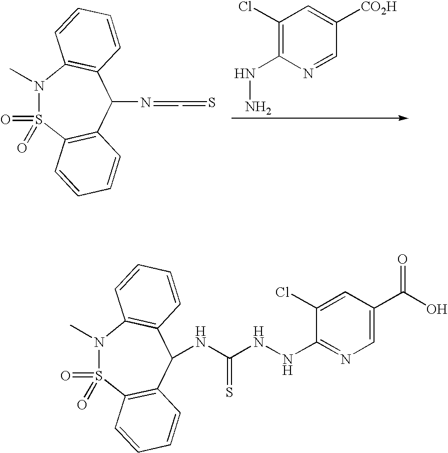

5-Chloro-6-hydrazino-3-pyridinecarboxylic acid (115 mg, 0.62 mmols, 1.1 eq) was added to a solution of 6,11-dihydro-11-isothiocyanato-6-methyl-5,5-dioxide-dibenzo[c,f][1,2]thiazepine (0.58 mmols, 1 eq.) in DMA (3 mL). The reaction was stirred overnight and then concentrated under reduced pressure. The residue was diluted in CH₂Cl₂ and was washed with saturated NaHCO₃, water and brine, dried and concentrated. The product was then used directly for the next step. MS (ESI) (M+1)⁺: 504.

### 21E: 5-Chloro-6-[2-[[(6,11-dihydro-6-methyl-5,5-dioxidodibenzo[c,f][1,2]thiazepin-11-yl)amino]thioxomethyl]hydrazino]-N-[(3R)-tetrahydro-2-oxo-3-thienyl]-3-pyridinecarboxamide

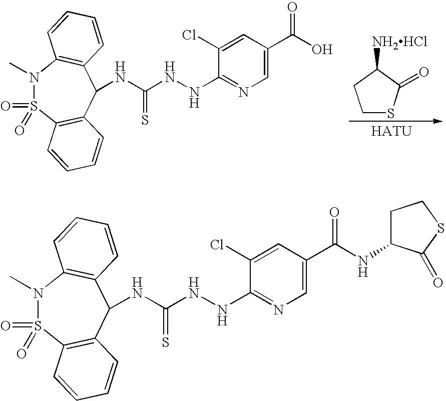

Following the general HATU coupling procedure of Example 1C: HATU (27 mg, 0.7 mmol) was added to a mixture of D-homocysteine thiolactone.HCl (11 mg, 0.07 mmol), 5-chloro-6-[2-[[(6,11-dihydro-6-methyl-5,5-dioxidodibenzo[c,f][1,2]thiazepin-11-yl)amino]thioxomethyl]hydrazino]-3-pyridinecarboxylic acid (30 mg, 0.06 mmol), and DIPEA (25 μL, 0.14 mmol) in DMA (3 ml). The mixture was stirred at room temperature for 2 hours and then DMA was removed under reduced pressure. The residue was diluted in CH₂Cl₂ and was washed successively with saturated NaHCO₃, water and brine, dried and concentrated. The product was then purified by flash chromatography (CH₂Cl₂-MeOH) to provide the title compound (11 mg, 31%). ¹H NMR (400 MHz, CD₃OD): δ 8.99 (bd, J=7.6 Hz, 2H), 8.23 (d, J=1.95 Hz, 1H), 8.16 (d, J=1.95 Hz, 1H), 8.00 (dd, J=7.62 and 2.93 Hz, 2H), 7.95 (dd, J=5.1 and 1.95 Hz, 2H), 7.92-7.84 (m, 2H), 7.82-7.76 (m, 2H), 7.62 (t, J=8.3 Hz, 2H), 7.59-7.49 (m, 2H), 7.45 (dd, J=9.7 and 3.4 Hz, 2H), 7.37-7.30 (m, 2H), 7.29-7.21 (m, 2H), 7.16 (dd, J=7.4 and 5.08 Hz, 2H), 6.86-6.74 (m, 2H), 4.74-4.66 (m, 2H), 3.48-3.29 (m, 4H), 2.96 (s, 3H). MS (ESI) (M+1)⁺: 603. HRMS(M+1)+ Calcd for C25H23ClN6O4S3: 603.0710; Found: 603.0806.

### Example 22

### 5-Chloro-6-[2-[[(10,11-dihydro-5H-benzo[4,5]cyclohepta[1,2-c]pyridin-5-yl)amino]carbonyl]hydrazino]-N-[(3R)-1-methyl-2-oxo-3-pyrrolidinyl]-3-pyridinecarboxamide

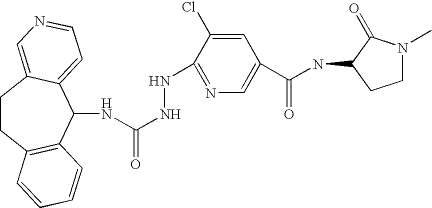

### 22A: 4-Methyl-3-(phenylethynyl)-pyridine

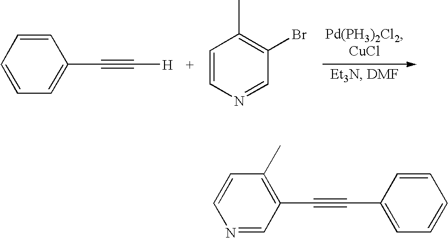

All the liquid reagents were degassed with argon for 15-20 minutes. A mixture of phenylacetylene (110 μL, 1 mmol, 3.6 eq.), Pd(Ph₃)₂Cl₂ (24 mg, 0.03 mmol), CuCl (14 mg, 0.14 mmol) and Et₃N (1 mL, 7.1 mmol) in DMF (1 ml) was stirred 15 minutes at room temperature. The yellow solution was then treated drop by drop with a solution of 3-bromo-4-methyl-pyridine (30 μL, 0.28 mmol) in DMF (0.5 ml) and then refluxed at 150° C. for 3 hours. The resulting brown solution was cooled and concentrated under reduced pressure then diluted in CH₂Cl₂ and washed successively with saturated aqueous NaHCO₃, water, and brine, dried and concentrated under reduced pressure. The product was purified by column chromatography (ether-hexane) to obtain the title compound (35 mg, 65%). ¹H NMR (400 MHz, CDCl₃): δ 8.69 (bs, 1H), 8.38 (bs, 1H), 7.57-7.51 (m, 2H), 7.39-7.33 (m, 3H), 7.16 (bs, 1H), 2.50 (s, 3H) ppm. MS (ESI) (M+1)⁺: 194.

### 22B: 4-Methyl-3-(2-phenylethyl)-pyridine

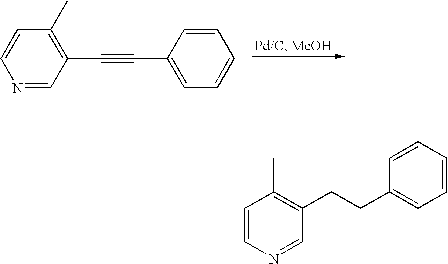

To a solution of 4-methyl-3-phenylethynyl)-pyridine in MeOH was added Pd/C and the mixture was stirred at room temperature for two days. The catalyst was then removed by filtration and the mixture concentrated under reduced pressure. The obtained product was directly used for the next step. ¹H NMR (400 MHz, CDCl₃): δ 8.30 (bs, 2H), 7.31-7.14 (m, 3H), 7.15 (d, J=7.2 Hz, 2H), 7.03 (bd, J=4.1 Hz, 1H), 2.95-2.80 (M, 4H), 2.23 (s, 3H) ppm. MS (ESI) (M+1)⁺: 198.

### 22C: 3-(2-Phenylethyl)-4-pyridinecarboxylic Acid

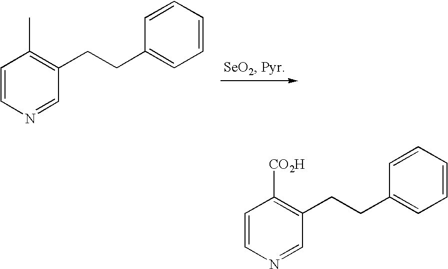

Following similar method reported in literature Villani, F. J., Daniels, P. J. L., Ellis, C. A., Mann, T. A., Wang, K.-C. J. Heterocyl. Chem. 1971, 73-78: SeO2 (4.8 g, 43.24 mmol) was added to a solution of 4-methyl-3-(2-phenylethyl)-pyridine (3.28 g, 16.62 mmol) in pyridine (21 mL) and the mixture was heated with stirring under reflux for 3 hours. Chloroform was then added and the mixture was filtered and then concentrated under reduced pressure. The residue was dissolved in dilute ammonium hydroxide and extracted with ether. The organic phase was discarded and the aq. phase was acidified with acetic acid and the product was allowed to crystallize. The acid was collected by filtration (2.3 g, 61/o). ¹H NMR (400 MHz, CD₃OD): δ 8.37 (d, J=5.1 Hz, 1H), 8.23 (s, 1H), 7.66 (d, J=5.3 Hz, 1H), 7.17-7.01 (m, 5H), 3.22-3.10 (m, 2H), 2.84-2.76 (m, 2H) ppm. MS (ESI) (M+1)⁺: 228.

### 22D: 10,11-Dihydro-5H-benzo[4,5]cyclohepta[1,2-c]pyridin-5-one

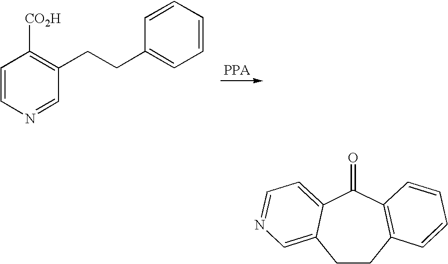

PPA (96 g) was added to the 3-(2-phenylethyl)-4-pyridinecarboxylic acid (2.3 g, 10.12 mmol) and the mixture was heated at 170° C. for 2 hours. The solution was cooled to about 60° C. and ice water was added with stirring. The mixture was then made basic by the gradual addition of solid NaOH and the aq. phase was extracted several times with AcOEt. The combined organic layers were washed with brine, dried and concentrated under reduced pressure to obtain the product which was purified by a short chromatography column to provide the title compound (1.9 g, 90%). ¹H NMR (400 MHz, CDCl₃): δ 8.63-8.56 (m, 2H), 7.99 (d, J=8.0 Hz, 1H), 7.79 (d, J=5.1 Hz, 1H), 7.46 (t, J=7.1 Hz, 1H), 7.33 (t, J=7.6 Hz, 1H), 7.24 (d, J=7.6 Hz, 1H), 3.25-3.15 (m, 4H) ppm. MS (ESI) (M+1)⁺: 210.

### 22E: 10,11-Dihydro-oxime, (5E)-5H-benzo[4,5]cyclohepta[1,2-c]pyridin-5-one

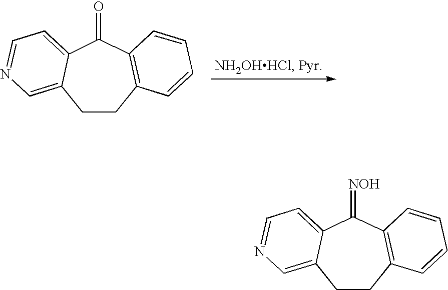

To a solution of 10,11-dihydro-5H-benzo[4,5]cyclohepta[1,2-c]pyridin-5-one (1.6 g, 7.65 mmol) in 70 mL pyridine was added hydroxylamine hydrochloride (3.5 g, 50.72 mmol) at rt. The mixture was then refluxed for 3 hours. More hydroxylamine hydrochloride 1.1 g (15.94 mmol) was added and the heating was continued overnight. The mixture was then cooled and condensed under reduced pressure. Saturated NaHCO₃ was then added and the aq. phase was extracted several times with CH₂Cl₂. The combined organic layers were washed with brine and then concentrated under reduced pressure to provide the oxime intermediate (1.40 g) which was used directly for the next step. MS (ESI) (M+1)⁺: 225.

### 22F: 10,11-Dihydro-5H-benzo[4,5]cyclohepta[1,2-c]pyridin-5-amine

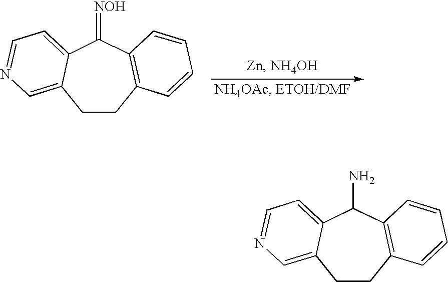

A solution of 10,11-dihydro-5H-benzo[4,5]cyclohepta[1,2-c]pyridin-5-one oxime (1.4 g, 6.25 mmol) in EtOH/DMF (42/7 mL) was treated with Zn (2.1 g, 32.30 mmol), NH₄OH (28 mL) and NH₄OAc (0.46 g, 5.97 mmol). The mixture was refluxed for one hour and then cooled to the room temperature and concentrated under reduced pressure. The residue was then diluted in CH₂Cl₂ and washed with a saturated solution of NaHCO₃. The aqeous phase was extracted several times with CH₂Cl₂. The combined organic phases were washed with brine, dried and concentrated under reduced pressure to provide 1.3 g of amine which was directly used for the next step. ¹H NMR (400 MHz, CDCl₃): δ 8.26 (bd, J=4.7, 1H), 8.16 (s, 1H), 7.30 (dd, J=5.2, 3.6 Hz, 1H), 7.26 (d, J=5.1 Hz, 1H), 7.14-7.03 (m, 3H), 5.43 (s, 1H), 3.25-3.15 (m, 2H), 3.15-3.07 (m, 2H), 1.54 (bs, 2H) ppm. MS (ESI) (M+1)⁺: 211.

### 22G: 5-Chloro-6-[2-[[(10,11-dihydro-5H-benzo[4,5]cyclohepta[1,2-c]pyridin-5-yl)amino]carbonyl]hydrazino]-3-pyridinecarboxylic Acid

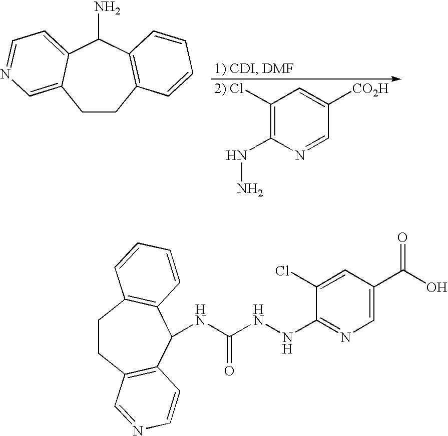

A solution of 10,11-dihydro-5H-benzo[4,5]cyclohepta[1,2-c]pyridin-S-amine (22 mg, 0.1 mmol) in DMF (0.7 ml) was added drop by drop to a stirred solution of CDI (16 mg, 0.1 mmol) in DMF (1 ml) at 0° C. and then allowed to warm to room temperature with stirring. After all the amine was consumed, 5-chloro-6-hydrazino-3-pyridinecarboxylic acid was added and stirring was continued for 2 hours. The mixture was then concentrated under reduced pressure and then diluted in CH₂Cl₂, washed with saturated NaHCO₃, water and brine, and then dried and concentrated. The dark-yellow residue was trituated with MeOH. The solid was collected and used directly for the next step. MS (ESI) (M+1)⁺: 424.

### 22H: 5-Chloro-6-[2-r[(10,11-dihydro-5H-benzo[4,5]cyclohepta[1,2-c]pyridin-5-yl)amino]carbonyl]hydrazino]-N-[(3R)-1-methyl-2-oxo-3-pyrrolidinyl]-3-pyridinecarboxamide

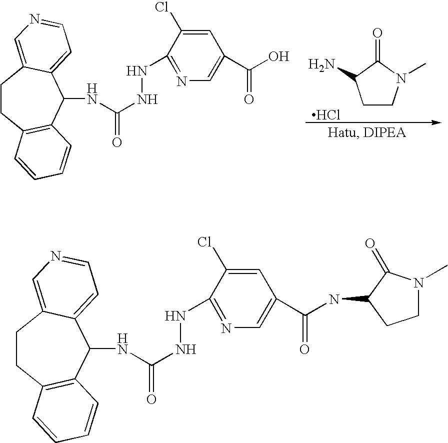

HATU (29 mg, 0.07 mmol) was added to a mixture 5-chloro-6-[2-[[(10,11-dihydro-5H-benzo[4,5]cyclohepta[1,2-c]pyridin-5-yl)amino]carbonyl]hydrazino]-3-pyridinecarboxylic acid (25 mg, 0.06 mmol), (3R)-3-amino-1-methyl-2-pyrrolidinone, monohydrochloride (11 mg, 0.07 mmol) and DIPEA (24 μL, 0.14 mmol) in 2 mL DMA. The mixture was stirred at room temperature for 2 hours and then DMA was removed under reduced pressure. The residue was diluted in CH₂Cl₂ and washed successively with saturated NaHCO₃, water, brine, dried and concentrated. The residue was then purified by flash chromatography (CH₂Cl₂-MeOH) to provide the title compound (6 mg). ¹H NMR (400 MHz, CD₃OD): 8.44 (s, 1H), 8.15 (bs, 2H), 7.98 (s, 1H), 7.37 (d, J=4.9 Hz, 1H), 7.25 (d, J=7.0 Hz, 1H), 7.15-7.01 (m, 2H), 6.41 (s, 1H), 4.59-4.49 (m, 1H), 3.35 (dd, J=9.0, 4.7 Hz, 3H), 3.30-2.97 (m, 3H), 2.79 (s, 3H), 2.42-2.32 (m, 1H), 2.02-1.87 (m, 1H), 1.20-1.10 (m, 1H) ppm. MS (ESI) (M+1)⁺: 520.

### Example 23

### 5-chloro-6-[2-[[(10,11-dihydro-5H-benzo[4,5]cyclohepta[1,2-c]pyridin-5-yl)amino]carbonyl]hydrazino]-N-[(3R)-tetrahydro-2-oxo-3-thienyl]-3-pyridinecarboxamide

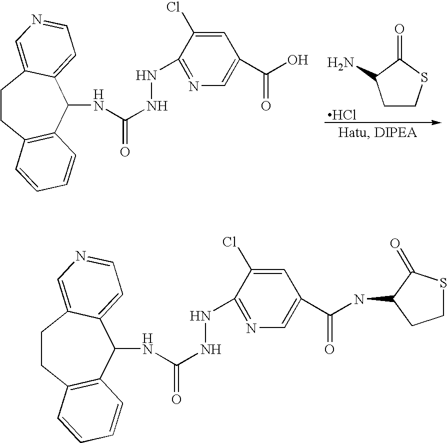

Following the general HATU coupling procedure of Example 1C: HATU (35 mg, 0.09 mmol) was added to a mixture of 5-Chloro-6-[2-[[(10,11-dihydro-5H-benzo [4,5]cyclohepta[1,2-c]pyridin-5-yl)amino]carbonyl]hydrazino]-3-pyridinecarboxylic acid (0.08 mmol), D-homocysteine thiolactone.HCl (20 mg, 0.13 mmol) and DIEPA (31 μL, 0.18 mmol) in 2 mL DMA. The mixture was stirred at rt for 2 hours and then DMA was removed under reduced pressure. The residue was then diluted in CH₂Cl₂ and washed with a saturated solution of NaHCO₃, water and a saturated solution of NaCl, dried and concentrated. The product was then purified by column chromatography (CH₂Cl₂-MeOH) to provide the title compound (6 mg). ¹H NMR (400 MHz, CD₃OD) δ: 8.44 (s, 1H), 8.23-8.13 (m, 2H), 8.00 (s, 1H), 7.37 (d, J=5.5 Hz, 1H), 7.25 (d, J=7.0 Hz, 1H), 7.14-7.02 (m, 2H), 6.41 (s, 1H), 4.59-4.49 (m, 1H), 3.35-2.95 (m, 6H), 2.57-2.47 (m, 1H), 2.25-2.12 (m, 1H), 1.20 (t, J=7.3 Hz, 1H) ppm. MS (ESI) (M+1)⁺: 523. HRMS(M+1)+ Calcd for C₂₅H₂₃ClN₆O₃S: 532.1319; Found: 523.1417.

### Example 24

### 5-chloro-6-[2-[[(6,11-dihydro-5H-benzo[5,6]cyclohepta[1,2-c]pyridin-11-yl)amino-3-thioxomethyl]hydrazino]-N-[(3R)-1-methyl-2-oxo-3-pyrrolidinyl]-3-pyridinecarboxamide

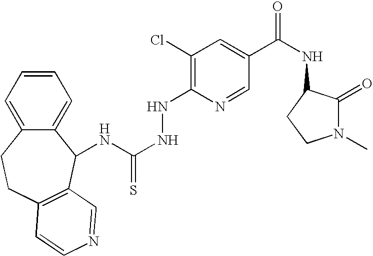

### 24A: 5,6-dihydro-11H-benzo[5,6]cyclohepta[1,2-c]pyridin-11-one, Oxime

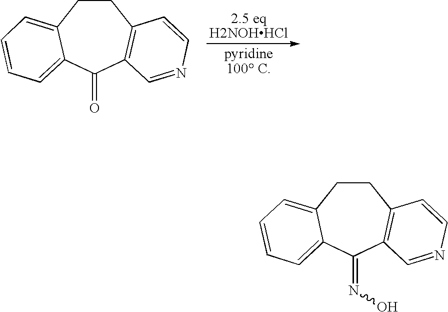

To a stirred solution of 5,6-dihydro-11H-benzo[5,6]cyclohepta[1,2-c]pyridin-1-one (2.7 g, 12.9 mmol) (prepared according to literature method described in Villani F. J., et al, J. Heterocyclic Chem. 1971, 8, 73, which is incorporated by reference herein for its disclosure of the preparation of 5,6-dihydro-1H-benzo[5,6]cyclohepta[1,2-c]pyridin-11-one) in pyridine (30 mL) at 100° C. was added NH₂OH.HCl (32.25 mmol, 2.24 g) and the resulting mixture was stirred at the same temperature overnight. After the reaction was completed, pyridine was removed in vacuo, the residue was added water and the precipitate was collected by filtration, washed with water, dried to give product as pale yellow powder 2.74 g (yield 95%), which was used without further purification. MS (ESI) (M+H)⁺=225.

### 24B: 6,11-dihydro-5H-benzo[5,6]cyclohepta[1,2-c]pyridin-11-amine

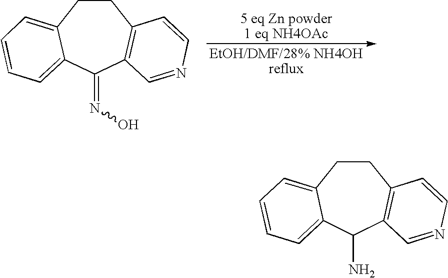

To a mixture of 5,6-dihydro-11H-benzo[5,6]cyclohepta[1,2-c]pyridin-1]-one oxime (2.24 g, 10 mmol) in EtOH (60 mL), DMF (10 mL) and 28% NH₄OH (40 mL) was added Zn powder (3.25 g, 50 mmol) followed by NH₄OAc (0.77 g, 10 mmol). The mixture was stirred at reflux for 3 h. After cooled to room temperature, Et₂O (200 mL) was added and stirred for 20 min, then 1N NaOH (50 mL) was added and stirred for another 10 min. After filtered through Celite, the organic layer was isolated; and the basic aqueous solution was extracted with Et₂O (3×50 mL). The combined organic layer was washed with water and then brine, dried over Na₂SO₄, evaporated to give white solid 1.93 g (92%), which can be used without further purification. MS (ESI) (M+H)⁺=211.

### 24C: 6,11-dihydro-11-isothiocyanato-5H-benzo[5,6]cyclohepta[1,2-c]pyridine

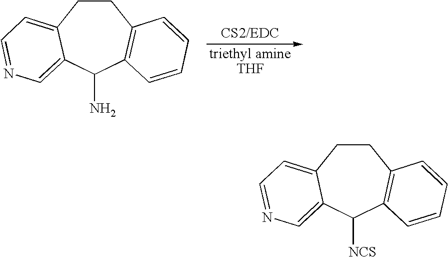

To a solution of 6,11-dihydro-5H-benzo[5,6]cyclohepta[1,2-c]pyridin-11-amine (210 mg, 1.0 mmol) in dry THF (12 mL) was added CS₂ (0.60 ml, 10 mmol) at −10° C., then EDC (383 mg, 2.0 mmol) was added in one portion. After being stirred for 15 minutes, TEA (279 μL, 2.0 mmol) was added dropwise. The mixture was allowed to slowly warm up to room temperature and stirred for 4 hours. After filtration, the filtrate was concentrated and the residue was taken up in ethyl acetate (20 mL), extracted with saturated NaHCO₃ (1×10 mL), water (1×10 mL) and then brine (1×10 mL), dried over Na₂SO₄, then concentrated in vacuo. The residue was purified by passing through a short pad of silica gel (30% EtOAc in DCM) to give the title compound as a pale yellow oil (238 mg, 94%). MS (ESI) (M+H)⁺=253.

### 24D: 5-chloro-6-[2-[[(6,11-dihydro-5H-benzo[5,6]cyclohepta[1,2-c]pyridin-11-yl)amino]thioxomethyl]hydrazino]-3-pyridinecarboxylic Acid

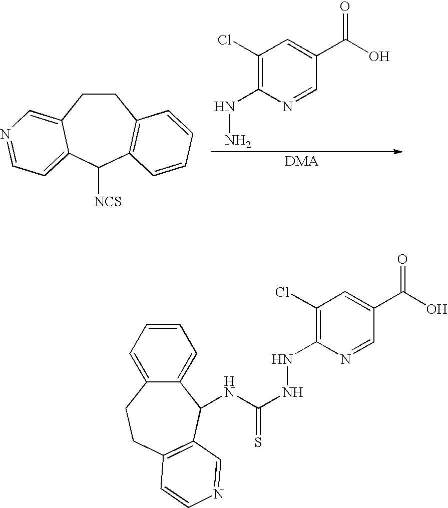

To a solution of 6,11-dihydro-11-isothiocyanato-5H-benzo[5,6]cyclohepta[1,2-c]pyridine (138 mg, 0.54 mmol) in DMA (3 mL) was added 5-chloro-6-hydrazino-3-pyridinecarboxylic acid (102 mg, 0.54 mmol), the mixture was stirred at room temperature for 2 hours, the solvent was concentrated in vacuo and water was added, the precipitate was collected by filtration, dried to give the title compound as off-white powders (225 mg, 94%). MS (ESI) (M+H)⁺=440.

### 24E: 5-chloro-6-[2-[[(6,11-dihydro-5H-benzo[5,6]cyclohepta[1,2-c]pyridin-11-yl)amino]thioxomethyl]hydrazino]-N-[(3R)-1-methyl-2-oxo-3-pyrrolidinyl]-3-pyridinecarboxamide

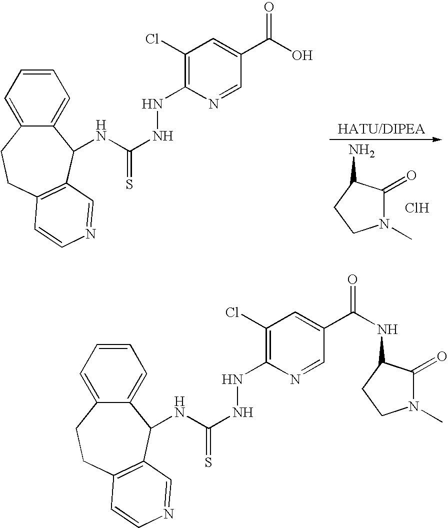

To a stirred solution of 5-chloro-6-[2-[[(6,11-dihydro-5H-benzo[5,6]cyclohepta[1,2-c]pyridin-11-yl)amino]thioxomethyl]hydrazino]-3-pyridinecarboxylic acid (265 mg, 0.6 mmol) in DMA (5 mL) was added HATU (276 mg, 0.72 mmol) followed by DIPEA (124 μL, 0.72 mmol). The mixture was stirred at room temperature for 10 min, and then (3R)-3-amino-1-methyl-2-pyrrolidinone hydrochloride (108 mg, 0.72 mmol) was added. The resulting mixture was stirred at room temperature for 4 h. The product mixture was concentrated in vacuo, the residue was taken up in DCM (50 mL), and washed with conc. NaHCO₃ (2×20 mL) and then brine (1×20 mL). The residue obtained after removal of the solvent was purified by flash chromatography (5˜10% MeOH in DCM) to provide the title compound (106 mg, 33%). ¹H-NMR (400 MHz, CD₃OD): δ 8.54(s, 1H), 8.52-8.46 (m, 1H), 8.19 (d, J=5 Hz, 1H), 8.06-7.98(m, 1H), 7.44-7.24(m, 2H), 7.23-7.05 (m, 4H), 4.73-4.56(m, 1H), 3.48-3.38 (m, 1H), 3.31-3.01(m, 4H), 2.85 (s, 3H), 2.55-2.36 (m, 1H), 2.13-1.95 (m, 1H) ppm. MS (ESI) (M+1)⁺=536. HRMS (M+H)⁺ Calcd for C₂₆H₂₆ClN₇O₂S: 536.1635; Found: 536.1736.

### Example 25

### 5-chloro-6-[2-[[(6,11-dihydro-5H-benzo[5,6]cyclohepta[1,2-c]pyridin-11-yl)amino]thioxomethyl]hydrazino]-N-[(3R)-tetrahydro-2-oxo-3-thienyl]-3-pyridinecarboxamide

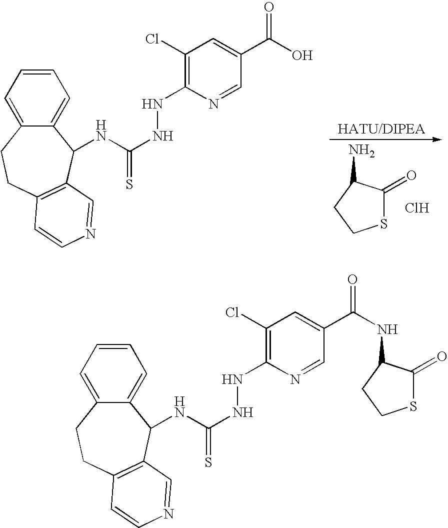

To a stirred solution of 5-chloro-6-[2-[[(6,11-dihydro-5H-benzo[5,6]cyclohepta[1,2-c]pyridin-11-yl)amino]thioxomethyl]hydrazino]-3-pyridinecarboxylic acid (198 mg, 0.45 mmol) in DMA (4 mL) was added HATU (207 mg, 0.54 mmol) followed by DIPEA (93 μL, 0.54 mmol). The mixture was stirred at room temperature for 10 min. and then D-homocysteine thiolactone hydrochloride (83 mg, 0.54 mmol) was added. The resulting mixture was stirred at room temperature for 4 h. The product mixture was concentrated in vacuo, the residue was taken up in DCM (40 mL), and washed with conc. NaHCO₃ (2×20 mL) and then brine (1×20 mL). The residue obtained after removal of the solvent was purified by flash chromatography (0˜5% MeOH in DCM) to provide the title compound (108 mg, 45%). ¹H-NMR (400 MHz, CD₃OD): δ 8.58(s, 1H), 8.52-8.48 (m, 1H), 8.29-8.21(m, 1H), 8.13-8.03(m, 1H), 7.45-7.32(m, 2H), 7.22-7.15(m, 3H), 7.13 (d, J=5 Hz, 1H), 4.93-4.80(m, 1H), 3.53-3.40 (m, 1H), 3.37-3.28(m, 1H), 3.28-3.06(m, 4H), 2.68-2.52 (m, 1H), 2.40-2.21 (m, 1H) ppm. HRMS (M+H)⁺ Calcd for C₅H₂₃ClN₆O₂S₂: 539.1091; Found: 539.1192.

### Example 26

### 5-chloro-6-[2-[6[(6,11-dihydro-7-methyl-5H-benzo[5,6]cyclohepta[1,2-c]pyridin-1-yl)amino]thioxomethyl]hydrazino]-N-[(3R)-1-methyl-2-oxa-3-pyrrolidinyl]-3-pyridinecarboxamide

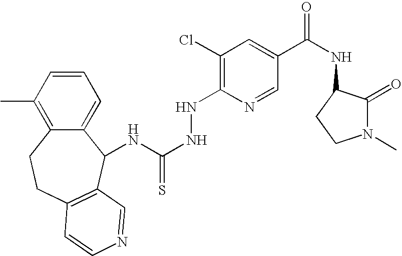

### 26A: Ethyl 4-[(E)-2-(2-methylphenyl)ethenyl]-3-pyridinecarboxylate

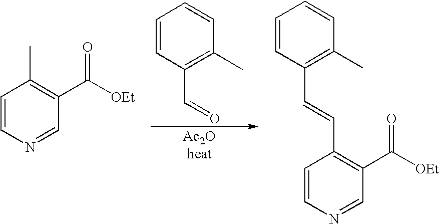

To a solution of ethyl 4-methylnicotinate (3.30 g, 20 mmol) in Ac₂O (6 mL) was added o-tolualdehyde (4.81 g, 40 mmol) and the mixture was stirred at 140° C. for 24 h under N₂. After cooled to room temperature, the mixture was concentrated in vacuo, water (12 mL) was added and the mixture was extracted with Et₂O (3×20 mL), the combined organic solution was extracted with 5% HCl (12 mL), and the aqueous solution was then neutralized with 28% NH₄OH to pH >9, then extracted with Et₂O (2×20 mL), dried over Na₂SO₄ and concentrated. The crude product (a dark brown oil, 3.8 g) was used directly for the next step without further purification. MS (ESI) (M+H)⁺=268.

### 26B: Ethyl 4-[2-(2-methylphenyl)ethyl]-3-pyridinecarboxylate

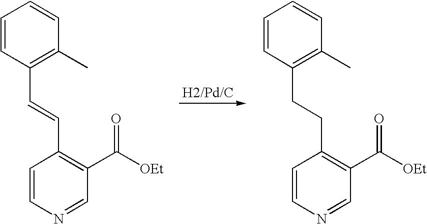

The solution of ethyl 4-[(E)-2-(2-methylphenyl)ethenyl]-3-pyridinecarboxylate (3.8 g) dissolved in MeOH (20 mL) was hydrogenated at 40 psi for 3 h in the presence of 10% Pd/C at room temperature. After work-up, the crude product was purified by passing through a short pad of silica gel (20% EtOAc in hexanes) to give the title compound as a light yellow oil (2.3 g, yield 42% for two steps). MS (ESI) (M+H)⁺=270.

### 26C: 4-[2-(2-methylphenyl)ethyl]-3-pyridinecarboxylic Acid

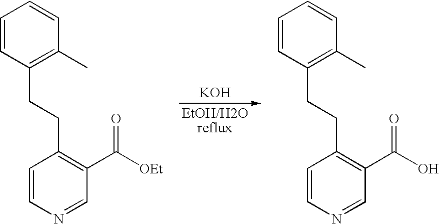

Ethyl 4-[2-(2-methylphenyl)ethyl]-3-pyridinecarboxylate (2.3 g, 8.5 mmol) was mixed with 12 mL of H₂O/EtOH (1:4). KOH (1.90 g, 34 mmol) was added and the mixture was stirred at reflux for 4 h. After cooled to room temperature, most solvents were removed and water (8 mL) was added. The solution was carefully neutralized to pH 56, the precipitate was collected and dried to give the title compound as white powders (1.8 g, 88%). MS (ESI) (M+H)⁺=242.

### 26D: 5,6-dihydro-7-methyl-11H-benzo[5,6]cyclohepta[1,2-c]pyridin-11-one

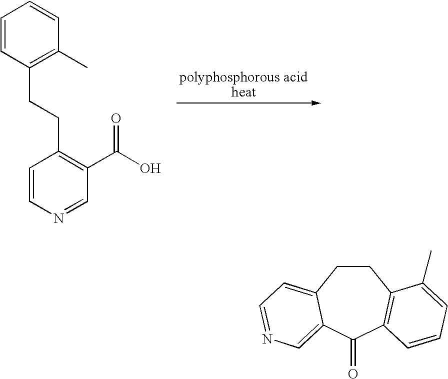

1.8 g of 4-[2-(2-Methylphenyl)ethyl]-3-pyridinecarboxylic acid (7.44 mmol) was added to stirred PPA (70 g) at 100° C. and the mixture was stirred at 160° C. for another 6 h. After cooling to 100° C., the mixture was poured into crashed ice (70 g) and the solution was carefully basified with KOH to pH>10. After extraction with EtOAc (4×40 mL), dried over Na₂SO₄ and evaporation, the title compound was obtained in 38% yield (0.63 g) and was used for the next step without further purification. (M+H)⁺=224.

### 26E: 5,6-dihydro-7-methyl-11H-benzo[5,6]cyclohepta[1,2-c]pyridin-1-one, Oxime

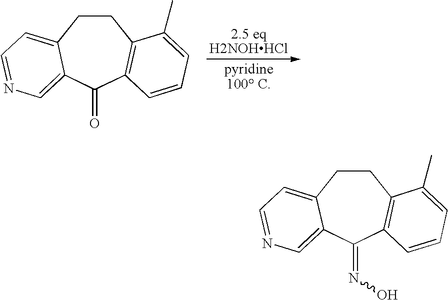

To a stirred solution of 5,6-dihydro-7-methyl-11H-benzo[5,6]cyclohepta[1,2-c]pyridin-11-one (0.63 g, 2.84 mmol) in pyridine (4 mL) at 100° C. was added H₂NOH.HCl (0.49 g, 7.1 mmol) and the resulting mixture was stirred at the same temperature overnight. After the reaction was completed, pyridine was removed in vacuo, the residue was added water and the precipitate was collected by filtration, washed with water, dried to give the title compound as pale yellow powders (0.63 g, 93%), which was used without further purification. MS (ESI) (M+H)⁺=239.

### 26F: 6,11-dihydro-7-methyl-5H-benzo[5,6]cyclohepta[1,2-c]pyridin-11-amine

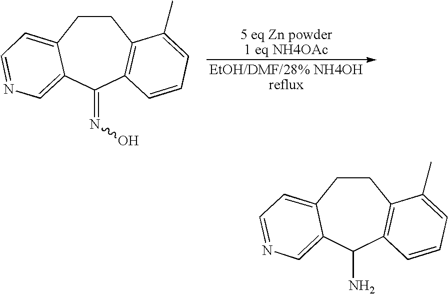

To a mixture of 5,6-dihydro-7-methyl-11H-benzo[5,6]cyclohepta[1,2-c]pyridin-11-one oxime (0.6 g, 2.5 mmol) in EtOH (15 mL), DMF (2.5 mL) and 28% NH₄H (10 mL) was added Zn powder (0.82 g, 12.5 mmol) followed by NH₄OAc (0.20 g, 2.5 mmol). The mixture was stirred at reflux for 3 h. After cooled to room temperature, Et₂O (50 mL) was added and, stirred for 20 min, then 1N NaOH (15 mL) was added and stirred for another 10 min. After filtered through Celite, the organic layer was isolated, and the basic aqueous solution was extracted with Et₂O (4×20 mL). The combined organic layer was washed with water and then brine, dried over Na₂SO₄, evaporated to give the title compound as white solids (0.53 g, 94%), which was used without further purification. MS (ESI) (M+H)⁺=225.

### 26G: 6,11-dihydro-7-methyl-11-isothiocyanato-5H-benzo[5,6]cyclohepta[1,2-c]pyridine

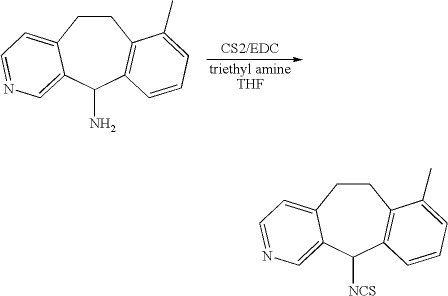

To a solution of 6,11-dihydro-7-methyl-5H-benzo[5,6]cyclohepta[1,2-c]pyridin-11-amine (22.4 mg, 0.1 mmol) in dry THF (1.2 mL) was added CS₂ (60 μL, 1.0 mmol) at −10° C., then EDC (38.2 mg, 0.2 mmol) was added in one portion. After being stirred for 15 minutes, TEA (28 μL, 0.2 mmol) was added dropwise. The mixture was allowed to slowly warm up to room temperature and stirred for 4 hours. After filtration, the filtrate was concentrated and the residue was taken up in ethyl acetate (10 mL), extracted with saturated NaHCO₃ (1×10 mL), water (1×10 mL) and then brine (1×10 mL), dried over Na₂SO₄, then concentrated in vacuo. The residue was purified by passing through a short pad of silica gel (30% EtOAc in DCM) to give the title compound as white solids (21 mg, 79%). MS (ESI) (M+H)⁺=267.

### 26H: 5,6-dichloro-N-[(3R)-1-methyl-2-oxo-3-pyrrolidinyl]-3-pyridinecarboxamide

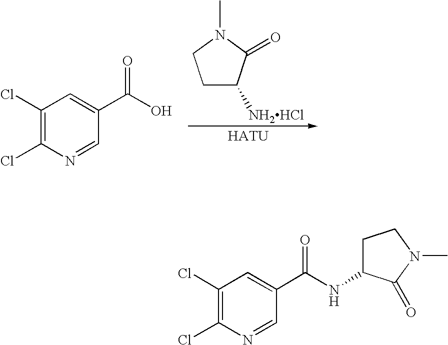

Following general HATU coupling procedure of Example 1C: The title compound (108 mg, 74%) was prepared from 5,6-dichloronicotic acid and (3R)-amino-1-methyl-2-pyrrolidinone HCl salt. 1HNMR (DMSO-d6): δ 9.07 (d, J=8.2 Hz, 1H), 8.76 (s, 1H), 8.45 (s, 1H), 4.55 (q, J=9.0 Hz, 1H), 3.25-3.35 (m, 2H), 2.73 (s, 3H), 2.25-2.35 (m, 1H), 1.85-1.95 (m, 1H) ppm. MS (ESI) (M+1)⁺: 289.

### 26I: 5-chloro-6-hydrazino-N-[(3R)-1-methyl-2-oxo-3-pyrrolidinyl]-3-pyridinecarboxamide

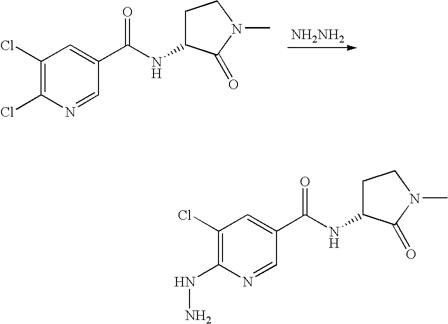 1 M NH₂NH₂ in EtOH (1 ml) was added to a solution of 5,6-dichloro-N-[(3R)-1-methyl-2-oxo-3-pyrrolidinyl]-3-pyridinecarboxamide (98 mg, 0.34 mmol) in EtOH (2 ml), the reaction mixture was heated at reflux for 8 h, allowed to cool to room temperature. The white solid was collected and washed with MeOH (3 ml) to afford the title compound (66 mg).

### 26J: 5-chloro-6-[2-[[(6,11-dihydro-7-methyl-5H-benzo[5,6]cyclohepta[1,2-c]pyridin-11-yl)amino]thioxomethyl]hydrazino]-N-[(3R)-1-methyl-2-oxo-3-pyrrolidinyl]-3-pyridinecarboxamide

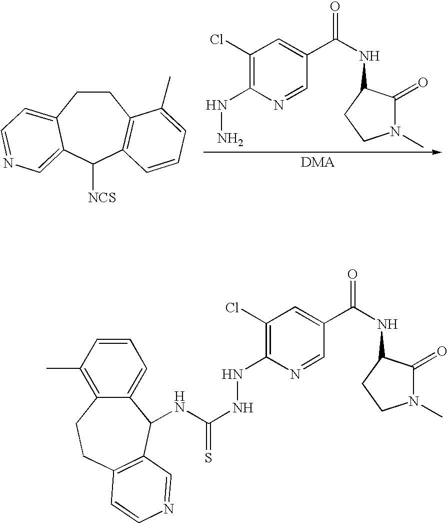

To a stirred solution of 6,11-dihydro-7-methyl-11-isothiocyanato-5H-benzo[5,6]cyclohepta[1,2-c]pyridine (21 mg, 0.08 mmol) in DMA (2 mL) was added 5-chloro-6-hydrazino-N-[(3R)-1-methyl-2-oxo-3-pyrrolidinyl]-3-pyridinecarboxamide (22.7 mg, 1.0 mmol). The mixture was stirred at room temperature for 2 hours and then concentrated in vacuo, the residue was purified by flash chromatography (5˜10% MeOH in DCM) to provide the title compound (15 mg, 34%). ¹H-NMR (400 MHz, CD₃OD): δ 8.60-8.55 (m, 1H), 8.52(s, 1H), 8.27-8.21 (m, 1H), 8.10(d, J=2 Hz, 1H), 7.40(d, J=2 Hz, 1H), 7.19(d, J=2 Hz, 1H), 7.14(d, J=2 Hz, 1H), 7.11-6.97(m, 2H), 4.73-4.60 (m, 1H), 3.52-3.39(m, 2H), 3.34-3.27(m, 4H), 2.88 (s, 1H), 2.54-2.42 (m, 1H), 2.12-1.96 (m, 1H) ppm. MS (ESI)(M+1)⁺=550. HRMS (M+H)⁺ Calcd for C₂₇H₂₈ClN₇O₂S: 550.1792; Found: 550.1763.

### Example 27

### 5-chloro-6-[2-[[(7-fluoro-6,11-dihydro-5H-benzo[5,6]cyclohepta[1,2-c]pyridin-11-yl)amino]thioxomethyl]hydrazino]-N-[(3R)-1-methyl-2-oxo-3-pyrrolidinyl]-3-pyridinecarboxamide

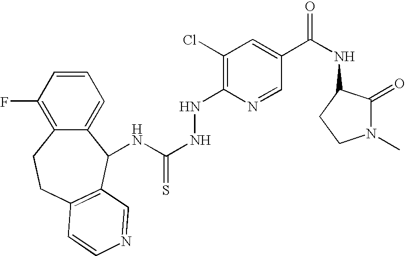

### 27A: Ethyl 4-[(E)-2-(2-fluorophenyl)ethenyl]-3-pyridinecarboxylate

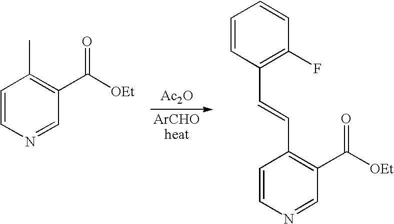

To a solution of ethyl 4-methylnicotinate (8.25 g, 50 mmol) in Ac₂O (16 mL) was added 2-flurobenzaldehyde (12.4 g, 100 mmol) and the mixture was stirred at 140° C. for 24 h under N₂. After cooled to room temperature, the mixture was concentrated in vacuo, water (35 mL) was added and the mixture was extracted with Et₂O (3×40 mL), the combined organic solution was extracted with 5% HCl (30 mL), and the aqueous solution was then neutralized with 28% NH₄OH to pH>9, then extracted with Et₂O (2×40 mL), dried over Na₂SO₄ and concentrated. The crude product (a dark brown, oil, ˜14 g) was used directly for the next step without further purification. MS (ESI) (M+H)⁺=274.

### 27B: ethyl 4-[2-(2-fluorophenyl)ethyl]-3-pyridinecarboxylate

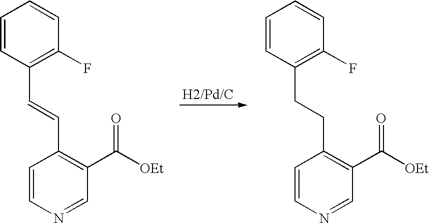

The solution of ethyl 4-[(E)-2-(2-fluorophenyl)ethenyl]-3-pyridinecarboxylate (˜14 g) dissolved in MeOH (50 mL) was hydrogenated at ˜40 psi for 3 h in the presence of 10% Pd/C at room temperature. After work-up, the crude product was purified by passing through a short pad of silica gel (20% EtOAc in hexanes) to give the title compound as a light yellow oil (7.2 g, yield 53% for two steps). ¹H-NMR (400 MHz, CDCl₃): δ 9.08 (s, 1H), 8.54(d, J=5 Hz, 1H), 7.25-6.93 (m, 5H), 4.40(q, J=8 Hz, 2H), 3.36-3.19(m, 2H), 3.02-2.81(m, 2H), 1.41(t, J=8 Hz, 3H) ppm. MS (ESI) (M+H)⁺=272.

### 27C: [2-(2-fluorophenyl)ethyl]-3-pyridinecarboxylic Acid

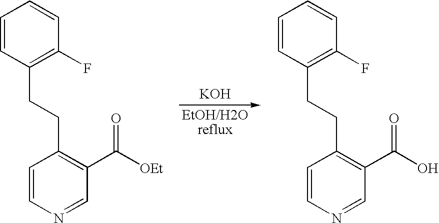

Ethyl 4-[2-(2-fluorophenyl)ethyl]-3-pyridinecarboxylate (7.1 g, 26 mmol) was mixed with 35 mL of H₂O/EtOH (1:4). KOH (5.83 g, 4 eq) was added and the mixture was stirred at reflux for 4 h. After cooled to room temperature, most solvents were removed and water (30 mL) was added. The solution was carefully neutralized to pH 56, the precipitate was collected and dried to give the title compound as white powders 6.3 g (yield 99%). MS (ESI) (M+H)⁺=246.

### 27D: 7-fluoro-5,6-dihydro-11H-benzo[5,6]cyclohepta[1,2-c]pyridin-11-one

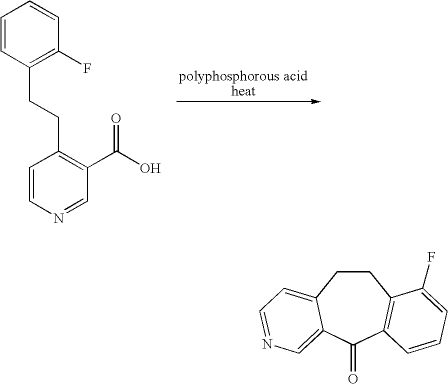

6.3 g of 4-[2-(2-Fluorophenyl)ethyl]-3-pyridinecarboxylic acid (25.7 mmol) was added to stirred PPA (240 g) at 100° C. and the mixture was stirred at 160° C. for another 6 h. After cooling to 100° C., the mixture was poured into crashed ice (200 g) and the solution was carefully basified with KOH to pH>10. After extraction with EtOAc (4×150 mL), dried over Na₂SO₄ and evaporation, the title compound was obtained in 92% yield (5.38 g) and was used for the next step without further purification. MS (ESI) (M+H)⁺=228.

### 27E: 7-fluoro-5,6-dihydro-11H-benzo[5,6]cyclohepta[1,2-c]pyridin-11-one, Oxime

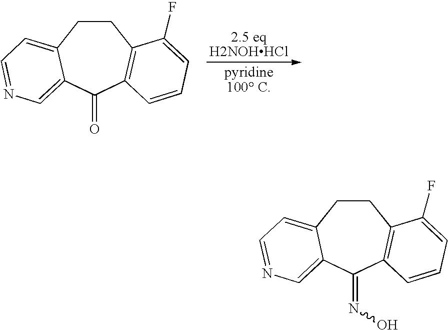

To a stirred solution of 7-fluoro-5,6-dihydro-11H-benzo[5,6]cyclohepta[1,2-c]pyridin-11-one (5.38 g, 23.7 mmol) in pyridine (30 mL) at 100° C. was added H₂NOH.HCl (4.12 g, 59.3 mmol) and the resulting mixture was stirred at the same temperature overnight. After the reaction was completed, pyridine was removed in vacuum, the residue was added water and the precipitate was collected by filtration, washed with water, dried to give the title compound as pale yellow powders (5.51 g, yield 96%), which was used without further purification. MS (ESI) (M+H)⁺=243.

### 27F: 7-fluoro-6,11-dihydro-5H-benzo[5,6]cyclohepta[1,2-c]pyridin-11-amine

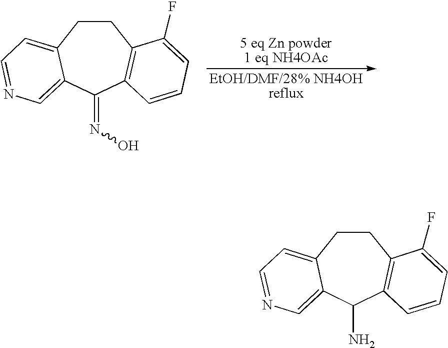

To a mixture of 7-fluoro-5,6-dihydro-11H-benzo[5,6]cyclohepta[1,2-c]pyridin-11-one oxime (1.21 g, 5 mmol) in EtOH (30 mL), DMF (5 mL) and 28% NH₄OH (20 mL) was added Zn powder (1.63 g, 25 mmol) followed by NH₄OAc (0.39 g, 5 mmol). The mixture was stirred at reflux for 3 h. After cooled to room temperature, Et₂O (100 mL) was added and stirred for 20 min, then 1N NaOH (30 mL) was added and stirred for another 10 min. After filtered through Celite, the organic layer was isolated, and the basic aqueous solution was extracted with Et₂O (4×50 mL). The combined organic layer was washed with water and then brine, dried over Na₂SO₄, evaporated to give the title compound as white solids (1.07 g, 94%), which was used without further purification. ¹H-NMR (400 MHz, CDCl₃): δ 8.55 (s, 1H), 8.39(d, J=5 Hz, 1H), 7.28-7.16 (m, 2H), 7.05(d, J=5 Hz, 1H), 7.00-6.91 (m, 1H), 5.40 (s, 1H), 3.69-3.46(m, 2H), 3.21-2.93(m, 2H), 1.85 (s, br, 2H) ppm. MS (ESI) (M+H)⁺=229.

### 27G: 7-fluoro-6,11-dihydro-11-isothiocyanato-5H-benzo[5,6]cyclohepta[1,2-c]pyridine

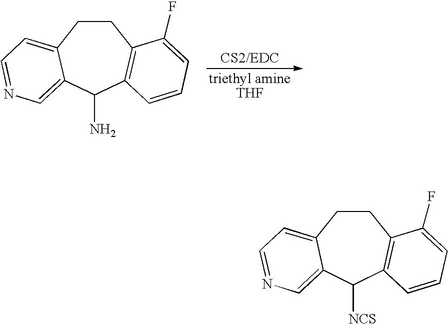

To a solution of 7-fluoro-6,11-dihydro-5H-benzo[5,6]cyclohepta[1,2-c]pyridin-11-amine (571 mg, 2.5 mmol) in dry THF (30 mL) was added CS₂ (1.5 mL, 25 mmol) at −10° C., then EDC (956 mg, 5.0 mmol) was added in one portion. After being stirred for 15 minutes, TEA (697 μL, 5.0 mmol) was added dropwise. The mixture was allowed to slowly warm up to room temperature and stirred for 4 hours. After filtration, the filtrate was concentrated and the residue was taken up in ethyl acetate (50 mL), extracted with saturated NaHCO₃ (1×0 mL), water (1×20 mL) and then brine (1×20 mL), dried over Na₂SO₄, then concentrated in vacuo. The residue was purified by passing through a short pad of silica gel (30% EtOAc in DCM) to give the title compound as white solids (578 mg, 86%). ¹H-NMR (400 MHz, CDCl₃): δ 8.62 (s, 1H), 8.49 (d, J=5 Hz, 1H), 7.25-7.18 (m, 2H), 7.12 (d, J=5 Hz, 1H), 7.08-7.02 (m, 1H), 6.22 (s, 1H), 3.48-3.35 (m, 2H), 3.18-3.05(m, 1H) ppm. MS (ESI) (M+H)⁺=271.

### 27H: 5-chloro-6-[2-[[(7-fluoro-6,11-dihydro-5H-benzo[5,6]cyclohepta[1,2-c]pyridin-11-yl)amino]thioxomethyl]hydrazino]-3-pyridinecarboxylic Acid

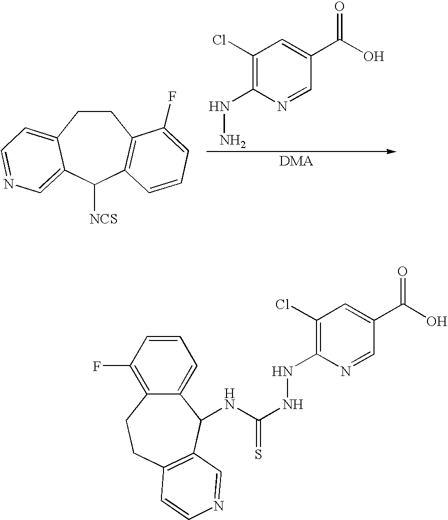

To a solution of 7-fluoro-6,11-dihydro-11-isothiocyanato-5H-benzo[5,6]cyclohepta[1,2-c]pyridine (270 mg, 1.0 mmol) in DMA (5 mL) was added 5-chloro-6-hydrazino-3-pyridinecarboxylic acid (188 mg, 1.0 mmol), the mixture was stirred at room temperature for 2 hours, the solvent was concentrated in vacuo and water was added, the precipitate was collected by filtration, dried to give the title compound as off-white powders (428 mg, 93%). MS (ESI) (M+H)+458.

### 27I: 5-chloro-6-[2-[[(7-fluoro-6,11-dihydro-5H-benzo[5,6]cyclohepta[1,2-c]pyridin-11-yl)amino]thioxomethyl]hydrazino]-N-[(3R)-1-methyl-2-oxo-3-pyrrolidinyl]-3-pyridinecarboxamide

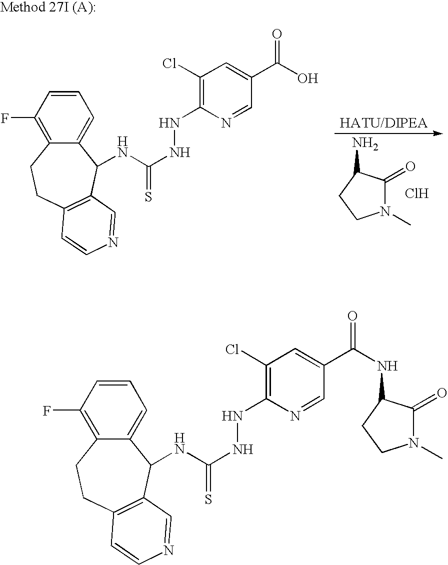

To a stirred solution of 5-chloro-6-[2-[[(7-fluoro-6,11-dihydro-5H-benzo[5,6]cyclohepta[1,2-c]pyridin-11-yl)amino]thioxomethyl]hydrazino]-3-pyridinecarboxylic acid (69 mg, 0.15 mmol) in DMA (2 mL) was added HATU (69 mg, 0.18 mmol) followed by DIPEA (31 μL, 0.18 mmol). The mixture was stirred at room temperature for 10 min, and then (3R)-3-amino-1-methyl-2-pyrrolidinone hydrochloride (27 mg, 0.18 mmol) was added. The resulting mixture was stirred at room temperature for 4 h. The product mixture was concentrated in vacuo, the residue was taken up in DCM (20 mL), and washed with conc. NaHCO₃ (2×10 mL) and then brine (1×10 mL). The residue obtained after removal of the solvent was purified by flash chromatography (5˜10% MeOH in DCM) to provide the title compound (43 mg, 52%). ¹H-NMR (400 MHz, CD₃OD): δ 8.60-8.46 (m, 2H), 8.24 (d, J=5 Hz, 1H), 8.09-7.98(m, 1H), 7.46-7.24(m, 1H), 7.26-7.05 (m, 3H), 4.71-4.55 (m, 1H), 3.52-3.33(m, 2H), 3.24-3.03(m, 4H), 2.85 (s, 3H), 2.54-2.36 (m, 1H), 2.13-1.92 (m, 1H) ppm. HRMS (M+H)⁺ Calcd for C₂₆H₂₅ClFN₇O₂S: 554.1541; Found: 554.1548.

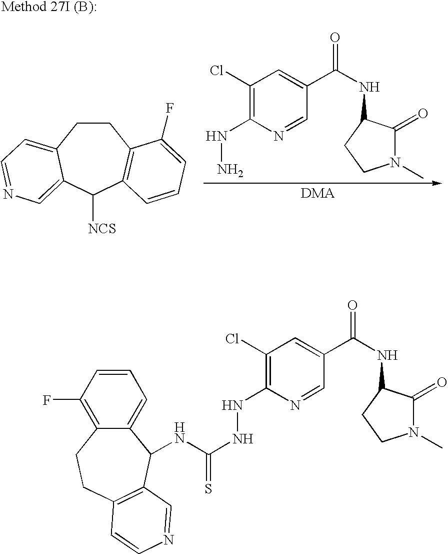

To a stirred solution of 7-fluoro-6,11-dihydro-11-isothiocyanato-5H-benzo[5,6]cyclohepta[1,2-c]pyridine (27 mg, 0.1 mmol) in DMA (2 mL) was added 5-chloro-6-hydrazino-N-[(3R)-1-methyl-2-oxo-3-pyrrolidinyl]-3-pyridinecarboxamide (28.4 mg, 1.0 mmol). The mixture was stirred at room temperature for 2 hours and then concentrated in vacuo, the residue was treated with small amount of MeOH and diethyl ether, the precipitate was collected by filtration and then dried to give the title compound as white solid (54 mg, 97%).

### Examples 28 and 29

The two diastereoisomers of Example 27 were produced via chiral separation from compound 27 using semi-preparative column (Chiralpak AD 21×250 mm column), with 23% isopronaol/77% hexane (containing 0.1% diethyl amine in each solvent) as a mobile phase (flow rate 9 ml/min, run of 120 min). Two diastereomers were successfully isolated:

### Example 28

Isomer A (earlier fraction): ¹H-NMR (400 MHz, CD₃OD): δ 8.45 (d, J=8 Hz, 2H), 8.18 (d, J=5 Hz, 1H), 7.98(s, 1H), 7.43-7.32(m, 1H), 7.26-7.08 (m, 3H), 6.87 (t, J=8 Hz, 1H), 4.56 (t, J=10 Hz, 1H), 3.47-3.38(m, 2H), 3.24-3.07(m, 4H), 2.79 (s, 3H), 2.51-2.40 (m, 1H), 2.10-2.96 (m, 1H) ppm. [α]²⁵_{D} +6.6° (c 0.098, methanol).

### Example 29

Isomer B (later fractions): ¹H-NMR (400 MHz, CD₃OD): δ 8.45 (s, 2H), 8.18 (d, J=5 Hz, 1H), 7.99-7.97 (m, 1H), 7.27(s, 1H), 7.19-7.00 (m, 3H), 6.88 (t, J=8 Hz, 1H), 4.56 (t, J=10 Hz, 1H), 3.39-3.30 (m, 2H), 3.16-2.99 (m, 4H), 2.79 (s, 3H), 2.44-2.31 (m, 1H), 2.04-1.88 (m, 1H) ppm. [α]²⁵_{D} −1.2° (c 0.102, methanol).

### Example 30

### 5-chloro-6-[2-[[(7-fluoro-6,11-dihydro-5H-benzo[5,6]cyclohepta[1,2-c]pyridin-11-yl)amino]thioxomethyl]hydrazino]-N-[(3R)-tetrahydro-2-oxo-3-thienyl]-3-pyridinecarboxamide

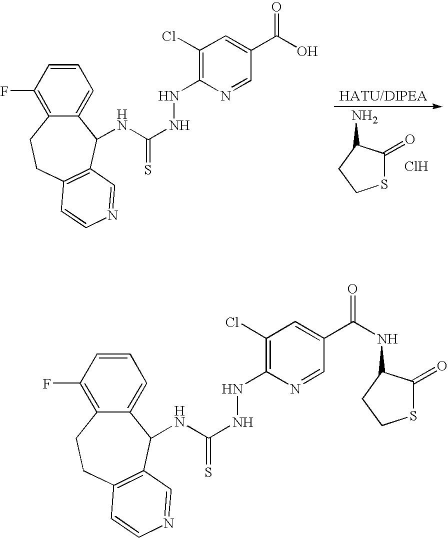

Following the general HATU coupling procedure of Example 1C: To a stirred solution of 5-chloro-6-[2-[[(7-fluoro-6,11-dihydro-5H-benzo[5,6]cyclohepta[1,2-c]pyridin-11-yl)amino]thioxomethyl]hydrazino]-3-pyridinecarboxylic acid (105 mg, 0.23 mmol) in DMA (3 mL) was added HATU (107 mg, 0.28 mmol) followed by DIPEA (48 μL, 0.28 mmol). The mixture was stirred at room temperature for 10 min, and then D-homocysteine thiolactone hydrochloride (43 mg, 0.28 mmol) was added. The resulting mixture was stirred at room temperature for 4 h. The product mixture was concentrated in vacuo, the residue was taken up in DCM (20 mL), and washed with conc. NaHCO₃ (2×10 mL) and then brine (1×10 mL). The residue obtained after removal of the solvent was purified by flash chromatography (05% MeOH in DCM) to provide the title compound (44 mg, 34%). ¹H-NMR (400 MHz, CD₃OD): δ 8.61-8.50 (m, 2H), 8.34-8.25 (m, 1H), 8.13-8.04 (m, 1H), 7.40(s, br, 1H), 7.28-7.20(m, 2H), 7.19-7.11 (m, 1H), 7.03-6.94(m, 1H), 4.92-4.79 (m, 1H), 3.52-3.40(m, 1H), 3.36-3.26(m, 1H), 3.26-3.08 (m, 4H), 2.71-2.56 (m, 1H), 2.40-2.22 (m, 1H) ppm. MS (ESI) (M+1)⁺=557. HRMS (M+H)⁺ Calcd for C₂₅H₂₂ClFN₆O₂S: 557.0996; Found: 557.0910. Anal. Calcd for C₂₅H₂₂ClFN₆O₂S.2.3H₂O: C, 50.17; H, 4.48; N, 14.04; Found: C, 50.28; H, 4.77; N, 14.07.

### Example 31

### 5-chloro-6-[2-[[(7-fluoro-6,11-dihydro-5H-benzo[5,6]cyclohepta[1,2-c]pyridin-11-yl)amino]thioxomethyl]hydrazino]-N-[(3R)-2-oxo-1-phenyl-3-pyrrolidinyl]-3-pyridinecarboxamide

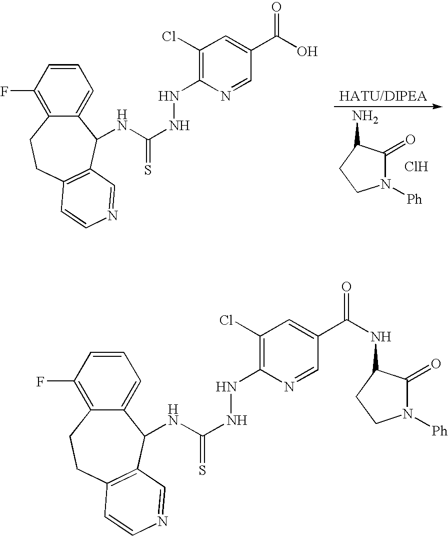

To a stirred solution of 5-chloro-6-[2-[[(7-fluoro-6,11-dihydro-5H-benzo[5,6]cyclohepta[1,2-c]pyridin-11-yl)amino]thioxomethyl]hydrazino]-3-pyridinecarboxylic acid (92 mg, 0.2 mmol) in DMA (3 mL) was added HATU (92 mg, 0.24 mmol) followed by DIPEA (41 μL, 0.24 mmol). The mixture was stirred at room temperature for 10 min, and then (3R)-3-amino-1-phenyl-2-pyrrolidinone hydrochloride (51 mg, 0.24 mmol) (made according to the procedure disclosed in Ian M. Bell et al., J. Med. Chem., 2001, 44, 2933, which is incorporated by reference herein for its disclosure in the preparation of (3R)-3-amino-1-phenyl-2-pyrrolidinone hydrochloride) was added. The resulting mixture was stirred at room temperature for 4 h. The product mixture was concentrated in vacuo, the residue was taken up in DCM (20 mL), and washed with concentrated NaHCO₃ (2×10 mL) and then brine (1×10 mL). The residue obtained after removal of the solvent was purified by flash chromatography (0˜5% MeOH in DCM) to provide the title compound (59 mg, 48%). ¹H-NMR (400 MHz, CD₃OD): δ 8.62-8.57 (m, 1H), 8.54(s, 1H), 8.28-8.24(m, 1H), 8.08 (d, J=2 Hz, 1H), 7.64(d, J=8 Hz, 211), 7.42-7.38(m, 31H), 7.26-7.10(m, 4H), 6.97(t, J=8 Hz, 1H), 4.94-4.84 (m, 1H), 3.99-3.82(m, 2H), 3.27-3.07(m, 4H), 2.65-2.53 (m, 1H), 2.29-2.15 (m, 1H) ppm. MS (ESI) (M+)⁺=616. HRMS (M+H)+Calcd for C₃₁H₂₇ClFN₇O₂S: 616.1698; Found: 616.1691. Anal. Calcd for C₃₁H₂₇ClFN₇O₂S.2H₂O: C, 57.09; H, 4.79; N, 15.03; Found: C, 56.98; H, 4.72; N, 14.94.

### Example 32

### 5-chloro-6-[2-[[(7-fluoro-6,11-dihydro-5H-benzo[5,6]cyclohepta[1,2-c]pyridin-11-yl)amino]thioxomethyl]hydrazino]-N-[[(2R)-tetrahydro-2-furanyl]methyl]-3-pyridinecarboxamide

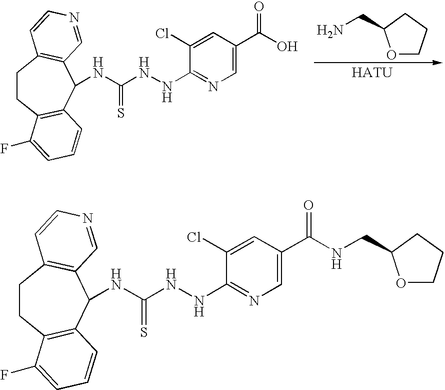

Following the general HATU coupling procedure of Example 1C: HATU (50 mg) was added to a solution of 5-chloro-6-[2-[[(1-fluoro-6,11-dihydro-5H-benzo[5,6]cyclohepta[1,2-c]pyridin-11-yl)amino]thioxomethyl]hydrazino]-3-pyridinecarboxylic acid (52 mg), (2R)-tetrahydro-2-furanmethanamine (15 mg) and DIPEA (0.1 ml) in DMA (3 ml), and the reaction mixture was stirred at room temperature for 2 h. DMA was stripped off in vacuo and the residue was treated with H₂O and the solid was collected and purified by prep-HPLC to give the title compound (15 mg) as a TFA salt. ¹H NMR(400 MHz, CD₃OD) δ: 8.83 (s, 1H), 8.61 (d, J=6 Hz, 1H), 8.57 (s, 1H), 8.14 (s, 1H), 7.89 (d, J=6 Hz, 1H), 7.43 (br, 1H), 7.20-7.33 (m, 2H), 7.05-7.10 (m, 1H), 4.05-5.15 (m, 1H), 3.89 (dd, J=7.2, 14.5 Hz, 1H), 3.79 (dd, J=10.0, 14.5 Hz, 1H), 3.30-3.60 (m, 5H), 3.10-3.20 (m, 1H), 1.98-2.08 (m, 1H), 1.88-1.97 (m, 1H), 1.59-1.70 (m, 1H) ppm. MS(ESI)(M+1)⁺=541. HRMS (M+1)⁺ Calcd for C₂₆H₂₆ClFN₆O₂S: 541.1589; Found: 541.1669.

### Example 33

### 5-chloro-6-[2-[[(7-fluoro-6,11-dihydro-5H-benzo[5,6]cyclohepta[1,2-c]pyridin-11-yl)amino]carbonyl]hydrazino]-N-[(3R)-tetrahydro-2-oxo-3-thienyl]-3-pyridinecarboxamide

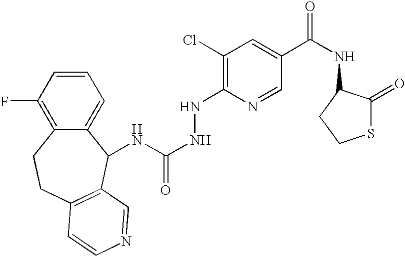

### 33A: 5-chloro-6-[2-[[(7-fluoro-6,11-dihydro-5H-benzo[5,6]cyclohepta[1,2-c]pyridin-11-yl)amino]carbonyl]hydrazino]-3-pyridinecarboxylic Acid

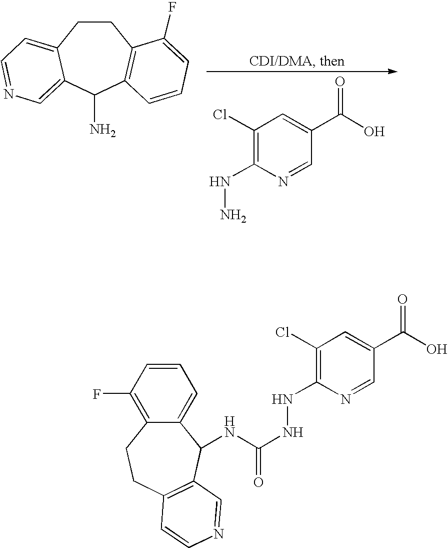

To a solution of CDI (24 mg, 0.15 mmol) in dry DMF (1.5 mL) was added a solution of 7-fluoro-6,11-dihydro-5H-benzo[5,6]cyclohepta[1,2-c]pyridin-11-amine (34.5 mg, 0.15 mmol) in dry DMF (1.5 mL) at 0° C. After being stirred for 1 hour, the mixture was allowed to warm up to room temperature and 5-chloro-6-hydrazino-3-pyridinecarboxylic acid (28 mg, 0.15 mmol) was added. The mixture was stirred at room temperature for another 2 hours. The solvent was concentrated in vacuo and the residue was treated with small amount of MeOH and diethyl ether, the white solid was collected and dried to afford the title comound (51 mg, 77%). MS (ESI) (M+H)⁺=442.

### 33B: 5-chloro-6-[2-[[(7-fluoro-6,11-dihydro-5H-benzo[5,6]cyclohepta[1,2-c]pyridin-11-yl)amino]carbonyl]hydrazino]-N-[(3R)-tetrahydro-2-oxo-3-thienyl]-3-pyridinecarboxamide

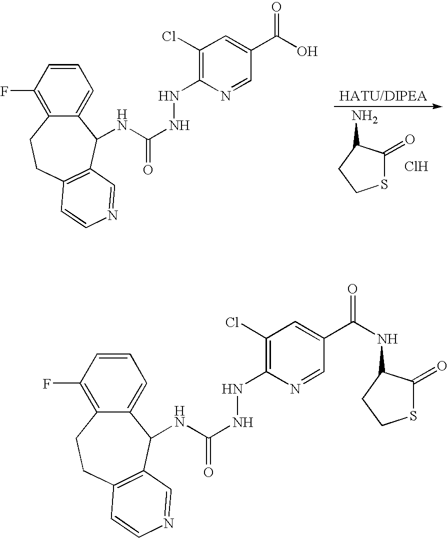

To a stirred solution of 5-chloro-6-[2-[[(7-fluoro-6,11-dihydro-5H-benzo[5,6]cyclohepta[1,2-c]pyridin-11-yl)amino]carbonyl]hydrazino]3-pyridinecarboxylic acid (51 mg, 0.115 mmol) in DMA (2 mL) was added HATU (54 mg, 0.14 mmol) followed by DIPEA (24 μL, 0.14 mmol). The mixture was stirred at room temperature for 10 min and then D-homocysteine thiolactone hydrochloride (22 mg, 0.14 mmol) was added. The resulting mixture was stirred at room temperature for 4 h. The product mixture was concentrated in vacuo, the residue was taken up in DCM (20 mL), and washed with conc. NaHCO₃ (2×10 mL) and then brine (1×10 mL). The residue obtained after removal of the solvent was purified by flash chromatography (05% MeOH in DCM) to provide the title compound (15 mg, 24%). ¹H-NMR (400 MHz, CD₃OD): δ 8.46(s, 1H), 8.43-8.39 (m, 1H), 8.20(d, J=5 Hz, 1H), 7.95(s, 1H), 7.20-7.04 (m, 3H), 6.91(t, J=8 Hz, 1H), 6.28(s, 1H), 4.86-4.72 (m, 1H), 3.42-3.30(m, 1H), 3.28-3.00(m, 5H), 2.52-2.47 (m, 1H), 2.28-2.12 (m, 1H) ppm. MS (ESI)(M+1)⁺=541. HRMS (M+H)⁺ Calcd for C₂₅H₂₂ClFN₆O₃S: 541.1225; Found: 541.1207.

### Example 34

### 5-chloro-6-[2-[[(7-fluoro-6,11-dihydro-5H-benzo[5,6]cyclohepta[1,2-c]pyridin-11-yl)amino]carbonyl]hydrazino]-N-[(3R)-1-methyl-2-oxo-3-pyrrolidinyl]-3-pyridinecarboxamide

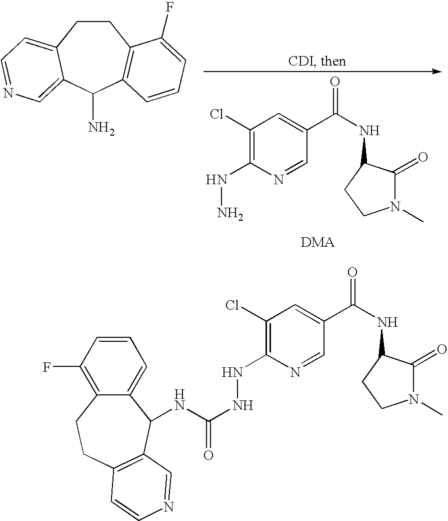

To a solution of CDI (31 mg, 0.19 mmol) in dry DMF (2 mL) was added a solution of 7-fluoro-6,11-dihydro-5H-benzo[5,6]cyclohepta[1,2-c]pyridin-11-amine (44 mg, 0.19 mmol) in dry DMF (2 mL) at 0° C. After being stirred for 1 hour, the mixture was allowed to warm up to room temperature and 5-chloro-6-hydrazino-N-[(3R)-1-methyl-2-oxo-3-pyrrolidinyl]-3-pyridinecarboxamide (54 mg, 0.19 mmol) was added. The mixture was stirred at room temperature for another 2 hours. The solvent was concentrated in vacuo, the residue was taken up into DCM (20 mL) and washed with water (1×10 mL), dried over Na₂SO₄, concentrated and purified by flash chromatography (5˜10% MeOH in DCM) to give the title compound as white solids (54 mg, 53%). ¹H-NMR (400 MHz, CD₃OD): δ 8.53(s, 1H), 8.46-8.42 (m, 1H), 8.28-8.30(m, 1H), 7.91 (d, J=2 Hz, 1H), 7.22 (d, J=8 Hz, 1H), 7.18-7.08(m, 2H), 6.94 (t, J=8 Hz, 1H), 6.32 (d, J=2 Hz, 1H), 4.63 (t, J=8 Hz, 1H), 3.44-3.36 (m, 2H), 3.31-3.23(m, 2H), 3.16-3.00(m, 2H), 2.84 (s, 3H), 2.50-2.36 (m, 1H), 2.09-1.90 (m, 1H) ppm. MS (ESI)(M+1)⁺=538. HRMS (M+H)⁺ Calcd for C₁₆H₂₅ClFN₇O₃: 538.1770; Found: 538.1729.

### Example 35

### 5-chloro-6-[2-[[(10,11-dihydro-5H-benzo[4,5]cyclohepta[1,2-b]pyridin-5-yl)amino]thioxomethyl]hydrazino]-N-[(3R)-1-methyl-2-oxo-3-pyrrolidinyl]-3-pyridinecarboxamide

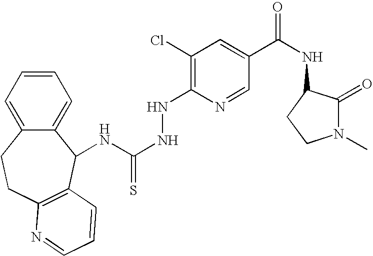

### 35A: 10,11-dihydro-5H-benzo[4,5]cyclohepta[1,2-b]pyridin-5-one, Oxime

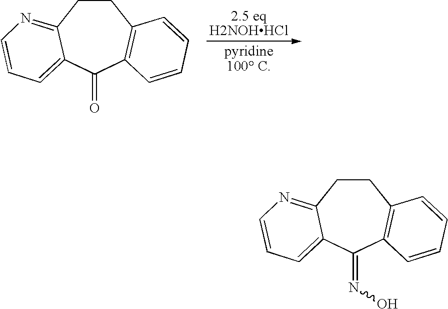

To a stirred solution of 10,11-dihydro-5H-benzo[4,5]cyclohepta[1,2-b]pyridin-5-one (837 mg, 4.0 mmol) (prepared according to Villani F. J., et al, J. Heterocyclic Chem., 1971, 8, 73) in pyridine (10 mL) at 100° C. was added H₂NOH.HCl (10 mmol, 695 mg) and the resulting mixture was stirred at the same temperature overnight. After the reaction was completed, pyridine was removed in vacuo, the residue was added water and the precipitate was collected by filtration, washed with water, dried to give product as pale yellow powder (843 mg, 94%), which was used without further purification. MS (ESI) (M+H)⁺=225.

### 35B: 10,11-dihydro-5H-benzo[4,5]cyclohepta[1,2-b]pyridin-5-amine

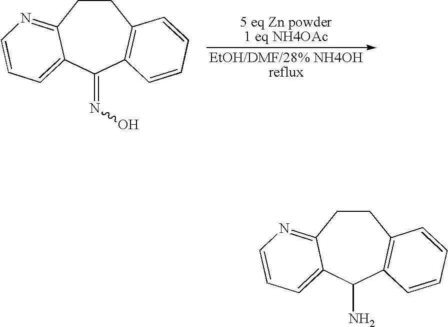

To a mixture of 10,11-dihydro-5H-benzo[4,5]cyclohepta[1,2-b]pyridin-5-one oxime (843 mg, 3.76 mmol) in EtOH (23 mL), DMF (3 mL) and 28% NH₄OH (15 mL) was added Zn powder (1.22 g, 18.8 mmol) followed by NH₄OAc (0.29 g, 3.76 mmol). The mixture was stirred at reflux for 3 h. After cooled to room temperature, Et₂O (80 mL) was added and stirred for 20 min, then 1N NaOH (20 mL) was added and stirred for another 10 min. After filtered through Celite, the organic layer was isolated, and the basic aqueous solution was extracted with Et₂O (3×20 mL). The combined organic layer was washed with water and then brine, dried over Na₂SO₄, evaporated to give the title compound as a white solid (700 mg, 88%), which was used without further purification. MS (ESI) (M+H)⁺=211.

### 35C: 10,11-dihydro-5-isothiocyanato-5H-benzo[4,5]cyclohepta[1,2-b]pyridine

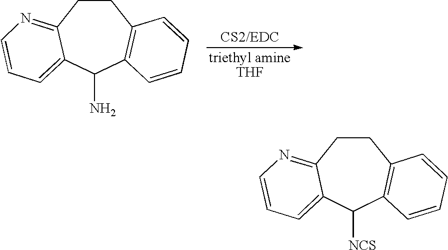

To a solution of 10,11-dihydro-5H-benzo[4,5]cyclohepta[1,2-b]pyridin-5-amine (210 mg, 1.0 mmol) in dry THF (12 μL) was added CS₂ (601 μL, 10 mmol) at −10° C., then EDC (383 mg, 2.0 mmol) was added in one portion. After being stirred for 15 minutes, TEA (279 μL, 2.0 mmol) was added dropwise. The mixture was allowed to slowly warm up to room temperature and stirred for 4 hours. After filtration, the filtrate was concentrated and the residue was taken up in ethyl acetate (20 mL), extracted with saturated NaHCO₃ (1×10 mL), water (1×10 mL) and then brine (1×10 mL), dried over Na₂SO₄, then concentrated in vacuo. The residue was purified by passing through a short pad of silica gel (30% EtOAc in DCM) to give the title compound as a pale yellow oil (230 mg, 91%). MS (ESI) (M+H)⁺=253.

### 35D: 5-chloro-6-[2-[[(10,11-dihydro-5H-benzo[4,5]cyclohepta[1,2-b]pyridin-5-yl)amino]thioxomethyl]hydrazino]-3-pyridinecarboxylic Acid

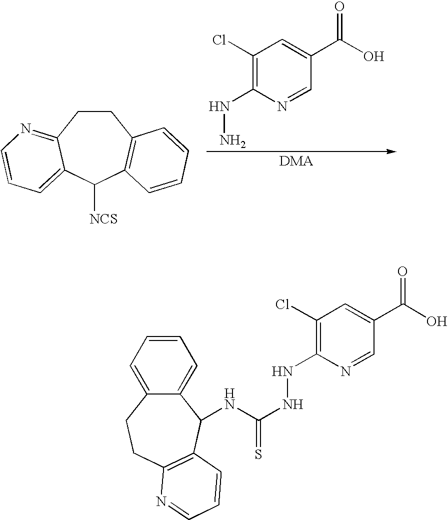

To a solution of 10,11-dihydro-5-isothiocyanato-5H-benzo[4,5]cyclohepta[1,2-b]pyridine (128 mg, 0.5 mmol) in DMA (3 mL) was added 5-chloro-6-hydrazino-3-pyridinecarboxylic acid (94 mg, 0.5 mmol), the mixture was stirred at room temperature for 2 hours, the solvent was concentrated in vacuo and water was added, the precipitate was collected by filtration, dried to give the title compound as off-white powders (211 mg, 96%). MS (ESI) (M+H)⁺=440.

### 35E: 5-chloro-6-[2-[[(10,11-dihydro-5H-benzo[4,5]cyclohepta[1,2-b]pyridin-5-yl)amino]thioxomethyl]hydrazino]-N-[(3R)-1-methyl-2-oxo-3-pyrrolidinyl]-3-pyridinecarboxamide

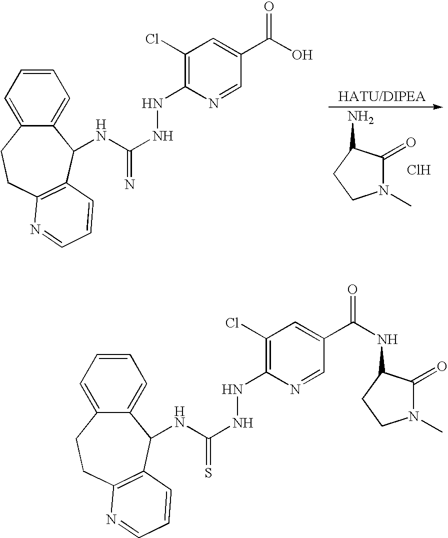

To a stirred solution of 5-chloro-6-[2-[[(10,11-dihydro-5H-benzo[4,5]cyclohepta[1,2-b]pyridin-5-yl)amino]thioxomethyl]hydrazino]-3-pyridinecarboxylic acid (88 mg, 0.2 mmol) in DMA (2 mL) was added HATU (92 mg, 0.24 mmol) followed by DIPEA (41 μL, 0.24 mmol). The mixture was stirred at room temperature for 10 min, and then (3R)-3-amino-1-methyl-2-pyrrolidinone hydrochloride (36 mg, 0.24 mmol) was added. The resulting mixture was stirred at room temperature for 4 h. The product mixture was concentrated in vacuo, the residue was taken up in DCM (20 mL), and washed with concentrated NaHCO₃ (2×10 mL) and then brine (1×10 mL). The residue obtained after removal of the solvent was purified by preparative TLC (8% MeOH in DCM) to provide the title compound (23 mg, 21%). ¹H-NMR (400 MHz, CD₃OD): δ 8.57(s, 1H), 8.33-8.25 (m, 1H), 8.17-8.09(m, 1H), 7.87 (d, J=5 Hz, 1H), 7.37(s, br, 1H), 7.33 (d, J=5 Hz, 1H), 7.24-7.10(m, 4H), 4.71-4.60(m, 1H), 3.52-3.41 (m, 2H), 3.39-3.06(m, 4H), 2.89 (s, 3H), 2.56-2.44 (m, 1H), 2.13-2.04 (m, 1H) ppm. HRMS (M+H)⁺ Calcd for C₂₆H₂₆ClN₇O₂S: 536.1635; Found: 536.1689.

### Example 36

### 5-chloro-6-[2-[[(9-fluoro-10,11-dihydro-5H-benzo[4,5]cyclohepta[1,2-b]pyridin-5-yl)amino]carbonyl]hydrazino]-N-[(3R)-1-methyl-2-oxo-3-pyrrolidinyl]-3-pyridinecarboxamide

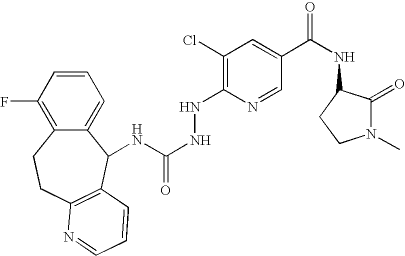

### 36A: 2-[(E)-2-(2-fluorophenyl)ethenyl]-3-pyridinecarboxylic Acid, Sodium Salt

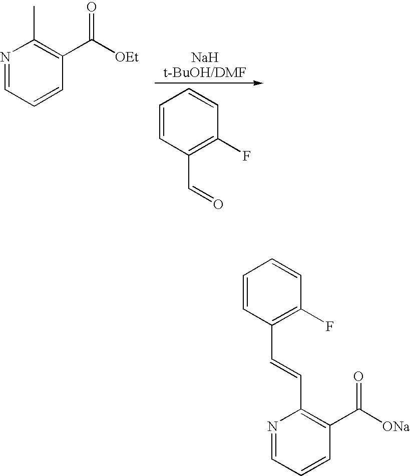

The mixture of NaH (2.64 g, 60% in mineral oil, 110 mmol), t-BuOH (8.62 mL, 90 mmol) in DMF (60 mL) was stirred at room temperature for 10 minutes and then warm up to 50° C. until gas evolution ceased. The mixture was cooled to 0° C. and a solution of ethyl 4-methylnicotinate (5.0 g, 30 mmol) in DMF (10 mL) was added. After being stirred at the same temperature for 1 hour, 2-fluorobenzaldehyde (3.86 mL, 36 mmol) in DMF (10 mL) was added and the mixture was warmed up to room temperature and stirred for overnight. After being concentrated, the residue was used for the next step without further purification. MS (ESI) (M+H)⁺=244.

### 36B: 2-[2-(2-fluorophenyl)ethyl]-3-pyridinecarboxylic Acid, Sodium Salt

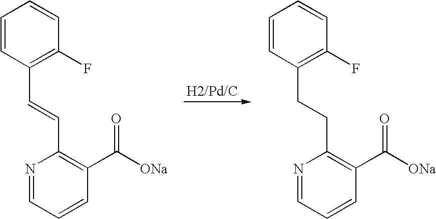

The above crude sodium salt of 2-[(E)-2-(2-fluorophenyl)ethenyl]-3-pyridinecarboxylic acid was dissolved in MeOH (100 mL) and the mixture was hydrogenated at 40 psi for 3 h in the presence of 10% Pd/C at room temperature. After filtration, the filtrate was concentrated dried in vacuo and the solid was used for the next step without further purification. MS (ESI) (M+H)⁺=246.

### 36C: 9-fluoro-10,11-dihydro-5H-benzo[4,5]cyclohepta[1,2-b]pyridin-5-one

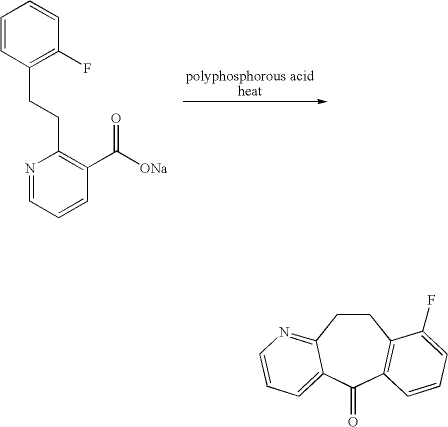

To the above crude sodium salt of 2-[2-(2-fluorophenyl)ethyl]-3-pyridinecarboxylic acid was added PPA (150 g) and the mixture was stirred at 160° C. for 6 h. After cooling to 100° C., the mixture was poured into crashed ice (100 g) and the solution was carefully basified with KOH to pH>10. After extraction with EtOAc (4×80 mL), dried over Na₂SO₄ and evaporation, the title compound was obtained as pale yellow solids (3.2 g, 47% for 3 steps) and was used for the next step without further purification. MS (ESI) (M+H)⁺=228.

### 36D: 9-fluoro-10,11-dihydro-5H-benzo[4,5]cyclohepta[1,2-b]pyridin-5-one, Oxime

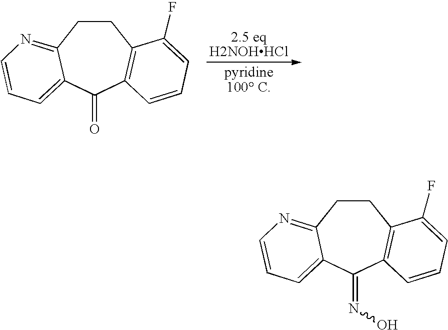

To a stirred solution of 9-fluoro-10,11-dihydro-5H-benzo[4,5]cyclohepta[1,2-b]pyridin-5-one (1.98 g, 8.68 mmol) in pyridine (10 mL) at 100° C. was added H₂NOH.HC (1.51 g, 21.7 mmol) and the resulting mixture was stirred at the same temperature overnight. After the reaction was completed, pyridine was removed in vacuum, the residue was added water and the precipitate was collected by filtration, washed with water, dried to give the title compound as pale yellow powders (2.05 g, 98%), which was used without further purification. MS (ESI) (M+H)⁺=243.

### 36E: 9-fluoro-10,11-dihydro-5H-benzo[4,5]cyclohepta[1,2-b]pyridin-5-amine

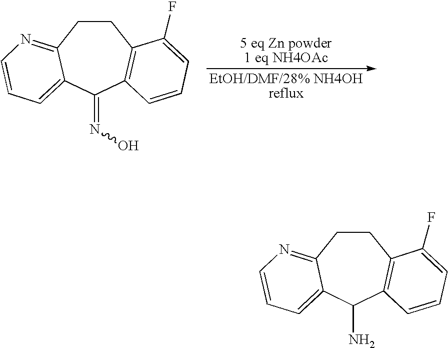

To a mixture of 9-fluoro-10,11-dihydro-5H-benzo[4,5]cyclohepta[1,2-b]pyridin-5-one oxime (2.05 g, 8.48 mmol) in EtOH (18 mL), DMF (8 mL) and 28% NH₄OH (32 mL) was added Zn powder (2.76 g, 42.3 mmol) followed by NH₄OAc (0.66 g, 8.48 mmol). The mixture was stirred at reflux for 3 h. After cooled to room temperature, Et₂O (120 mL) was added and stirred for 20 min, then 1N NaOH (48 mL) was added and stirred for another 10 min. After filtered through Celite, the organic layer was isolated, and the basic aqueous solution was extracted with Et₂O (4×60 mL). The combined organic layer was washed with water and then brine, dried over Na₂SO₄, evaporated to give the title compound as white solids (1.83 g, 95%), which was used without further purification. MS (ESI) (M+H)⁺=229.

### 36F: 5-chloro-6-[2-[[(9-fluoro-10,11-dihydro-5H-benzo[4,5]cyclohepta[1,2-b]pyridin-5-yl)amino]carbonyl]hydrazino]-N-[(3R)-1-methyl-2-oxo-3-pyrrolidinyl]-3-pyridinecarboxamide

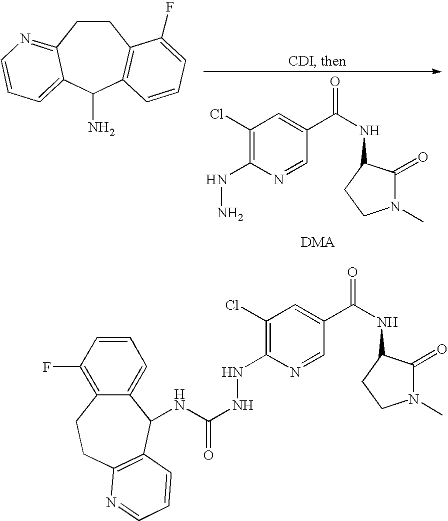

To a solution of CDI (15.4 mg, 0.095 mmol) in dry DMF (1 mL) was added a solution of 9-fluoro-10,11-dihydro-5H-benzo[4,5]cyclohepta[1,2-b]pyridin-5-amine (22 mg, 0.095 mmol) in dry DMF (1 mL) at 0° C. After being stirred for 1 hour, the mixture was allowed to warm up to room temperature and 5-chloro-6-hydrazino-N-[(3R)-1-methyl-2-oxo-3-pyrrolidinyl]-3-pyridinecarboxamide (27 mg, 0.095 mmol) was added. The mixture was stirred at room temperature for another 2 hours. The solvent was concentrated in vacuo, the residue was taken up into DCM (20 mL) and washed with water (1×10 mL), dried over Na₂SO₄, concentrated and purified by flash chromatography (5-10% MeOH in DCM) to give the title compound as white solids (29 mg, 54%). ¹H-NMR (400 MHz, CD₃OD): δ8.62-8.58 (m, 2H), 8.49-8.52(m, 1H), 8.11(d, J=2 Hz, 1H), 7.82(t, J=8 Hz, 1H), 7.30-7.22(m, 2H), 7.13-7.04(m, 1H), 6.53(s, 1H), 4.70-4.60 (m, 1H), 3.60-3.35 (m, 6H), 2.55-2.41 (m, 1H), 2.12-1.98 (m, 1H) ppm. MS (ESI)(M+1)⁺=538. HRMS (M+1)⁺ Calcd for C₂₆H₂₅ClFN₇O₃: 538.1769; Found: 538.1727. Anal. Calcd for C₂₆H₂₅ClFN₇O₃.0.2H₂O.2.5TFA: C, 45.04; H, 3.40; N, 11.86; Found: C, 45.08; H, 3.41; N, 11.80.

### Example 37

### 5-chloro-6-[2-[[(9-fluoro-10,11-dihydro-5H-benzo[4,5]cyclohepta[1,2-b]pyridin-5-yl)amino]carbonyl]hydrazino]-N-[(3R)-tetrahydro-2-oxo-3-furanyl]-3-pyridinecarboxamide

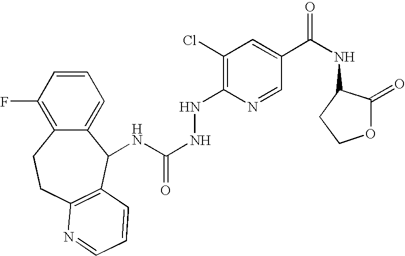

### 37A: 5-chloro-6-[2-[[(9-fluoro-10,11-dihydro-5H-benzo[4,5]cyclohepta[1,2-b]pyridin-5-yl)amino]carbonyl]hydrazino]-3-pyridinecarboxylic Acid

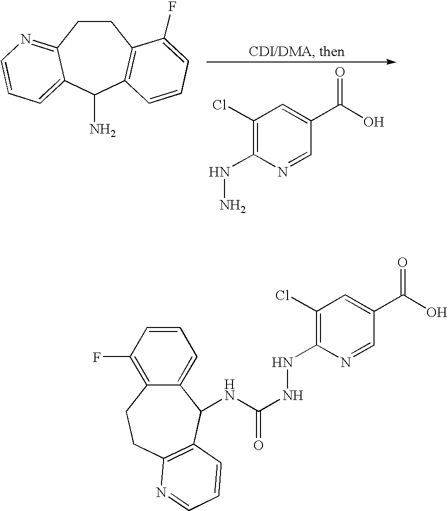

To a solution of CDI (61 mg, 0.38 mmol) in dry DMF (4 mL) was added a solution of 9-fluoro-10,11-dihydro-5H-benzo[4,5]cyclohepta[1,2-b]pyridin-5-amine (87 mg, 0.38 mmol) in dry DMF (4 mL) at 0° C. After being stirred for 1 hour, the mixture was allowed to warm up to room temperature and 5-chloro-6-hydrazino-3-pyridinecarboxylic acid (71 mg, 0.38 mmol) was added. The mixture was stirred at room temperature for another 2 hours. The solvent was concentrated in vacuo, the residue was treated with small amount of MeOH and diethyl ether, the solid was collected and dried to give the title compound (139 mg, 83%). MS (ESI) (M+H)⁺=442.

### 37B: 5-chloro-6-[2-[[(9-fluoro-10,11-dihydro-5H-benzo[4,5]cyclohepta[1,2-b]pyridin-5-yl)amino]carbonyl]hydrazino]-N-[(3R)-tetrahydro-2-oxo-3-furanyl]-3-pyridinecarboxamide

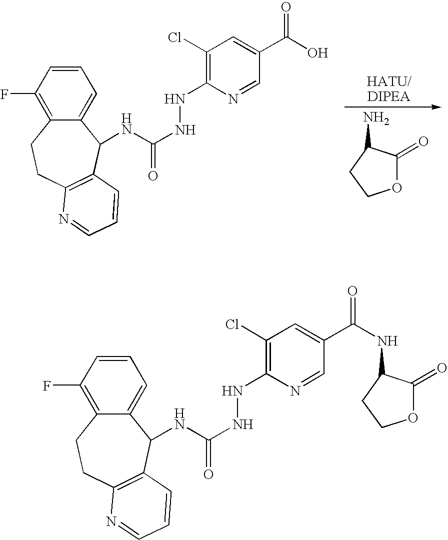

To a stirred solution of 5-chloro-6-[2-[[(9-fluoro-10,11-dihydro-5H-benzo[4,5]cyclohepta[1,2-b]pyridin-S-yl)amino]carbonyl]hydrazino]-3-pyridinecarboxylic acid (84 mg, 0.19 mmol) in DMA (2 mL) was added HATU (87 mg, 0.23 mmol) followed by DIPEA (39 μL, 0.23 mmol). The mixture was stirred at room temperature for 10 min, and then (R)-(+)-α-amino-ω-butyrolactone hydrochloride (32 mg, 0.23 mmol) was added. The resulting mixture was stirred at room temperature for 4 h. The product mixture was concentrated in vacuo, the residue was taken up in DCM (20 mL), and washed with concentrated NaHCO₃ (2×10 mL) and then brine (1×10 mL). The residue obtained after removal of the solvent was purified by reverse-phase HPLC (10˜40% MeCN in water) to provide the title compound (37 mg, 31%) as a TFA salt. ¹H-NMR (400 MHz, CD₃OD): δ 8.59 (s, 1H), 8.57(s, 1H), 8.55-8.51(m, 1H), 8.10(d, 1 Hz, 1H), 7.82-7.77 (m, 1H), 7.30-7.20(m, 2H), 7.13-7.04(m, 1H), 6.54(s, 1H), 4.81-4.75 (m, 1H), 4.52-4.48(m, 1H), 4.39-4.31(m, 1H), 3.62-3.36 (m, 4H), 2.65-2.56 (m, 1H), 2.46-2.36 (m, 1H) ppm. MS (ESI) (M+1)⁺=525. Calcd for C₂₅H₂₂ClFN₆O₄: HRMS (M+H)⁺ 525.1453; Found: 525.1307.

### Example 38

### 5-chloro-6-[2-[[(7-fluoro-10,11-dihydro-5H-benzo[4,5]cyclohepta[1,2-b]pyridin-5-yl)amino]thioxomethyl]hydrazino]-N-[(3R)-1-methyl-2-oxo-3-pyrrolidinyl]-3-pyridinecarboxamide

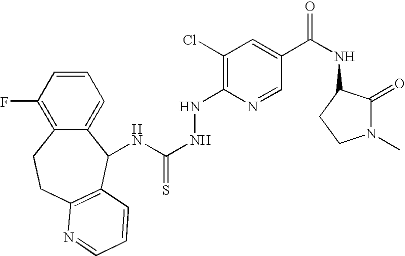

### 38A: 2-[(E)-2-(4-fluorophenyl)ethenyl]-3-pyridinecarboxylic Acid, Sodium Salt

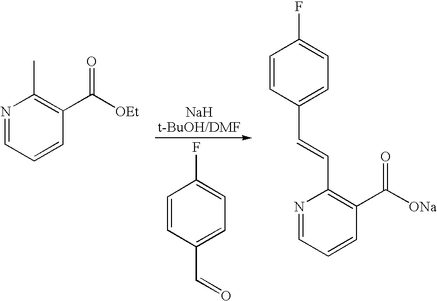

The mixture of NaH (1.32 g, 60% in mineral oil, 55 mmol), t-BuOH (4.31 mL, 45 mmol) in DMF (30 mL) was stirred at room temperature for 10 minutes and then warm up to 50° C. until the formation of air bubbles ceased. The mixture was cooled to 0° C. and a solution of ethyl 4-methylnicotinate (2.5 g, 15 mmol) in DMF (5 mL) was added. After being stirred at the same temperature for 1 hour, 4-fluorobenzaldehyde (1.93 mL, 18 mmol) in DMF (5 mL) was added and the mixture was warmed up to room temperature and stirred for overnight. After being concentrated, the residue was used for the next step without further purification MS (ESI) (M+H)⁺=244.

### 38B: 2-[2-(4-fluorophenyl)ethyl]-3-pyridinecarboxylic Acids Sodium Salt

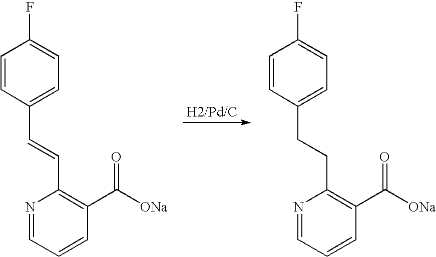

The above sodium salt of 2-[(E)-2-(4-fluoro phenyl)ethenyl]-3-pyridinecarboxylic acid was dissolved in MeOH (60 mL) and the mixture was hydrogenated at 40 psi for 3 h in the presence of 10% Pd/C at room temperature. After filtration, the filtrate was concentrated dried in vacuo and the solid was used for the next step without further purification. MS (ESI) (M+H)⁺=246.

### 38C: 7-fluoro-10,11-dihydro-5H-benzo[4,5]cyclohepta[1,2-b]pyridin-5-one

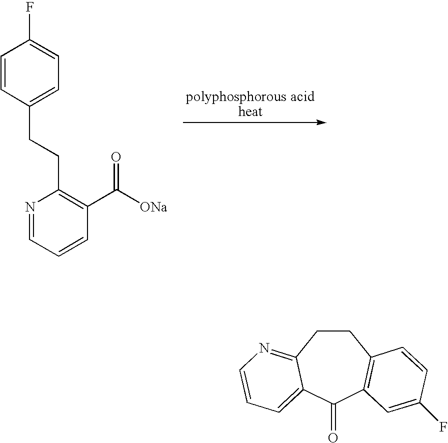

To the above sodium salt of 2-[2-(2-fluorophenyl)ethyl]-3-pyridinecarboxylic acid was added PPA (100 g) and the mixture was stirred at 160° C. for 6 h. After cooling to 100° C., the mixture was poured into crushed ice (100 g) and the solution was carefully basified with KOH to pH>10. After extraction with EtOAc (4×50 mL), dried over Na₂SO₄ and evaporation, the title compound was obtained as pale yellow solids (2.1 g, 61% for 3 steps) and was used for the next step without further purification. MS (ESI) (M+H)⁺=228.

### 38D: 7-fluoro-10,11-dihydro-5H-benzo[4,5]cyclohepta[1,2-b]pyridin-5-one, Oxime

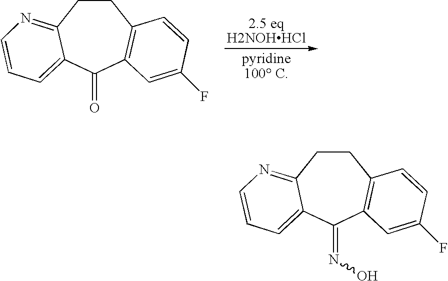

To a stirred solution of 7-fluoro-10,11-dihydro-5H-benzo[4,5]cyclohepta[1,2-b]pyridin-5-one (1.9 g, 8.36 mmol) in pyridine (10 mL) at 100° C. was added H₂NOH.HCl (1.45 g, 20.9 mmol) and the resulting mixture was stirred at the same temperature overnight. After the reaction was completed, pyridine was removed in vacuum, the residue was added water and the precipitate was collected by filtration, washed with water, dried to give the title compound as pale yellow powders (2.01 g, 99%), which was used without further purification. MS (ESI) (M+H)⁺=243.

### 38E: 7-fluoro-10,11-dihydro-5H-benzo[4,5]cyclohepta[1,2-b]pyridin-5-amine

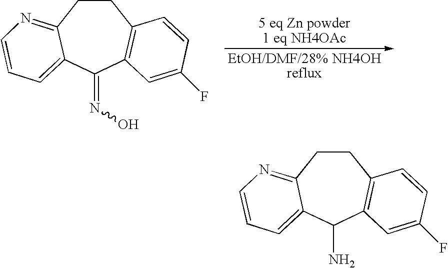

To a mixture of 7-fluoro-10,11-dihydro-5H-benzo[4,5]cyclohepta[1,2-b]pyridin-5-one oxime (2.01 g, 8.31 mmol) in EtOH (18 mL), DMF (8 mL) and 28% NH₄OH (32 mL) was added Zn powder (2.71 g, 41.5 mmol) followed by NH₄OAc (0.65 g, 8.31 mmol). The mixture was stirred at reflux for 3 h. After cooled to room temperature, Et₂O (120 mL) was added and stirred for 20 min, then 1N NaOH (48 mL) was added and stirred for another 10 min. After filtered through Celite, the organic layer was isolated, and the basic aqueous solution was extracted with Et₂O (4×60 mL). The combined organic layer was washed with water and then brine, dried over Na₂SO₄, evaporated to give the title compound as white solids (1.84 g, 97%), which was used without further purification. MS (ESI) (M+H)⁺=229.

### 38F: 7-fluoro-10,11-dihydro-5-isothiocyanato-5H-benzo[4,5]cyclohepta[1,2-b]pyridine

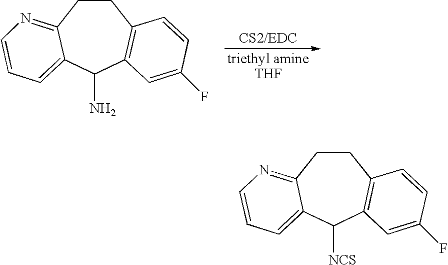

To a solution of 7-fluoro-6,11-dihydro-5H-benzo[5,6]cyclohepta[1,2-c]pyridin-11-amine (228 mg, 1.0 mmol) in dry THF (8 mL) was added CS₂ (0.6 mL, 10 mmol) at 10° C., then EDC (383 mg, 2.0 mmol) was added in one portion. After being stirred for 15 minutes, TEA (279 μL, 2.0 mmol) was added dropwise. The mixture was allowed to slowly warm up to room temperature and stirred for 4 hours. After filtration, the filtrate was concentrated and the residue was taken up in ethyl acetate (30 mL), extracted with saturated NaHCO₃ (1×10 mL), water (1×10 mL) and then brine (1×10 mL), dried over Na₂SO₄, then concentrated in vacuo. The residue was purified by passing through a short pad of silica gel (30% EtOAc in DCM) to give the title compound as white solids (229 mg, 85%). MS (ESI) (M+H)⁺=271.

### 38G: 5-chloro-6-[2-[[(7-fluoro-10,11-dihydro-5H-benzo[4,5]cyclohepta[1,2-b]pyridin-5-yl)amino]thioxomethyl]hydrazino]-N-[(3R)-1-methyl-2-oxo-3-pyrrolidinyl]-3-pyridinecarboxamide

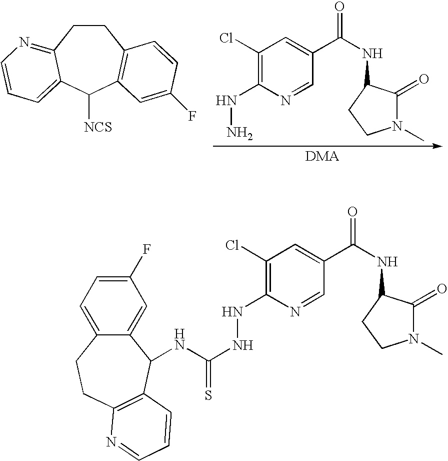

To a stirred solution of 7-fluoro-10,11-dihydro-5-isothiocyanato-5H-benzo[4,5]cyclohepta[1,2-b]pyridine (27 mg, 0.1 mmol) in DMA (2 mL) was added 5-chloro-6-hydrazino-N-[(3R)-1-methyl-2-oxo-3-pyrrolidinyl]-3-pyridinecarboxamide (28.4 mg, 1.0 mmol). The mixture was stirred at room temperature for 2 hours and then concentrated in vacuo, the residue was purified by flash chromatography (5˜10% MeOH in DCM) to give the title compound as white solids (18 mg, yield 32%). ¹H-NMR (400 MHz, CD₃OD): δ 8.62(s, 1H), 8.31(d, J=5 Hz, 1H), 8.15(d, J=2 Hz, 1H), 7.90 (d, J=8 Hz, 1H), 7.53(s, br, 1H), 7.29-7.20(m, 2H), 7.13 (d, J=8 Hz, 1H), 6.96-6.90(m, 1H), 4.70-4.61(m, 1H), 3.48-3.42 (m, 2H), 3.42-3.05(m, 4H), 2.89 (s, 3H), 2.53-2.43 (m, 1H), 2.11-2.00 (m, 1H) ppm. MS (M+H)⁺=554. HRMS (M+1)⁺ Calcd for C₂₆H₂₅ClFN₇O₂S: 554.1541; Found: 554.1533.

### Example 39

### 5-chloro-6-[2-[[(7-fluoro-10,11-dihydro-5H-benzo[4,5]cyclohepta[1,2-b]pyridin-5-yl)amino]thioxomethyl]hydrazino]-N-[(3R)-2-oxo-1-phenyl-3-pyrrolidinyl]-3-pyridinecarboxamide

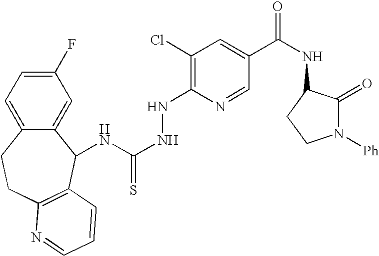

### 39A: 5-chloro-6-[2-[[(7-fluoro-10,11-dihydro-5H-benzo[4,5]cyclohepta[1,2-b]pyridin-5-yl)amino]thioxomethyl]hydrazino]-3-pyridinecarboxylic Acid

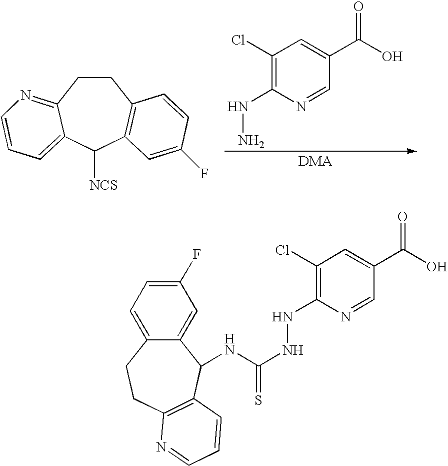

To a solution of 7-fluoro-10,11-dihydro-5-isothiocyanato-5H-benzo[4,5]cyclohepta[1,2-b]pyridine (141 mg, 0.52 mmol) in DMA (3 mL) was added 5-chloro-6-hydrazino-3-pyridinecarboxylic acid (98 mg, 0.52 mmol), the mixture was stirred at room temperature for 2 hours, the solvent was concentrated in vacuo and water was added, the precipitate was collected by filtration, dried to give the title compound as off-white powders (213 mg, 90%). MS (ESI) (M+H)⁺=458.

### 39B: 5-chloro-6-[2-[[(7-fluoro-10,11-dihydro-5H-benzo[4,5]cyclohepta[1,2-b]pyridin-5-yl)amino]thioxomethyl]hydrazino]-N-[(3R)-2-oxo-1-phenyl-3-pyrrolidinyl]-3-pyridinecarboxamide

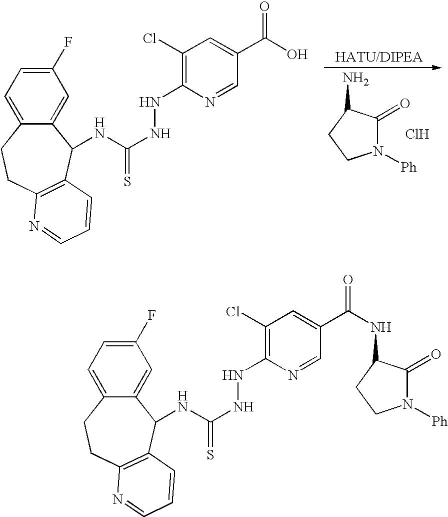

To a stirred solution of 5-chloro-6-[2-[[(7-fluoro-10,11-dihydro-5H-benzo[4,5]cyclohepta[1,2-b]pyridin-5-yl)amino]thioxomethyl]hydrazino]3-pyridinecarboxylic acid (115 mg, 0.25 mmol) in DMA (4 mL) was added HATU (114 mg, 0.3 mmol) followed by DIPEA (51 μL, 0.3 mmol). The mixture was stirred at room temperature for 10 min. and then (3R)-3-amino-1-phenyl-2-pyrrolidinone hydrochloride (64 mg, 0.3 mmol) was added. The resulting mixture was stirred at room temperature for 4 h. The product mixture was concentrated in vacuo, the residue was taken up in DCM (20 mL), and washed with conc. NaHCO₃ (2×10 mL) and then brine (1×10 mL). The residue obtained after removed of the solvent was purified by flash chromatography (0˜5% MeOH in DCM) to provide the title compound (67 mg, 44°/O). ¹H-NMR (400 MHz, CD₃OD): δ 8.64 (s, 1H), 8.25 (d, J=5 Hz, 1H), 80.10 (d, J=2 Hz, 1H), 7.81 (d, J=5 Hz, 1H), 7.63 (d, J=8 Hz, 2H), 7.49(s, br, 1H), 7.37(t, J=8 Hz, 2H), 7.26-7.06 (m, 4H), 6.97-6.83(m, 1H), 4.98-4.82 (m, 1H), 4.01-3.70(m, 2H), 3.46-3.25(m, 2H), 3.24-3.01(m, 2H), 2.65-2.46 (m, 1H), 2.29-2.11 (m, 1H) ppm. MS (ESI) (M+H)⁺=616. HRMS (M+1)⁺ Calcd for C₃₁H₂₇ClFN₇O₂S: 616.1698; Found: 616.1641.

### Example 40

### 5-chloro-6-[2-[[(7-fluoro-6,11-dihydro-5H-benzo[5,6]cyclohepta[1,2-c]pyridin-11-yl)amino]thioxomethyl]hydrazino]-N-(2-methoxy-3-pyridinyl)-3-pyridinecarboxamide

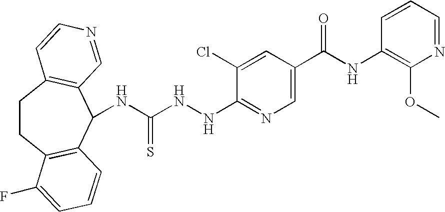

### 40A: 5,6-dichloro-N-(2-methoxy-3-pyridinyl)-3-pyridinecarboxamide

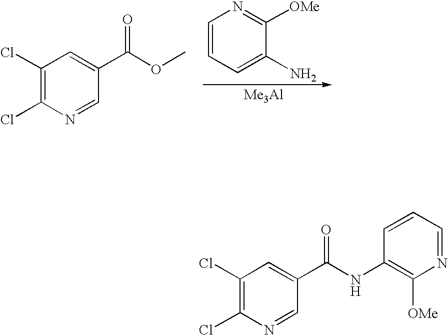 2 M Me₃Al in heptane (0.5 ml, 1.0 mmol) was added dropwise to a solution of 2-methoxy-3-aminopyridine (124 mg, 1.0 mmol) in CH₂Cl₂ (5 ml) at 0° C. The mixture was stirred at room temperature for 30 min, then methyl 5,6-dichloro-3-pyridinecarboxylate (204 mg, 1.00 mmol) from Experiment 15A was added. The mixture was stirred at room temperature for 3 h, quenched with H₂O (1 ml), diluted with CH₂Cl₂ (30 ml), washed with 10% citric acid (5 ml), saturated NaHCO₃, brine, dried over Na₂SO₄, concentrated in vacuo to give 5,6-dichloro-N-(2-methoxy-3-pyridinyl)-3-pyridinecarboxamide (303 mg, quantitatively). MS (ESI) (M+1)⁺=298 (300).

### 40B: 5-chloro-6-hydrazino-N-(2-methoxy-3 pyridinyl)-3-pyridinecarboxamide

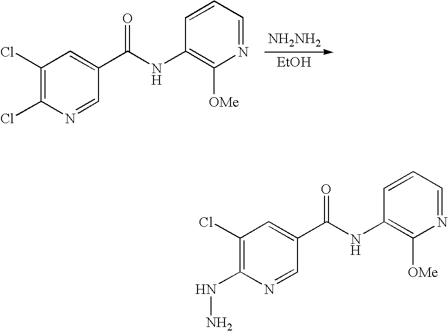

5,6-dichloro-N-(2-methoxy-3-pyridinyl)-3-pyridinecarboxamide (300 mg, 1 mmol) from step A and hydrazine monohydrate (0.1 ml) in EtOH (5 ml) was heated at reflux for 2 h, allowed to cool to room temperature, and the solid was collected (102 mg). MS (ESI)(M+1)⁺=294.

### 40C: 5-chloro-6-[2-[[(7-fluoro-6,11-dihydro-5H-benzo[5,6]cyclohepta[1,2-c]pyridin-11-yl)amino]thioxomethyl]hydrazino]-N-(2-methoxy-3-pyridinyl)-3-pyridinecarboxamide

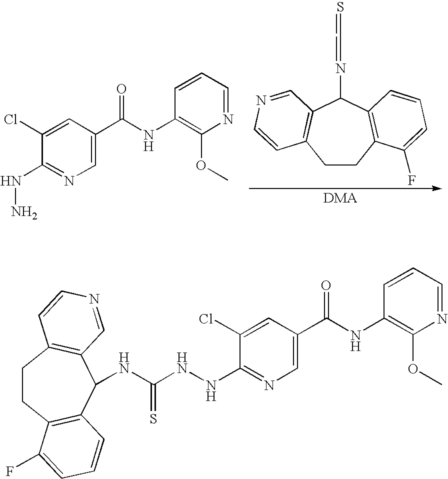

7-fluoro-6,11-dihydro-11-isothiocyanato-5H-benzo[5,6]cyclohepta[1,2-c]pyridine (27 mg, 0.10 mmol) from 4970X and 5-chloro-6-hydrazino-N-(2-methoxy-3-pyridinyl)-3-pyridinecarboxamide (29 mg, 0.10 mmol) from 920B were dissolved in DMA (1 ml), and the reaction mixture was stirred at room temperature for 3 h, and H₂O (10 ml) was added, and the solid was collected and dried. The crude product was further purified on silica gel (eluent MeOH:CH₂Cl₂ 1:25) to give 5-chloro-6-[2-[[(7-fluoro-6,11-dihydro-5H-benzo[5,6]cyclohepta[1,2-c]pyridin-11-yl)amino]thioxomethyl]hydrazino]-N-(2-methoxy-3-pyridinyl)-3-pyridinecarboxamide (47 mg, 83%). ¹HNMR(400 MHz, CDCl₃): δ 8.58-8.62(m, 3H), 8.33 (s, 1H), 8.32 (s, 1H), 8.11(s, 1H), 8.00-8.10(br, 1H), 7.91(d, J=1.7 Hz, 1H), 7.89(d, J=1.7 Hz, 1H), 7.21(d, J=7.6 Hz, 1H), 7.05-7.15(m, 2H), 7.01(d, J=6.1 Hz, 1H), 6.88-6.98(m, 2H), 4.05 (s, 3H), 3.14 (m, 4H) ppm. MS(ESI) (M+1)⁺=564. HRMS (M+1)⁺ Calcd for C₂₇H₂₃ClFN₇O₂S: 564.1385; Found: 564.1302.

### Example 41

### 6-[2-[[(7-fluoro-6,11-dihydro-5H-benzo[5,6]cyclohepta[1,2-c]pyridin-11-yl)amino]thioxomethyl]hydrazino]-N-[(3R)-1-methyl-2-oxo-3-pyrrolidinyl]-3-pyridinecarboxamide

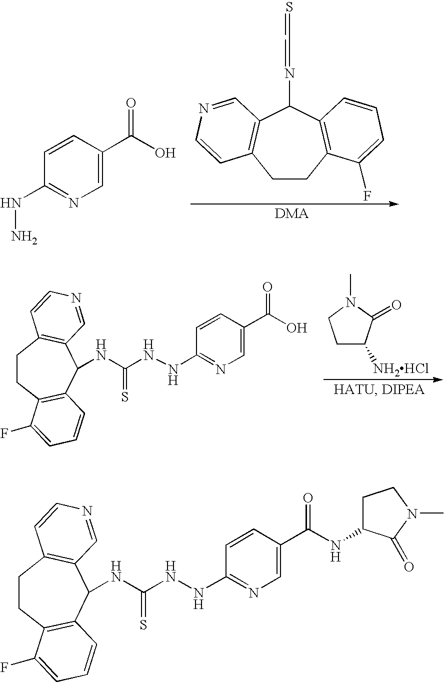

7-fluoro-6,11-dihydro-11-isothiocyanato-5H-benzo[5,6]cyclohepta[1,2-c]pyridine (27 mg, 0.10 mmol) and 6-hydrazinonicotinic acid (15 mg, 0.10 mmol) prepared from hydrazine and nicotinic acid were dissolved in DMA (2 ml), the reaction mixture was stirred at room temperature for 16 h, and the acid intermediate as its DMA solution was prepared. MS(ESI, M+1)⁺: 423.

(3R)-3-amino-1-methyl-2-pyrrolidinone hydrochloride (30 mg), DIPEA (0.1 ml), HATU (65 mg) was added successively to the above acid intermediate. The mixture was stirred at room temperature for 2 h, the reaction mixture was subject to preparative LCMS without work-up and the desired product (10 mg) was obtained as TFA salt.

¹HNMR(400 MHz, CD₃OD): δ 8.81 (m, 1H), 8.64 (s, 1H), 8.59 (d, J=5.9 Hz, 1H), 8.07 (m, 1H), 7.85 (d, J=3.9 Hz, 1H), 7.37 (br, 1H), 7.22 (m, 2H), 7.04-7.10 (m, 1H), 6.77 (d, J=8.8 Hz, 1H), 4.67 (m, 1H), 3.40-3.50 (m, 4H), 3.33 (m, 1H), 3.15 (m, 1H), 2.90 (s, 3H), 2.40-2.50 (m, 1H), 2.00-2.10 (m, 1H) ppm. MS (ESI)(M+1)⁺=520. HRMS (M+1)⁺ Calcd for C₂₆H₂₆FN₇O₂S: 520.1931; Found: 520.1981.

### Example 42

### 5-chloro-6-[[[[(7-fluoro-6,11-dihydro-5H-benzo[5,6]cyclohepta[1,2-c]pyridin-11-yl)amino]thioxomethyl]amino]methyl]-N-[(3R)-tetrahydro-2-oxo-3-thienyl]-3-pyridinecarboxamide

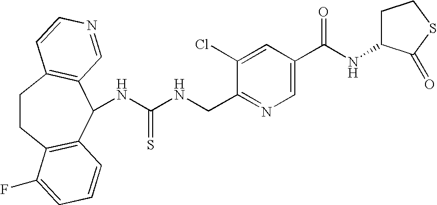

### 42A: 5-chloro-6-[[[[(7-fluoro-6,11-dihydro-5H-benzo[5,6]cyclohepta[1,2-c]pyridin-1-yl)amino]thioxomethyl]amino]methyl]-3-pyridinecarboxylic Acid, Methyl Ester

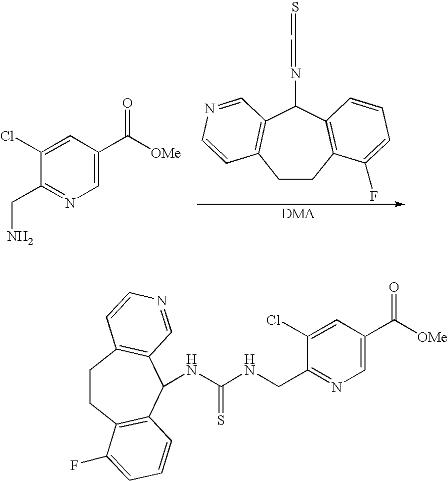

7-fluoro-6,11-dihydro-11-isothiocyanato-5H-benzo[5,6]cyclohepta[1,2-c]pyridine (270 mg, 1.0 mmol) and methyl 6-(aminomethyl)-5-chloro-3-pyridinecarboxylate (220 mg, ˜1.10 mmol) from Example 15C was dissolved in DMA (6 ml), the reaction mixture was stirred at room temperature overnight, and DMA was stripped off in vacuo. The residue was subjected to flash chromatography on silica gel (EtOAc:CH₂Cl₂ 1:3) to give the desired thiourea compound (220 mg).

### 42B: 5-chloro-6-[[[[(7-fluoro-6,11-dihydro-5H-benzo[5,6]cyclohepta[1,2-c]pyridin-11-yl)amino]thioxomethyl]amino]methyl]-3-pyridinecarboxylic Acid

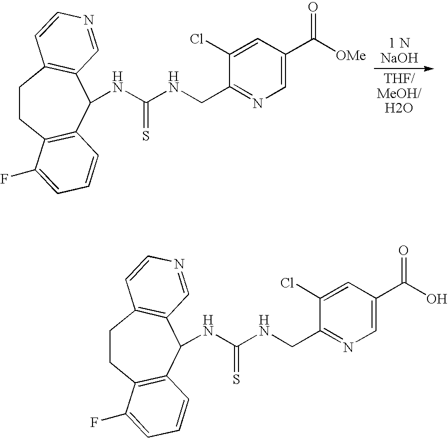

1 N NaOH (1 ml, 1.0 mmol) was added to a solution of 5-chloro-6-[[[[(7-fluoro-6,11-dihydro-5H-benzo[5,6]cyclohepta[1,2-c]pyridin-11-yl)amino]thioxomethyl]amino]methyl]-3-pyridinecarboxylic acid methyl ester (220 mg, 0.468 mmol) in a solvent mixture of MeOH/THF/H₂O (7.5 ml, 1:1:1), the reaction mixture was heated at 50° C. for 30 min, then concentrated in vacuo, and then re-dissolved H₂O (10 ml). 1N HCl (1 ml) was added, the precipitate was collected and dried to afford the acid intermediate (210 mg), MS (M+1)⁺=457.

### 42C: 5-chloro-6-[[[[(7-fluoro-6,11-dihydro-5H-benzo[5,6]cyclohepta[1,2-c]pyridin-11-yl)amino]thioxomethyl]amino]methyl]-N-[(3R)-tetrahydro-2-oxo-3-thienyl]-3-pyridinecarboxamide

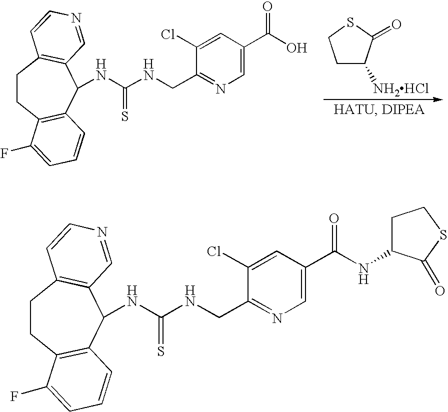

Following general HATU coupling procedure of 1C: HATU (42 mg, 0.11 mmol) was added to a mixture of 5-chloro-6-[[[[(7-fluoro-6,11-dihydro-5H-benzo[5,6]cyclohepta-[1,2-c]pyridin-11-yl)amino]thioxomethyl]amino]methyl]-3-pyridinecarboxylic acid (46 mg, 0.10 mmol) from step B, (D)-homocysteine thiolactone hydrochloride (22 mg), DIPEA (0.1 ml) in DMA (3 ml) at room temperature. The reaction mixture was stirred at room temperature for 2 h, and then DMA was removed under reduced pressure. H₂O (10 ml) was added, and the precipitate was collected and dried. The product was purified on silica gel (EtOAc:CH₂Cl₂ 1:3 to 5% MeOH in CH₂Cl₂) to give the title compound (48 mg, 86%). ¹HNMR (400 MHz, CD₃OD): δ 8.87 (br.s, 1H), 8.64 (s, 1H), 8.34 (d, J=5.1 Hz, 1H), 8.23 (d, J=1.9 Hz, 1H), 7.33 (d, J=5.1 Hz, 1H), 7.31 (d, J=7.6 Hz, 1H), 7.19-7.26 (m, 1H), 7.10 (br.s, 1H), 7.00-7.06 (m, 1H), 4.97 (s, 1H), 4.84-4.90 (m, 2H), 3.40-3.50 (m, 1H), 3.10-3.35 (m, 4H), 2.60-2.67 (m, 1H), 2.23-2.33 (m, 1H) ppm. MS (ESI)(M+1)⁺: 556. HRMS (M+1)⁺ Calcd for C₂₆H₂₃ClFN₅O₂S₂: 556.1044; Found: 556.1091.

### Example 43

### 5-chloro-6-[[[[(7-fluoro-6,11-dihydro-5H-benzo[5,6]cyclohepta[1,2-c]pyridin-11-yl)amino]thioxomethyl]amino]methyl]-N-[(3R)-1-methyl-2-oxo-3-pyrrolidinyl]-3-pyridinecarboxamide

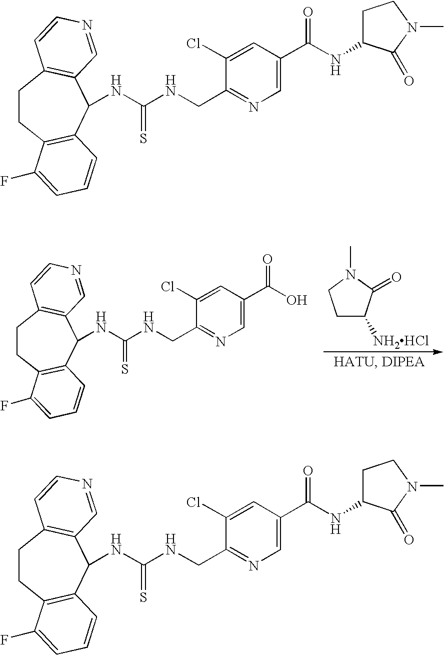

Following general HATU coupling procedure of Example 1C: HATU (95 mg, 0.25 mmol) was added to a mixture of 5-chloro-6-[[[[(7-fluoro-6,11-dihydro-5H-benzo[5,6]cyclohepta-[1,2-c]pyridin-11-yl)amino]thioxomethyl]amino]methyl]-3-pyridinecarboxylic acid (92 mg, 0.20 mmol), (3R)-3-amino-1-methyl-2-pyrrolidinone hydrochloride (60 mg), DIPEA (0.2 ml) in DMA (5 ml) at room temperature. The reaction mixture was stirred at room temperature for 2 h, and then DMA was removed under reduced pressure. H₂O (5 ml) was added, and the precipitate was collected and dried. The product was purified by preparative HPLC and lypholized to give the title compound (25 mg) as its TFA salt. ¹HNMR (CD₃OD): δ 8.97 (br.s, 1H), 8.85 (s, 1H), 8.59 (d, J=5.5 Hz, 1H), 8.23 (m, 1H), 7.89 (d, J=5.9 Hz, 1H), 7.21-7.44 (m, 3H), 7.05-7.20 (m, 1H), 6.97 (s, 1H), 4.93 (s, 1H), 4.68 (m, 1H), 3.78 (m, 1H), 3.44 (m, 4H), 3.12 (m, 1H), 2.89 (s, 3H), 2.40-2.55 (m, 1H), 2.00-2.10 (m, 1H) ppm. MS (ESI) (M+1)⁺=553. HRMS (M+1)⁺ Calcd for C₂₇H₂₆ClFN₆O₂S: 553.1589; Found: 553.1575. CHN Calcd for C₂₇H₂₆ClFN₆O₂S.2CF₃CO₂H.0.5H₂O: C, 47.12; H, 3.70; N, 10.64. Found: C, 46.70; H, 3.63; N, 11.14.

### Example 44

### 5-chloro-6-[[[[(7-fluoro-6,11-dihydro-5H-benzo[5,6]cyclohepta[1,2-c]pyridin-11-yl)amino]carbonyl]amino]methyl]-N-[(3R)-1-methyl-2-oxo-3-pyrrolidinyl]-3-pyridinecarboxamide

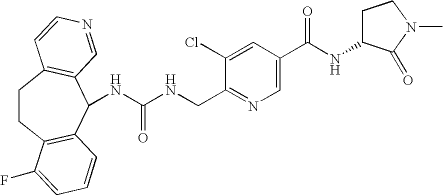

### 44A: 5-chloro-6-[[[[(7-fluoro-6,11-dihydro-5H-benzo[5,6]cyclohepta[1,2-c]pyridin-11-yl)amino]carbonyl]amino]methyl]-3-pyridinecarboxylic Acid, Methyl Ester

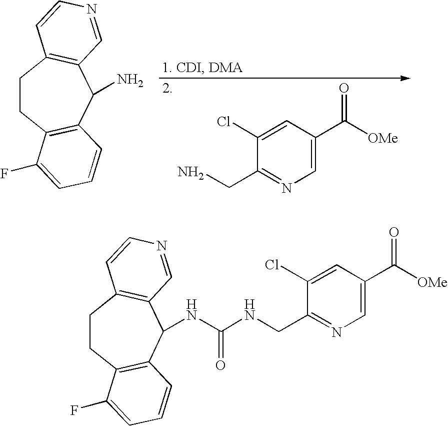

7-fluoro-6,11-dihydro-5H-benzo[5,6]cyclohepta[1,2-c]pyridin-11-amine (120 mg, 0.52 mmol) was added to a solution of CDI (90 mg, 0.55 mmol) in DMF (5 ml) at room temperature and the reaction mixture was stirred at room temperature for 3 h. And then methyl 6-(aminomethyl)-5-chloro-3-pyridinecarboxylate (120 mg, mmol) was added and the reaction mixture was heated at 70° C. for 3 h, DMF was evaporated in vacuo, and the residue was dissolved in CH₂Cl₂ (30 ml), washed with H₂O (2×5 ml), dried, and concentrated in vacuo. The product was purified on silica gel (5% MeOH in CH₂Cl₂) to give the title compound (150 mg, 63%). MS(ESI) (M+1)⁺=455.

### 44B 5-chloro-6-[[[[(7-fluoro-6,11-dihydro-5H-benzo[5,6]cyclohepta[1,2-c]pyridin-11-yl)amino]carbonyl]amino]methyl]-3-pyridinecarboxylic Acid

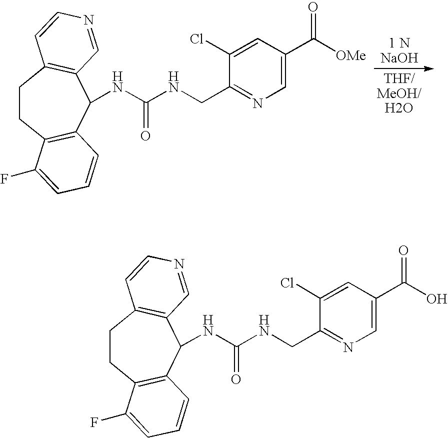

1 N NaOH (0.8 ml, 0.8 mmol) was added to a solution of 5-chloro-6-[[[[(7-fluoro-6,11-dihydro-5H-benzo[5,6]cyclohepta[1,2-c]pyridin-11-yl)amino]carbonyl]amino]methyl]-3-pyridinecarboxylic acid methyl ester (150 mg, 0.330 mmol) from 928A in a solvent mixture of MeOH/THF/H₂O (7.5 ml, 1:1:1), the reaction mixture was heated at 50° C. for 30 min, then concentrated in vacuo, and then re-dissolved H₂O (5 ml). 1N HCl (0.8 ml) was added, the precipitate was collected and dried to afford the acid intermediate (120 mg), MS (M+1)⁺=445.

### 44C: 5-chloro-6-[[[[(7-fluoro-6,11-dihydro-5H-benzo[5,6]cyclohepta[1,2-c]pyridin-11-yl)amino]carbonyl]amino]methyl]-N-[(3R)-1-methyl-2-oxo-3-pyrrolidinyl]-3-pyridinecarboxamide

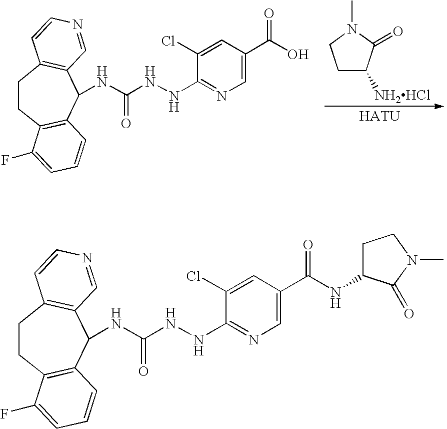

Following the general HATU coupling procedure in Example 1C: The title compound (42 mg, 52%) was obtained from 5-chloro-6-[[[[(7-fluoro-6,11-dihydro-5H-benzo[5,6]cyclohepta[1,2-c]pyridin-11-yl)amino]carbonyl]amino]methyl]-3-pyridinecarboxylic acid and (3R)-3-amino-1-methyl-2-pyrrolidinone hydrochloride.

¹H (400 MHz, CD₃OD): δ 8.87 (s, 1H), 8.70-8.80 (m, 2H), 8.52 (br, 1H), 8.22 (s, 1H), 7.70 (br, 1H), 7.20-7.35 (m, 2H), 7.08 (dd, J=7.6, 8.0 Hz, 1H), 6.31 (s, 1H), 4.68 (m, 1H), 4.59 (s, 2H), 3.67 (m, 1H), 3.30-3.50 (m, 4H), 3.10-3.20 (m, 1H), 2.89 (s, 3H), 2.45-2.55 (m, 1H), 1.95-2.10 (m, 1H) ppm. MS(ESI)(M+)⁺=537. HRMS (M+1)⁺ Calcd for C₂₇H₂₆ClFN₆O₃: 537.1817; Found: 537.1875.

### Example 45

### [5-chloro-6-[2-[[(7-fluoro-6,11-dihydro-5H-benzo[5,6]cyclohepta[1,2-c]pyridin-11-yl)amino]thioxomethyl]hydrazino]-3-pyridinyl]methyl-carbamic Acid, Methyl Ester

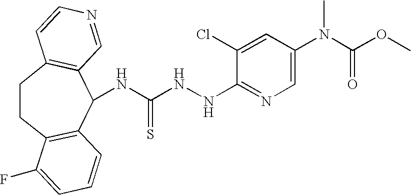

### 45A: (5,6-dichloro-3-pyridinyl)-carbamic Acid, 1,1-dimethylethyl Ester

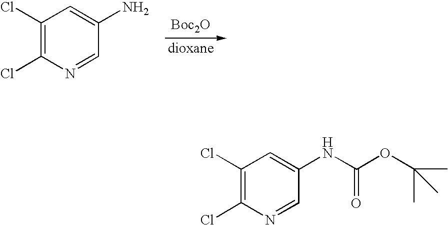

Boc₂O (3.50 g, 16.0 mmol) was added to a solution of 5,6-dichloro-3-pyridinamine (2.00 g, 12.34 mmol) in dioxane, and the mixture was heated at reflux for 8 h, allowed to cool to room temperature. The solvent was stripped off at the reduced pressure, and the residue was dissolved in CH₂Cl₂ (50 ml) and washed with saturated NaHCO₃, brine, dried over Na₂SO₄, concentrated in vacuo. The product was purified on silica gel (CH₂Cl₂:EtOAc 1:2) to give the title compound (1.05 g), MS(ESI)(M+1)⁺=263.

### 45B: 5,6-dichloro-N-methyl-3-pyridinamine

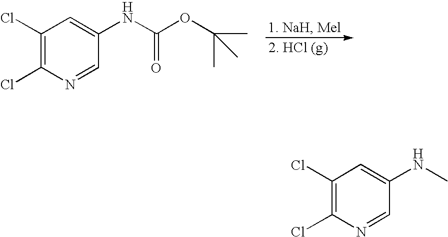

60% NaH (240 mg, 6.0 mmol) was added to the solution of (5,6-dichloro-3-pyridinyl)-carbamic acid 1,1-dimethylethyl ester (1.00 g, 3.80 mmol) in THF (30 ml) at 0° C., and the reaction mixture was stirred at this temperature for 30 min, then MeI (0.40 ml, 6.4 mmol) was added, and the mixture was stirred at room temperature for 2 h. THF was distilled in vacuo and the residue was dissolved in CH₂Cl₂, and washed with brine, dried over Na₂SO₄. Removal of solvent gave the crude N-Me product that was used without purification, MS (ESI) (M+1)⁺=277. The N-Methyl compound was dissolved in EtOAc (30 ml), and HCl(g) was bubbled into the solution for 10 min, and the mixture was stirred at room temperature for 2 h and EtOAc was evaporated in vacuo to give the title compound (640 mg).

### 45C: (5-chloro-6-hydrazino-3-pyridinyl)methyl-carbamic Acid, Methyl Ester

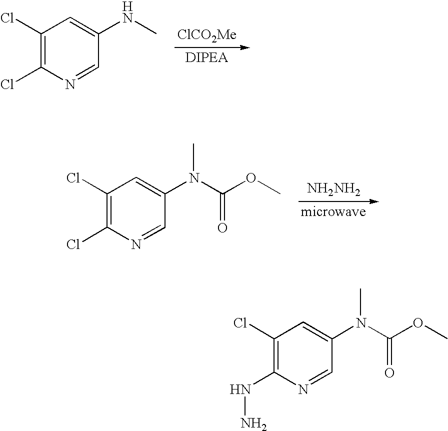

Methyl chloroformate (40 mg, 0.42 mmol) was added to a mixture of 5,6-dichloro-N-methyl-3-pyridinamine (36 mg, 0.20 mmol) and DIPEA (0.15 ml) in CH₂Cl₂ (1 ml) at 0° C. The reaction mixture was stirred at room temperature overnight, diluted with CH₂Cl₂ 10 ml), washed with NaHCO₃, brine and dried. Removal of solvent gave the methyl carbamate intermediate. 1 M NH2NH2 solution in pentanol (2 ml) was added to the intermediate, and the reaction mixture was heated in a Smith station microwave instrument at 180° C. for 10 min. Removal of solvent and excess hydrazine afforded the title compound which was used without purification. MS (ESI) (M+1)⁺=231.

### 45D: [5-chloro-6-[2-[[(7-fluoro-6,11-dihydro-5H-benzo[5,6]cyclohepta[1,2-c]pyridin-11-yl)amino]thioxomethyl]hydrazino]-3-pyridinyl]methyl-carbamic Acid, Methyl Ester

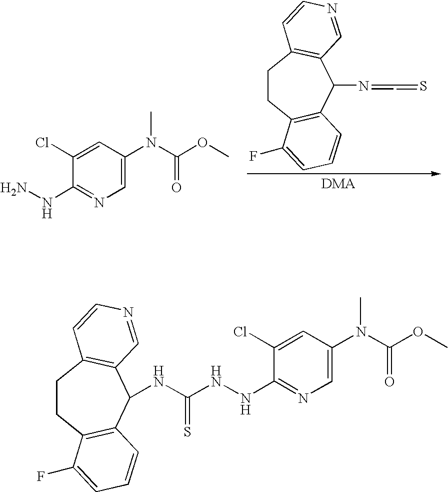

The mixture of 7-fluoro-6,11-dihydro-11-isothiocyanato-5H-benzo[5,6]cyclohepta[1,2-c]pyridine (54 mg, 0.20 mmol) and methyl(5-chloro-6-hydrazino-3-pyridinyl)methyl-carbamate (0.2 mmol) from 925C in DMF (2 ml), and the mixture was stirred at room temperature overnight and purified on prep-LCMS to give the title compound (25 mg) as its TEA salt. ¹HNMR (CD₃OD): δ 8.83 (d, J=4.1 Hz, 1H), 8.45-8.53 (m, 1H), 7.76 (dd, J=6.0, 7.3 Hz, 1), 6.90-7.40 (m, 6H), 3.19 (s, 3H), 3.15 (s, 3H), 2.90-3.60 (m, 4H) ppm. MS (ESI) (M+1)⁺=501. HRMS (M+1)⁺ Calcd for, C₂₃H₂₂ClFN₆O₂S: 501.1276. Found: 501.1211.

### Example 46

### N-[5-chloro-6-[2-[[(7-fluoro-6,11-dihydro-5H-benzo[5,6]cyclohepta[1,2-c]pyridin-11-yl)amino)thioxomethyl]hydrazino]-3-pyridinyl]-N-methyl-cyclopropanecarboxamide

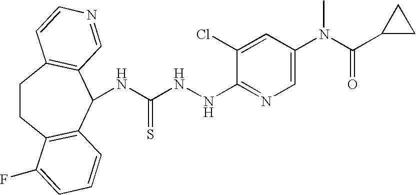

### 46A: N-(5-chloro-6-hydrazino-3-pyridinyl)-N-methyl-cyclopropanecarboxamide

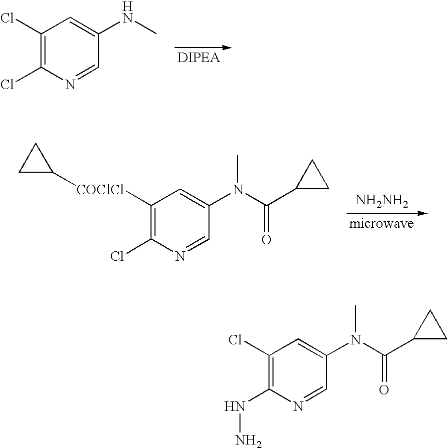

Following the procedure of Example 45C: cyclopropanecarbonyl chloride (40 mg, 0.40 mmol) was added to a mixture of 5,6-dichloro-N-methyl-3-pyridinamine (36 mg, 0.20 mmol) and DIPEA (0.15 ml) in CH₂Cl₂ (1 ml) at 0° C. The reaction mixture was stirred at room temperature overnight, diluted with CH₂Cl₂ 10 ml), washed with NaHCO₃, brine and dried. Removal of solvent gave the amide intermediate, MS(ESI)(M+1)⁺=245. 1-M NH₂NH₂ solution in pentanol (2 ml) was added to the intermediate, and the reaction mixture was heated in a Smith station microwave at 180° C. for 10 min. Removal of solvent and excess hydrazine afforded the title compound that was used without purification. MS (ESI) (M+1)⁺=241.

### 46B: N-[5-chloro-6-[2-[[(7-fluoro-6,11-dihydro-5H-benzo[5,6]cyclohepta[1,2-c]pyridin-11-yl)amino]thioxomethyl]hydrazino]-3-pyridinyl]-N-methyl-cyclopropanecarboxamide

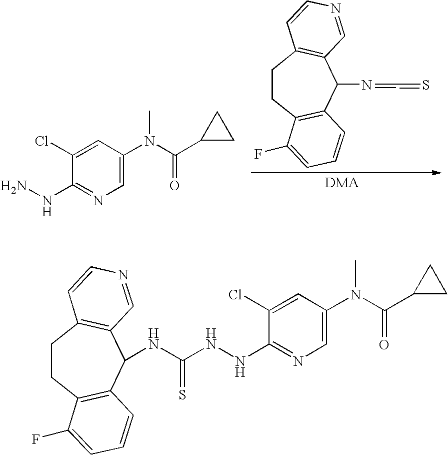

Following the procedure Example 45D: The mixture of 7-fluoro-6,11-dihydro-11-isothiocyanato-5H-benzo[5,6]cyclohepta[1,2-c]pyridine (54 mg, 0.20 mmol) and N-(5-chloro-6-hydrazino-3-pyridinyl)-N-methyl-cyclopropanecarboxamide (0.2 mmol) from 923A in DMF (2 ml), and the mixture was stirred at room temperature overnight and purified on prep-LCMS to give the title compound (26 mg) as its TFA salt. ¹HNMR (400 MHz, CD₃OD): δ 8.75 (s, 1H), 8.40-8.52(m, 2H), 7.98 (s, 1H), 7.60-7.75 (m, 2H), 6.90-7.25 (m, 4H), 3.14 (s, 3H), 3.00-3.50 (m, 4H), 0.50-1.25 (m, 5H) ppm. (MS(ESI)(M+1)⁺=511. HRMS(M+1)⁺ Calcd for C₂₅H₂₄ClFN₆OS: 511.1483. Found: 511.1412.

### Example 47

### N-[5-chloro-6-[2-[[(7-fluoro-6,11-dihydro-5H-benzo[5,6]cyclohepta[1,2-c]pyridin-11-yl)amino]thioxomethyl]hydrazino]-3-pyridinyl]-N-methyl-3-pyridinecarboxamide

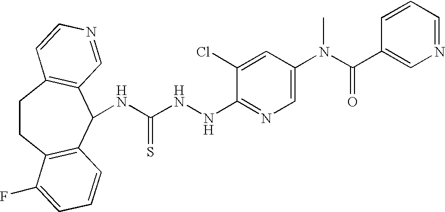

### 47A: N-(5-chloro-6-hydrazino-3-pyridinyl)-N-methyl-3-pyridinecarboxamide

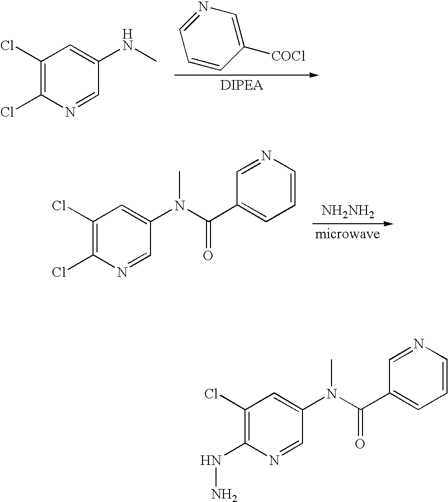

Following the procedure of Example 45C: nicotinoyl chloride hydrochloride (71 mg, 0.40 mmol) was added to a mixture of 5,6-dichloro-N-methyl-3-pyridinamine (36 mg, 0.20 mmol) and DIPEA (0.25 ml) in CH₂Cl₂ (1 ml) at 0° C. The reaction mixture was stirred at room temperature overnight, diluted with CH₂Cl₂ 10 ml), washed with NaHCO₃, brine and dried. Removal of solvent gave the amide intermediate, MS(ESI)(M+1)⁺=282. 1 M NH₂NH₂ solution in pentanol (2 ml) was added to the intermediate, and the reaction mixture was heated in a Smith station microwave at 180° C. for 10 min. Removal of solvent and excess hydrazine afforded the title compound that was used without purification. MS (ESI) (M+1)⁺=278.

### 47B: N-[5-chloro-6-[2-[[(7-fluoro-6,11-dihydro-5H-benzo[5,6]cyclohepta[1,2-c]pyridin-11-yl)amino]thioxomethyl]hydrazino]-3-pyridinyl]-N-methyl-3-pyridinecarboxamide

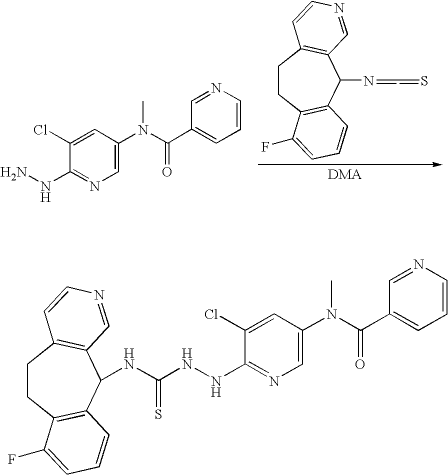

Following the procedure of Example 45D: The mixture of 7-fluoro-6,11-dihydro-11-isothiocyanato-5H-benzo[5,6]cyclohepta[1,2-c]pyridine (54 mg, 0.20 mmol) and N-[5-chloro-6-[2-[[(7-fluoro-6,11-dihydro-5H-benzo[5,6]cyclohepta[1,2-c]pyridin-11-yl)amino]thioxomethyl]hydrazino]-3-pyridinyl]-N-methyl-3-pyridinecarboxamide (−0.2 mmol) from step 47A in DMF (2 ml), and the mixture was stirred at room temperature overnight and purified on prep-LCMS to give the title compound (27 mg).

¹HNMR(400 MHz, CD₃OD): δ 8.78 (s, 1H), 8.62 (d, J=5.8 Hz, 1H), 8.47 (br, 1H), 7.80-8.00 (m, 4H), 7.00-7.50 (m, 7H), 3.43 (s, 3H), 3.25-3.50 (m, 3H), 3.05-3.20 (m, 1H) ppm. MS(ESI)(M+1)⁺=548. HRMS(M+1)⁺ Calcd for C₂₇H₂₃ClFN₇OS: 548.1435; Found: 548.1475.

### Example 48

### 2-[3-chloro-5-[[[(3R)-1-methyl-2-oxo-3-pyrrolidinyl]amino]sulfonyl]-2-pyridinyl]-N-(7-fluoro-6,11-dihydro-5H-benzo[5,6]cyclohepta[1,2-c]pyridin-11-yl)-hydrazinecarbothioamide

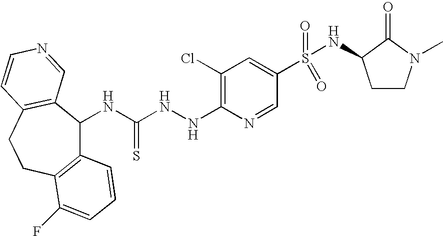

### 48A: 5,6-dichloro-N-[(3R)-1-methyl-2-oxo-3-pyrrolidinyl]-3-pyridinesulfonamide

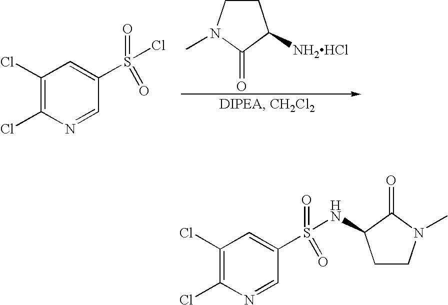

A solution of 5,6-dichloro-3-pyridinesulfonyl chloride (Prepared according to: M. W. Crawley, European Patent Application No. EP0147105A2 (1985)) (34 mg, 0.14 mmol) in CH₂Cl₂ (3 mL) was added to a mixture of (3R)-3-amino-1-methyl-2-pyrrolidinone hydrochloride (60 mg, 0.40 mmol) and DIPEA (0.20 mL,) in CH₂Cl₂ (3 ml) at −30° C., the reaction mixture was stirred at this temperature for 30 min, allowed to warm to room temperature, diluted with EtOAc (20 mL), washed with 5% HCl, H₂O, and brine, dried over Na₂SO₄. Removal of solvent gave the title compound (15 mg), MS(ESI) (M+1)⁺: 324.

### 48B: 5-chloro-6-hydrazino-N-[(3R)-1-methyl-2-oxo-3-pyrrolidinyl]-3-pyridinesulfonamide

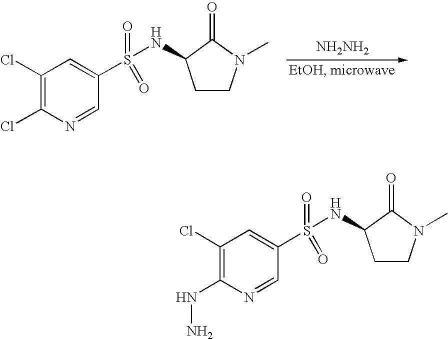

A solution of 5,6-dichloro-N-[(3R)-1-methyl-2-oxo-3-pyrrolidinyl]-3-pyridinesulfonamide (15 mg, 0.046 mmol), and 1M NH₂NH₂ in pentanol (0.30 ml, 0.30 mmol) in EtOH (0.50 ml) was heated in microwave for 10 min, allowed to cool to room temperature. The solid was collected and the mother liquid was concentrated in vacuo and lipholized. The product was combined to give the title compound (12 mg, 80%). MS(ESI) (M+1)+=320.

### 48C: 2-[3-chloro-5-[[[(3R)-1-methyl-2-oxo-3-pyrrolidinyl]amino]sulfonyl]-2-pyridinyl]-N-(7-fluoro-6,11-dihydro-5H-benzo[5,6]cyclohepta[1,2-c]pyridin-11-yl)-hydrazinecarbothioamide

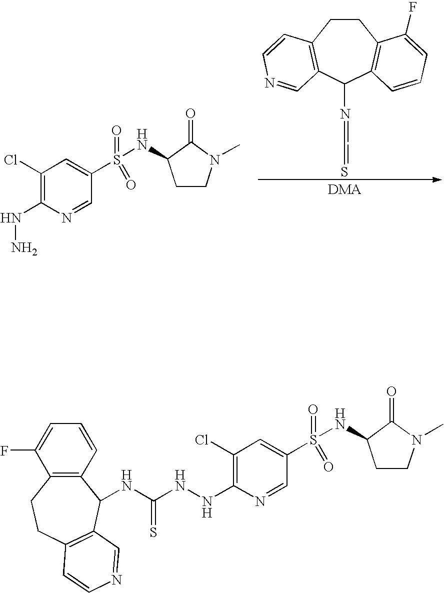

5-chloro-6-hydrazino-N-[(3R)-1-methyl-2-oxo-3-pyrrolidinyl]-3-pyridinesulfonamide (6.4 mg, 0.02 mmol) and 7-fluoro-6,11-dihydro-11-isothiocyanato-5H-benzo[5,6]cyclohepta[1,2-c]pyridine (5.4 mg, 0.02 mmol) was dissolved in DMA (1 ml) at room temperature and the reaction mixture was stirred at room temperature overnight. DMA was stripped off and the residue was lipholized to afford the title compound (12 mg, quantitatively). ¹HNMR (CD₃OD): δ 8.55 (s, 1H), 8.51 (d, J=2 Hz, 1H), 8.33 (m, 2H), 8.08 (m, 1H), 6.90-7.55 (m, 6H), 4.06-4.16 (m, 1H), 3.00-3.50 (m, 6H), 2.68 (s, 1.5H), 2.65 (s, 1.5H) (2.65 and 2.68 peaks from the NMe of the two diastereoisomers), 2.32-2.44 (m, 1H), 1.75-1.90 (m, 1H) ppm. MS(ESI)(M+1)⁺=590. HRMS(M+1)⁺ Calcd for C₂₅H₂₅ClFN₇O₃S₂: 590.1211; Found: 590.1141.

### Example 49

### 5-chloro-6-[2-([(7-fluoro-6,11-dihydro-5H-benzo[5,6]cyclohepta[1,2-c]pyridin-11-yl)amino]thioxomethyl]-1-methylhydrazino]-N-[(3R)-1-methyl-2-oxo-3-pyrrolidinyl]-3-pyridinecarboxamide

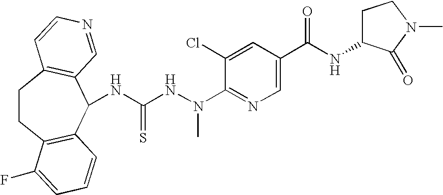

### 49A: 5-chloro-6-(1-methylhydrazino)-3-pyridinecarboxylic Acid

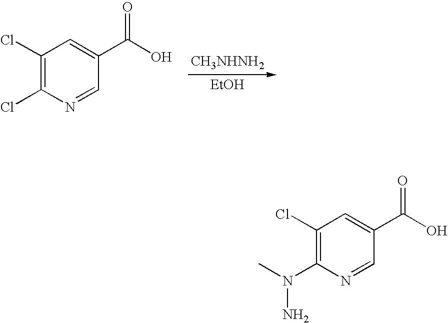

The mixture of 5,6-dichloronicotic acid (2.00 g, 10.42 mmol) and methylhydrazine (2.50 ml) in EtOH (15 ml) was heated at reflux for 3 days, then EtOH was striped off, and then H₂O (10 ml) was added, and the solution was neutralized with HOAc to pH˜7. The precipitate was collected and dried to give the title compound (1.80 g, 86%).

### 49B: 5-chloro-6-[2-[[(7-fluoro-6,11-dihydro-5H-benzo[5,6]cyclohepta[1,2-c]pyridin-1-yl)amino]thioxomethyl]-1-methylhydrazino]-N-[(3R)-1-methyl-2-oxo-3-pyrrolidinyl]-3-pyridinecarboxamide

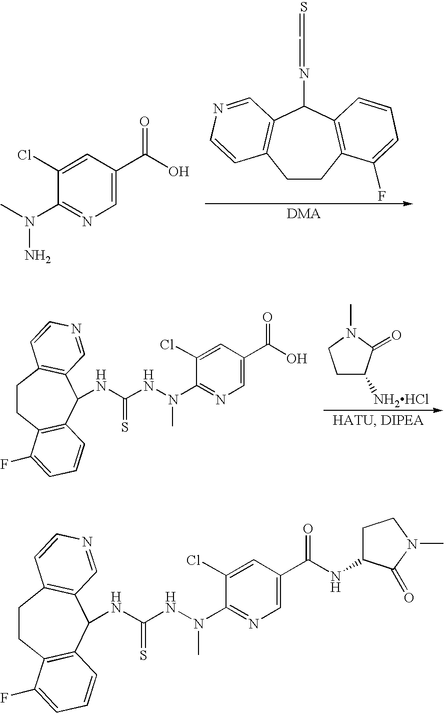

7-fluoro-6,11-dihydro-11-isothiocyanato-5H-benzo[5,6]cyclohepta[1,2-c]pyridine (41 mg, 0.15 mmol) and 5-chloro-6-(1-methylhydrazino)-3-pyridinecarboxylic acid (30 mg, 0.15 mmol) were dissolved in DMA (3 ml), the reaction mixture was stirred at room temperature for 16 h, and the acid intermediate as its DMA solution was prepared. MS(ESI, M+1)⁺: 427.

(3R)-3-amino-1-methyl-2-pyrrolidinone hydrochloride (45 mg), DIPEA (0.15 ml), HATU (65 mg) was added successively to the above acid intermediate. The mixture was stirred at room temperature for 2 h, DMA was stripped off in vacuo, and the residue was treated with H₂O (5 ml), the solid was collected and washed with H₂O (2×5 ml), dried in vacuo to give the title compound (53 mg). ¹HNMR(CD₃OD): δ 8.75 (s, 1H), 8.62 (d, J=3.9 Hz, 1H), 8.55 (d, J=4.3 Hz, 1H), 8.28-8.40 (m, 2H), 8.03 (s, 1H), 7.48 (m, 1H), 6.90-7.40 (m, 5H), 4.56 (dd, J=9.0, 9.1 Hz, 1H), 3.00-3.50 (m, 6H), 3.10 (s, 3H), 2.79 (s, 3H), 3.30-2.45 (m, 1H), 1.85-2.05 (m, 1H) ppm. MS (M+1)⁺=568. HRMS (M+1)⁺ Calcd for C₂₇H₂₇ClFN₇O₂S: 568.1698; Found: 568.1772.

### Example 50

### 5-Chloro-6-[2-[[(7-fluoro-6,11-dihydro-5H-benzo[5,6]cyclohepta[1,2-c]pyridin-11-yl)amino]carbonyl]hydrazino]-N-[(3S)-1-methyl-5-oxo-3-pyrrolidinyl]-3-pyridinecarboxamide

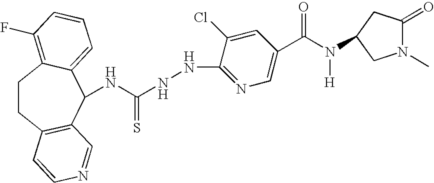

### 50A: 2-[[(1,1-Dimethylethoxy)carbonyl]amino]-3-(methylamino)-3-oxo-phenylmethyl Ester, (2S)-propanoic Acid

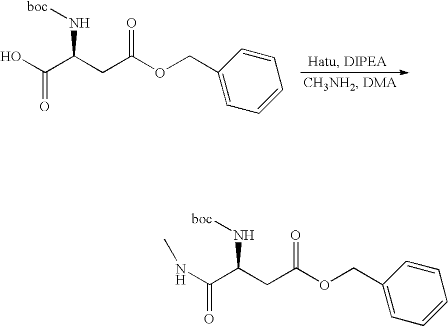

HATU (7.05 g, 18.55 mmol) was added to a mixture of Boc-L-Asp(OBz)-OH (5 g, 15.46 mmol), CH₃NH₂.HCl (3.3 g, 48.87 mmol) and DIPEA (13.42 mL, 77.07 mmol) in DMA (20 ml). The mixture was stirred at room temperature overnight and then DMA was removed under reduced pressure. The residue was then diluted in CH₂Cl₂ and washed with saturated NaHCO₃, water and brine, dried and concentrated. The product was then purified by column chromatography (CH₂Cl₂-MeOH) to afford the title product (2.10 g, 40%). ¹H NMR (400 MHz, CDCl₃): δ 7.41-7.30 (m, 5H), 6.41 (bs, 1H), 5.64 (bs, 1H), 5.17-5.07 (m, 2H), 4.84 (s, 1H), 3.11-3.02 (m, 1H), 2.77 (d, J=4.9 Hz, 3H), 2.69 (dd, J=17.3 and 6.1 Hz, 1H), 1.43 (s, 9H) ppm. MS (ESI) (M+1)⁺: 337.

### 50B: N-[(1,1-Dimethylethoxy)carbonyl]-3-(methylamino)-3-thioxo-phenylmethyl Ester-L-alanine

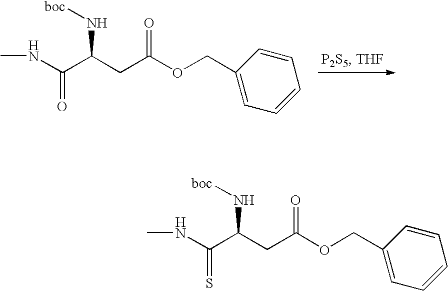

A mixture of 0.2 g (0.59 mmols, 1 eq) of 2-[[(1,1-dimethylethoxy)carbonyl]amino]-3-(methylamino)-3-oxo-phenylmethyl ester, (2S)-propanoic acid and phosphorus pentasulfide (0.41 g, 0.92 mmols, 1.6 eq.) in THF (5 mL) was heated to 60° C. for 1 hour. The mixture was then cooled to the room temperature and AcOEt was added. The organic phase was then washed with saturated NaHCO₃, water and brine, dried and concentrated. The product was directly used for the next step. MS (ESI) (M+1)⁺: 353.

### 50C: [(3S)-1-Methyl-5-oxo-3-pyrrolidinyl]-carbamic Acid, 1,1-dimethylethyl Ester

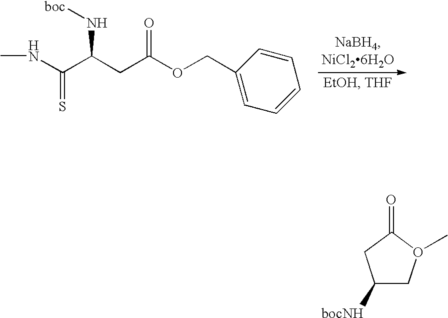

NiCl₂.6H₂O (0.21 g, 5.6 mmol) and N-[(1,1-dimethylethoxy)carbonyl]-3-(methylamino)-3-thioxo-phenylmethyl ester-L-alanine (85 mg, 0.24 mmol) were dissolved in EtOH/THF (1/1 mL) and the mixture was cooled at 0° C. NaBH₄ (0.11 g, 2.9 mmol) was then added and the reaction was stirred 30 minutes. The mixture was then filtered over celite and the filtrate evaporated under reduced pressure. The residue was dissolved in CH₂Cl₂ and was washed with 5% NaHCO₃. The organic layer was separated and washed with water and brine, dried and concentrated under reduced pressure. The crude product was then purified by column chromatography (CH₂Cl₂-MeOH). ¹H NMR (400 MHz, CDCl₃): δ 4.93 (bs, 1H), 4.26 (bs, 1H), 3.71-3.62 (m, 1H), 3.21 (dd, J=10.5 and 3.8 Hz, 1H), 2.82 (s, 3H), 2.71 (dd, J=17.3 and 8.3 Hz, 1H), 2.22 (dd, J=17.0 and 4.5 Hz, 1H), 1.42 (s, 9H) ppm. MS (ESI) (M+1)⁺: 215.

### 50D: 4-Amino-1-methyl-monohydrochloride, (4S)-2-pyrrolidinone

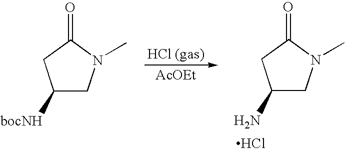

A solution of [(3S)-1-Methyl-5-oxo-3-pyrrolidinyl]-1,1-dimethylethyl ester-carbamic acid (0.14 g, 0.65 mmols) in AcOEt (3 mL) was treated with HCl (gas) at 0° C. for 1 hour. The stirring was then continued for 3 hours. The solvent was removed under reduced pressure and the crude product was directly used for the next step.

### 50E: 5,6-Dichloro-N-[(3S)-1-methyl-5-oxo-3-pyrrolidinyl]-3-pyridinecarboxamide

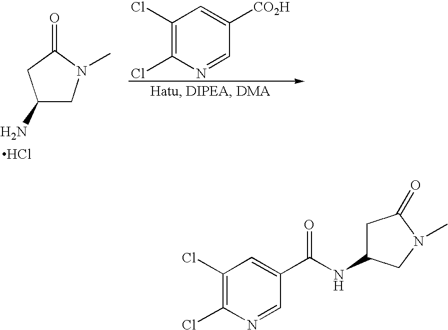

HATU (0.46 g, 1.21 mmols, 1.2 eq.) was added to a mixture of 5,6-dichloronicotinic acid (0.23 g, 1.20 mmols, 1 eq.), 4-amino-1-methyl-monohydrochloride,(4S)-2-pyrrolidinone (0.3 g, 1.99 mmols, 1 eq.) and DIPEA (0.8 mL, 4.59 mmols, 2.3 eq.) in 10 mL DMA. The mixture was stirred at rt overnight and then DMA was removed under reduced pressure. The residue was then diluted in CH₂Cl and washed with saturated NaHCO₃, water and brine, dried and concentrated. The crude product was then purified by column chromatography (CH₂Cl₂-MeOH) to afford the title compound (0.14 g, 40%). MS (ESI) (M+1)⁺: 289.

### 50F: 5-Chloro-6-hydrazino-N-[(3S)-1-methyl-5-oxo-3-pyrrolidinyl]-3-pyridinecarboxamide

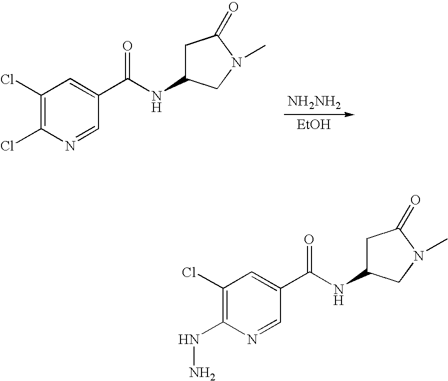

A solution of 5,6-dichloro-N-[(3S)-1-methyl-2-oxo-3-pyrrolidinyl]-3-pyridinecarboxamide (80 mg, 0.28 mmol) in EtOH (1.5 mL) was treated with hydrazine.monohydrate (60 μL, 4.4 eq.) and the mixture was refluxed for 3 hours. The reaction was then cooled to room temperature and the solid was filtered and washed with EtOH to provide the title compound (76 mg, 96%).

### 50G: 5-Chloro-6-[2-[[(7-fluoro-6,11-dihydro-5H-benzo[5,6]cyclohepta[1,2-c]pyridin-11-yl)amino]carbonyl]hydrazino]-N-[(3S)-1-methyl-5-oxo-3-pyrrolidinyl]-3-pyridinecarboxamide

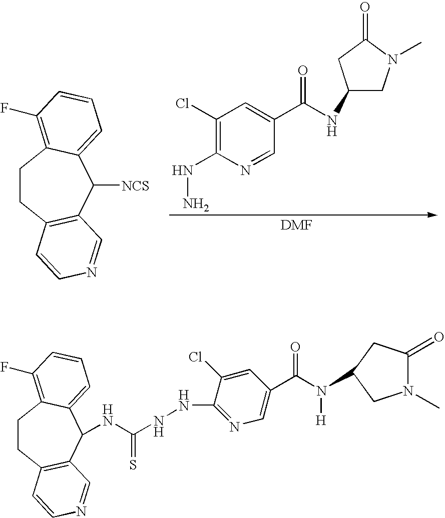

To a solution of 7-fluoro-6,11-dihydro-11-isothiocyanato-5H-benzo[5,6]cyclohepta[1,2-c]pyridine (54 mg, 0.2 mmols, 1 eq) in 3 mL DMF was added 5-chloro-6-hydrazino-N-[(3S)-1-methyl-5-oxo-3-pyrrolidinyl]-3-pyridinecarboxamide (76 mg, 0.2 mmols, 1 eq) and the mixture was stirred overnight. The solvent was then removed under reduced pressure and the residue was then diluted in CH₂Cl₂ and washed with a saturated NaHCO₃, water and brine, dried and concentrated under reduced pressure. The product was then purified by column chromatography (CH₂Cl₂-MeOH) to afford the title product (22 mg, 27%). ¹H NMR (400 MHz, CDCl₃): δ 7.27 (bs, 1H), 7.14 (bs, 1H), 7.05 (d, J=6.0 Hz, 1H), 6.59 (s, 1H), 6.34 (d, J=6.0 Hz, 1H), 5.99 (s, 1H), 5.75-5.63 (m, 2H), 5.50 (t, J=8.5 Hz, 1H), 3.15-3.03 (m, 1H), 2.27 (dd, J=10.5 and 7.4 Hz, 1H), 2.02-1.84 (m, 3H), 1.79-1.60 (m, 2H), 1.30 (s, 3H), 1.30-1.18 (m, 2H), 0.96 (dd, J=17.4 and 4.5 Hz, 1H) ppm. MS (ESI) (M+1)⁺: 555.

1. A compound of formula (I), pharmaceutically acceptable salts thereof, diasteriomers thereof, enantiomers thereof, or mixtures thereof: 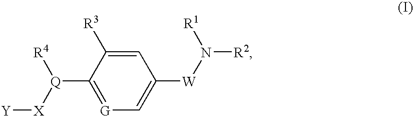
wherein
R¹ and R² are independently selected from hydrogen, optionally substituted C₁₋₁₂acyl, optionally substituted C₁₋₁₂alkyl-oxycarbonyl, optionally substituted C₁₋₁₂alkyl, optionally substituted C₁₋₁₂heteroalkyl, optionally substituted C₃₋₁₂cycloalkyl, optionally substituted C₆₋₁₂aryl and optionally substituted C₂₋₁₂heterocyclyl;
W is a linking group that separates the groups linked thereto by one or two atoms;
G is N or CH;
R³ is halogen, hydrogen or C₁₋₆alkyl;
Q is N or CH;
R⁴ is —H or optionally substituted hydrocarbyl;
X is a divalent group including first nitrogen atom and a second nitrogen atom, wherein a first group linked to X is linked to the first nitrogen and a second group linked to X is linked to the second nitrogen atom, and wherein the first and second nitrogen atoms are separated by either one carbon atom, or two carbon atoms wherein said two carbon atoms form a double bond therebetween; and
Y is a group that includes an optionally substituted seven-membered ring and two optionally substituted aromatic rings, wherein each of the aromatic rings is independently fused with said seven-membered ring, and wherein each of said seven-membered ring and aromatic rings, independently, optionally, contains one or more heteroatoms:

2. A compound of formula (I), pharmaceutically acceptable salts thereof, diasteriomers thereof, enantiomers thereof, or mixtures thereof, 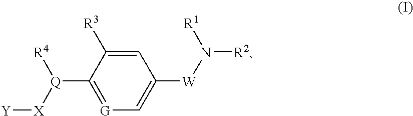
wherein
R¹ and R² are independently selected from hydrogen, optionally substituted acyl, optionally substituted alkyl-oxycarbonyl, optionally substituted alkyl, optionally substituted heteroalkyl, optionally substituted cycloalkyl, optionally substituted aryl; optionally substituted heterocyclyl; optionally substituted aryl-C₁₋₆alkyl, and optionally substituted heterocyclyl-C₁₋₆alkyl;
W is a linking group selected from —C(═O)—, —C(═O)O— and —S(═O)₂—;
G is N or CH;
R³ is halogen, or hydrogen
Q is N or CH;
R⁴ is —H, optionally substituted hydrocarbyl, a single bond, or a divalent group;
X is represented by (i), (ii), (iii), (iv), (v), (vi), (vii), (viii), (ix), (x), (xi), (xii), (xiii), (xiv), (xv), (xvi), or (xvii) below: 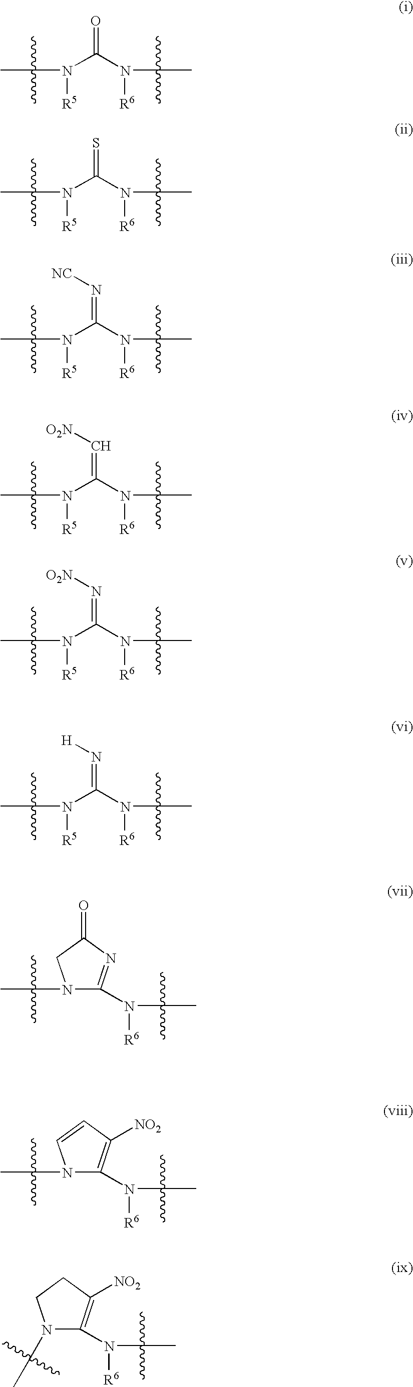 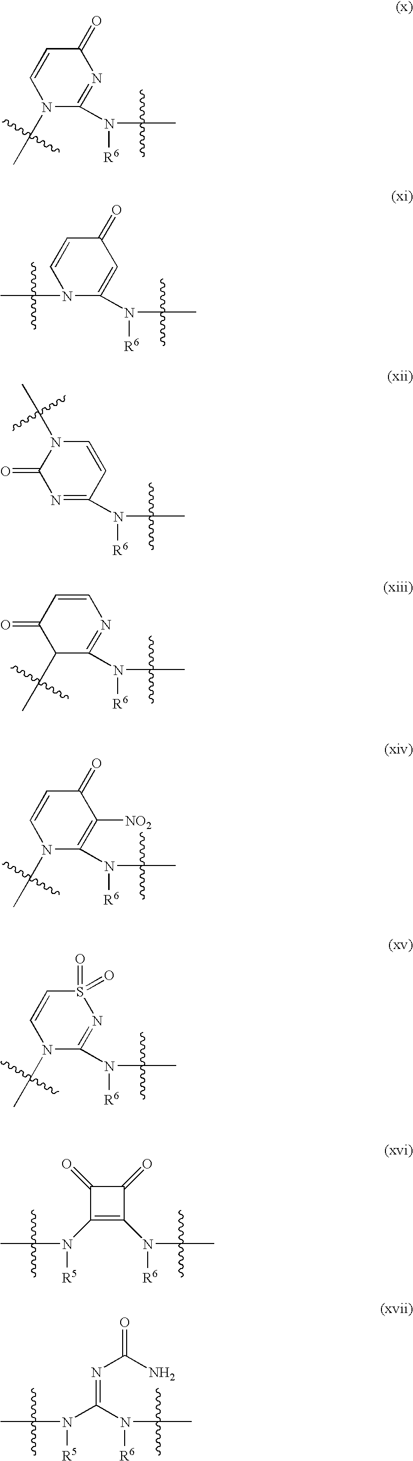
wherein R⁵ is selected from —H or optionally substituted alkyl, or a divalent C₀₋₆group together with R⁴ to form a portion of a ring, wherein said divalent C₀₋₆ group optionally contains one or more heteroatoms;
R⁶ is independently selected from —H or optionally substituted alkyl;
Y is represented by formula (1H) below: 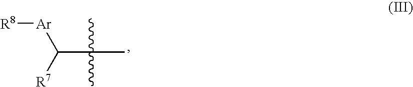
wherein
R⁷ is optionally substituted aryl, optionally substituted heteroaryl, optionally substituted aryl-C₁₋₆alkyl, optionally substituted heteroaryl-C₁₋₆alkyl, —C(═O)O—R⁹, —C(═O)NHR⁹, —C(═O)NR⁹R¹⁰, —SO₂NHR⁹, —SO₂NR⁹R¹⁰, —R¹¹NH₂, —R¹¹NHR¹², —R¹¹NR¹²R¹³, —R¹¹OH, —R¹¹OR¹², —R¹¹SH, —R¹¹SR¹², or a divalent C₀₋₆group which together with R⁸ forms a portion of a ring,
R⁸ is —H, halogen, optionally substituted R¹², —OR¹², —SR¹², —S(═O)R¹², —SO₂R¹², —C(═O)R¹², or a divalent C₀₋₆group which together with the divalent R⁷ forms the portion of the ring,
wherein R⁹ and R¹⁰ are independently C₁₋₁₂hydrocarbyl, R¹¹ is C₁₋₆alkylene, R¹² and R¹³ are independently C₁₋₆alkyl; and
Ar is optionally substituted arylene, optionally substituted heteroarylene, optionally substituted arylene-C₁₋₆alkyl, or optionally substituted heteroarylene-C₁₋₆alkyl.

3. A compound according to claim 2, wherein
R¹ is selected from C₁₋₆alkyl, C₃₋₆cycloalkyl, aryl, aryl-C₁₋₆alkyl or heterocyclyl, heterocyclyl-C₁₋₆alkyl, wherein said C₁₋₈alkyl and C₃₋₈cycloalkyl are optionally, independently, subsituted by —R²⁰, —C(═O)R²⁰, oxo (═O), sulfo (═S), —OH, —OR²⁰, phenyl, halogen, heterocyclyl, —NH₂, —NHR²⁰, —NR²⁰R²¹, —C(═O)NH₂, —C(═O)NHR²⁰, —C(═O)NR²⁰R²¹ and —C(═O)OR²⁰, wherein said aryl is optionally substituted by —R²⁰, —C(═O)R²⁰, —OH, —OR²⁰, phenyl, halogen, heterocyclyl, —NH₂, NHR²⁰, —NR²⁰R²¹, —C(═O)NH₂, —C(═O)NHR²⁰, —C(═O)NR²⁰R²¹ and —C(═O)OR²⁰, wherein said heterocyclyl is a five or six-membered heterocyclyl, wherein said heterocyclyl is optionally substituted by —R²⁰, aryl, heteroaryl, —NH₂, —NHR²⁰, —NR²⁰R²¹, —C(═O)NH₂, —C(═O)NHR²⁰, —C(═O)NR²⁰R²¹, —C(═O)OR²⁰, or oxo (═O);
R² is selected from C₁₋₆alkyl, C₃₋₆cycloalkyl, wherein said C₁₋₆alkyl and C₃₋₆cycloalkyl are optionally, independently, subsituted by R²⁰, —C(═O)R²⁰, oxo (═O), sulfo (═S), —OH, —OR²⁰, phenyl, halogen, heterocyclyl, —NH₂, —NHR²⁰, R²⁰R²¹, —C(═O)NH₂, —C(═O)NHR²⁰, —(═O)NR²⁰R²¹ and —C(═O)OR²⁰;
wherein R²⁰ and R²¹ are independently C₁₋₆alkyl;
R³ is selected from bromo, chloro and fluoro;
R⁴ is —H, optionally substituted (C₁-C₆)alkyl, or a divalent group together with R⁵ of X to form a portion of a first ring;
W is —C(═O)—, or —S(═O)₂—;
G is N or CH;
Q is CH or N;
X is selected from Formulas (i) and (ii), below: 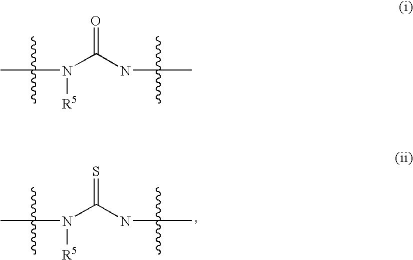
wherein R⁵ is —H, optionally substituted C₁₋₆alkyl, a bond or a divalent group wherein said bond or divalent group together with R⁴ forms the portion of the first ring, wherein said first ring is selected from optionally substituted Formulas (a), (b) and (c), 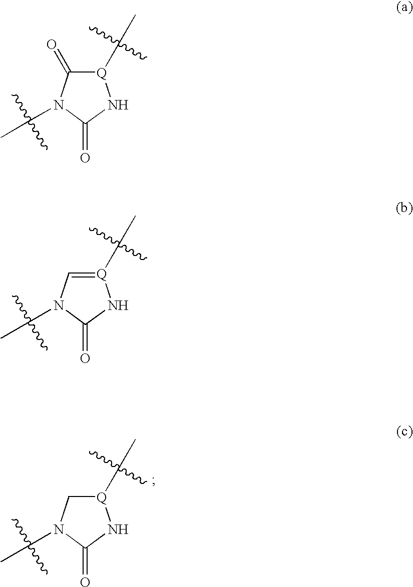
wherein when R⁴ or R⁵ is substituted, substituents of R⁴ or R⁵ are selected from: —OH, NH2, —O(C₁-C₃)alkyl, —CN, oxo (═O), —C(═O)O(C₁-C₄)alkyl and halogen.
Y is represented by formula (III) below: 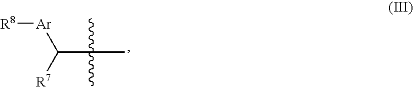
R⁷ is optionally substituted aryl, optionally substituted heterocyclyl, or optionally substituted arylene which together with R⁸ forms a portion of a second ring;
R⁸ is R²², —OR²², —SR²², —S(═O)R²², —SO₂R²², (═O)R²², or a optionally substituted divalent C₀₋₆group which together with the divalent R⁷ forms the portion of the second ring;
wherein when R⁷ or R⁸ is substituted, substituents of R⁷ or R⁸ are halogen, nitro, cyano, R²², —C(═O)R²², —C(═O)OR²², —OH, —OR²², C(═O)NHR²², —C(═O)NR²²R²³, —SO₂NH₂, —SO₂NHR²² or —SO₂NR²²R²³;
Ar is optionally substituted arylene, optionally substituted heteroarylene, optionally substituted arylene-C₁₋₆alkyl, or optionally substituted heteroarylene-C₁₋₆alkyl; and
wherein R²² and R²³ are independently C₁₋₆alkyl.

4. A compound according to claim 2, wherein
R¹ is selected from C₁₋₆alkyl, C₃₋₆cycloalkyl, aryl, aryl-C₁₋₆alkyl or heterocyclyl, heterocyclyl-C₁₋₆alkyl, wherein said C₁₋₆alkyl and C₃₋₆cycloalkyl and aryl are optionally, independently, subsituted by —OH, —C(═O)OR²⁴, —OR²⁴ or —NR²⁴R²⁵, wherein said heterocyclyl is derived from pyrrolidinone, five-membered lactone, five-membered thiolactone, pyrrolidine, tetrahyrofuran, thiophan, sulfolane, piperidine, piperazine, morpholine, thiomorpholine, dioxane, tetrahydropyran or tetrahydrothiopyran by removing a hydrogen therefrom, wherein said heterocyclyl is optionally substituted by oxo (═O);
R² is selected from C₁₋₆alkyl, C₃₋₆cycloalkyl, wherein said C₁₋₆alkyl and C₃₋₆cycloalkyl are optionally, independently, subsituted by —OH, —C(═O)OR²⁴, —OR²⁴ and —NR²⁴R²⁵;
wherein R²⁴ and R²⁵ are independently C₁₋₆alkyl;
R³ is chloro;
R⁴ is —H;
G is N or CH;
Q is N or CH;
W is —C(═O)—;
X is selected from Formulas (i) and (ii), below: 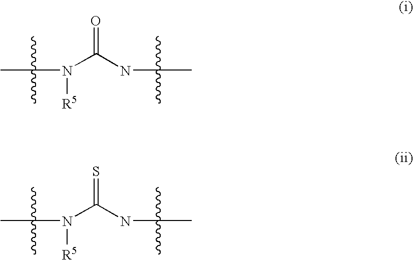
wherein R⁵ is —H or C₁₋₆alkyl;
Y is selected from formulas (d), (e), (f), (g), (h), (j) and (k), below: 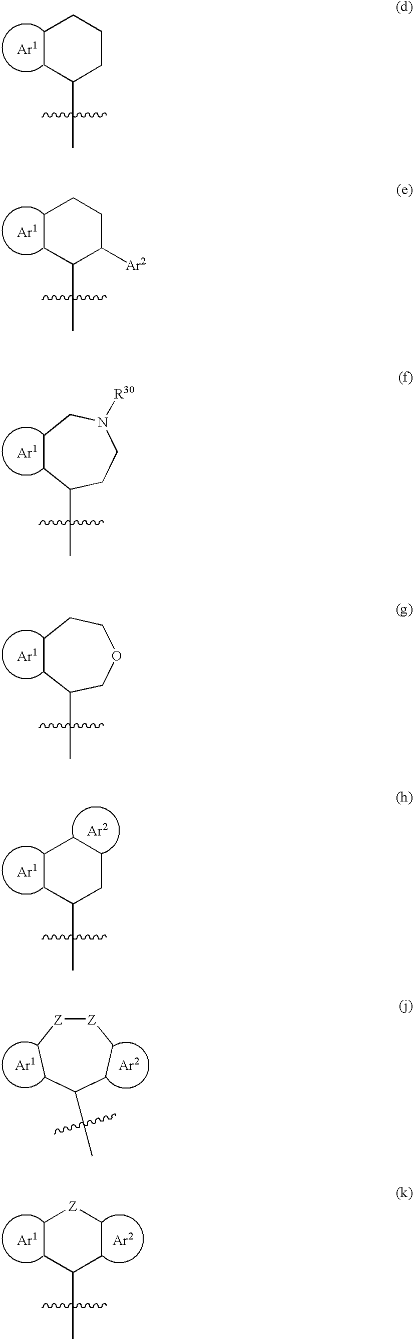
wherein Z is selected from —C—, —C(═O)—, —O—, —N(-alkyl)-, —NH—, —S—, —S(═O)— and —SO₂—; Ar¹ and Ar² are, independently, optionally substituted aryl, or optionally subsitituted heteroaryl; R³⁰ is a C₁₋₆hydrocarbyl; and when Ar¹ or Ar² is represented by a three-quarter cycle attached to a ring structure, Ar¹ or Ar² is fused with said ring structure.

5. A compound according to claim 4, wherein
R¹ is selected from a group derived from dihydrothiophene-2-one, pyrrolidinone, five-membered lactone, or five-membered thiolactone by removing one hydrogen therefrom, wherein said group is optionally substituted by C₁₋₃alkyl or phenyl, and —CH₂C(═O)OC₂H₅;
R is —H or —CH₃;
R⁵ is —H; and
Y is selected from structures (l), (m), (n), (O), (p), (q), (r), (s), (t), (u), (v), (w), (x), (y), (z), (a1), (b1), (c1), (d1), (e1), (f1), (g1) and (h1) below, 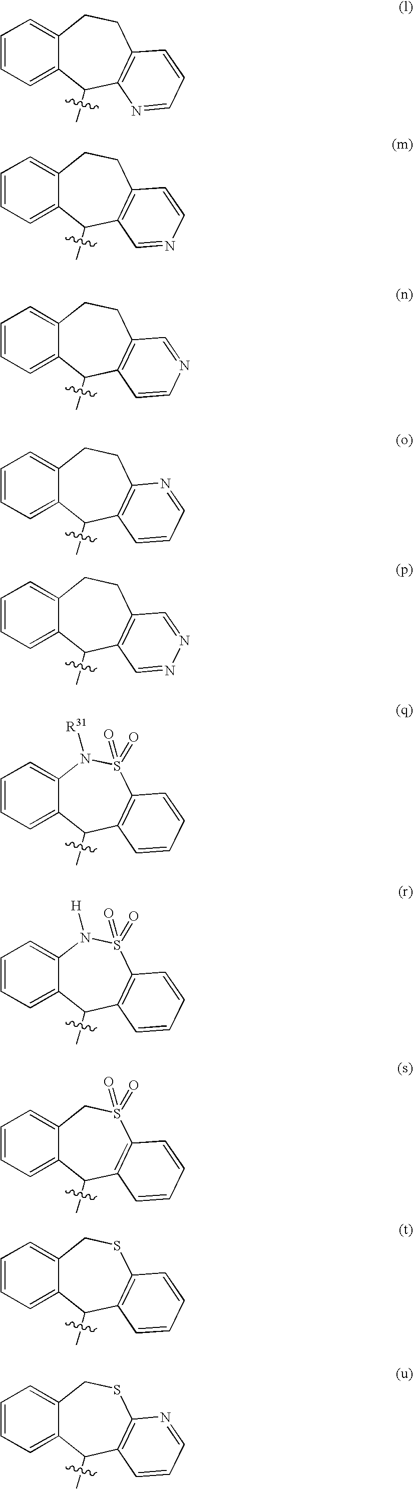 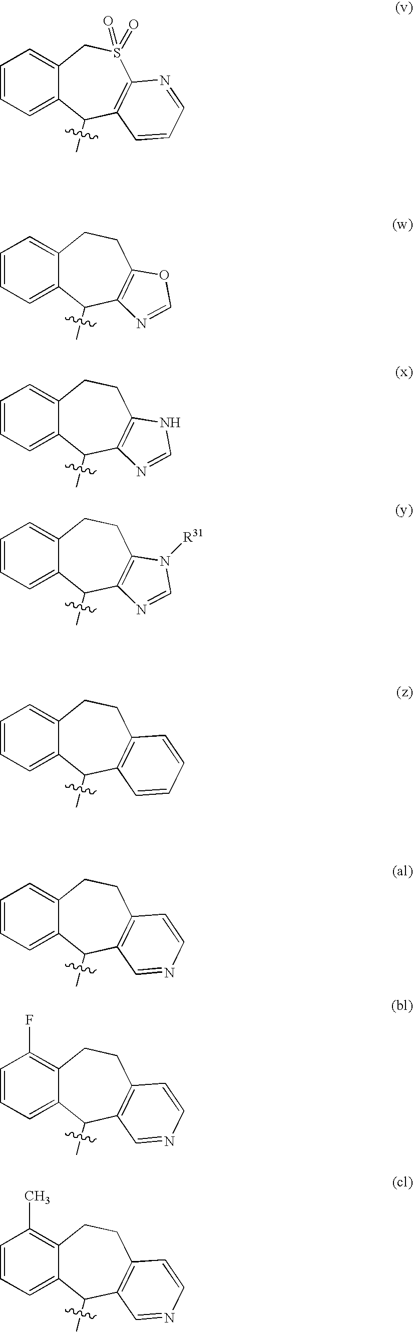 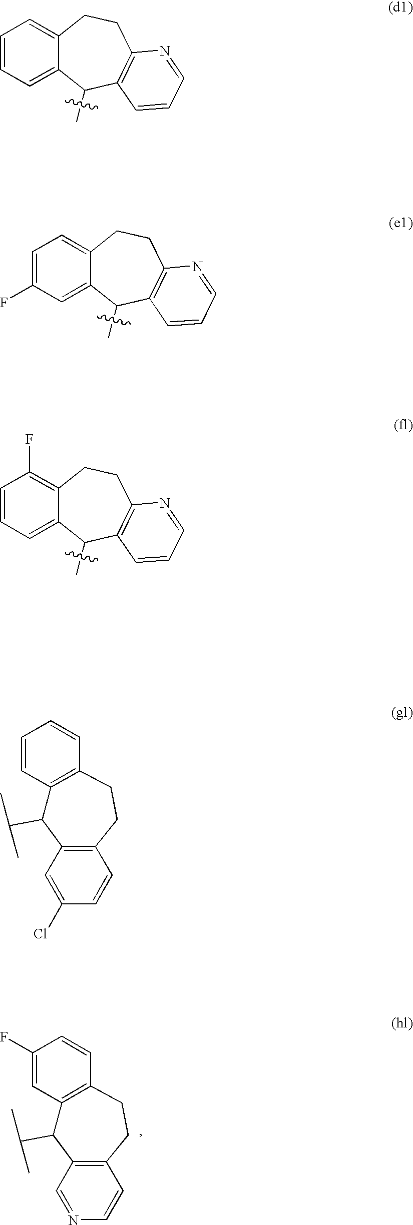
and wherein R³¹ is a C₁₋₆alkyl.

6. A compound of formula (I), pharmaceutically acceptable salts thereof, diasteriomers thereof, enantiomers thereof, or mixtures thereof: 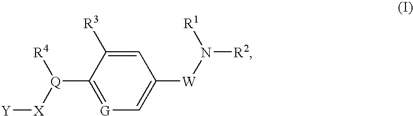
wherein
R¹ and R² are independently selected from hydrogen, optionally substituted C₁₋₁₂acyl, optionally substituted C₁₋₁₂alkyl-oxycarbonyl, optionally substituted C₁₋₁₂alkyl, optionally substituted C₁₋₁₂heteroalkyl, optionally substituted C₃₋₁₂cycloalkyl, optionally substituted C₆₋₁₂aryl and optionally substituted C₂₋₁₂heterocyclyl;
W is a linking group selected from (═O)—, —C(═O)O— and —S(═O)₂—;
G is N or CH;
R³ is halogen, or hydrogen
Q is N or CH;
R⁴ is —H, or optionally substituted hydrocarbyl;
X is selected from Formulas (i) and (ii), below 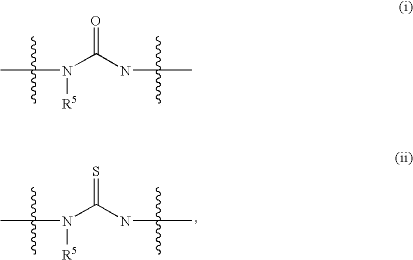
wherein R⁵ is —H, or optionally substituted C₁₋₆alkyl; and
Y is optionally subsitituted aryl, or optionally substituted heteroaryl.

7. A compound selected from:
N-[[5-chloro-6-[2-[[(10,11-dihydro-5H-dibenzo[a,d]cyclo-hepten-5-yl)amino]thioxomethyl]-hydrazino]-3-pyridinyl]-carbonyl]-N-methyl-glycine ethyl ester;
N-[[5-chloro-6-[2-[[(10,11-dihydro-5H-dibenzo[a,d]cyclo-hepten-5-yl)amino]carbonyl]-hydrazino]-3-pyridinyl]carbonyl]-N-methyl-glycine ethyl ester;
5-chloro-6-[2-[[(10,11-dihydro-5H-dibenzo[a,d]-cyclohepten-5-yl)amino]thioxomethyl]hydrazino]-N-(tetrahydro-2-oxo-3-thienyl)-3-pyridine-carboxamide;
5-chloro-6-[2-[[(10,11-dihydro-5H-dibenzo[a,d]-cyclohepten-5-yl)amino]thioxomethyl]hydrazino]-N-(2-hydroxyethyl)-N-(phenyl-methyl)-3-pyridinecarboxamide;
5-chloro-6-[2-[[(10,11-dihydro-5H-dibenzo[a,d]-cyclohepten-5-yl)amino]carbonyl]hydrazino]-N-(tetrahydro-2-oxo-3-thienyl)-3-pyridinecarboxamide;
N-[3-chloro-4-[[[[(10,11-dihydro-5H-dibenzo[a,d]cyclohepten-5-yl)amino]carbonyl]amino]methyl]benzoyl]-N-methyl-glycine ethyl ester;
3-chloro-4-[[[[(10,11-dihydro-5H-dibenzo[a,d]cyclo-hepten-5-yl)amino]carbonyl]amino]methyl]-N-[[(2S)-1-ethyl-2-pyrrolidinyl]-methyl]-benzamide;
3-chloro-4-[[[[(10,11-dihydro-5H-dibenzo[a,d]cyclo-hepten-5-yl)-amino]carbonyl]amino]methyl]-N-[3-(2-methyl-1-piperidinyl)-propyl]-benzamide;
3-chloro-4-[[[[(10,11-dihydro-5H-dibenzo[a,d]-cyclohepten-5-yl)amino]carbonyl]amino]methyl]-N-[2-(1-methyl-2-pyrrolidinyl)-ethyl]-benzamide;
5-chloro-N-(tetrahydro-2-oxo-3-thienyl)-6-[2-[[[(1S,2R)-1,2,3,4-tetrahydro-2-phenyl-1-naphthalenyl]amino]thioxomethyl]hydrazino]-3-pyridinecarboxamide;
5-Chloro-6-[2-[[[(4-chloro-phenyl)-phenyl-methyl]-amino]-thioxomethyl]-hydrazino]-N-(tetrahydro-2-oxo-3-thienyl)-3-pyridine-carboxamide;
5-chloro-6-[2-[[(10,11-dihydro-5H-dibenzo[a,d]cyclohepten-5-yl)amino]thioxomethyl]hydrazino]-N-[(3R)-tetrahydro-2-oxo-3-thienyl]-3-pyridinecarboxamide;
5-chloro-6-[2-[[(1,2-diphenyl-ethyl)amino]thioxomethyl]hydrazino]-N-(tetrahydro-2-oxo-3-thienyl)-3-pyridinecarboxamide;
5-chloro-6-[2-[[(10,11-dihydro-5H-dibenzo[a,d]cyclohepten-5-yl)amino]thioxomethyl]hydrazino]-N-((3S)-tetrahydro-2-oxo-3-thienyl)-3-pyridinecarboxamide;
5-chloro-6-[2-[[(10,11-dihydro-5H-dibenzo[a,d]cyclohepten-5-yl)amino]thioxomethyl]hydrazino]-N-(tetrahydro-2-oxo-3-furanyl)-3-pyridinecarboxamide;
N-[[5-chloro-6-[2-[[(10,11-dihydro-5h-dibenzo[a,d]cyclo-hepten-5-yl)amino]thioxomethyl]-hydrazino]-3-pyridinyl]sulfonyl]-n-methyl-glycine, ethyl ester;
N-[[5-chloro-6-[2-[[(10,11-dihydro-5H-dibenzo[a,d]cyclo-hepten-5-yl)amino]carbonyl]-hydrazino]-3-pyridinyl]sulfonyl]-N-methyl-glycine, ethyl ester;
5-chloro-6-[2-[[(10,11-dihydro-5H-dibenzo[a,d]cyclohepten-5-yl)amino]carbonyl]hydrazino]-N-[[(2S)-1-ethyl-2-pyrrolidinyl]-methyl]-3-pyridinecarboxamide;
N-[[3-(aminomethyl)cyclohexyl]-methyl]-5-chloro-6-[2-[[(10,11-dihydro-5H-dibenzo[a,d]cyclo-hepten-5-yl)amino]carbonyl]-hydrazino]-3-pyridinecarboxamide;
N-[[3-(aminomethyl)phenyl]-methyl]-5-chloro-6-[2-[[(10,11-dihydro-5H-dibenzo[a,d]cyclo-hepten-5-yl)amino]carbonyl]-hydrazino]-3-pyridinecarboxamide;
5-chloro-6-[2-[[(10,11-dihydro-5H-dibenzo[a,d]cyclohepten-5-yl)amino]carbonyl]hydrazino]-N-[2-(1-methyl-2-pyrrolidinyl)ethyl]-3-pyridinecarboxamide;
5-chloro-N-[2-(diethylamino)-ethyl]-6-[2-[[(10,11-dihydro-5H-dibenzo[a,d]cyclohepten-5-yl)-amino]carbonyl]hydrazino]-3-pyridinecarboxamide;
5-chloro-N-[4-(diethylamino)-1-methylbutyl]-6-[2-[[(10,11-dihydro-5H-dibenzo[a,d]cyclo-hepten-5-yl)amino]carbonyl]-hydrazino]-3-pyridinecarboxamide;
5-chloro-6-[2-[[(10,11-dihydro-5H-dibenzo[a,d]cyclohepten-5-yl)-amino]carbonyl]hydrazino]-N-[2-(1-pyrrolidinyl)ethyl]-3-pyridine-carboxamide;
5-chloro-6-[2-[[(10,11-dihydro-5H-dibenzo[a,d]cyclohepten-5-yl)amino]carbonyl]hydrazino]-N-[3-(2-methyl-1-piperidinyl)-propyl]-3-pyridinecarboxamide;
5-chloro-6-[2-[[(10,11-dihydro-5H-dibenzo[a,d]cyclohepten-5-yl)amino]carbonyl]hydrazino]-N-[3-(dimethylamino)propyl]-3-pyridinecarboxamide;
5-chloro-6-[2-[[(10,11-dihydro-5H-dibenzo[a,d]cyclohepten-5-yl)amino]carbonyl]hydrazino]-N-[[(2R)-1-ethyl-2-pyrrolidinyl]-methyl]-3-pyridinecarboxamide;
3-[[[5-chloro-6-[2-[[(10,11-dihydro-5H-dibenzo[a,d]cyclo-hepten-5-yl)amino]carbonyl]-hydrazino]-3-pyridinyl]carbonyl]-amino]-1-pyrrolidinecarboxylic acid, 1,1-dimethylethyl ester;
5-chloro-6-[2-[[(10,11-dihydro-5H-dibenzo[a,d]cyclohepten-5-yl)amino]thioxomethyl]hydrazino]-N-[[(2S)-1-ethyl-2-pyrrolidinyl]-methyl]-3-pyridinecarboxamide;
5-chloro-6-[2-[[(10,11-dihydro-5H-dibenzo[a,d]cyclohepten-5-yl)amino]thioxomethyl]hydrazino]-N-[3-(4-morpholinyl)propyl]-3-pyridinecarboxamide;
N-[[3-(aminomethyl)cyclohexyl]-methyl]-5-chloro-6-[2-[[(10,11-dihydro-5H-dibenzo[a,d]cyclo-hepten-5-yl)amino]thioxomethyl]-hydrazino]-3-pyridinecarboxamide;
5-chloro-6-[2-[[(10,11-dihydro-5H-dibenzo[a,d]cyclohepten-5-yl)amino]thioxomethyl]hydrazino]-N-[3-(4-methyl-1-piperazinyl)-propyl]-3-pyridinecarboxamide;
5-chloro-6-[2-[[(10,11-dihydro-5H-dibenzo[a,d]cyclohepten-5-yl)amino]thioxomethyl]hydrazino]-N-[2-(1-methyl-2-pyrrolidinyl)-ethyl]-3-pyridinecarboxamide;
5-chloro-N-[2-(diethylamino)-ethyl]-6-[2-[[(10,11-dihydro-5H-dibenzo[a,d]cyclohepten-5-yl)-amino]thioxomethyl]hydrazino]-3-pyridinecarboxamide;
5-chloro-6-[2-[[(10,11-dihydro-5H-dibenzo[a,d]cyclohepten-5-yl)amino]thioxomethyl]hydrazino]-N-[2-(1-pyrrolidinyl)ethyl]-3-pyridinecarboxamide;
5-chloro-6-[2-[[(10,11-dihydro-5H-dibenzo[a,d]cyclohepten-5-yl)amino]thioxomethyl]hydrazino]-N-[3-(2-methyl-1-piperidinyl)-propyl]-3-pyridinecarboxamide;
5-chloro-6-[2-[[(10,11-dihydro-5H-dibenzo[a,d]cyclohepten-5-yl)amino]thioxomethyl]hydrazino]-N-[((2R)-1-ethyl-2-pyrrolidinyl)-methyl]-3-pyridinecarboxamide;
N-[[5-chloro-6-[2-[[(10,11-dihydro-5H-dibenzo[a,d]cyclo-hepten-5-yl)amino]thioxomethyl]-hydrazino]-3-pyridinyl]carbonyl]-N-methyl-glycine, methyl ester;
5-chloro-6-[2-[[(10,11-dihydro-5H-dibenzo[a,d]cyclohepten-5-yl)amino]carbonyl]hydrazino]-N-(2-hydroxyethyl)-N-(phenyl-methyl)-3-pyridinecarboxamide;
N-[[5-chloro-6-[2-[[(10,11-dihydro-5H-dibenzo[a,d]cyclo-hepten-5-yl)amino]thioxomethyl]-hydrazino]-3-pyridinyl]carbonyl]-glycine, ethyl ester;
5-chloro-6-[2-[[(10,11-dihydro-5H-dibenzo[a,d]cyclohepten-5-yl)amino]thioxomethyl]hydrazino]-N-[2-[[2-(dimethylamino)ethyl]-methylamino]-ethyl]-3-pyridine-carboxamide;
2-[2-chloro-4-[[(2-hydroxyethyl)-(phenylmethyl)amino]carbonyl]phenyl]-N-(10,11-dihydro-5H-dibenzo[a,d]cyclohepten-5-yl)-hydrazinecarboxamide;
3-[[[5-chloro-6-[2-[[(10,11-dihydro-5H-dibenzo[a,d]cyclo-hepten-5-yl)amino]carbonyl]-hydrazino]-3-pyridinyl]-carbonyl]-amino]-benzoic acid, ethyl ester;
5-chloro-N-(4,4-diethoxybutyl)-6-[2-[[(10,11-dihydro-5H-dibenzo-[a,d]cyclohepten-5-yl)amino]-carbonyl]hydrazino]-3-pyridine-carboxamide;
3-pyridinecarboxamide, 5-chloro-6-[2-[[(10,11-dihydro-5H-dibenzo-[a,d]cyclohepten-5-yl)amino]-thioxomethyl]hydrazino]-N-methyl-N-[2-(2-pyridinyl)ethyl]-;
5-chloro-6-[2-[[(10,11-dihydro-5H-dibenzo[a,d]cyclohepten-5-yl)amino]carbonyl]hydrazino]-N-[2-(1-piperidinyl)ethyl]-3-pyridinecarboxamide;
3-pyridinecarboxamide, 5-chloro-6-[2-[[(10,11-dihydro-5H-dibenzo-[a,d]cyclohepten-5-yl)amino]-carbonyl]hydrazino]-N-methyl-N-[2-(2-pyridinyl)ethyl]-;
5-chloro-6-[2-[[(10,11-dihydro-5H-dibenzo[a,d]cyclohepten-5-yl)-amino]carbonyl]hydrazino]-N-[2-(dimethylamino)ethyl]-N-(phenyl-methyl)-3-pyridinecarboxamide;
N-[3-chloro-4-[2-[[(10,11-dihydro-5H-dibenzo[a,d]cyclo-hepten-5-yl)amino]carbonyl]-hydrazino]benzoyl]-N-methyl-glycine, ethyl ester;
2-[3-chloro-5-[[4-(3-chloro-phenyl)-1-piperazinyl]carbonyl]-2-pyridinyl]-N-(10,11-dihydro-5H-dibenzo[a,d]cyclohepten-5-yl)-hydrazinecarboxamide;
(α¹S)-α-[[[2-[3-chloro-5-[[(2-hydroxyethyl)(phenylmethyl)amino]carbonyl]-2-pyridinyl]-hydrazino]thioxomethyl]amino]-benzeneacetic acid, 1,1-dimethylethyl ester;
5-chloro-6-[2-[[(10,11-dihydro-5H-dibenzo[a,d]cyclohepten-5-yl)amino]thioxomethyl]hydrazino]-N-[2-(1-piperidinyl)ethyl]-3-pyridinecarboxamide;
N-butyl-5-chloro-N-(cyano-methyl)-6-[2-[[(10,11-dihydro-5H-dibenzo[a,d]cyclohepten-5-yl)-amino]carbonyl]hydrazino]-3-pyridinecarboxamide;
5-chloro-6-[2-[[(10,11-dihydro-5H-dibenzo[a,d]cyclohepten-5-yl)amino]thioxomethyl]hydrazino]-N-[2-(dimethylamino)ethyl]-N-(phenylmethyl)-3-pyridine-carboxamide;
5-chloro-6-[2-[[(10,11-dihydro-5H-dibenzo[a,d]cyclohepten-5-yl)amino]thioxomethyl]hydrazino]-N-(3-fluorophenyl)-3-pyridine-carboxamide;
5-chloro-6-[2-[[(10,11-dihydro-5H-dibenzo[a,d]cyclohepten-5-yl)amino]carbonyl]hydrazino]-N-[3-(dimethylamino)-2,2-dimethyl-propyl]-3-pyridinecarboxamide;
5-chloro-6-[2-[[(10,11-dihydro-5H-dibenzo[a,d]cyclohepten-5-yl)-amino]carbonyl]hydrazino]-N-[2-(dimethylamino)-2-(4-methoxy-phenyl)ethyl]-3-pyridine-carboxamide;
3-[[[5-chloro-6-[2-[[(10,11-dihydro-5H-dibenzo[a,d]cyclo-hepten-5-yl)amino]thioxomethyl]-hydrazino]-3-pyridinyl]carbonyl]-amino]-benzoic acid, ethyl ester;
5-chloro-6-[2-[[(10,11-dihydro-5H-dibenzo[a,d]cyclohepten-5-yl)amino]carbonyl]hydrazino]-N-[[4-[2-(dimethylamino)ethoxy]-phenyl]methyl]-3-pyridine-carboxamide;
5-chloro-6-[2-[[(10,11-dihydro-5H-dibenzo[a,d]cyclohepten-5-yl)amino]thioxomethyl]hydrazino]-N-(3-iodophenyl)-3-pyridine-carboxamide;
5-chloro-6-[2-[[(10,11-dihydro-5H-dibenzo[a,d]cyclohepten-5-yl)amino]thioxomethyl]hydrazino]-N-[(3R)-tetrahydro-2-oxo-3-furanyl]-3-pyridinecarboxamide;
N-[[5-Chloro-6-[[[[(10,11-dihydro-5H-dibenzo[a,d]cyclo-hepten-5-yl)amino]carbonyl]-amino]methyl]-3-pyridinyl]-carbonyl]-N-methyl-glycine ethyl ester;
5-Chloro-6-[[[[(10,11-dihydro-5H-dibenzo[a,d]cyclohepten-5-yl)-amino]carbonyl]amino]methyl]-N-(tetrahydro-2-oxo-3-thienyl)-3-pyridinecarboxamide;
3-Chloro-4-[[[[(10,11-dihydro-5H-dibenzo[a,d]cyclohepten-5-yl)-amino]carbonyl]amino]methyl]-N-[3-(4-methyl-1-piperazinyl)-propyl]-benzamide;
3-Chloro-4-[[[[(10,11-dihydro-5H-dibenzo[a,d]cyclohepten-5-yl)-amino]carbonyl]amino]methyl]-N-(tetrahydro-1,1-dioxido-3-thienyl)-benzamide;
3-Chloro-4-[[[[(10,11-dihydro-5H-dibenzo[a,d]cyclohepten-5-yl)-amino]carbonyl]amino]methyl]-N-(3-methyl-5-isothiazolyl)-benzamide;
5-chloro-6-[2-[[(6,11-dihydro-5H-benzo[5,6]cyclohepta[1,2-c]pyridin-11-yl)amino]thioxomethyl]hydrazino]-N-[(3R)-tetrahydro-2-oxo-3-thienyl]-3-pyridinecarboxamide;
5-chloro-6-[2-[[(10,11-dihydro-5H-benzo[4,5]cyclohepta[1,2-b]pyridin-5-yl)amino]thioxomethyl]hydrazino]-N-[(3R)-tetrahydro-2-oxo-3-thienyl]-3-pyridinecarboxamide;
5-chloro-6-[2-[[(7-fluoro-6,11-dihydro-5H-benzo[5,6]cyclohepta[1,2-c]pyridin-11-yl)amino]thioxomethyl]hydrazino]-N-[(3R)-tetrahydro-2-oxo-3-thienyl]-3-pyridinecarboxamide;
5-chloro-6-[2-[[(7-fluoro-6,11-dihydro-5H-benzo[5,6]cyclohepta[1,2-c]pyridin-11-yl)amino]carbonyl]hydrazino]-N-[(3R)-tetrahydro-2-oxo-3-thienyl]-3-pyridinecarboxamide;
5-chloro-6-[2-[[(6,11-dihydro-5,5-dioxidodibenzo[b,e]thiepin-11-yl)amino]thioxomethyl]hydrazino]-N-[(3R)-tetrahydro-2-oxo-3-thienyl]-3-pyridinecarboxamide;
5-chloro-6-[2-[[(6,11-dihydrodibenzo[b,e]thiepin-11-yl)amino]thioxomethyl]hydrazino]-N-(tetrahydro-2-oxo-3-thienyl)-3-pyridinecarboxamide;
5-chloro-6-[2-[[(10,11-dihydro-5H-benzo[4,5]cyclohepta[1,2-c]pyridin-5-yl)amino]carbonyl]hydrazino]-N-(tetrahydro-2-oxo-3-thienyl)-3-pyridinecarboxamide;
5-chloro-6-[[[[(7-fluoro-6,11-dihydro-5H-benzo[5,6]cyclohepta[1,2-c]pyridin-11-yl)amino]thioxomethyl]amino]methyl]-N-[(3R)-tetrahydro-2-oxo-3-thienyl]-3-pyridinecarboxamide;
5-chloro-6-[[[[(7-fluoro-6,11-dihydro-5H-benzo[5,6]cyclohepta[1,2-c]pyridin-11-yl)amino]thioxomethyl]amino]methyl]-N-[(3R)-1-methyl-2-oxo-3-pyrrolidinyl]-3-pyridinecarboxamide;
5-chloro-6-[2-[[(7-fluoro-6,11-dihydro-5H-benzo[5,6]cyclohepta[1,2-c]pyridin-11-yl)amino]thioxomethyl]hydrazino]-N-[(3R)-1-methyl-2-oxo-3-pyrrolidinyl]-3-pyridinecarboxamide;
5-chloro-6-[2-[[(7-fluoro-6,11-dihydro-5H-benzo[5,6]cyclohepta[1,2-c]pyridin-11-yl)amino]carbonyl]hydrazino]-N-[(3R)-1-methyl-2-oxo-3-pyrrolidinyl]-3-pyridinecarboxamide;
5-chloro-6-[2-[[(6,11-dihydro-7-methyl-5H-benzo[5,6]cyclohepta[1,2-c]pyridin-11-yl)amino]thioxomethyl]hydrazino]-N-[(3R)-1-methyl-2-oxo-3-pyrrolidinyl]-3-pyridinecarboxamide;
5-chloro-6-[2-[[(6,11-dihydro-5H-benzo[5,6]cyclohepta[1,2-c]pyridin-11-yl)amino]thioxomethyl]hydrazino]-N-[(3R)-1-methyl-2-oxo-3-pyrrolidinyl]-3-pyridinecarboxamide;
5-chloro-6-[[[[(7-fluoro-6,11-dihydro-5H-benzo[5,6]cyclohepta[1,2-c]pyridin-1-yl)amino]carbonyl]amino]methyl]-N-[(3R)-1-methyl-2-oxo-3-pyrrolidinyl]-3-pyridinecarboxamide;
5-chloro-6-[2-[[(3-chloro-10,11-dihydro-5H-dibenzo[a,d]cyclohepten-5-yl)amino]thioxomethyl]hydrazino]-N-[(3R)-1-methyl-2-oxo-3-pyrrolidinyl]-3-pyridinecarboxamide;
5-chloro-6-[2-[[(2-chloro-10,11-dihydro-5H-dibenzo[a,d]cyclohepten-5-yl)amino]thioxomethyl]hydrazino]-N-[(3R)-1-methyl-2-oxo-3-pyrrolidinyl]-3-pyridinecarboxamide;
6-[2-[[(7-fluoro-6,11-dihydro-5H-benzo[5,6]cyclohepta[1,2-c]pyridin-11-yl)amino]thioxomethyl]hydrazino]-N-[(3R)-1-methyl-2-oxo-3-pyrrolidinyl]-3-pyridinecarboxamide;
5-chloro-6-[2-[[(7-fluoro-6,11-dihydro-5H-benzo[5,6]cyclohepta[1,2-c]pyridin-11-yl)amino]thioxomethyl]-1-methylhydrazino]-N-[(3R)-1-methyl-2-oxo-3-pyrrolidinyl]-3-pyridinecarboxamide;
5-chloro-6-[2-[[(9-fluoro-6,11-dihydro-5H-benzo[5,6]cyclohepta[1,2-c]pyridin-11-yl)amino]thioxomethyl]hydrazino]-N-[(3R)-1-methyl-2-oxo-3-pyrrolidinyl]-3-pyridinecarboxamide;
5-chloro-6-[2-[[(7-fluoro-10,11-dihydro-5H-benzo[4,5]cyclohepta[1,2-b]pyridin-5-yl)amino]thioxomethyl]hydrazino]-N-[(3R)-1-methyl-2-oxo-3-pyrrolidinyl]-3-pyridinecarboxamide;
5-chloro-6-[2-[[(9-fluoro-10,11-dihydro-5H-benzo[4,5]cyclohepta[1,2-b]pyridin-5-yl)amino]carbonyl]hydrazino]-N-[(3R)-1-methyl-2-oxo-3-pyrrolidinyl]-3-pyridinecarboxamide;
5-chloro-6-[2-[[(10,11-dihydro-5H-benzo[4,5]cyclohepta[1,2-c]pyridin-5-yl)amino]carbonyl]hydrazino]-N-(1-methyl-2-oxo-3-pyrrolidinyl)-3-pyridinecarboxamide;
2-[3-chloro-5-[[[(3R)-1-methyl-2-oxo-3-pyrrolidinyl]amino]sulfonyl]-2-pyridinyl]-N-(7-fluoro-6,11-dihydro-5H-benzo[5,6]cyclohepta[1,2-c]pyridin-11-yl)-hydrazinecarbothioamide;
2-[3-chloro-5-[[[(3R)-1-methyl-2-oxo-3-pyrrolidinyl]amino]sulfonyl]-2-pyridinyl]-N-(10,11-dihydro-5H-dibenzo[a,d]cyclohepten-5-yl)-hydrazinecarbothioamide;
5-chloro-6-[2-[[(10,11-dihydro-5H-dibenzo[a,d]cyclohepten-5-yl)amino]carbonyl]hydrazino]-N-[(3R)-1-methyl-2-oxo-3-pyrrolidinyl]-3-pyridinecarboxamide;
5-chloro-6-[2-[[(10,11-dihydro-5H-dibenzo[a,d]cyclohepten-5-yl)amino]thioxomethyl]hydrazino]-N-[(3R)-1-methyl-2-oxo-3-pyrrolidinyl]-3-pyridinecarboxamide;
5-chloro-6-[2-[[(6,11-dihydro-5H-benzo[5,6]cyclohepta[1,2-c]pyridin-1-yl)amino]thioxomethyl]hydrazino]-N-[(3R)-1-methyl-5-oxo-3-pyrrolidinyl]-3-pyridinecarboxamide;
5-chloro-6-[2-[[(6,11-dihydro-5H-benzo[5,6]cyclohepta[1,2-c]pyridin-11-yl)amino]thioxomethyl]hydrazino]-N-[(3S)-1-methyl-5-oxo-3-pyrrolidinyl]-3-pyridinecarboxamide;
5-chloro-6-[2-[[(9-fluoro-10,11-dihydro-5H-benzo[4,5]cyclohepta[1,2-b]pyridin-5-yl)amino]carbonyl]hydrazino]-N-[(3R)-tetrahydro-2-oxo-3-furanyl]-3-pyridinecarboxamide;
5-chloro-6-[2-[[(7-fluoro-10,11-dihydro-5H-benzo[4,5]cyclohepta[1,2-b]pyridin-5-yl)amino]thioxomethyl]hydrazino]-N-[(3R)-tetrahydro-2-oxo-3-furanyl]-3-pyridinecarboxamide;
5-chloro-6-[2-[[(10,11-dihydro-5H-dibenzo[a,d]cyclohepten-5-yl)amino]thioxomethyl]hydrazino]-N-(2-oxo-1-phenyl-3-pyrrolidinyl)-3-pyridinecarboxamide;
5-chloro-6-[2-[[(7-fluoro-6,11-dihydro-5H-benzo[5,6]cyclohepta[1,2-c]pyridin-11-yl)amino]thioxomethyl]hydrazino]-N-[(3R)-2-oxo-1-phenyl-3-pyrrolidinyl]-3-pyridinecarboxamide;
5-chloro-6-[2-[[(10,11-dihydro-5H-dibenzo[a,d]cyclohepten-5-yl)amino]carbonyl]hydrazino]-N-(2-oxo-1-phenyl-3-pyrrolidinyl)-3-pyridinecarboxamide;
N-[[5-chloro-6-[[[[(7-fluoro-6,11-dihydro-5H-benzo[5,6]cyclohepta[1,2-c]pyridin-11-yl)amino]thioxomethyl]amino]methyl]-3-pyridinyl]carbonyl]-N-methyl-glycine, ethyl ester;
N-[[5-chloro-6-[2-[[(7-fluoro-6,11-dihydro-5H-benzo[5,6]cyclohepta[1,2-c]pyridin-11-yl)amino]thioxomethyl]hydrazino]-3-pyridinyl]carbonyl]-glycine, ethyl ester;
5-chloro-6-[2-[[(10,11-dihydro-5H-benzo[4,5]cyclohepta[1,2-b]pyridin-5-yl)amino]thioxomethyl]hydrazino]-N-[(3R)-1-methyl-2-oxo-3-pyrrolidinyl]-3-pyridinecarboxamide;
5-chloro-6-[2-[[(7-fluoro-10,11-dihydro-5H-benzo[4,5]cyclohepta[1,2-b]pyridin-5-yl)amino]thioxomethyl]hydrazino]-N-[(3R)-2-oxo-1-phenyl-3-pyrrolidinyl]-3-pyridinecarboxamide;
5-chloro-6-[2-[[(6,11-dihydro-5H-benzo[5,6]cyclohepta[1,2-c]pyridin-11-yl)amino]carbonyl]hydrazino]-N-[(3R)-tetrahydro-2-oxo-3-thienyl]-3-pyridinecarboxamide;
5-chloro-6-[2-[[(7-fluoro-6,11-dihydro-5H-benzo[5,6]cyclohepta[1,2-c]pyridin-11-yl)amino]thioxomethyl]hydrazino]-N-[(3R)-1-methyl-5-oxo-3-pyrrolidinyl]-3-pyridinecarboxamide;
5-chloro-6-[2-[[(7-fluoro-6,11-dihydro-5H-benzo[5,6]cyclohepta[1,2-c]pyridin-11-yl)amino]thioxomethyl]hydrazino]-N-[[(2R)-tetrahydro-2-furanyl]methyl]-3-pyridinecarboxamide;
5-chloro-6-[2-[[(7-fluoro-6,11-dihydro-5H-benzo[5,6]cyclohepta[1,2-c]pyridin-11-yl)amino]thioxomethyl]hydrazino]-N-[[(2S)-tetrahydro-2-furanyl]methyl]-3-pyridinecarboxamide;
5-chloro-N-[2-(diethylamino)ethyl]-6-[2-[[(7-fluoro-6,11-dihydro-5H-benzo[5,6]cyclohepta[1,2-c]pyridin-11-yl)amino]thioxomethyl]hydrazino]-3-pyridinecarboxamide;
5-chloro-N-[2-(dimethylamino)ethyl]-6-[2-[[(7-fluoro-6,11-dihydro-5H-benzo[5,6]cyclohepta[1,2-c]pyridin-11-yl)amino]thioxomethyl]hydrazino]-3-pyridinecarboxamide;
5-chloro-6-[2-[[(7-fluoro-6,11-dihydro-5H-benzo[5,6]cyclohepta[1,2-c]pyridin-11-yl)amino]thioxomethyl]hydrazino]-N-[(1R*,2R*)-2-hydroxycyclopentyl]-3-pyridinecarboxamide;
5-chloro-6-[2-[[(7-fluoro-6,11-dihydro-5H-benzo[5,6]cyclohepta[1,2-c]pyridin-11-yl)amino]thioxomethyl]hydrazino]-N-(2-methoxyphenyl)-3-pyridinecarboxamide;
5-chloro-6-[2-[[(7-fluoro-6,11-dihydro-5H-benzo[5,6]cyclohepta[1,2-c]pyridin-11-yl)amino]thioxomethyl]hydrazino]-N-(2-methoxy-3-pyridinyl)-3-pyridinecarboxamide;
N-[5-chloro-6-[2-[[(7-fluoro-6,11-dihydro-5H-benzo[5,6]cyclohepta[1,2-c]pyridin-11-yl)amino]thioxomethyl]hydrazino]-3-pyridinyl]-N-methyl-acetamide; [5-chloro-6-[2-[[(7-fluoro-6,11-dihydro-5H-benzo[5,6]cyclohepta[1,2-c]pyridin-11-yl)amino]thioxomethyl]hydrazino]-3-pyridinyl]methyl-carbamic acid, methyl ester;
N-[5-chloro-6-[2-[[(7-fluoro-6,11-dihydro-5H-benzo[5,6]cyclohepta[1,2-c]pyridin-1-yl)amino]thioxomethyl]hydrazino]-3-pyridinyl]-N,3-dimethyl-butanamide;
N-[5-chloro-6-[2-[[(7-fluoro-6,11-dihydro-5H-benzo[5,6]cyclohepta[1,2-c]pyridin-11-yl)amino]thioxomethyl]hydrazino]-3-pyridinyl]-N-methyl-cyclopropanecarboxamide;
N-[5-chloro-6-[2-[[(7-fluoro-6,11-dihydro-5H-benzo[5,6]cyclohepta[1,2-c]pyridin-11-yl)amino]thioxomethyl]hydrazino]-3-pyridinyl]-N-methyl-3-pyridinecarboxamide;
N-[5-chloro-6-[2-[[(7-fluoro-6,11-dihydro-5H-benzo[5,6]cyclohepta[1,2-c]pyridin-11-yl)amino]thioxomethyl]hydrazino]-3-pyridinyl]-N-methyl-4-pyridinecarboxamide;
N-[5-chloro-6-[2-[[(7-fluoro-6,11-dihydro-5H-benzo[5,6]cyclohepta[1,2-c]pyridin-11-yl)amino]thioxomethyl]hydrazino]-3-pyridinyl]-3-fluoro-N-methyl-benzamide;
N-[5-chloro-6-[2-[[(7-fluoro-6,11-dihydro-5H-benzo[5,6]cyclohepta[1,2-c]pyridin-11-yl)amino]thioxomethyl]hydrazino]-3-pyridinyl]-4-fluoro-N-methyl-benzamide;
5-chloro-6-[2-[[(10,11-dihydro-5H-benzo[4,5]cyclohepta[1,2-b]pyridin-5-yl)amino]thioxomethyl]hydrazino]-N-(2-hydroxyethyl)-N-(phenylmethyl)-3-pyridinecarboxamide;
2-[3-chloro-5-[[(2-hydroxyethyl)(phenylmethyl)amino]sulfonyl]-2-pyridinyl]-N-(10,11-dihydro-5H-dibenzo[a,d]cyclohepten-5-yl)-hydrazinecarbothioamide;
2-[3-chloro-5-[[(2-hydroxyethyl)(phenylmethyl)amino]sulfonyl]-2-pyridinyl]-N-(10,11-dihydro-5H-dibenzo[a,d]cyclohepten-5-yl)-hydrazinecarboxamide;
2-[3-chloro-5-[[[(1-ethyl-2-pyrrolidinyl)methyl]amino]sulfonyl]-2-pyridinyl]-N-[7-ethenyl-8,9-dihydro-6-[(1Z)-1-propenyl]-5H-benzocyclohepten-5-yl]-hydrazinecarbothioamide;
5-chloro-6-[2-[[(9-fluoro-6,11-dihydro-5H-benzo[5,6]cyclohepta[1,2-c]pyridin-11-yl)amino]thioxomethyl]hydrazino]-N-[2-(1-methyl-2-pyrrolidinyl)ethyl]-3-pyridinecarboxamide;
5-chloro-6-[2-[[(9-fluoro-6,11-dihydro-5H-benzo[5,6]cyclohepta[1,2-c]pyridin-11-yl)amino]thioxomethyl]hydrazino]-N-(2-hydroxyethyl)-N-(phenylmethyl)-3-pyridinecarboxamide;
N-[[5-chloro-6-[2-[[(6,11-dihydrodibenzo[b,e]thiepin-1-yl)amino]thioxomethyl]hydrazino]-3-pyridinyl]carbonyl]-N-methyl-glycine, ethyl ester;
N-[[5-chloro-6-[2-[[(6,11-dihydro-5H-benzo[5,6]cyclohepta[1,2-c]pyridin-11-yl)amino]thioxomethyl]hydrazino]-3-pyridinyl]carbonyl]-N-methyl-glycine, ethyl ester;
5-chloro-N-[2-(diethylamino)ethyl]-6-[2-[[(6,11-dihydrodibenzo[b,e]thiepin-11-yl)amino]thioxomethyl]hydrazino]-3-pyridinecarboxamide;
5-chloro-N-[(1-ethyl-2-pyrrolidinyl)methyl]-6-[2-[[(9-fluoro-6,11-dihydro-5H-benzo[5,6]cyclohepta[1,2-c]pyridin-11-yl)amino]thioxomethyl]hydrazino]-3-pyridinecarboxamide;
5-chloro-N-[2-(diethylamino)ethyl]-6-[2-[[(9-fluoro-6,11-dihydro-5H-benzo[5,6]cyclohepta[1,2-c]pyridin-11-yl)amino]thioxomethyl]hydrazino]-3-pyridinecarboxamide;
5-chloro-N-[2-(diethylamino)ethyl]-6-[2-[[(6,11-dihydro-5H-benzo[5,6]cyclohepta[1,2-c]pyridin-11-yl)amino]thioxomethyl]hydrazino]-3-pyridinecarboxamide;
5-chloro-6-[2-[[(6,11-dihydro-5H-benzo[5,6]cyclohepta[1,2-c]pyridin-1-yl)amino]thioxomethyl]hydrazino]-N-[2-(1-pyrrolidinyl)ethyl]-3-pyridinecarboxamide;
5-chloro-6-[2-[[(9-fluoro-6,11-dihydro-5H-benzo[5,6]cyclohepta[1,2-c]pyridin-1-yl)amino]thioxomethyl]hydrazino]-N-(tetrahydro-2-oxo-3-furanyl)-3-pyridinecarboxamide;
5-chloro-6-[2-[[(6,11-dihydro-5H-benzo[5,6]cyclohepta[1,2-c]pyridin-11-yl)amino]thioxomethyl]hydrazino]-N-[(1-ethyl-2-pyrrolidinyl)methyl]-3-pyridinecarboxamide;
5-chloro-6-[2-[[(6,11-dihydro-5H-benzo[5,6]cyclohepta[1,2-c]pyridin-11-yl)amino]thioxomethyl]hydrazino]-N-(tetrahydro-2-oxo-3-furanyl)-3-pyridinecarboxamide;
5-chloro-6-[2-[[(6,11-dihydro-5H-benzo[5,6]cyclohepta[1,2-c]pyridin-11-yl)amino]thioxomethyl]hydrazino]-N-(2-hydroxyethyl)-N-(phenylmethyl)-3-pyridinecarboxamide;
5-chloro-6-[2-[[(9-fluoro-6,11-dihydro-5H-benzo[5,6]cyclohepta[1,2-c]pyridin-11-yl)amino]thioxomethyl]hydrazino]-N-[3-(4-methyl-1-piperazinyl)propyl]-3-pyridinecarboxamide;
5-chloro-6-[2-[[(6,11-dihydro-5H-benzo[5,6]cyclohepta[1,2-c]pyridin-11-yl)amino]thioxomethyl]hydrazino]-N-[3-(4-methyl-1-piperazinyl)propyl]-3-pyridinecarboxamide;
5-chloro-6-[2-[[(9-fluoro-6,11-dihydro-5H-benzo[5,6]cyclohepta[1,2-c]pyridin-11-yl)amino]thioxomethyl]hydrazino]-N-methyl-N-[2-(2-pyridinyl)ethyl]-3-pyridinecarboxamide;
5-chloro-N-[[(2S)-1-ethyl-2-pyrrolidinyl]methyl]-6-[2-[[[4-(4-propylcyclohexyl)phenyl]amino]thioxomethyl]hydrazino]-3-pyridinecarboxamide;
5-chloro-N-[[(2R)-1-ethyl-2-pyrrolidinyl]methyl]-6-[2-[[[4-(4-propylcyclohexyl)phenyl]amino]thioxomethyl]hydrazino]-3-pyridinecarboxamide;
5-chloro-6-[2-[[(9-chloro-6,11-dihydro-5H-benzo[5,6]cyclohepta[1,2-c]pyridin-11-yl)amino]thioxomethyl]hydrazino]-N-(2-hydroxyethyl)-N-(phenylmethyl)-3-pyridinecarboxamide;
5-chloro-6-[2-[[(9-fluoro-6,11-dihydro-5H-benzo[5,6]cyclohepta[1,2-c]pyridin-11-yl)amino]thioxomethyl]hydrazino]-N-[2-(1-pyrrolidinyl)ethyl]-3-pyridinecarboxamide;
5-chloro-6-[2-[[(6,11-dihydro-5H-benzo[5,6]cyclohepta[1,2-c]pyridin-11-yl)amino]thioxomethyl]hydrazino]-N-methyl-N-[2-(2-pyridinyl)ethyl]-3-pyridinecarboxamide;
5-chloro-6-[2-[[(6,11-dihydrodibenzo[b,e]thiepin-11-yl)amino]thioxomethyl]hydrazino]-N-[3-(4-methyl-1-piperazinyl)propyl]-3-pyridinecarboxamide;
5-chloro-N-[2-(1-methyl-2-pyrrolidinyl)ethyl]-6-[2-[[[4-(4-propylcyclohexyl)phenyl]amino]thioxomethyl]hydrazino]-3-pyridinecarboxamide;
2-[3-chloro-5-[[[(1-ethyl-2-pyrrolidinyl)methyl]amino]sulfonyl]-2-pyridinyl]-N-[4-(4-propylcyclohexyl)phenyl]-hydrazinecarbothioamide;
5-chloro-6-[2-[[(6,11-dihydrodibenzo[b,e]thiepin-11-yl)amino]thioxomethyl]hydrazino]-N-[2-(1-methyl-2-pyrrolidinyl)ethyl]-3-pyridinecarboxamide;
5-chloro-6-[2-[[(6,11-dihydrodibenzo[b,e]thiepin-11-yl)amino]thioxomethyl]hydrazino]-N-[(1-ethyl-2-pyrrolidinyl)methyl]-3-pyridinecarboxamide;
5-chloro-6-[2-[[(9-chloro-6,11-dihydro-5H-benzo[5,6]cyclohepta[1,2-c]pyridin-11-yl)amino]thioxomethyl]hydrazino]-N-[3-(4-methyl-1-piperazinyl)propyl]-3-pyridinecarboxamide;
5-chloro-6-[2-[[(6,11-dihydro-6-methyl-5,5-dioxidodibenzo[c,f][1,2]thiazepin-11-yl)amino]thioxomethyl]hydrazino]-N-(tetrahydro-2-oxo-3-thienyl)-3-pyridinecarboxamide;
5-chloro-N-[(1-ethyl-2-pyrrolidinyl)methyl]-6-[2-[[[4-(4-propylcyclohexyl)phenyl]amino]thioxomethyl]hydrazino]-3-pyridinecarboxamide;
5-chloro-6-[2-[[(6,11-dihydro-5H-benzo[5,6]cyclohepta[1,2-c]pyridin-11-yl)amino]thioxomethyl]hydrazino]-N-[2-(1-methyl-2-pyrrolidinyl)ethyl]-3-pyridinecarboxamide;
5-chloro-6-[2-[[(6,11-dihydrodibenzo[b,e]thiepin-11-yl)amino]thioxomethyl]hydrazino]-N-[2-(1-pyrrolidinyl)ethyl]-3-pyridinecarboxamide;
2-[3-chloro-5-[[[(1-ethyl-2-pyrrolidinyl)methyl]amino]sulfonyl]-2-pyridinyl]-N-(10,11-dihydro-5H-dibenzo[a,d]cyclohepten-5-yl)-hydrazinecarboxamide;
5-chloro-N-[(3R)-1-methyl-2-oxo-3-pyrrolidinyl]-6-[2-[[[(1R,2R)-1,2,3,4-tetrahydro-2-phenyl-1-naphthalenyl]amino]thioxomethyl]hydrazino]-3-pyridinecarboxamide;
5-chloro-6-[2-[[(9-chloro-6,11-dihydro-5H-benzo[5,6]cyclohepta[1,2-c]pyridin-11-yl)amino]thioxomethyl]hydrazino]-N-[2-(1-methyl-2-pyrrolidinyl)ethyl]-3-pyridinecarboxamide;
2-[3-chloro-5-[[(2-hydroxyethyl)(phenylmethyl)amino]sulfonyl]-2-pyridinyl]-N-(diphenylmethyl)-hydrazinecarbothioamide;
5-chloro-6-[2-[[(6,11-dihydro-6-methyl-5,5-dioxidodibenzo[c,f][1,2]thiazepin-11-yl)amino]thioxomethyl]hydrazino]-N-(1-methyl-2-oxo-3-pyrrolidinyl)-3-pyridinecarboxamide;
and pharmaceutically acceptable salts thereof.

8. A compound according to any one of claims 1-7 for use as a medicament.

9. The use of a compound according to any one of claims 1-7 in the manufacture of a medicament for the therapy of pain.

10. A pharmaceutical composition comprising a compound according to any one of claims 1-7 and a pharmaceutically acceptable carrier.

11. A method for the therapy of pain in a warm-blooded animal, comprising the step of administering to said animal in need of such therapy a therapeutically effective amount of a compound according to any one of claims 1-7.

12. A compound according to any one of claims 1-7 for use as an antagonist of bradykinin at the B2 receptor.

13. A method for preparing compounds of Formula I, 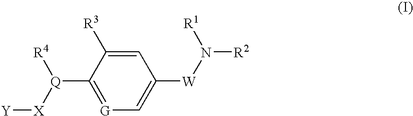
comprising the steps of:
a) reacting a compound of general formula IV, with an isocyanate (Y—NCO) or thioisocyanate (Y—NCS), 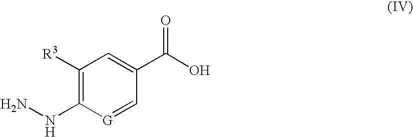
to give a compound of Formula V; 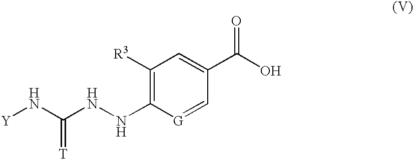
b) reacting the compound of Formula V with an amine HNR¹R² in the presence of an amide coupling reagent and an acid scavenger to yield the compound of formula I, Wherein
R¹ and R² are independently selected from hydrogen, optionally substituted acyl, optionally substituted alkyl-oxycarbonyl, optionally substituted alkyl, optionally substituted heteroalkyl, optionally substituted cycloalkyl, optionally substituted aryl; optionally substituted heterocyclyl; optionally substituted aryl-C₁₋₆alkyl, and optionally substituted heterocyclyl-C₁₋₆alkyl;
T is S or O;
X is represented by formula (i) or (ii), 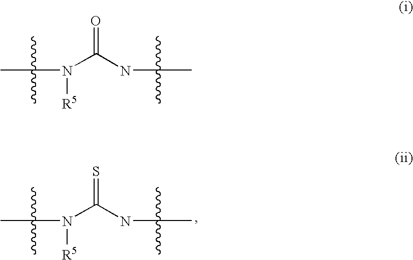
wherein R⁵ is —H, or optionally substituted C₁₋₆alkyl;
Q is N
R⁴ is —H;
G is CH or N;
R³ is halogen;
Y is a group that includes an optionally substituted seven-membered ring and two optionally substituted aromatic rings, wherein each of the aromatic rings is independently fused with said seven-membered ring, and wherein each of said seven-membered ring and aromatic rings, independently, optionally, contains one or more heteroatoms; and
W is —C(═O)—.

14. A method of preparing a compound of formula VI comprising the step of reacting a compound of formula VII with a compound of Y—NCO or Y—NCS: 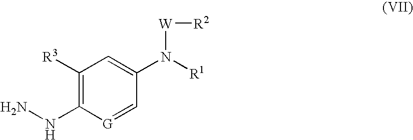
to form the compound of formula VI: 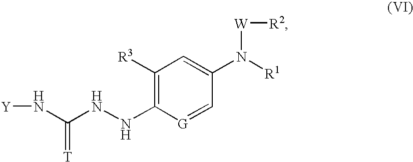
wherein
R¹ and R² are independently selected from hydrogen, optionally substituted acyl, optionally substituted alkyl-oxycarbonyl, optionally substituted alkyl, optionally substituted heteroalkyl, optionally substituted cycloalkyl, optionally substituted aryl; optionally substituted heterocyclyl; optionally substituted aryl-C₁₋₆alkyl, and optionally substituted heterocyclyl-C₁₋₆alkyl;
T is S or O;
X is represented by formula (i) or (ii), 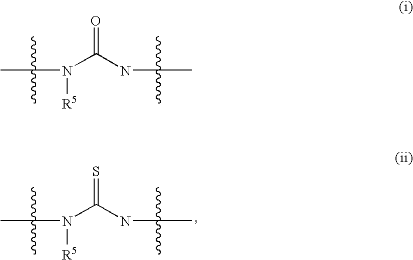
wherein R⁵ is —H, or optionally substituted C₁₋₆alkyl;
G is CH or N;
R³ is halogen;
Y is a group that includes an optionally substituted seven-membered ring and two optionally substituted aromatic rings, wherein each of the aromatic rings is independently fused with said seven-membered ring, and wherein each of said seven-membered ring and aromatic rings, independently, optionally, contains one or more heteroatoms; and
W is —C(═O)—.

15. A compound of formula (V): 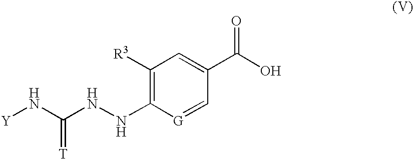
wherein
G is N or CH;
T is O or S;
R³ is halogen, hydrogen or C₁₋₆alkyl; and
Y is a group that includes an optionally substituted seven-membered ring and two optionally substituted aromatic rings, wherein each of the aromatic rings is independently fused with said seven-membered ring, and wherein each of said seven-membered ring and aromatic rings, independently, optionally, contains one or more heteroatoms.

16. A compound of formula (VII): 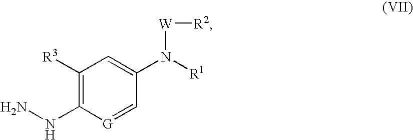
wherein
R¹ and R² are independently selected from hydrogen, optionally substituted C₁₋₁₂acyl, optionally substituted C₁₋₁₂alkyl-oxycarbonyl, optionally substituted C₁₋₁₂alkyl, optionally substituted C₁₋₁₂heteroalkyl, optionally substituted C₃₋₁₂cycloalkyl, optionally substituted C₆₋₁₂aryl and optionally substituted C₂₋₁₂heterocyclyl;
W is —C(═O)— or —S(═O)₂—;
G is N or CH; and
R³ is halogen, hydrogen or C₁₋₆alkyl.

17. A compound of Y—NCO or Y—NCS,
wherein Y is a group that includes an optionally substituted seven-membered ring and two optionally substituted aromatic rings, wherein each of the aromatic rings is independently fused with said seven-membered ring, and wherein each of said seven-membered ring and aromatic rings, independently, optionally, contains one or more heteroatoms.

18. A compound of formula (II), pharmaceutically acceptable salts thereof, diasteriomers thereof, enantiomers thereof, or mixtures thereof: 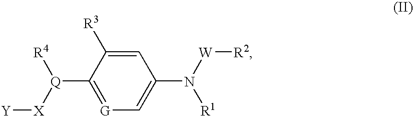
wherein
R¹ and R² are independently selected from hydrogen, optionally substituted C₁₋₁₂acyl, optionally substituted C₁₋₁₂alkyl-oxycarbonyl, optionally substituted C₁₋₁₂alkyl, optionally substituted C₁₋₁₂heteroalkyl, optionally substituted C₃₋₁₂cycloalkyl, optionally substituted C₆₋₁₂aryl and optionally substituted C₂₋₁₂heterocyclyl;
W is a linking group that separates the groups linked thereto by one or two atoms;
G is N or CH;
R³ is halogen, hydrogen or C₁₋₆alkyl;
Q is N or CH;
R⁴ is —H or optionally substituted hydrocarbyl;
X is a divalent group including first nitrogen atom and a second nitrogen atom, wherein a first group linked to X is linked to the first nitrogen and a second group linked to X is linked to the second nitrogen atom, and wherein the first and second nitrogen atoms are separated by either one carbon atom, or two carbon atoms wherein said two carbon atoms form a double bond therebetween; and
Y is a group that includes an optionally substituted seven-membered ring and two optionally substituted aromatic rings, wherein each of the aromatic rings is independently fused with said seven-membered ring, and wherein each of said seven-membered ring and aromatic rings, independently, optionally, contains one or more heteroatoms.

19. A compound of formula (II), pharmaceutically acceptable salts thereof, diasteriomers thereof, enantiomers thereof, or mixtures thereof, 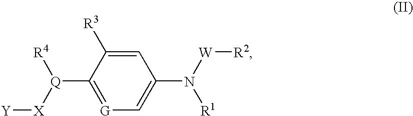
wherein
R¹ and R² are independently selected from hydrogen, optionally substituted acyl, optionally substituted alkyl-oxycarbonyl, optionally substituted alkyl, optionally substituted heteroalkyl, optionally substituted cycloalkyl, optionally substituted aryl; optionally substituted heterocyclyl; optionally substituted aryl-C₁₋₆alkyl, and optionally substituted heterocyclyl-C₁₋₆alkyl;
W is a linking group selected from —C(═O)—, —C(═O)O— and —S(═O)₂—;
G is N or CH;
R³ is halogen, or hydrogen
Q is N or CH;
R⁴ is —H, optionally substituted hydrocarbyl, a single bond, or a divalent group;
X is represented by (i), (ii), (iii), (iv), (v), (vi), (vii), (viii), (ix), (x), (xi), (xii), (xiii), (xiv), (xv), (xvi), or (xvii) below: 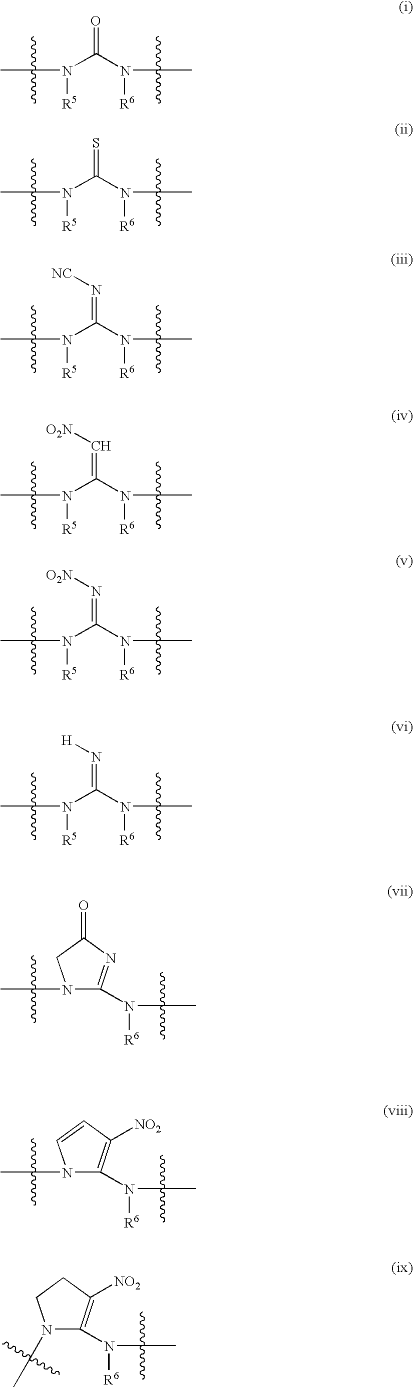 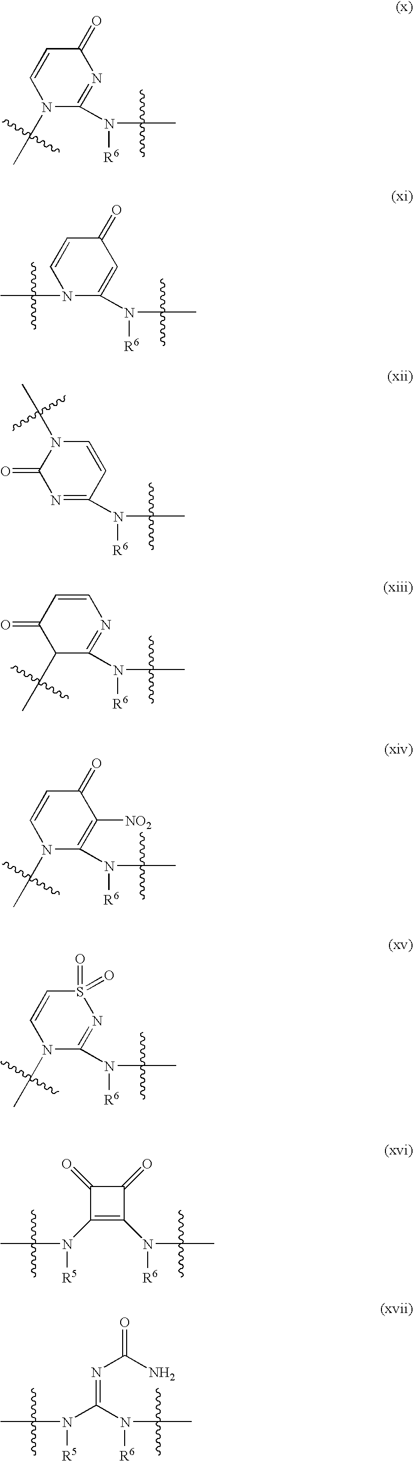
wherein R⁵ is selected from —H or optionally substituted alkyl, or a divalent C₀₋₆group together with R⁴ to form a portion of a ring, wherein said divalent C₀₋₆ group optionally contains one or more heteroatoms;
R⁶ is independently selected from —H or optionally substituted alkyl;
Y is represented by formula (R) below: 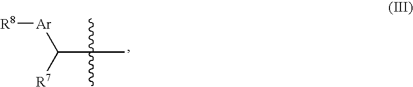
wherein
R⁷ is optionally substituted aryl, optionally substituted heteroaryl, optionally substituted aryl-C₁₋₆alkyl, optionally substituted heteroaryl-C₁₋₆alkyl, —C(═O)O—R⁹, —C(═O)NHR⁹, —C(═O)NR⁹R¹⁰, —SO₂NHR⁹, —SO₂NR⁹R¹¹, —R¹¹NH₂, —R¹¹R¹², —R¹¹NR¹²R¹³, —R¹¹OH, —R¹¹OR¹², —R¹¹SH, —R¹¹SR¹², or a divalent C₀₋₆group which together with R⁸ forms a portion of a ring,
R⁸ is —H, halogen, optionally substituted R¹², —OR¹², —SR¹², —S(═O)R¹², —SO₂R¹², —C(═O)R¹², or a divalent C₀₋₆group which together with the divalent R⁷ forms the portion of the ring,
wherein R⁹ and R¹⁰ are independently C₁₋₁₂hydrocarbyl, R¹¹ is C₁₋₆alkylene, R¹² and R¹³ are independently C₁₋₆alkyl; and
Ar is optionally substituted arylene, optionally substituted heteroarylene, optionally substituted arylene-C₁₋₆alkyl, or optionally substituted heteroarylene-C₁₋₆alkyl.

20. A compound according to claim 19, wherein
R¹ is selected from C₁₋₆alkyl, C₃₋₆cycloalkyl, aryl, aryl-C₁₋₆alkyl or heterocyclyl, heterocyclyl-C₁₋₆alkyl, wherein said C₁₋₈alkyl and C₃₋₈cycloalkyl are optionally, independently, subsituted by —R²⁰, —C(═O)R²⁰, oxo (═O), sulfo (═S), —OH, —OR²⁰, phenyl, halogen, heterocyclyl, —NH₂, —NHR²⁰, —NR²⁰R²¹, —C(═O)NH₂, —C(═O)NHR²⁰, —C(═O)NR²⁰R²¹ and —C(═O)OR²⁰, wherein said aryl is optionally substituted by —R²⁰, —C(═O)R²⁰, —OH, —OR²⁰, phenyl, halogen, heterocyclyl, —NH₂, NHR²⁰, N²⁰R²¹, —C(═O)NH₂, —C(═O)NHR²⁰, —C(═O)NR²⁰R²¹ and —C(═O)OR²⁰, wherein said heterocyclyl is a five or six-membered heterocyclyl, wherein said heterocyclyl is optionally substituted by —R²⁰, aryl, heteroaryl, —NH₂, —NHR²⁰, —NR²⁰R²¹, —C(═O)NH₂, —C(═O)NHR²⁰, —C(═O)NR²⁰R²¹, —C(═O)OR²⁰, or oxo (═O);
R² is selected from C₁₋₆alkyl, C₃₋₆cycloalkyl, wherein said C₁₋₆alkyl and C₃₋₆cycloalkyl are optionally, independently, subsituted by R²⁰, —C(═O)R²⁰, oxo (═O), sulfo (═S), —OH, —OR²⁰, phenyl, halogen, heterocyclyl, —NH₂, —NHR²⁰, NR²⁰R²¹, —C(═O)NH₂, —C(═O)NHR²⁰, C(═O)NR²⁰R²¹ and —C(═O)OR²⁰;
wherein R²⁰ and R²¹ are independently C₁₋₆alkyl;
R³ is selected from bromo, chloro and fluoro;
R⁴ is —H, optionally substituted (C₁-C₆)alkyl, or a divalent group together with R⁵ of X to form a portion of a first ring;
W is —C(═O)—, or —S(═O)₂—;
G is N or CH;
Q is CH or N;
X is selected from Formulas (i) and (ii), below: 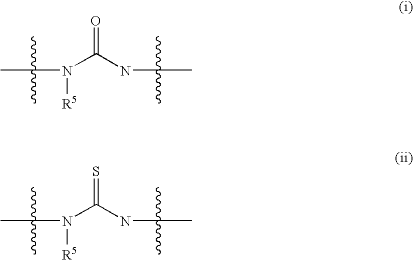
wherein R⁵ is —H, optionally substituted C₁₋₆alkyl, a bond or a divalent group wherein said bond or divalent group together with R⁴ forms the portion of the first ring, wherein said first ring is selected from optionally substituted Formulas (a), (b) and (c), 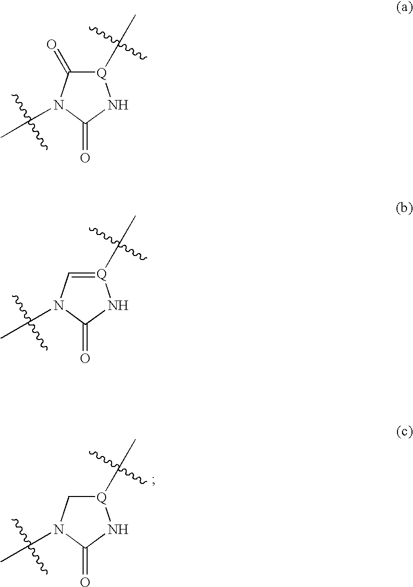
wherein when R⁴ or R⁵ is substituted, substituents of R⁴ or R⁵ are selected from: —OH, NH2, —O(C₁-C₃)alkyl, —CN, oxo (═O), —C(═O)O(C₁-C₄)alkyl and halogen.
Y is represented by formula (II) below: 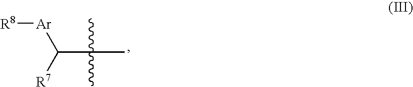
R⁷ is optionally substituted aryl, optionally substituted heterocyclyl, or optionally substituted arylene which together with R⁸ forms a portion of a second ring;
R⁸ is R²², —OR²², —SR²², —S(═O)R²², —SO₂R²², —C(═O)R²², or a optionally substituted divalent C₀₋₆group which together with the divalent R⁷ forms the portion of the second ring;
wherein when R⁷ or R⁸ is substituted, substituents of R⁷ or R⁸ are halogen, nitro, cyano, R²², —C(═O)R²², —C(═O)OR²², —OH, —OR²², —C(═O)NH₂, —C(═O)NHR²², —C(═O)NR²²R²³, —SO₂NH₂, —SO₂NHR²² or —SO₂NR²²R²³;
Ar is optionally substituted arylene, optionally substituted heteroarylene, optionally substituted arylene-C₁₋₆alkyl, or optionally substituted heteroarylene-C₁₋₆alkyl; and
wherein R²² and R²³ are independently C₁₋₆alkyl.

21. A compound according to claim 19, wherein
R¹ is selected from C₁₋₆alkyl, C₃₋₆cycloalkyl, aryl, aryl-C₁₋₆alkyl or heterocyclyl, heterocyclyl-C₁₋₆alkyl, wherein said C₁₋₆alkyl and C₃₋₆cycloalkyl and aryl are optionally, independently, subsituted by —OH, —C(═O)OR²⁴, —OR²⁴ or —NR²⁴R²⁵, wherein said heterocyclyl is derived from pyrrolidinone, five-membered lactone, five-membered thiolactone, pyrrolidine, tetrahyrofuran, thiophan, sulfolane, piperidine, piperazine, morpholine, thiomorpholine, dioxane, tetrahydropyran or tetrahydrothiopyran by removing a hydrogen therefrom, wherein said heterocyclyl is optionally substituted by oxo (═O);
R² is selected from C₁₋₆alkyl, C₃₋₆cycloalkyl, wherein said C₁₋₆alkyl and C₃₋₆cycloalkyl are optionally, independently, subsituted by —OH, —C(═O)OR²⁴, —OR²⁴ and —NR²⁴R²⁵;
wherein R²⁴ and R²⁵ are independently C₁₋₆alkyl;
R³ is chloro;
R⁴ is —H;
G is N or CH;
Q is N or CH;
W is —C(═O)—;
X is selected from Formulas (i) and (ii), below: 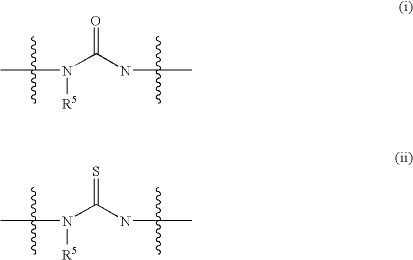
wherein R⁵ is —H or C₁₋₆alkyl;
Y is selected from formulas (d), (e), (f), (g), (h), (j) and (k), below: 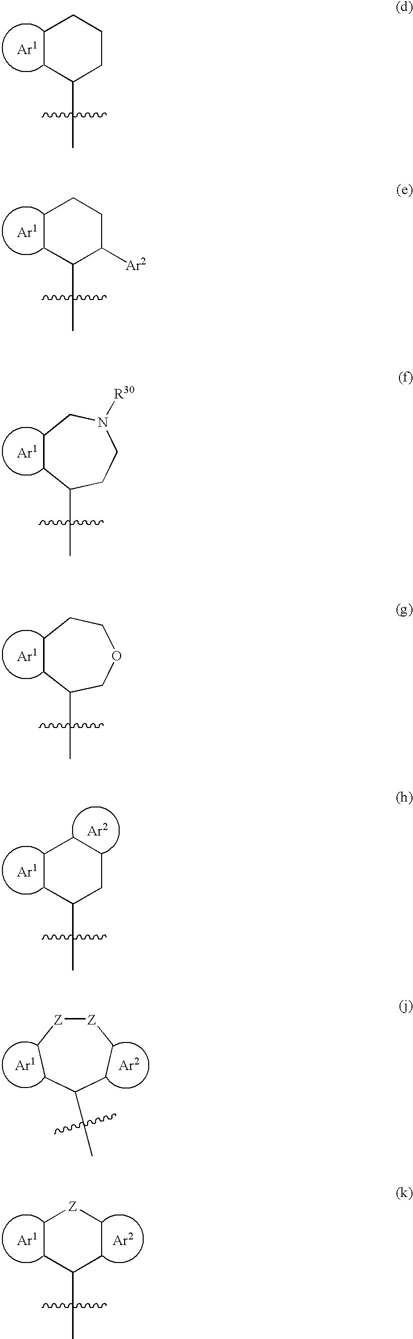
wherein Z is selected from —C—, —C(═O)—, —O—, —N(-alkyl)-, —NH—, —S—, —S(═O)— and —SO₂—; Ar¹ and Ar² are, independently, optionally substituted aryl, or optionally subsitituted heteroaryl; R³⁰ is a C₁₋₆hydrocarbyl; and when Ar¹ or Ar² is represented by a three-quarter cycle attached to a ring structure, Ar¹ or Ar² is fused with said ring structure.

22. A compound according to claim 21, wherein
R¹ is C₁₋₃alkyl;
R² is C₁₋₆alkyl optionally substituted by halo or heteroaryl, or aryl optionally substituted by halo or heteroaryl;
R⁵ is —H; and
Y is selected from structures (l), (m), (n), (O), (p), (q), (r), (s), (t), (u), (v), (w), (x), (y), (z), (a1), (b1), (c1), (d1), (e1), (f1), (g1) and (h1) below, 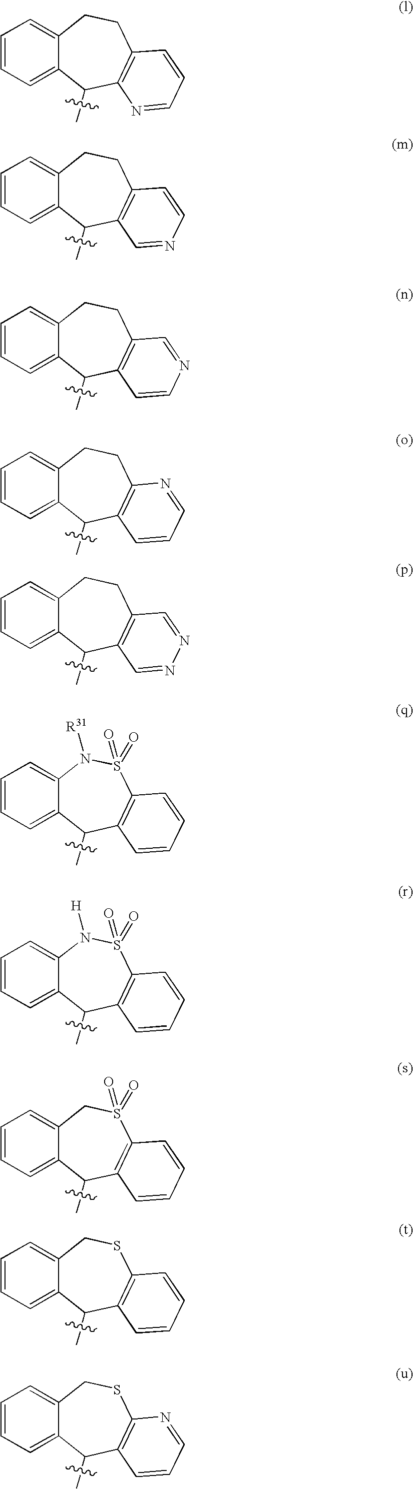 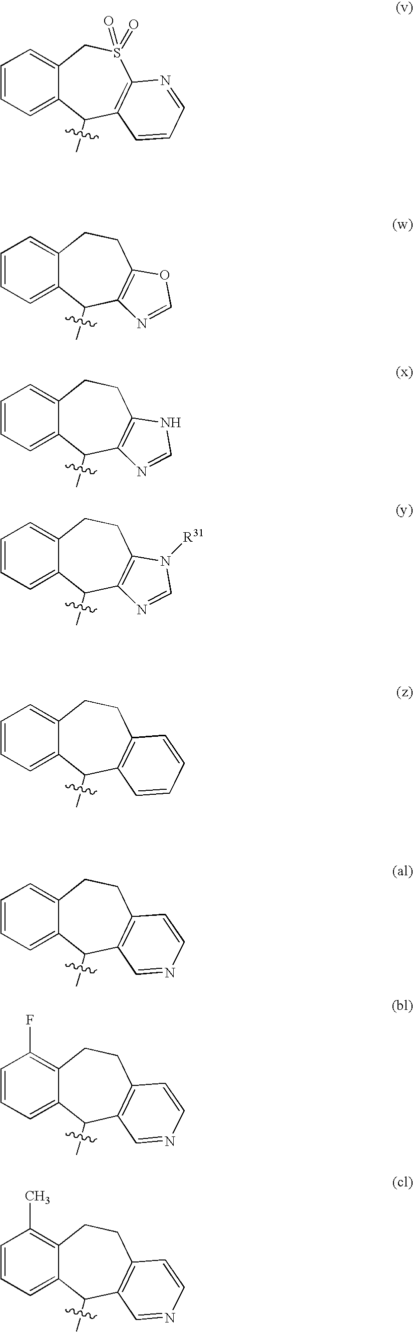 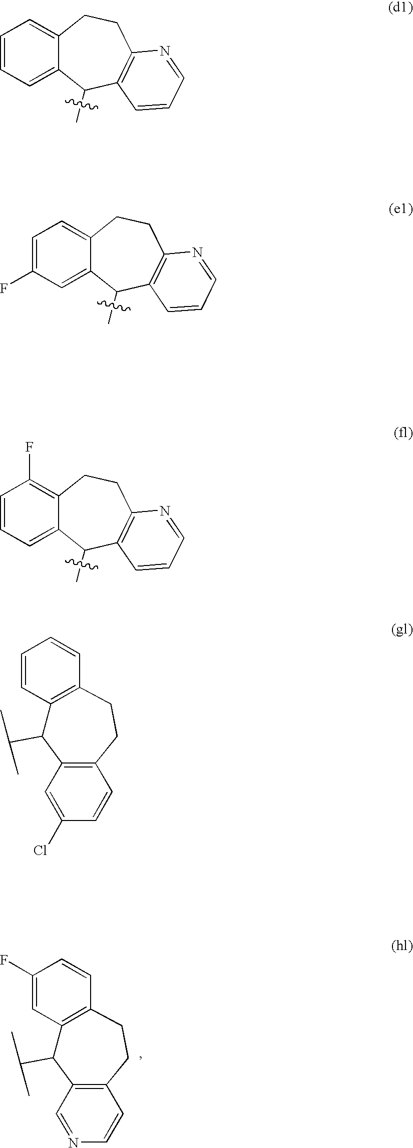
and wherein R³¹ is a C₁₋₆alkyl.

23. A compound according to any one of claims 18-22 for use as a medicament.

24. The use of a compound according to any one of claims 18-22 in the manufacture of a medicament for the therapy of pain.

25. A pharmaceutical composition comprising a compound according to any one of claims 18-22 and a pharmaceutically acceptable carrier.

26. A method for the therapy of pain in a warm-blooded animal, comprising the step of administering to said animal in need of such therapy a therapeutically effective amount of a compound according to any one of claims 18-22.

27. A compound according to any one of claims 18-22 for use as an antagonist of bradykinin at the B2 receptor.

28. A method of preparing a compound of formula VI comprising the step of reacting a compound of formula VII with a compound of Y—NCO or Y—NCS: 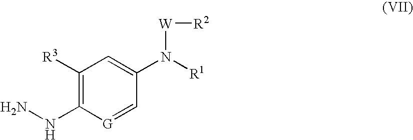
to form the compound of formula VI: 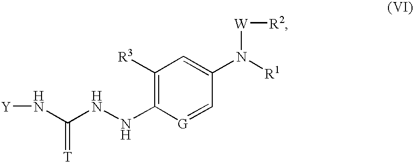
wherein
R¹ and R² are independently selected from hydrogen, optionally substituted acyl, optionally substituted alkyl-oxycarbonyl, optionally substituted alkyl, optionally substituted heteroalkyl, optionally substituted cycloalkyl, optionally substituted aryl; optionally substituted heterocyclyl; optionally substituted aryl-C₁₋₆alkyl, and optionally substituted heterocyclyl-C₁₋₆alkyl;
T is S or O;
X is represented by formula (i) or (ii), 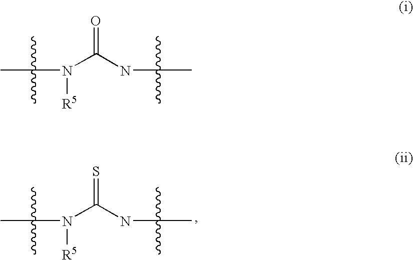
wherein R⁵ is —H, or optionally substituted C₁₋₆alkyl;
G is CH or N;
R³ is halogen;
Y is represented by formula (III) below: 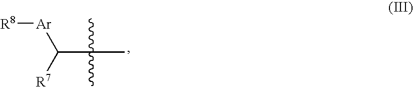
wherein
R⁷ is optionally substituted aryl, optionally substituted heteroaryl, optionally substituted aryl-C₁₋₆alkyl, optionally substituted heteroaryl-C₁₋₆alkyl, —C(═O)O—R⁹, —C(═O)NHR⁹, —C(═O)NR⁹R¹⁰, —SO₂NHR⁹, —SO₂NR⁹R¹⁰, —R¹¹NH₂, —R¹ NHR², —R¹¹NR¹²R¹³, —R¹¹OH, —R¹¹OR¹², —R¹¹SH, —R¹¹SR¹², or a divalent C₀₋₆group which together with R⁸ forms a portion of a ring,
R⁸ is —H, halogen, optionally substituted R¹², —OR¹², —SR¹², —S(═O)R¹², —SO₂R¹², —C(═O)R¹², or a divalent C₀₋₆group which together with the divalent R⁷ forms the portion of the ring,
wherein R⁹ and R¹⁰ are independently C₁₋₁₂hydrocarbyl, R¹¹ is C₁₋₆alkylene, R¹² and R¹³ are independently C₁₋₆alkyl; and
Ar is optionally substituted arylene, optionally substituted heteroarylene, optionally substituted arylene-C₁₋₆alkyl, or optionally substituted heteroarylene-C₁₋₆alkyl; and
W is —C(═O)—.

29. A compound of formula (VII): 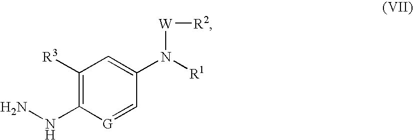
wherein
R¹ and R² are independently selected from hydrogen, optionally substituted C₁₋₁₂acyl, optionally substituted C₁₋₁₂alkyl-oxycarbonyl, optionally substituted C₁₋₁₂alkyl, optionally substituted C₁₋₁₂heteroalkyl, optionally substituted C₃₋₁₂cycloalkyl, optionally substituted C₆₋₁₂aryl and optionally substituted C₂₋₁₂heterocyclyl;
W is —C(═O)— or —S(═O)₂—;
G is N or CH; and
R³ is halogen, hydrogen or C₁₋₆alkyl.

30. A compound of Y—NCO or Y—NCS,
Y is represented by formula (III) below: 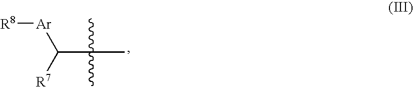
wherein
R⁷ is optionally substituted aryl, optionally substituted heteroaryl, optionally substituted aryl-C₁₋₆alkyl, optionally substituted heteroaryl-C₁₋₆alkyl, —C(═O)O—R⁹, C(═O)NHR⁹, —C(═O)NR⁹R¹⁰, —SO₂NHR⁹, —SO₂NR⁹R¹⁰, —R¹¹NH₂, —R¹¹NHR², —R¹¹NR¹²R¹³, —R¹¹OH, —R¹¹OR¹², —R¹¹SH, —R¹¹SR¹², or a divalent C₀₋₆group which together with R³ forms a portion of a ring,
R⁸ is —H, halogen, optionally substituted R², —OR², —SR², —S(═O)R², —SO₂R¹², —C(═O)R², or a divalent C₀₋₆group which together with the divalent R⁷ forms the portion of the ring,
wherein R⁹ and R¹⁰ are independently C₁₋₁₂hydrocarbyl, R¹¹ is C₁₋₆alkylene, R¹² and R¹³ are independently C₁₋₆alkyl; and
Ar is optionally substituted arylene, optionally substituted heteroarylene, optionally substituted arylene-C₁₋₆alkyl, or optionally substituted heteroarylene-C₁₋₆alkyl.
